(19) United States
(12) Patent Application Publication
(10) Pub. No.: US 2026/0068522 A1
(43) Pub. Date: Mar. 5, 2026

(54) ORGANIC COMPOUNDS AND ORGANIC LIGHT-EMITTING DIODE COMPRISING THE SAME

(71) Applicant: MATERIAL SCIENCE CO., LTD., Gyeonggi-do (KR)

(72) Inventors: Tae Wan LEE, Gyeonggi-do (KR); Seung Uk CHO, Gyeonggi-do (KR); Ha Ni JEON, Gyeonggi-do (KR); Da Hee PARK, Gyeonggi-do (KR); Sang Mee KIM, Gyeonggi-do (KR)

(21) Appl. No.: 19/318,658

(22) Filed: Sep. 4, 2025

(30) Foreign Application Priority Data
Sep. 5, 2024 (KR) 10-2024-0120477
Sep. 3, 2025 (KR) 10-2025-0124986

(51) Int. Cl. H10K 85/60; C07B 59/00; C07D 307/91; C07D 405/12; C07D 407/12; C07D 409/12; H10K 50/15

(52) U.S. Cl. CPC H10K 85/636; C07B 59/002; C07D 307/91; C07D 405/12; C07D 407/12; C07D 409/12; H10K 85/633; H10K 50/156; H10K 85/626; H10K 85/6574; H10K 85/6576

(57) ABSTRACT

Provided is a novel organic compound and an organic light-emitting diode including the same.

An organic light-emitting diode according to one exemplary embodiment of the present invention includes a first electrode, a second electrode facing the first electrode, and one or more organic material layers disposed between the first electrode and the second electrode, and at least one of the one or more organic material layers is a hole transport layer or a hole transport auxiliary layer, which contains the organic compound according to the present invention, represented by Formula 1.

### CROSS-REFERENCE TO RELATED APPLICATION

This application claims priority to Korean Patent Application No. 10-2024-0120477 filed on Sep. 5, 2024, and Korean Patent Application No. 10-2025-0124986 filed on Sep. 3, 2025, all the benefits accruing therefrom under 35 U.S.C. § 119, the contents of which are incorporated by reference in their entirety.

### BACKGROUND

The present invention relates to an organic compound and an organic light-emitting diode comprising the same.

As compared with other flat panel display elements such as a liquid crystal display device (LCD), a plasma display panel (PDP), and an electric field emission display (FED), an organic light-emitting diode (OLED) has a simple structure, various advantages in the manufacturing process, high brightness, excellent viewing angle characteristics, fast response speed, and a low operation voltage, and thus is being actively developed and commercialized to be used as a light source for a flat-panel display such as a wall-mounted TV or a backlight of a display, lighting, a billboard, or the like.

An organic light-emitting diode is composed of an organic material layer between two electrodes. The organic light-emitting diode is an element that uses the principle that electrons and holes are injected into an emission layer from two electrodes, respectively, to generate excitons due to combination of electrons and holes, and light is generated when the generated excitons fall from an excited state to a ground state.

The organic light-emitting diode may include at least one emission layer. In general, an organic light-emitting diode including a plurality of emission layers includes emission layers that emit light having peak wavelengths different from each other, which makes it possible to realize a specific color through a combination of light having peak wavelengths different from each other.

Structures of such an organic light-emitting diode may be divided into a top light-emitting element structure and a bottom light-emitting element structure. The top light-emitting element emits light generated in an emission layer toward a translucent first electrode by using a reflective second electrode (cathode). On the other hand, the bottom light-emitting element emits light that is generated in the emission layer by using the reflective first electrode and then reflected at the first electrode toward the transparent second electrode, that is, in the direction of the driving thin film transistor.

### DOCUMENTS OF RELATED ART

### Patent Document

Chinese Published Patent CN116354941A (published on 2023 Jun. 30)

### SUMMARY

The present disclosure provides a novel organic compound and an organic light-emitting diode comprising the same.

The exemplary embodiment according to the present invention may be used to achieve other objects not specifically mentioned, in addition to the above-described object.

The object of the present invention is not limited to the above-mentioned object, and other objects and advantages of the present invention, which are not mentioned, will be understood by the following description and will be more clearly understood by the exemplary embodiment according to the present invention.

In addition, it will be easy to see that the objects and advantages of the present invention can be realized by the means disclosed in the exemplary embodiment according to the present invention and a combination thereof.

In accordance with an exemplary embodiment of the present invention, provide is an organic compound represented by Formula 1.

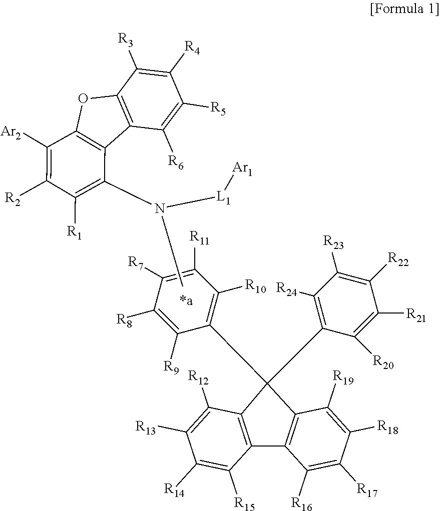

In Formula 1 above,
L₁ is selected from the group consisting of a single bond, a substituted or unsubstituted arylene group having 6 to 30 carbon atoms, and a substituted or unsubstituted heteroarylene group having 1 to 30 carbon atoms,
Ar₁ and Ar₂ are the same as or different from each other and are each independently a substituted or unsubstituted aryl group having 6 to 30 carbon atoms or a substituted or unsubstituted heteroaryl group having 1 to 30 carbon atoms,
R₁ to R₂₄ are the same as or different from each other and are each independently one selected from the group consisting of a hydrogen atom, a deuterium atom, a substituted or unsubstituted aryl group having 6 to 30 carbon atoms, and a substituted or unsubstituted heteroaryl group having 1 to 30 carbon atoms,
when L₁, Ar₁, Ar₂, and R₁ to R₂₄ are substituted, substituents are the same as or different from each other and may be each independently one or more selected from the group consisting of a deuterium atom, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, an heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms, and when the substituents are plural, the substituents may be the same as or different from each other and may be bonded to an adjacent group to form a substituted or unsubstituted ring, and
*a represents a bonding position on a phenylene, and one of R₇ to R₁₁ represents a single bond bonded to *a.

In accordance with an exemplary embodiment of the present invention, an organic light-emitting diode includes a first electrode, a second electrode facing the first electrode, and one or more layer of organic material layers disposed between the first electrode and the second electrode, where at least one of the one or more layers of the organic material layers is a hole transport layer or a hole transport auxiliary layer, which contains the organic compound represented by Formula 1 above.

The organic compound, according to the present invention, represented by Formula 1, may achieve excellent hole transport characteristics.

In addition, the organic light-emitting diode according to the present invention may improve the operation voltage, efficiency, and lifespan characteristics of the organic light-emitting diode by including a hole transport layer and/or a hole transport auxiliary layer, which contains the organic compound according to the present invention, represented by Formula 1.

In addition, in a case where the organic compound according to the present invention, which is represented by Formula 1, is used as a substance for a hole transport auxiliary layer, it is possible to provide an energy level that is suitable for a hole transport auxiliary layer to take a role in transferring holes from the hole transport layer to the emission layer and blocking electrons coming from the emission layer.

In addition, even in a case of being combined with a emission layer of any color, the organic light-emitting diode according to the present invention makes it possible to excellently realize the color coordinates targeted by the emission layer, by including a hole transport layer and/or a hole transport auxiliary layer, which contains the organic compound according to the present invention which is represented by Formula 1.

The effect of the present specification is not limited to the effect described above, and the other effects which are not mentioned will be clearly understood by those skilled in the art from the description below.

### DETAILED DESCRIPTION OF EMBODIMENTS

The above-described purposes, features, and advantages will be described in detail below, and accordingly, those skilled in the art to which the present invention belongs may easily implement the technical idea of the present invention.

In describing the present invention, in a case where it is determined that a detailed description of a publicly known technology related to the present invention may unnecessarily obscure the gist of the present invention, the detailed description thereof will be omitted.

In the present specification, when the word such as “includes”, “has”, “consists of”, “is disposed”, or “is equipped with” is used regarding a component, another portion may also be added, unless “only” is used. Unless there is a specifically explicit description, when a component is expressed in a singular, it also includes a plural form.

In the present specification, in interpreting a component, it is interpreted as including the error range even in a case where there is no additional explicit description.

In the present specification, the phrase that any component is disposed on an “upper part (or lower part) of” or “on (or below)” a component may not only mean that the any configuration is disposed in contact with the upper surface (or lower surface) of the component but also mean that another component is interposed between the component and the any component disposed on (below) the component.

The term “halogen group” used in the present specification includes fluorine, chlorine, bromine, and iodine.

The term “alkyl group” used in the present specification means both of a linear alkyl radical and a branched alkyl radical. Unless particularly limited, the alkyl group contains 1 to 10 carbon atoms and may include, methyl, ethyl, propyl, isopropyl, butyl, secondary butyl, isobutyl, ter-butyl, pentyl, isoamyl, hexyl, and the like, but is not limited thereto. In addition, the alkyl group may undergo any substitution.

The term “cycloalkyl group” used in the present specification means a cyclic alkyl radical. Unless particularly limited, the cycloalkyl group contains 3 to 10 carbon atoms and may include cyclopropyl, cyclobutyl, cyclopentyl, cyclohexyl, norbonyl, adamantyl, and the like, but is not limited thereto. In addition, the cycloalkyl group may undergo any substitution.

The term “alkenyl group” used in the present specification means both a linear alkenyl radical and a branched alkenyl radical, which has one or more carbon-carbon double bonds. Unless particularly limited, the alkenyl group contains 2 to 30 carbon atoms and may include vinyl, allyl, isopropenyl, 2-butenyl, and the like and the like, but is not limited thereto. In addition, the alkenyl group may undergo any substitution.

The term “cycloalkenyl group” used in the present specification means a cyclic alkenyl radical. Unless particularly limited, the cycloalkenyl group contains 3 to 20 carbon atoms, and in addition, the cycloalkenyl group may undergo any substitution.

The term “alkynyl group” used in the present specification means both a linear alkynyl radical and a branched alkynyl radical, which has one or more carbon-carbon triple bonds. Unless particularly limited, the alkynyl group contains 2 to 30 carbon atoms and may include ethynyl, 2-propynyl, and the like, but is not limited thereto. In addition, the alkynyl group may undergo any substitution.

The term “cycloalkynyl group” used in the present specification means a cyclic alkynyl radical. Unless particularly limited, the alkynyl group contains 3 to 20 carbon atoms, and in addition, the alkynyl group may undergo any substitution.

The term “aralkyl group” or “arylalkyl group” used in the present specification are used interchangeably and means an alkyl group having an aromatic group as a substituent, and in addition, the aralkyl group (arylalkyl group) may undergo any substitution.

The term “aryl group” or “aromatic group” used in the present specification is used in the same meaning, and the aryl group includes both a single ring group and a condensed ring group. The condensed ring may include a “condensed ring” that is two or more rings in which two adjacent rings have two common carbon atoms. In addition, it may also include a form in which two or more rings are simply attached or condensed to each other. Unless particularly limited, the aryl group contains 6 to 30 carbon atoms and may include phenyl, naphthyl, anthracenyl, phenanthrenyl, fluorenyl, dimethylfluorenyl, diphenylfluorenyl, spirofluorenyl, and the like; however, the aryl group is not limited thereto. In addition, the aryl group may undergo any substitution.

The term “heteroaryl group” or “heteroaromatic group” used in the present specification is used in the same meaning, and the heteroaryl group includes both a single ring group and a condensed ring group. The condensed ring may include a “condensed ring” that is two or more rings in which two adjacent rings have two common carbon atoms or heteroatoms. In addition, it may also include a form in which two or more rings are simply attached or condensed to each other. Unless particularly limited, the heteroaryl group contains 1 to 30 carbon atoms, and in a case where the heteroaryl group has 1 or 2 carbon atoms, an additional hetero element may be contained to form a ring. In addition, the heteroaryl group may contain 1 to 30 carbon atoms, and in this case, one or more carbon atoms in the ring are substituted with a heteroatom such as oxygen (O), nitrogen (N), sulfur (S), or selenium (Se), and the heteroaryl group may include a 6-membered monocyclic ring such as pyridinyl, pyrazinyl, pyrimidinyl, pyridazinyl, or triazinyl, and a polycyclic ring such as phenoxathiinyl, indolyl, indolyl, purinyl, quinolyl, isoquinolyl, benzoxyzolyl, benzothiazolyl, benzimidazolyl, benzofuranyl, dibenzofuranyl, benzothiophenyl, dibenzothiophenyl, phenylcarbazolyl, 9-phenylcarbazolyl, or carbazolyl, as well as 2-furanyl, N-imidazolyl, 2-isoxazolyl, 2-pyridinyl, 2-pyrimidinyl, 2-pyrimidinyl, and the like; however, the heteroaryl group is not limited thereto. In addition, the heteroaryl group may undergo any substitution.

The term “heterocycle group” used in the present specification means a heterocycle group in which one or more carbon atoms among carbon atoms constituting an aryl group, a cycloalkyl group, a cycloalkenyl group, a cycloalkynyl group, an arylalkyl group, an arylamino group, or the like are substituted with a heteroatom such as oxygen (O), nitrogen (N), sulfur (S), or selenium (Se), and with reference to the definition above, it includes a heteroaryl group, a heterocycloalkyl group, a heterocycloalkenyl group, a heterocycloalkynyl group, a heteroarylalkyl group, a heteroarylamino group, and the like. In addition, the heteroaryl group may undergo any substitution.

Unless particularly limited, the term “carbon ring” used in the present specification may be used as a term that includes both “a cycloalkyl group” which is an alicyclic ring group and “an aryl group (aromatic group) which is an aromatic ring group.

The terms “heteroalkyl group” and “heteroarylalkyl group”, which are used in the present specification, mean those in which one or more carbon atoms among carbon atoms constituting an alkyl group and an arylalkyl group are substituted with a heteroatom such as oxygen (O), nitrogen (N), sulfur (S), or selenium (Se), and in addition, the heteroalkyl group and the heteroarylalkyl group may undergo any substitution.

The term “alkylamino group”, “arylalkylamino group”, “arylamino group”, or “heteroarylamino group”, which is used in the present specification, means a group in which an amino group (or amine group) is substituted with the alkyl group, the arylalkyl group, the aryl group, or the heteroaryl group, and the alkylamino group, the arylalkylamino group, the arylamino group, or the heteroarylamino group” includes all of a primary amino group (or amine group), a secondary amino group (or amine group), and a tertiary amino group (or amine group). In addition, the alkylamino group, the arylalkylamino group, the arylamino group, and the heteroarylamino group may undergo any substitution.

The terms “alkylsilyl group,” “arylsilyl group,” “alkoxy group”, “aryloxy group”, “alkylthio group”, and “arylthio group”, which are used in the present specification, mean groups in which a silyl group, an oxy group, and a thio group are substituted with the alkyl group and the aryl group, respectively, and in addition, the alkylsilyl group, the arylsilyl group, the alkoxy group, the aryloxy group, the alkylthio group, and the arylthio group may undergo any substitution.

The terms “arylene group”, “arylalkylene group”, “heteroarylene group”, and “heteroarylalkylene group”, which are used in the present specification, mean those in which each of the aryl group, arylalkyl group, a heteroaryl group, and a heteroarylalkyl group is a divalent substituent that further includes one substitution. In addition, the arylene group, the arylalkylene group, the heteroarylene group, and the heteroarylalkylene group may undergo any substitution.

The term “substitution” used in the present specification means that a hydrogen (H) atom bonded to a carbon atom, a nitrogen atom, or the like of the compound according to the present invention is changed to a substituent other than the hydrogen atom, and in a case where there are a plurality of substituents, the respective substituents may be the same as or different from each other.

The substituents may be each independently selected from the group consisting of a deuterium atom, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, an heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms.

In the present specification, the reference for the unit is based on weight (wt) unless specifically described. For example, in a case where “%” is described, it is interpreted as % by weight (wt %).

Hereinafter, an organic compound according to the present invention and an organic light-emitting diode comprising the same will be described in detail.

According to one exemplary embodiment of the present invention, in Formula 1, L₁ may be a single bond or a substituted or unsubstituted arylene group having 6 to 15 carbon atoms. For example, L₁ may be a single bond or a substituted or unsubstituted phenylene.

In Formula 1, Ar₁ may be a substituted or unsubstituted aryl group having 6 to 30 carbon atoms, or a substituted or unsubstituted heteroaryl group having 1 to 30 carbon atoms, which contains at least one or more heteroatoms selected from the group consisting of O, S, and N. For example, Ar₁ may be a substituted or unsubstituted phenyl group, a substituted or unsubstituted naphthyl group, a substituted or unsubstituted phenanthrenyl group, a substituted or unsubstituted dibenzofuranyl group, a substituted or unsubstituted dibenzothiophenyl group, a substituted or unsubstituted carbazolyl group, a substituted or unsubstituted 9-phenylcarbazolyl group, a substituted or unsubstituted dimethylfluorenyl group, or a substituted or unsubstituted diphenylfluorenyl group.

Ar₂ may be a substituted or unsubstituted aryl group having 6 to 15 carbon atoms. For example, Ar₂ may be a substituted or unsubstituted phenyl group, a substituted or unsubstituted naphthyl group, and a substituted or unsubstituted phenanthrenyl group.

The substituents of L₁, Ar₁, Ar₂, and R₁ to R₂₄ are the same as or different from each other, and may be each independently one or more selected from the group consisting of a deuterium atom, a methyl group, an ethyl group, a propyl group, a butyl group, a pentyl group, a hexyl group, a cyclopropyl group, a cyclobutyl group, a cyclopentyl group, a cyclohexyl group, an adamantyl group, a phenyl group, a biphenyl group, a naphthyl group, a phenyl-naphthyl group, an anthracenyl group, a phenanthrenyl group, a dimethylfluorenyl group, a diphenylfluorenyl group, a spirofluorenyl group, a benzofuranyl group, a benzothiophenyl group, a dibenzofuranyl group, a dibenzothiophenyl group, a carbazolyl group, a phenylcarbazolyl group, and a 9-phenylcarbazolyl group.

According to one exemplary embodiment of the present invention, in Formula 1, Ar₂ may be one of groups represented by Formula 2 to Formula 5 below.

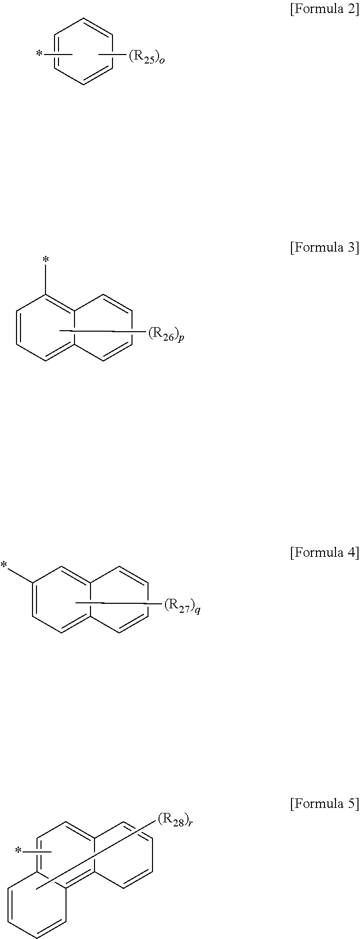

In Formula 2 to Formula 5 above, R₂₅ to R₂₈ are the same as or different from each other and are each independently one selected from the group consisting of a hydrogen atom, a deuterium atom, a substituted or unsubstituted arylene group having 6 to 30 carbon atoms, and a substituted or unsubstituted heteroarylene group having 1 to 30 carbon atoms,

o is an integer of 1 to 5,

p is an integer of 1 to 7,

q is an integer of 1 to 7,

r is an integer of 1 to 9, and

* means a moiety for bonding.

According to one exemplary embodiment of the present invention, in a case where Ar₂ is such a group that is represented by Formula 2, Ar₁ may be a substituted or unsubstituted aryl group having 6 to 30 carbon atoms, or a substituted or unsubstituted heteroaryl group having 1 to 30 carbon atoms, which contains at least one heteroatom selected from the group consisting of O, S, and N. For example, Ar₁ may be a substituted or unsubstituted phenyl group, a substituted or unsubstituted naphthyl group, a substituted or unsubstituted phenanthrenyl group, a substituted or unsubstituted dibenzofuranyl group, a substituted or unsubstituted dibenzothiophenyl group, a substituted or unsubstituted carbazolyl group, a substituted or unsubstituted 9-phenylcarbazolyl group, or a substituted or unsubstituted diphenylfluorenyl group.

According to one exemplary embodiment of the present invention, in a case where Ar₂ is such a group that is represented by Formula 3 or Formula 4, Ar₁ may be a substituted or unsubstituted aryl group having 6 to 30 carbon atoms, or a substituted or unsubstituted heteroaryl group having 1 to 30 carbon atoms, which contains at least one heteroatom selected from the group consisting of O and S. For example, Ar₁ may be a substituted or unsubstituted phenyl group, a substituted or unsubstituted naphthyl group, a substituted or unsubstituted phenanthrenyl group, a substituted or unsubstituted dibenzofuranyl group, a substituted or unsubstituted dibenzothiophenyl group, a substituted or unsubstituted dimethylfluorenyl group, or a substituted or unsubstituted diphenylfluorenyl group.

According to one exemplary embodiment of the present invention, in a case where Ar₂ is such a group that is represented by Formula 5, An may be a substituted or unsubstituted aryl group having 6 to 30 carbon atoms, or a substituted or unsubstituted heteroaryl group having 1 to 30 carbon atoms, which contains at least one heteroatom selected from the group consisting of O, S, and N. For example, Ar₁ may be a substituted or unsubstituted phenyl group, a substituted or unsubstituted naphthyl group, a substituted or unsubstituted phenanthrenyl group, a substituted or unsubstituted dibenzofuranyl group, a substituted or unsubstituted dibenzothiophenyl group, a substituted or unsubstituted carbazolyl group, a substituted or unsubstituted 9-phenylcarbazolyl group, a substituted or unsubstituted dimethylfluorenyl group, or a substituted or unsubstituted diphenylfluorenyl group.

According to one exemplary embodiment of the present invention, Formula 1 may be represented by Formula 6 or Formula 7 below.

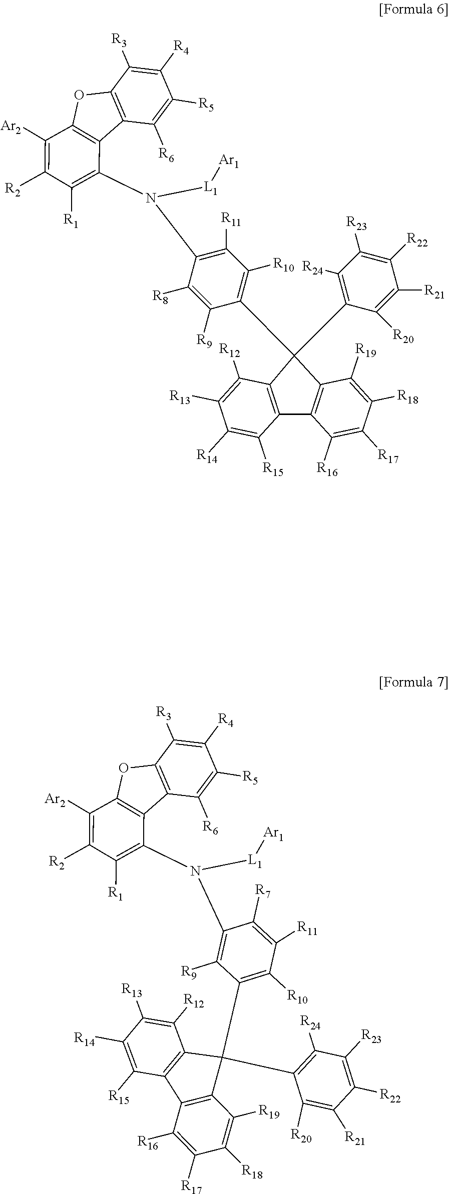

In Formula 6 or Formula 7 above,
definitions of L₁, Ar₁, Ar₂, and R₁ to R₂₄ and substituents thereof are the same as those described in Formula 1 above.

Ar₁ may be one of groups represented by Formula 8 to Formula 11 below.

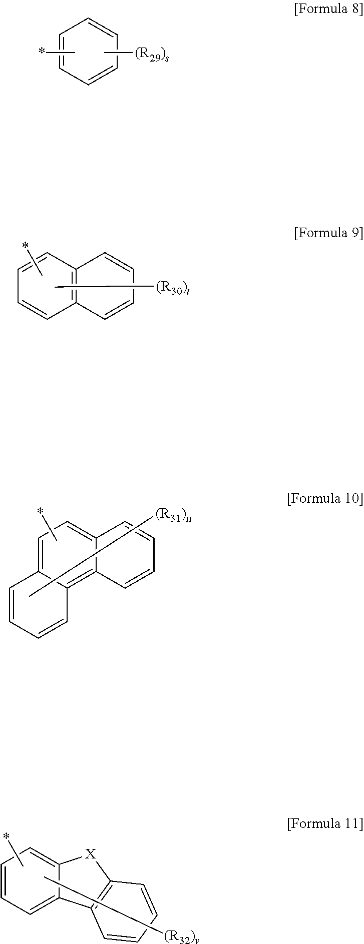

In Formula 8 to Formula 11 above, R₂₉ to R₃₂ are the same as or different from each other and are each independently one selected from the group consisting of a hydrogen atom, a deuterium atom, a substituted or unsubstituted aryl group having 6 to 30 carbon atoms, and a substituted or unsubstituted heteroaryl group having 1 to 30 carbon atoms,
s is an integer of 1 to 5, t and v are each an integer of 1 to 7, and u is an integer of 1 to 9.

In Formula 11 above, X may be one of O, S, NR₃₃, or CR₃₄R₃₅. According to one exemplary embodiment of the present invention, in a case where Ar₂ is such a group that is represented by Formula 2 or Formula 5, X may be O, S, CR₃₄R₃₅, or NR₃₃. According to one exemplary embodiment of the present invention, in a case where Ar₂ is such a group that is represented by Formula 3 or Formula 4, X may be O, S, or CR₃₄R₃₅.

According to one exemplary embodiment of the present invention, in a case where Ar₂ is such a group that is represented by Formula 5, X may be O, S, NR₃₃, or CR₃₄R₃₅.

R₃₃ to R₃₅ are the same as or different from each other and are ones that are each independently selected from the group consisting of a hydrogen atom, a deuterium atom, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms. In this case, in a case where Ar₂ is such a group that is represented by Formula 2, R₃₄ and R₃₅ are the same as or different from each other and may be each independently a substituted or unsubstituted aryl group having 6 to 30 carbon atoms or a substituted or unsubstituted heteroaryl group having 1 to 30 carbon atoms.

* means a moiety for bonding.

According to one exemplary embodiment of the present invention, R₁ to R₂₄ and R₂₆ to R₂₈ are the same as or different from each other and may each independently be a hydrogen atom or a deuterium atom.

According to one exemplary embodiment of the present invention, L₁ may be a single bond or one of substituents represented by F1 to F3 below. The dotted lines in the following structure means linking portions to be linked to substituents different from each other. Dn refers to the number of substituted deuterium atoms in the following F1 to F3 structures, and in this case, n, which refers to the number of deuterium atoms, is an integer equal to or larger than 0, and an upper limit thereof is the number of hydrogen atoms that may be substituted in the corresponding structure. For example, in a case where n is 0 in F1, it means

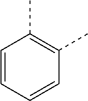

(indicated by D₀), and in a case where n is 1, it means

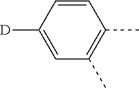

(indicated by D₁), in which one of the four hydrogen atoms in the phenyl group is substituted with a deuterium atom.

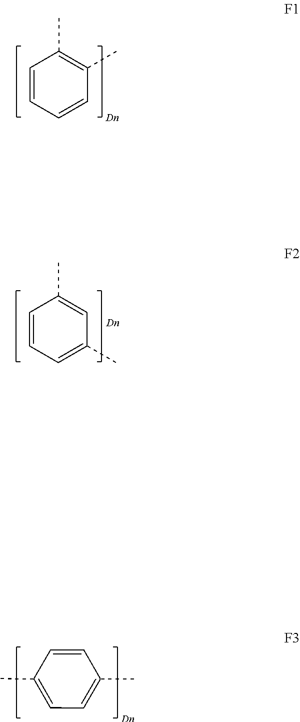

According to one exemplary embodiment of the present invention, Ar₁ may be a substituted or unsubstituted aryl group having 6 to 30 carbon atoms or a substituted or unsubstituted heteroaryl group having 1 to 30 carbon atoms.

According to one exemplary embodiment of the present invention, Ar₁ may be a substituted or unsubstituted aryl group having 6 to 30, 6 to 25, 6 to 15, 6 to 12, or 6 to 10 carbon atoms, or a substituted or unsubstituted heteroaryl group having 3 to 30, 3 to 18, or 3 to 12 carbon atoms.

According to one exemplary embodiment of the present invention, An may be a substituted or unsubstituted phenyl group, a substituted or unsubstituted biphenyl group, a substituted or unsubstituted ter-phenyl group, a substituted or unsubstituted naphthyl group, a substituted or unsubstituted anthryl group, a substituted or unsubstituted phenanthrenyl group, a substituted or unsubstituted fluorenyl group, a substituted or unsubstituted diphenylfluorenyl group, a substituted or unsubstituted naphthyl-phenyl group, a substituted or unsubstituted phenanthryl-phenyl group, a substituted or unsubstituted naphthyl-biphenyl group, a substituted or unsubstituted dibenzofuranyl group, a substituted or unsubstituted dibenzothiophenyl group, a substituted or unsubstituted carbazole group, a substituted or unsubstituted 9-phenylcarbazole group, a substituted or unsubstituted dibenzofuranyl-phenyl group, a substituted or unsubstituted dibenzothiophenyl-phenyl group, a substituted or unsubstituted carbazole-phenyl group, or a substituted or unsubstituted 9-phenyl carbazole-phenyl group. For example, Ar₁ may be a substituted or unsubstituted phenyl group, a substituted or unsubstituted biphenyl group, a substituted or unsubstituted naphthyl group, a substituted or unsubstituted phenanthrenyl group, a substituted or unsubstituted fluorenyl group, a substituted or unsubstituted diphenylfluorenyl group, a substituted or unsubstituted naphthyl-phenyl group, a substituted or unsubstituted phenanthryl-phenyl group, or a substituted or unsubstituted naphthyl-biphenyl group.

Alternatively, Ar₁ may be a deuterium-substituted or unsubstituted phenyl group, a deuterium-substituted or unsubstituted biphenyl group, a deuterium-substituted or unsubstituted naphthyl group, a deuterium-substituted or unsubstituted phenanthrenyl group, a deuterium-substituted or unsubstituted fluorenyl group, a deuterium-substituted or unsubstituted diphenylfluorenyl group, a deuterium-substituted or unsubstituted naphthyl-phenyl group, a deuterium-substituted or unsubstituted phenanthrenyl-phenyl group, or a deuterium-substituted or unsubstituted naphthyl-biphenyl group.

According to one exemplary embodiment of the present invention, Ar₁ may be one of substituents represented by M1 to M68 below.

In the following M1 to M68, * indicates a position for bonding, and Dn refers to the number of substituted deuterium atoms in the following M1 to M68 structures, and in this case, n, which refers to the number of deuterium atoms, is an integer equal to or larger than 0, and an upper limit thereof is the number of hydrogen atoms that may be substituted in the corresponding structure. For example, in a case where n is 0 in M1, it means

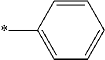 [SMILES: *c1ccccc1]

and in a case where n is 1, it means

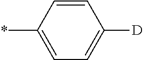 [SMILES: *c1ccc([2H])cc1]

in which one of the five hydrogen atoms in the phenyl group is substituted with a deuterium atom.

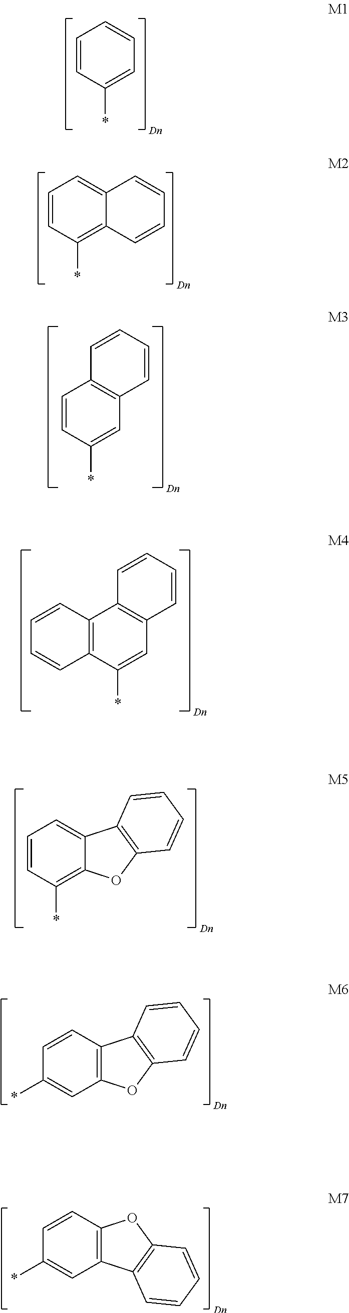

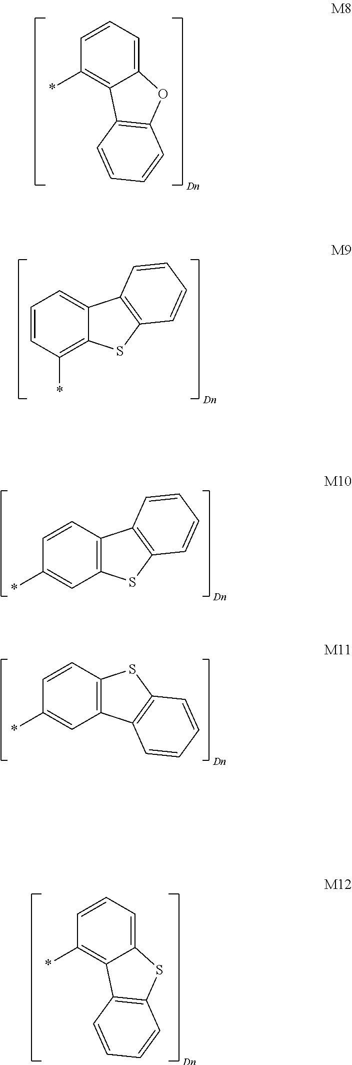

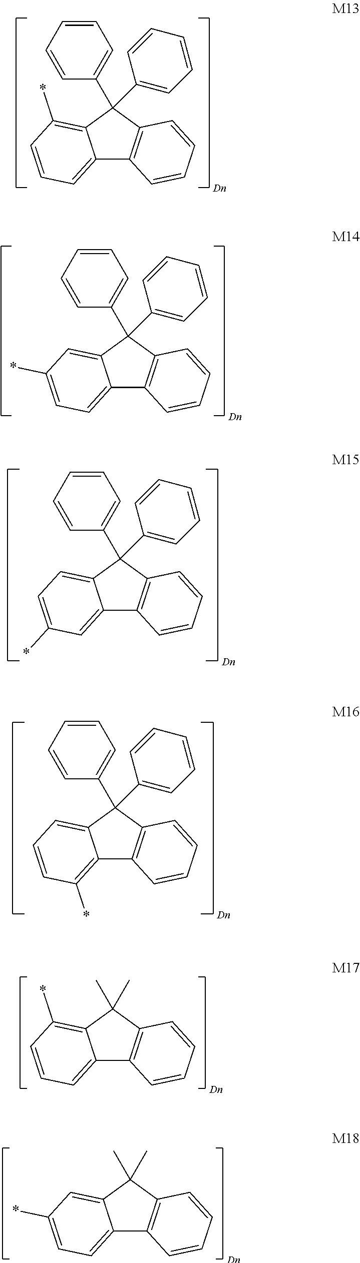

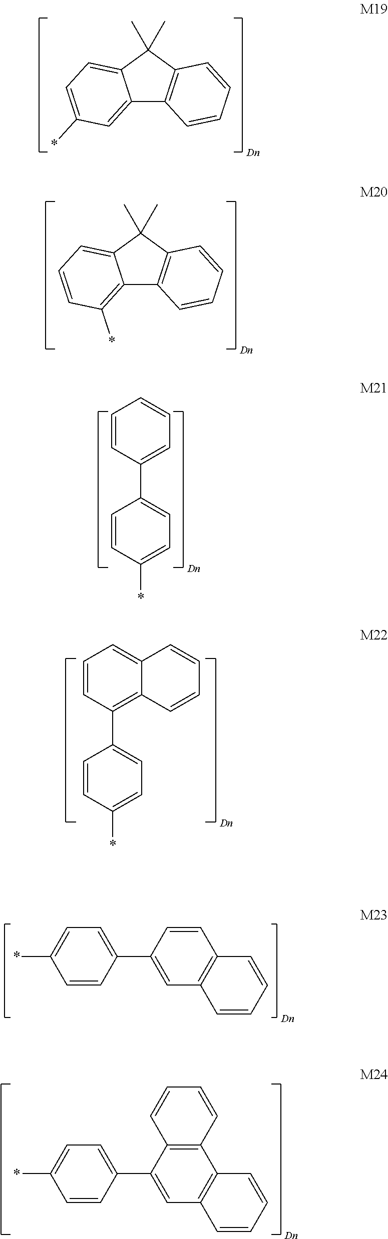

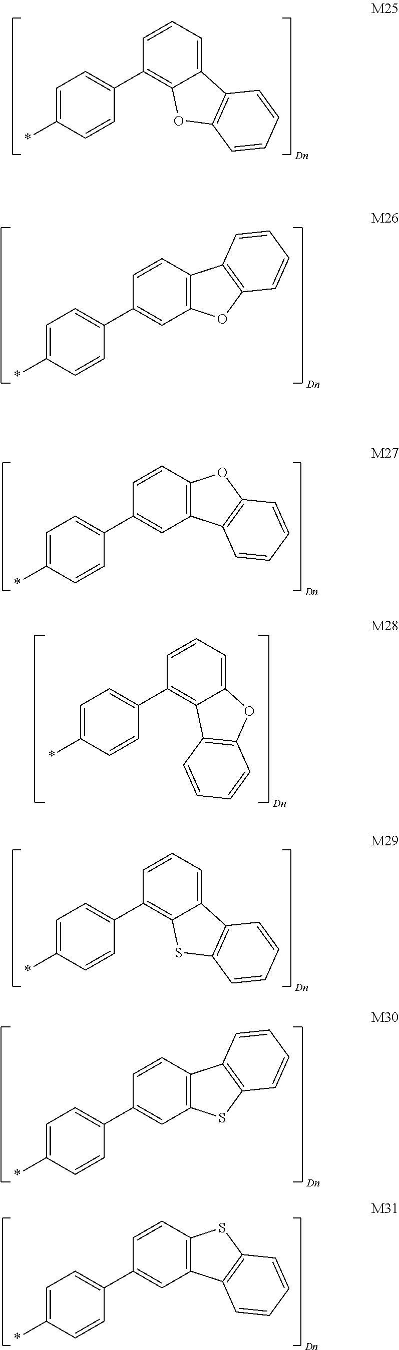

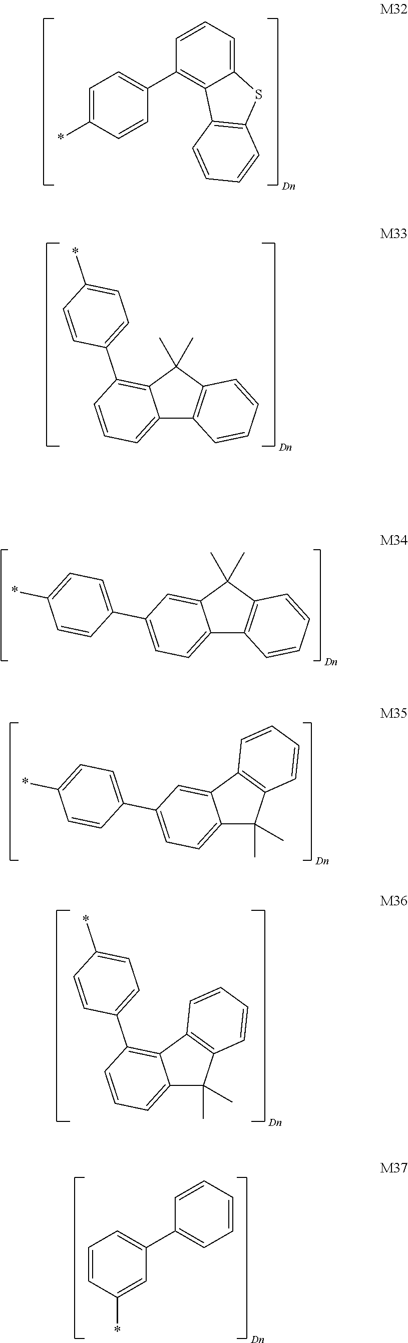

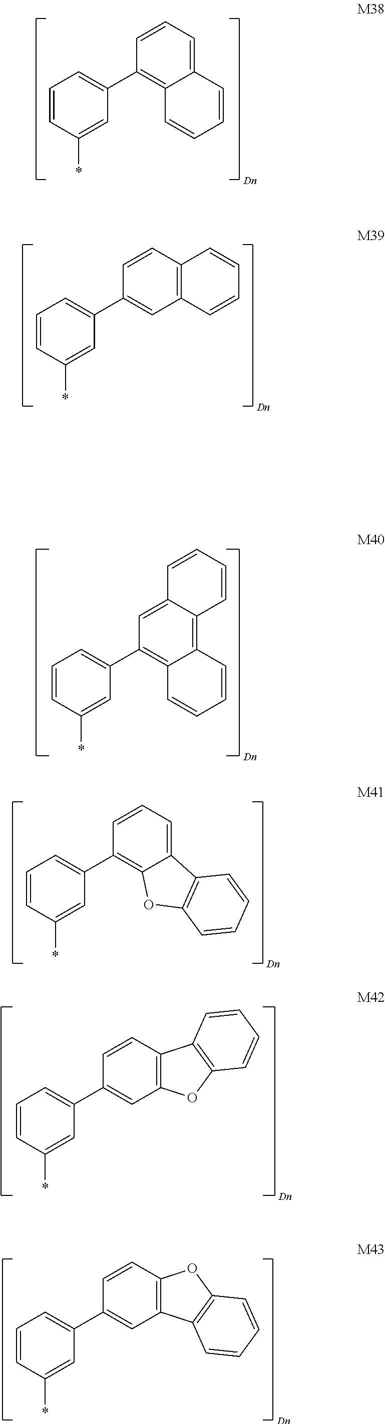

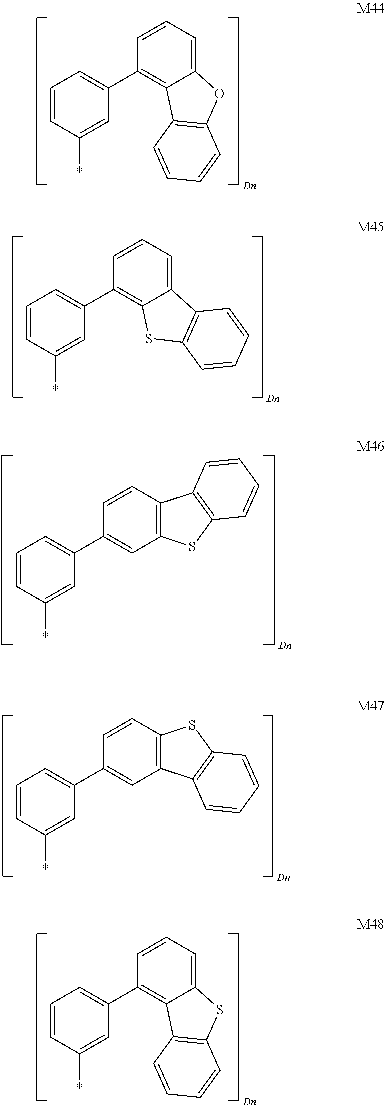

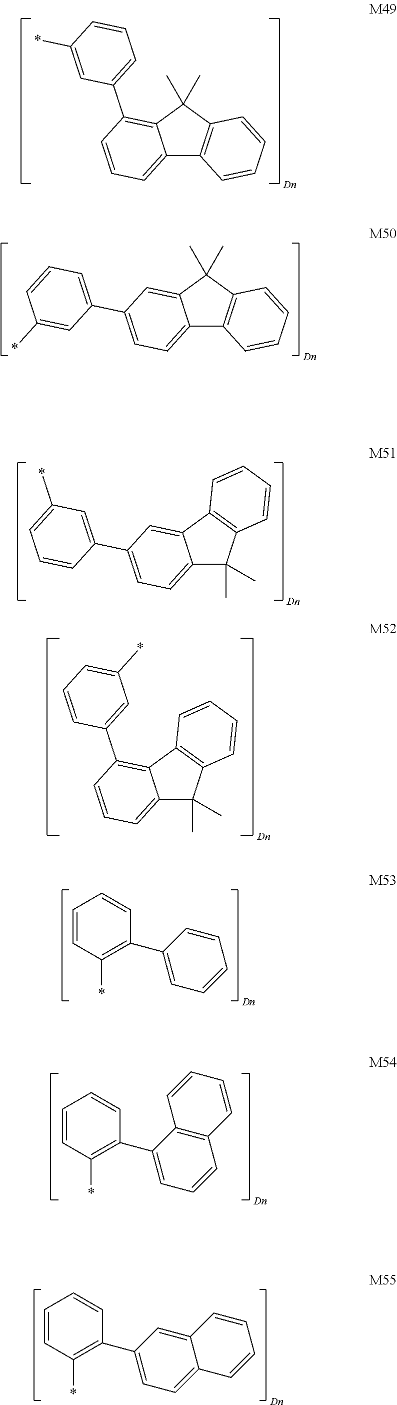

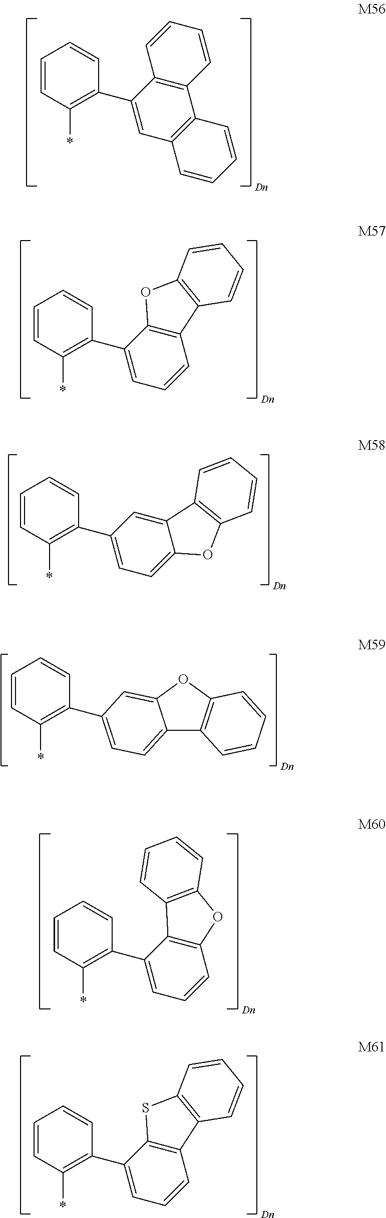

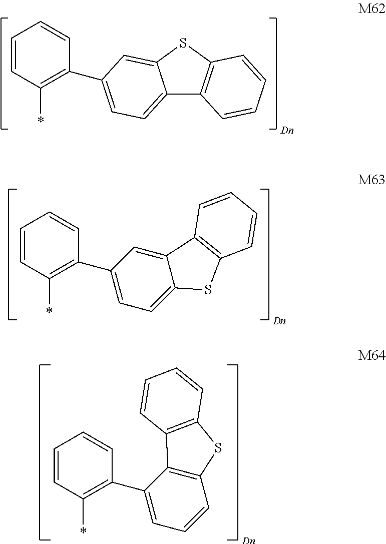

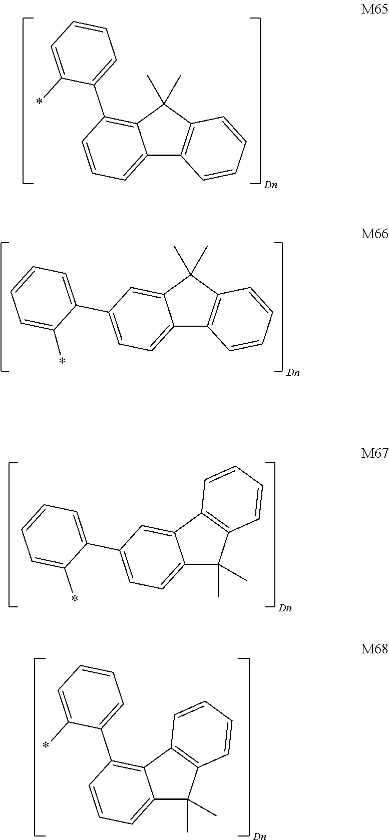

According to one exemplary embodiment of the present invention, in a case where an aryl amine is bonded at a position 1 or 8 of dibenzofuran represented by Formula A below and a group represented by Formulae 3 to 5 is bonded as Ar₂ at a position 4 or 5, the compound according to the present invention makes it possible to provide an energy level that is more suitable for a hole transport auxiliary layer to take a role in transferring holes from the hole transport layer to the emission layer and blocking electrons coming from the emission layer.

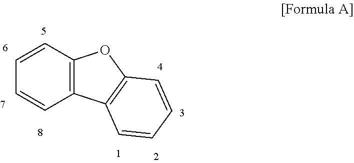 [SMILES: c1ccc2c(c1)oc1ccccc12]

According to one exemplary embodiment of the present invention, in the compound according to the present invention, in a case where Ar₂ is selected from Formulae 3 to 5, Ar₁ may be a substituted or unsubstituted aryl group having 6 to 30 carbon atoms or a substituted or unsubstituted heteroaryl group having 1 to 30 carbon atoms. For example, in a case where Ar₂ is selected from Formula 3 or 4 and Ar₁ is a substituted or unsubstituted aryl group having 6 to 30, 6 to 25, 6 to 15, 6 to 12, or 6 to 10 carbon atoms, the element performance may be excellent. For example, in a case where Ar₂ is selected from Formula 3 and Ar₁ is a substituted or unsubstituted aryl group having 6 to 30, 6 to 25, 6 to 15, 6 to 12, or 6 to 10 carbon atoms, the stability of the compound itself is high and hole mobility is also excellent, and thus the charge balance of the entire element is improved, which may provide a more excellent element performance.

According to one exemplary embodiment of the present invention, in the compound according to the present invention, in a case where Ar₂ is selected from Formulae 3 to 5, L₁ may be a single bond, or may be in a form of ortho, which is the above-described F1 structure, a form of meta, which is the above-described F2 structure, or a form of para, which is the above-described F3 structure, and for example, in a case where L₁ is a single bond, the element performance may be more excellent.

According to one exemplary embodiment of the present invention, the compound represented by Formula 1 may be selected from the group consisting of the following compounds; however, the compound is not limited thereto.

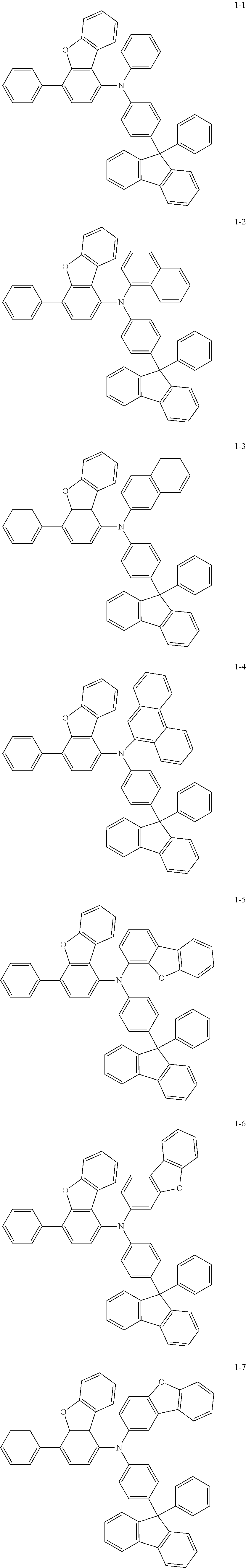

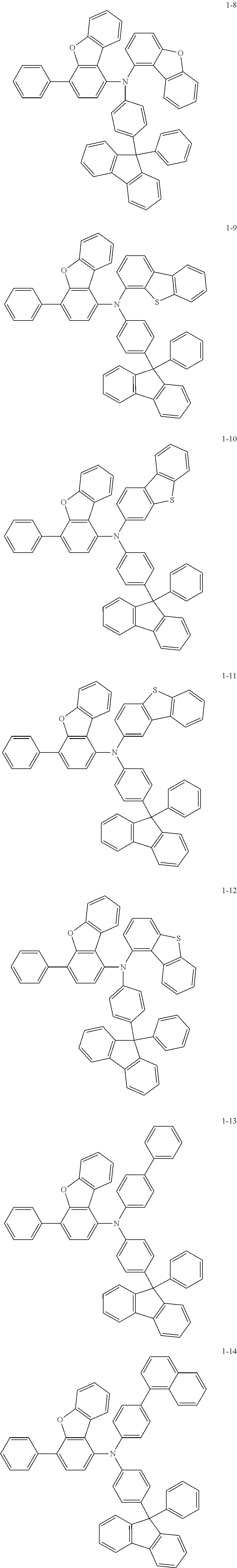

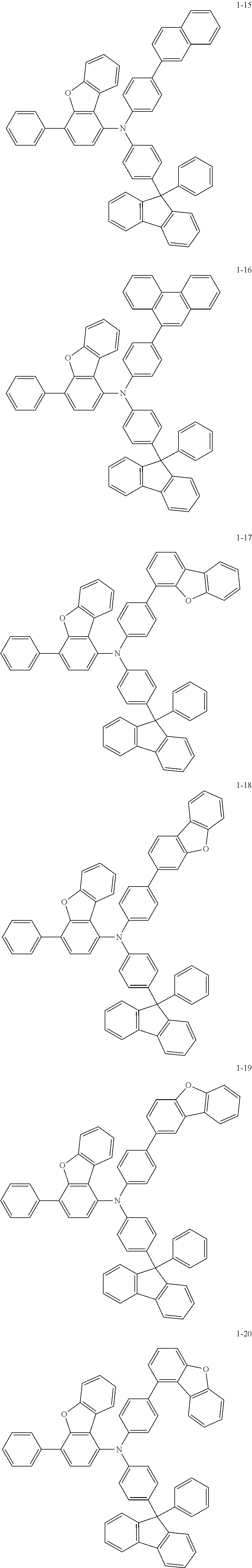

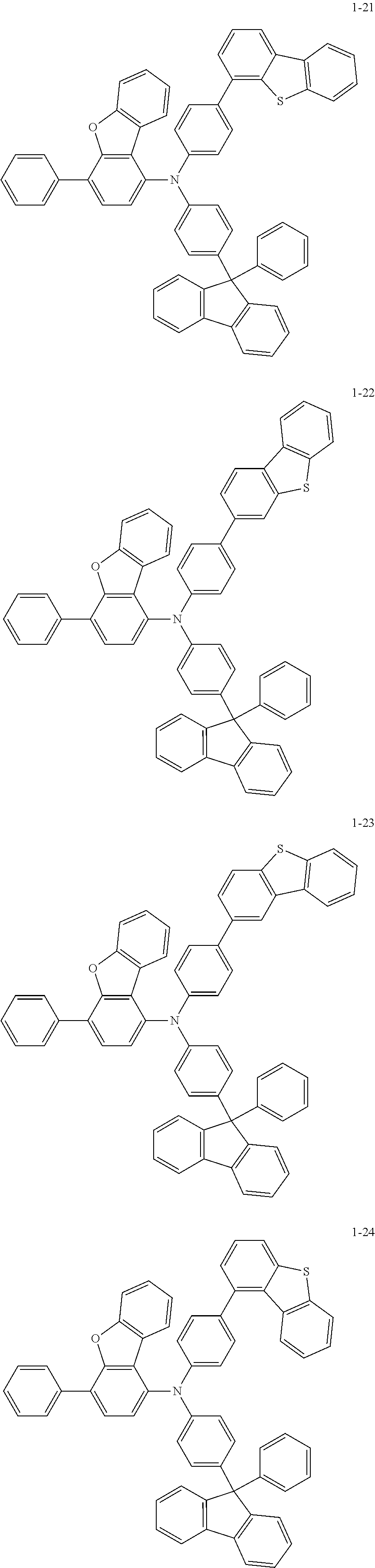 [SMILES: c1ccc(-c2ccc(N(c3ccc(-c4cccc5c4sc4ccccc45)cc3)c3ccc(C4(c5ccccc5)c5ccccc5-c5ccccc54)cc3)c3c2oc2ccccc23)cc1 ; c1ccc(-c2ccc(N(c3ccc(-c4ccc5c(c4)sc4ccccc45)cc3)c3ccc(C4(c5ccccc5)c5ccccc5-c5ccccc54)cc3)c3c2oc2ccccc23)cc1 ; c1ccc(-c2ccc(N(c3ccc(-c4ccc5sc6ccccc6c5c4)cc3)c3ccc(C4(c5ccccc5)c5ccccc5-c5ccccc54)cc3)c3c2oc2ccccc23)cc1 ; c1ccc(-c2ccc(N(c3ccc(-c4cccc5sc6ccccc6c45)cc3)c3ccc(C4(c5ccccc5)c5ccccc5-c5ccccc54)cc3)c3c2oc2ccccc23)cc1]

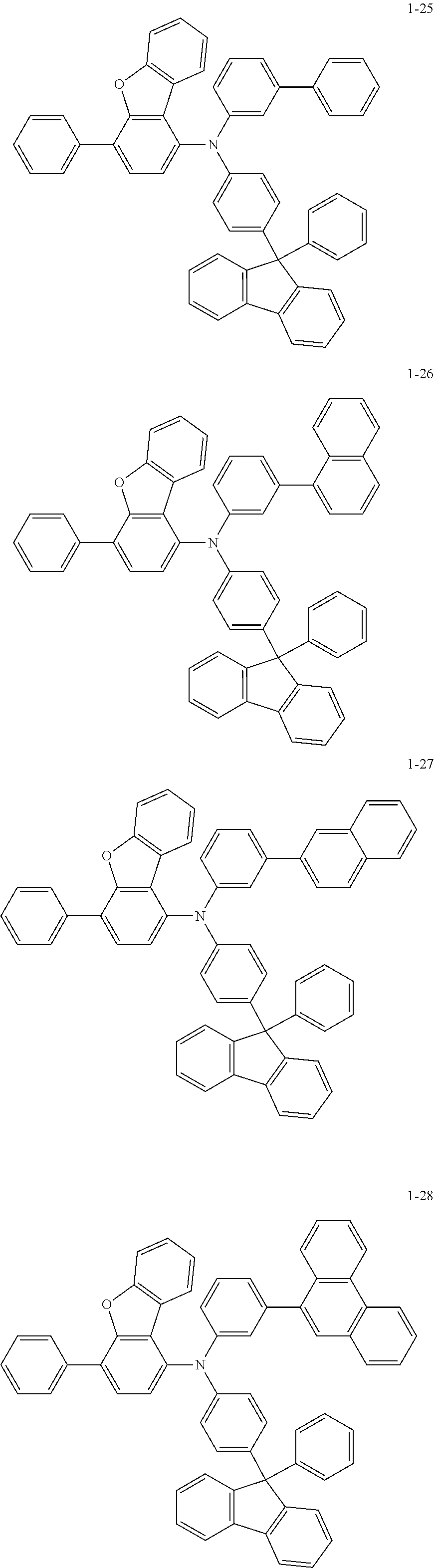 [SMILES: c1ccc(-c2cccc(N(c3ccc(C4(c5ccccc5)c5ccccc5-c5ccccc54)cc3)c3ccc(-c4ccccc4)c4oc5ccccc5c34)c2)cc1 ; c1ccc(-c2ccc(N(c3ccc(C4(c5ccccc5)c5ccccc5-c5ccccc54)cc3)c3cccc(-c4cccc5ccccc45)c3)c3c2oc2ccccc23)cc1 ; c1ccc(-c2ccc(N(c3ccc(C4(c5ccccc5)c5ccccc5-c5ccccc54)cc3)c3cccc(-c4ccc5ccccc5c4)c3)c3c2oc2ccccc23)cc1 ; c1ccc(-c2ccc(N(c3ccc(C4(c5ccccc5)c5ccccc5-c5ccccc54)cc3)c3cccc(-c4cc5ccccc5c5ccccc45)c3)c3c2oc2ccccc23)cc1]

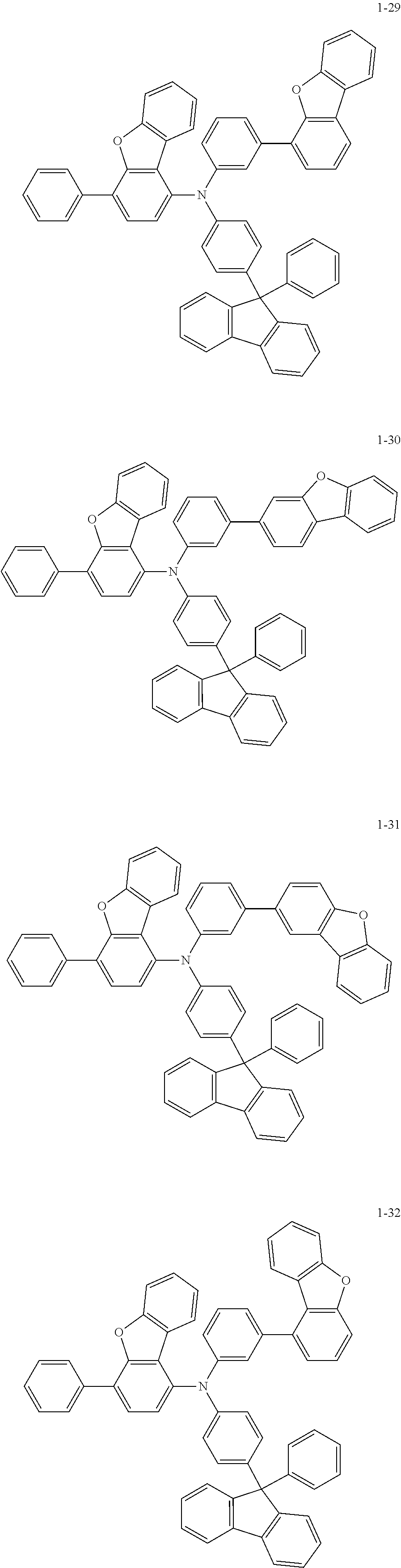 [SMILES: c1ccc(-c2ccc(N(c3ccc(C4(c5ccccc5)c5ccccc5-c5ccccc54)cc3)c3cccc(-c4cccc5c4oc4ccccc45)c3)c3c2oc2ccccc23)cc1 ; c1ccc(-c2ccc(N(c3ccc(C4(c5ccccc5)c5ccccc5-c5ccccc54)cc3)c3cccc(-c4ccc5c(c4)oc4ccccc45)c3)c3c2oc2ccccc23)cc1 ; c1ccc(-c2ccc(N(c3ccc(C4(c5ccccc5)c5ccccc5-c5ccccc54)cc3)c3cccc(-c4ccc5oc6ccccc6c5c4)c3)c3c2oc2ccccc23)cc1 ; c1ccc(-c2ccc(N(c3ccc(C4(c5ccccc5)c5ccccc5-c5ccccc54)cc3)c3cccc(-c4cccc5oc6ccccc6c45)c3)c3c2oc2ccccc23)cc1]

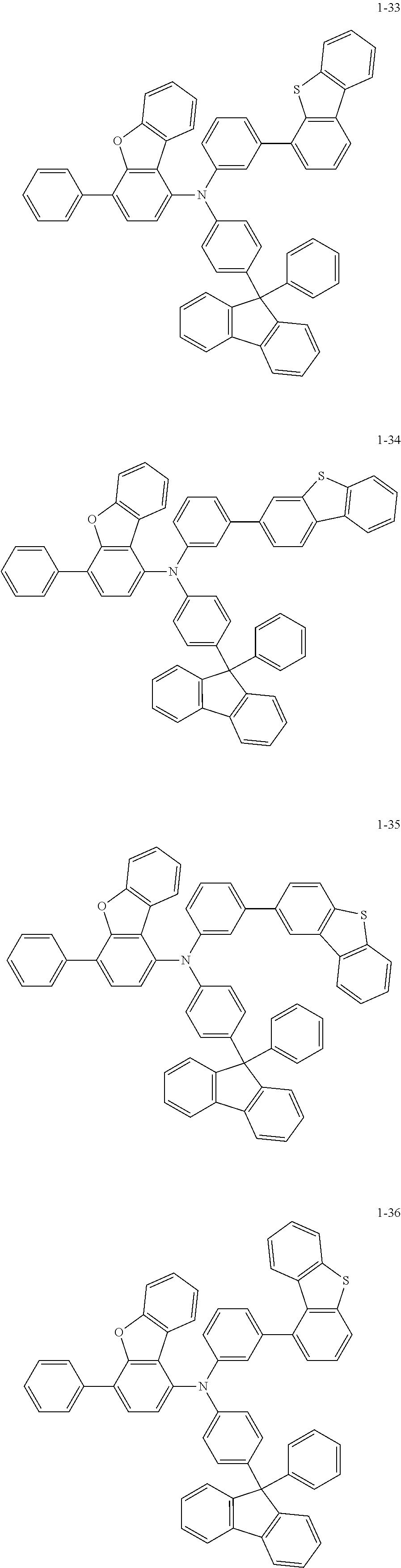 [SMILES: c1ccc(-c2ccc(N(c3ccc(C4(c5ccccc5)c5ccccc5-c5ccccc54)cc3)c3cccc(-c4cccc5c4sc4ccccc45)c3)c3c2oc2ccccc23)cc1 ; c1ccc(-c2ccc(N(c3ccc(C4(c5ccccc5)c5ccccc5-c5ccccc54)cc3)c3cccc(-c4ccc5c(c4)sc4ccccc45)c3)c3c2oc2ccccc23)cc1 ; c1ccc(-c2ccc(N(c3ccc(C4(c5ccccc5)c5ccccc5-c5ccccc54)cc3)c3cccc(-c4ccc5sc6ccccc6c5c4)c3)c3c2oc2ccccc23)cc1 ; c1ccc(-c2ccc(N(c3ccc(C4(c5ccccc5)c5ccccc5-c5ccccc54)cc3)c3cccc(-c4cccc5sc6ccccc6c45)c3)c3c2oc2ccccc23)cc1]

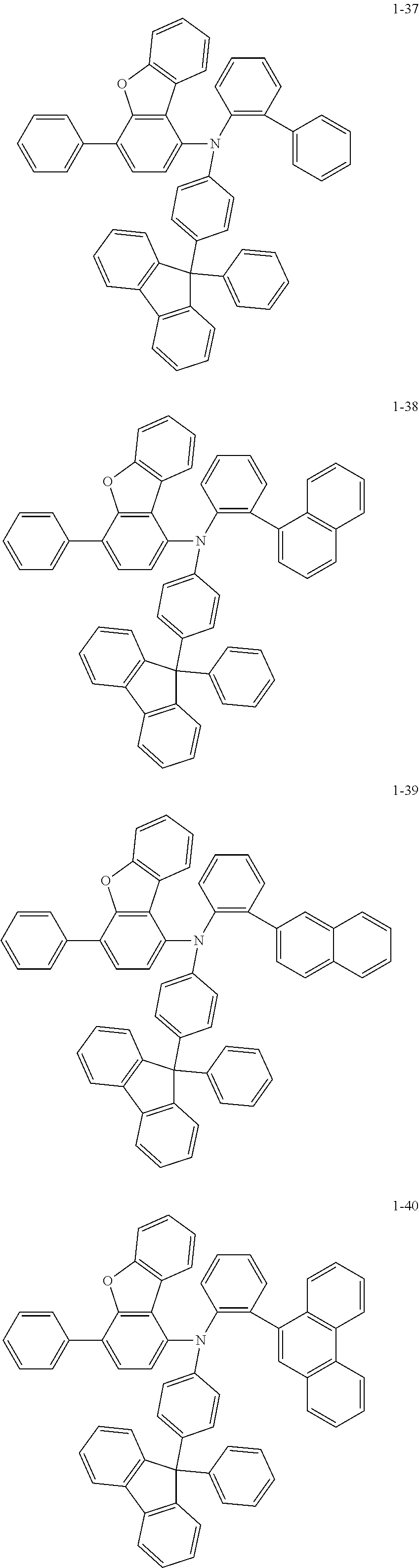

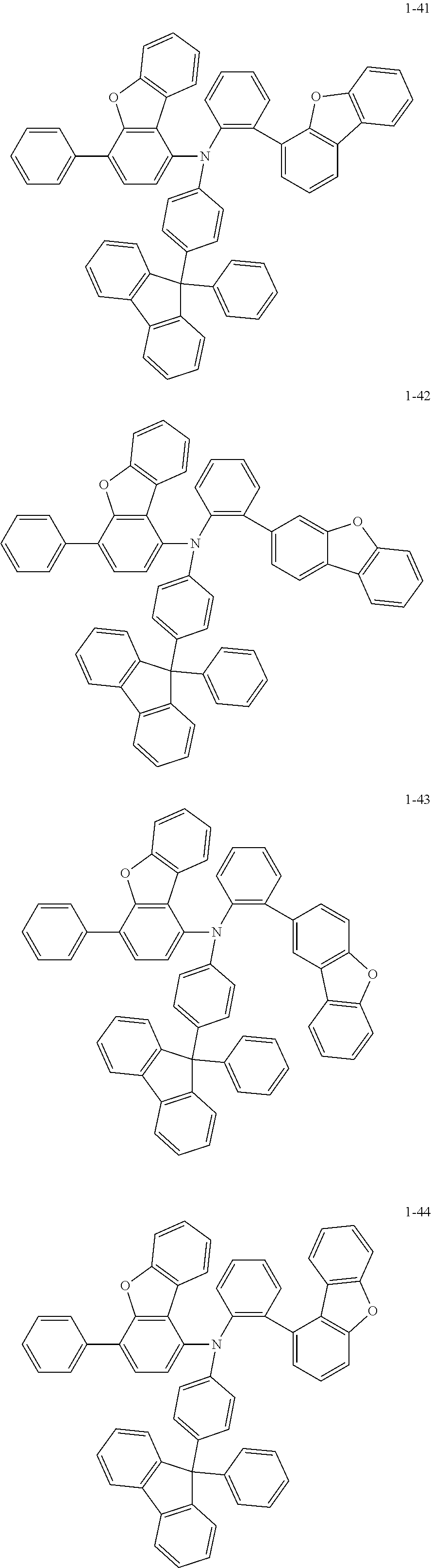

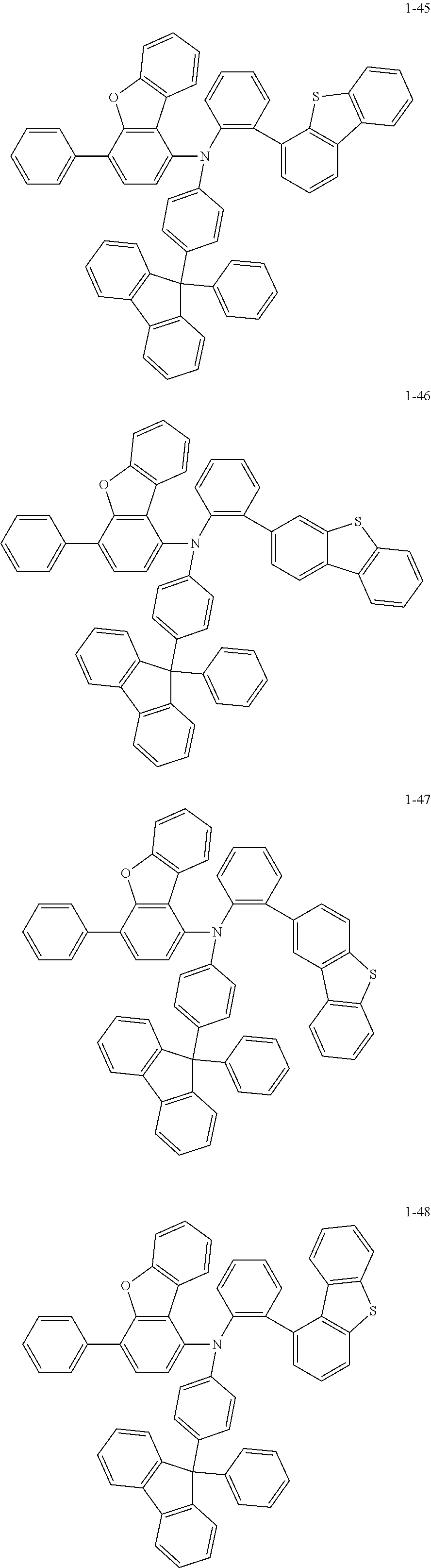

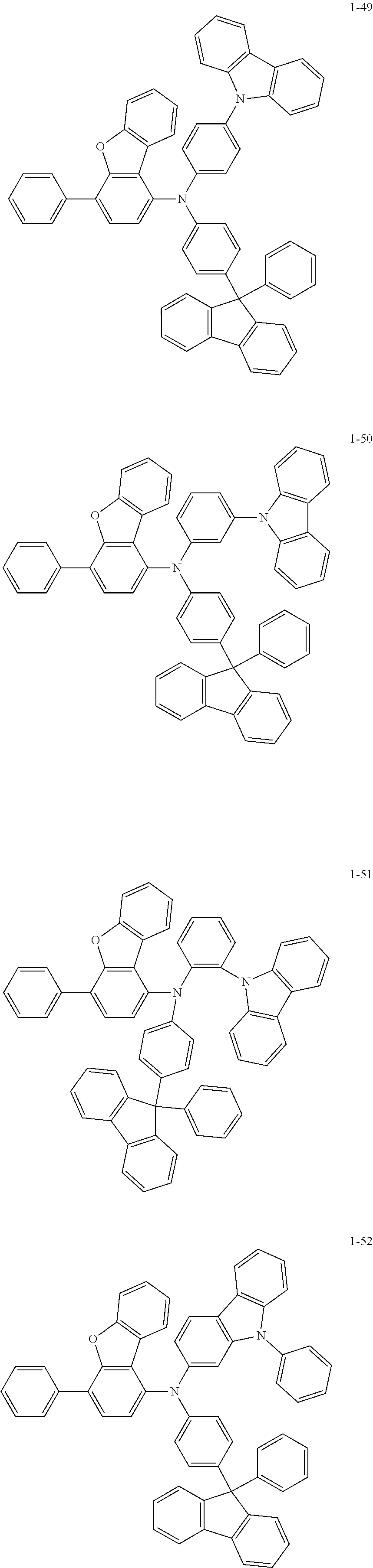 [SMILES: c1ccc(-c2ccc(N(c3ccc(-n4c5ccccc5c5ccccc54)cc3)c3ccc(C4(c5ccccc5)c5ccccc5-c5ccccc54)cc3)c3c2oc2ccccc23)cc1 ; c1ccc(-c2ccc(N(c3ccc(C4(c5ccccc5)c5ccccc5-c5ccccc54)cc3)c3cccc(-n4c5ccccc5c5ccccc54)c3)c3c2oc2ccccc23)cc1 ; c1ccc(-c2ccc(N(c3ccc(C4(c5ccccc5)c5ccccc5-c5ccccc54)cc3)c3ccccc3-n3c4ccccc4c4ccccc43)c3c2oc2ccccc23)cc1 ; c1ccc(-c2ccc(N(c3ccc(C4(c5ccccc5)c5ccccc5-c5ccccc54)cc3)c3ccc4c5ccccc5n(-c5ccccc5)c4c3)c3c2oc2ccccc23)cc1]

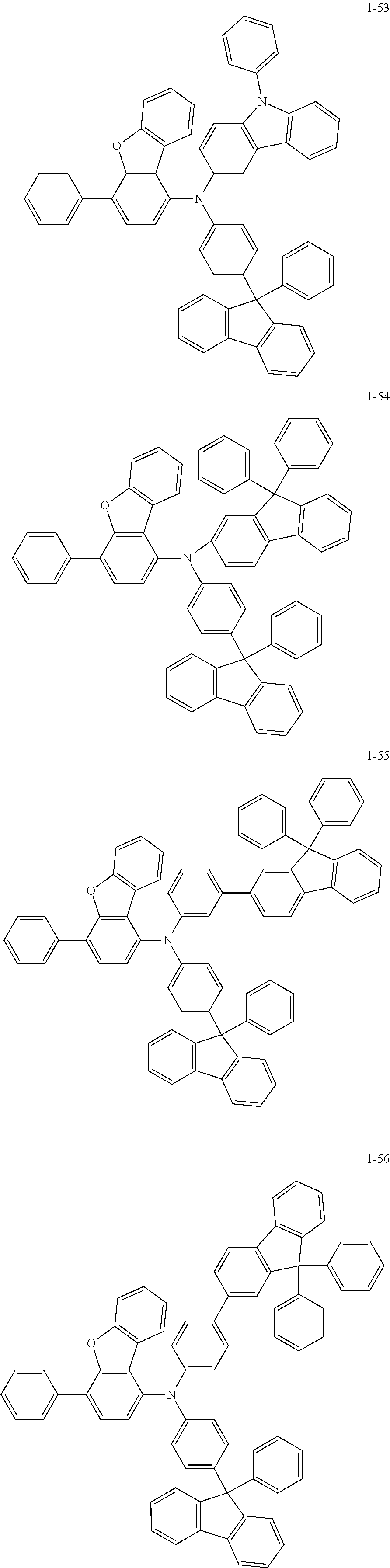 [SMILES: c1ccc(-c2ccc(N(c3ccc(C4(c5ccccc5)c5ccccc5-c5ccccc54)cc3)c3ccc4c(c3)c3ccccc3n4-c3ccccc3)c3c2oc2ccccc23)cc1 ; c1ccc(-c2ccc(N(c3ccc(C4(c5ccccc5)c5ccccc5-c5ccccc54)cc3)c3ccc4c(c3)C(c3ccccc3)(c3ccccc3)c3ccccc3-4)c3c2oc2ccccc23)cc1 ; c1ccc(-c2ccc(N(c3ccc(C4(c5ccccc5)c5ccccc5-c5ccccc54)cc3)c3cccc(-c4ccc5c(c4)C(c4ccccc4)(c4ccccc4)c4ccccc4-5)c3)c3c2oc2ccccc23)cc1 ; c1ccc(-c2ccc(N(c3ccc(-c4ccc5c(c4)C(c4ccccc4)(c4ccccc4)c4ccccc4-5)cc3)c3ccc(C4(c5ccccc5)c5ccccc5-c5ccccc54)cc3)c3c2oc2ccccc23)cc1]

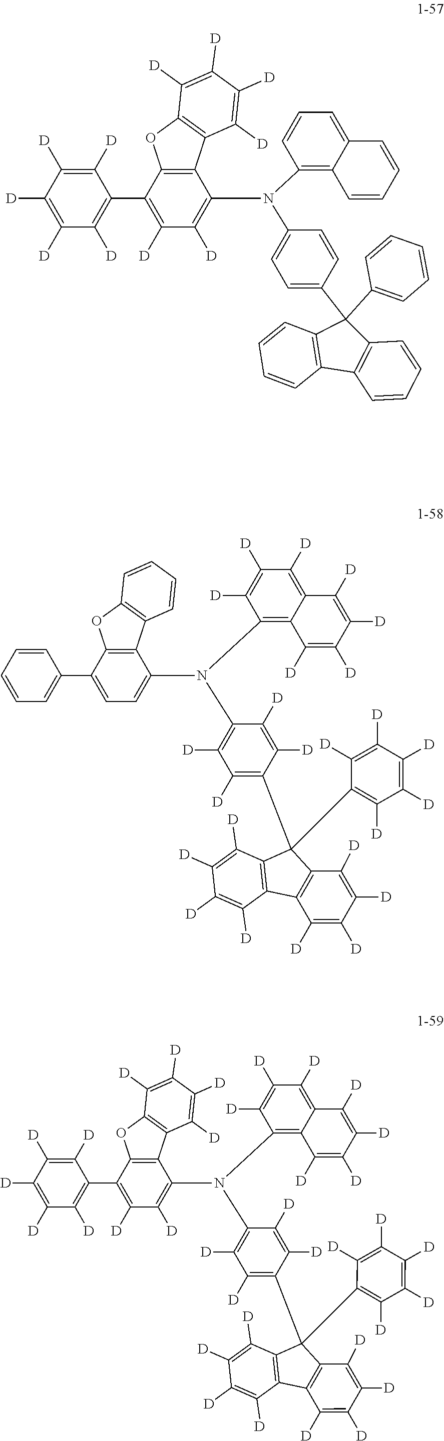

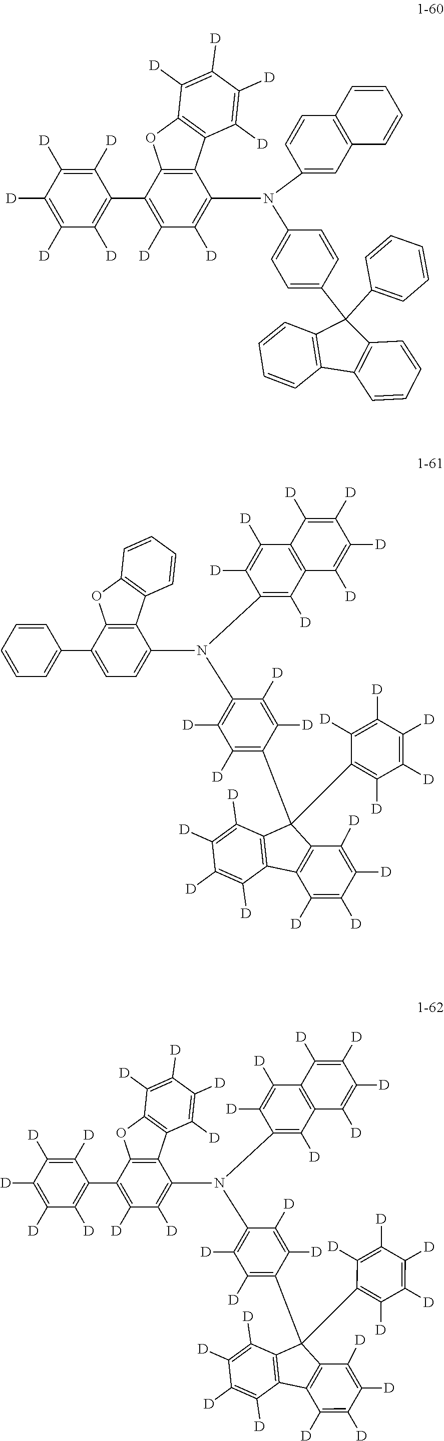

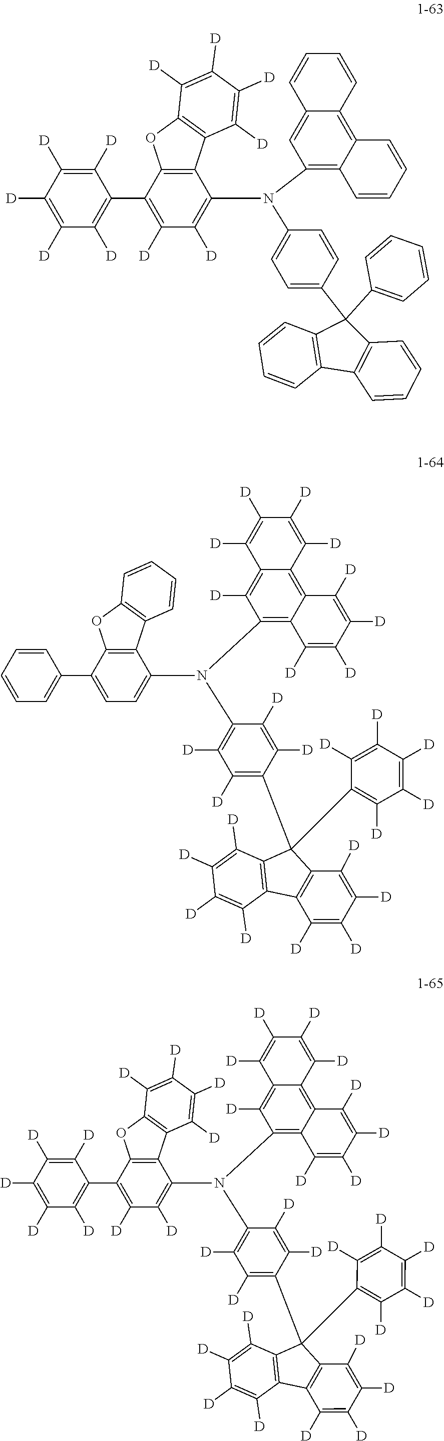

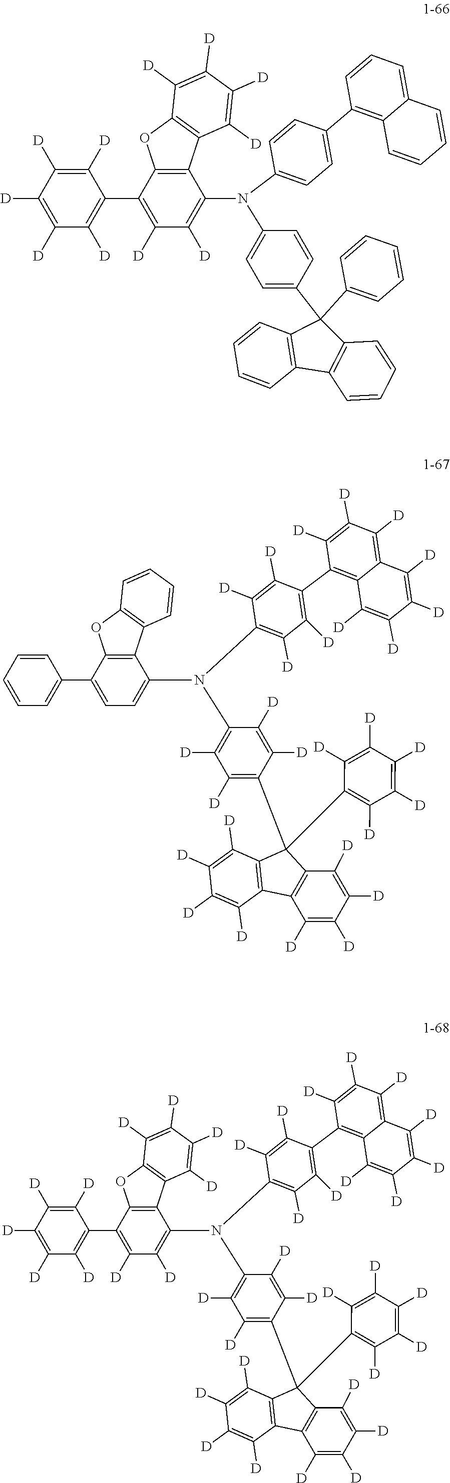

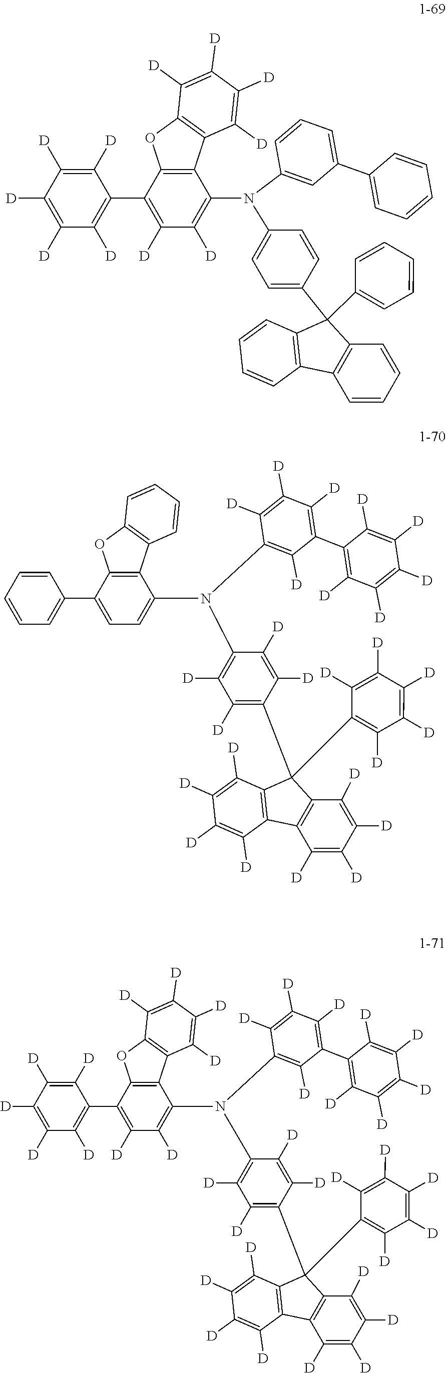

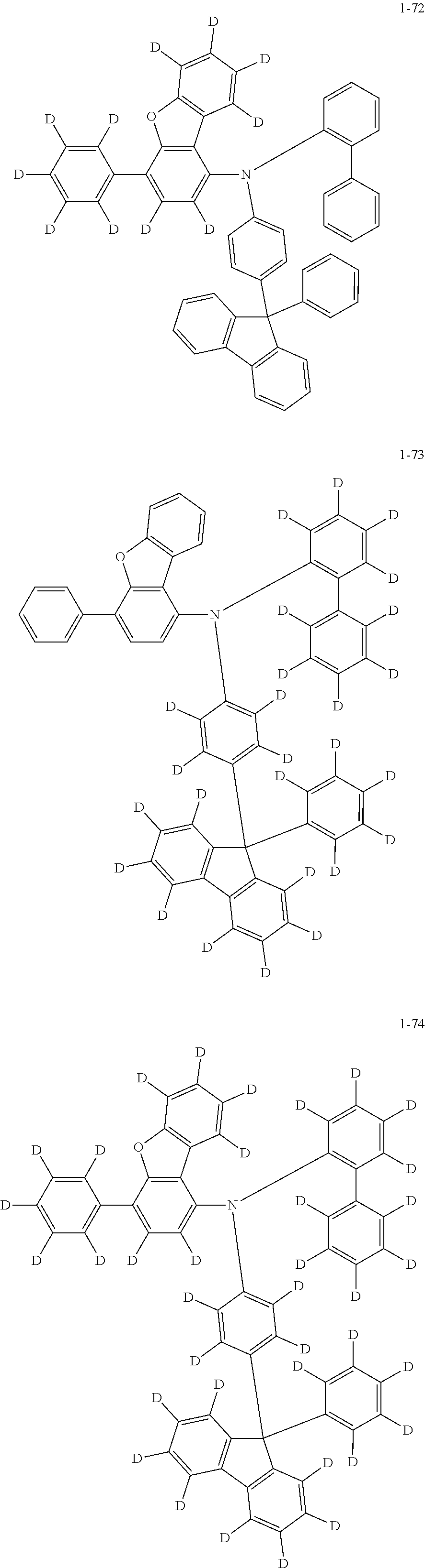

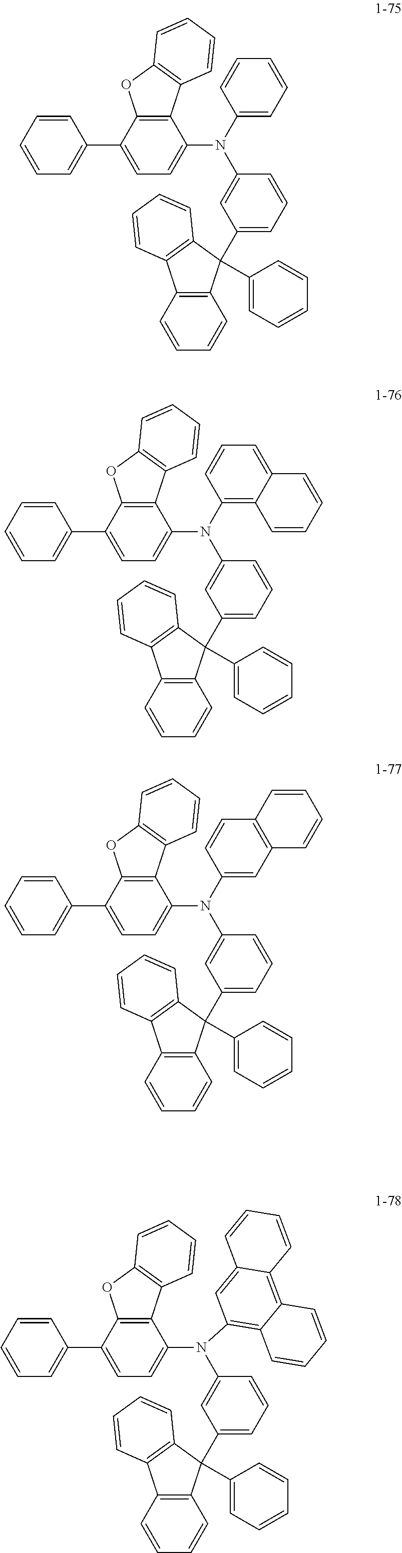

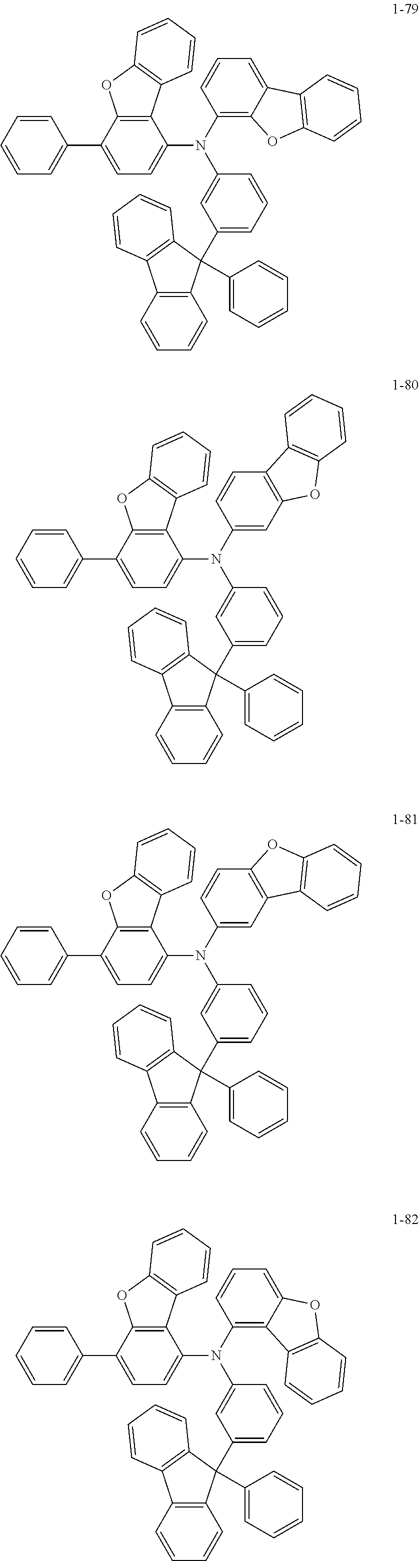 [SMILES: c1ccc(-c2ccc(N(c3cccc(C4(c5ccccc5)c5ccccc5-c5ccccc54)c3)c3cccc4c3oc3ccccc34)c3c2oc2ccccc23)cc1 ; c1ccc(-c2ccc(N(c3cccc(C4(c5ccccc5)c5ccccc5-c5ccccc54)c3)c3ccc4c(c3)oc3ccccc34)c3c2oc2ccccc23)cc1 ; c1ccc(-c2ccc(N(c3cccc(C4(c5ccccc5)c5ccccc5-c5ccccc54)c3)c3ccc4oc5ccccc5c4c3)c3c2oc2ccccc23)cc1 ; c1ccc(-c2ccc(N(c3cccc(C4(c5ccccc5)c5ccccc5-c5ccccc54)c3)c3cccc4oc5ccccc5c34)c3c2oc2ccccc23)cc1]

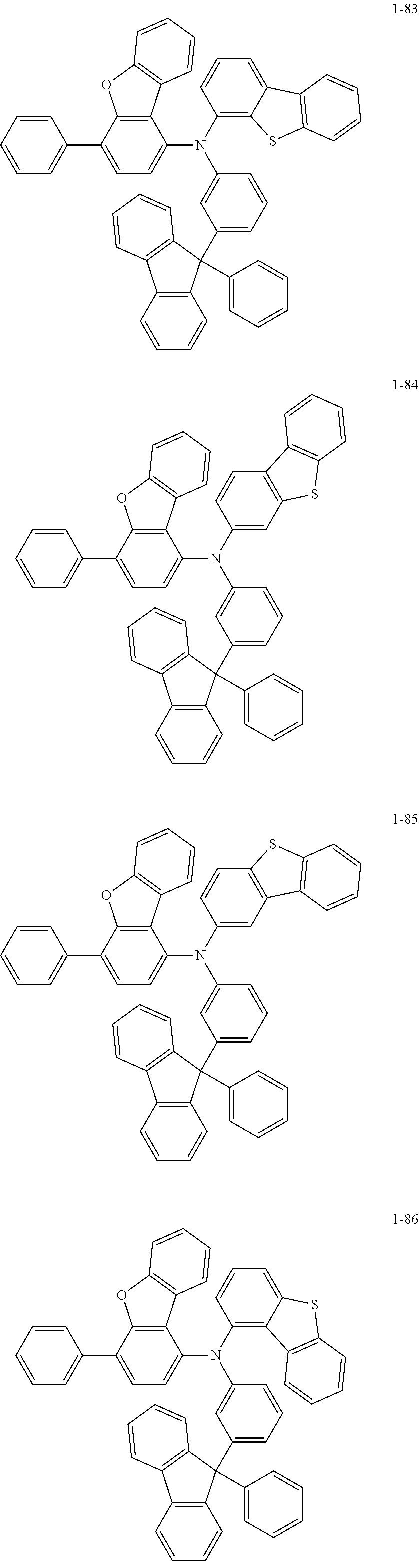 [SMILES: c1ccc(-c2ccc(N(c3cccc(C4(c5ccccc5)c5ccccc5-c5ccccc54)c3)c3cccc4c3sc3ccccc34)c3c2oc2ccccc23)cc1 ; c1ccc(-c2ccc(N(c3cccc(C4(c5ccccc5)c5ccccc5-c5ccccc54)c3)c3ccc4c(c3)sc3ccccc34)c3c2oc2ccccc23)cc1 ; c1ccc(-c2ccc(N(c3cccc(C4(c5ccccc5)c5ccccc5-c5ccccc54)c3)c3ccc4sc5ccccc5c4c3)c3c2oc2ccccc23)cc1 ; c1ccc(-c2ccc(N(c3cccc(C4(c5ccccc5)c5ccccc5-c5ccccc54)c3)c3cccc4sc5ccccc5c34)c3c2oc2ccccc23)cc1]

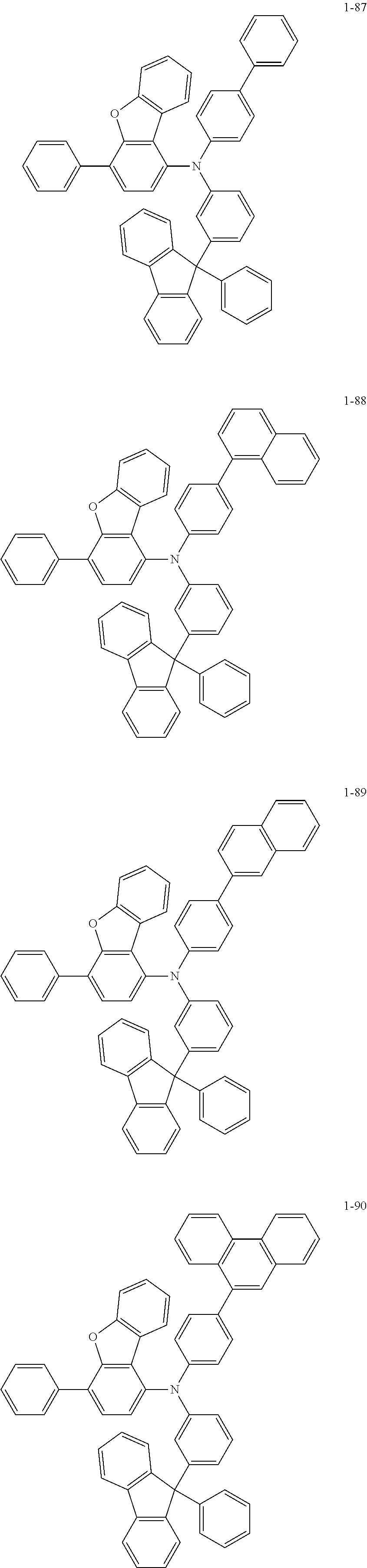 [SMILES: c1ccc(-c2ccc(N(c3cccc(C4(c5ccccc5)c5ccccc5-c5ccccc54)c3)c3ccc(-c4ccccc4)c4oc5ccccc5c34)cc2)cc1 ; c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1 ; c1ccc(-c2ccc(N(c3ccc(-c4ccc5ccccc5c4)cc3)c3cccc(C4(c5ccccc5)c5ccccc5-c5ccccc54)c3)c3c2oc2ccccc23)cc1 ; c1ccc(-c2ccc(N(c3ccc(-c4cc5ccccc5c5ccccc45)cc3)c3cccc(C4(c5ccccc5)c5ccccc5-c5ccccc54)c3)c3c2oc2ccccc23)cc1]

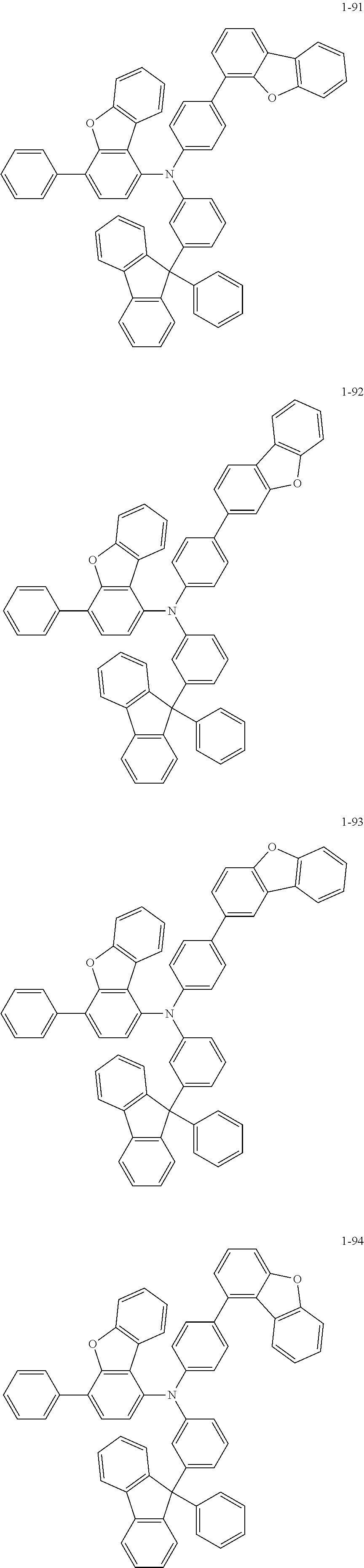 [SMILES: c1ccc(-c2ccc(N(c3ccc(-c4cccc5c4oc4ccccc45)cc3)c3cccc(C4(c5ccccc5)c5ccccc5-c5ccccc54)c3)c3c2oc2ccccc23)cc1 ; c1ccc(-c2ccc(N(c3ccc(-c4ccc5c(c4)oc4ccccc45)cc3)c3cccc(C4(c5ccccc5)c5ccccc5-c5ccccc54)c3)c3c2oc2ccccc23)cc1 ; c1ccc(-c2ccc(N(c3ccc(-c4ccc5oc6ccccc6c5c4)cc3)c3cccc(C4(c5ccccc5)c5ccccc5-c5ccccc54)c3)c3c2oc2ccccc23)cc1 ; c1ccc(-c2ccc(N(c3ccc(-c4cccc5oc6ccccc6c45)cc3)c3cccc(C4(c5ccccc5)c5ccccc5-c5ccccc54)c3)c3c2oc2ccccc23)cc1]

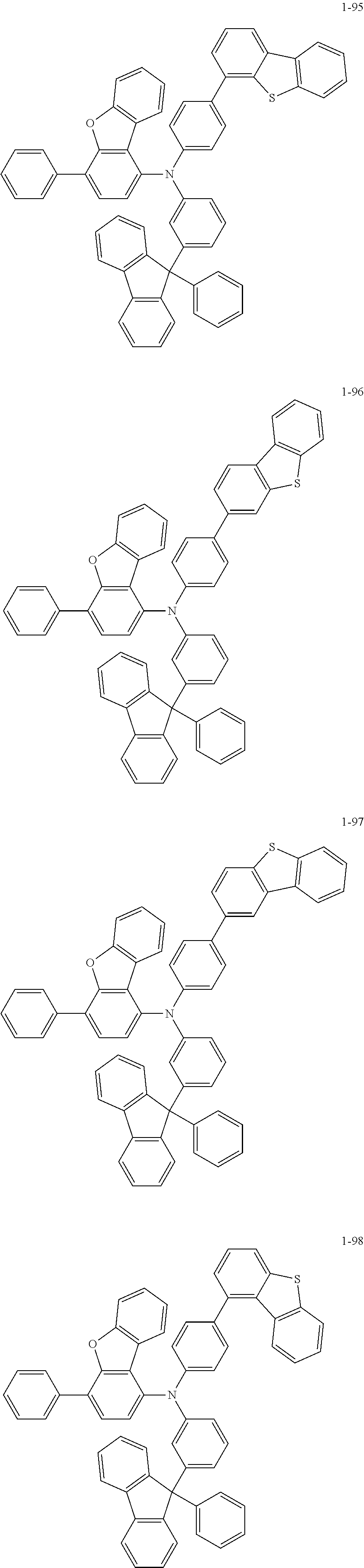 [SMILES: c1ccc(-c2ccc(N(c3ccc(-c4cccc5c4sc4ccccc45)cc3)c3cccc(C4(c5ccccc5)c5ccccc5-c5ccccc54)c3)c3c2oc2ccccc23)cc1 ; c1ccc(-c2ccc(N(c3ccc(-c4ccc5c(c4)sc4ccccc45)cc3)c3cccc(C4(c5ccccc5)c5ccccc5-c5ccccc54)c3)c3c2oc2ccccc23)cc1 ; c1ccc(-c2ccc(N(c3ccc(-c4ccc5sc6ccccc6c5c4)cc3)c3cccc(C4(c5ccccc5)c5ccccc5-c5ccccc54)c3)c3c2oc2ccccc23)cc1 ; c1ccc(-c2ccc(N(c3ccc(-c4cccc5sc6ccccc6c45)cc3)c3cccc(C4(c5ccccc5)c5ccccc5-c5ccccc54)c3)c3c2oc2ccccc23)cc1]

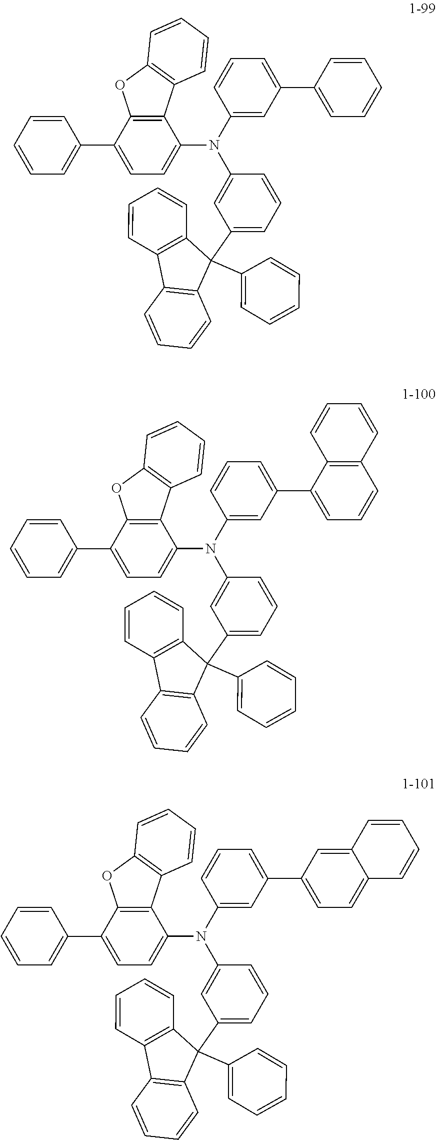 [SMILES: c1ccc(-c2cccc(N(c3cccc(C4(c5ccccc5)c5ccccc5-c5ccccc54)c3)c3ccc(-c4ccccc4)c4oc5ccccc5c34)c2)cc1 ; c1ccc(-c2ccc(N(c3cccc(-c4cccc5ccccc45)c3)c3cccc(C4(c5ccccc5)c5ccccc5-c5ccccc54)c3)c3c2oc2ccccc23)cc1 ; c1ccc(-c2ccc(N(c3cccc(-c4ccc5ccccc5c4)c3)c3cccc(C4(c5ccccc5)c5ccccc5-c5ccccc54)c3)c3c2oc2ccccc23)cc1]

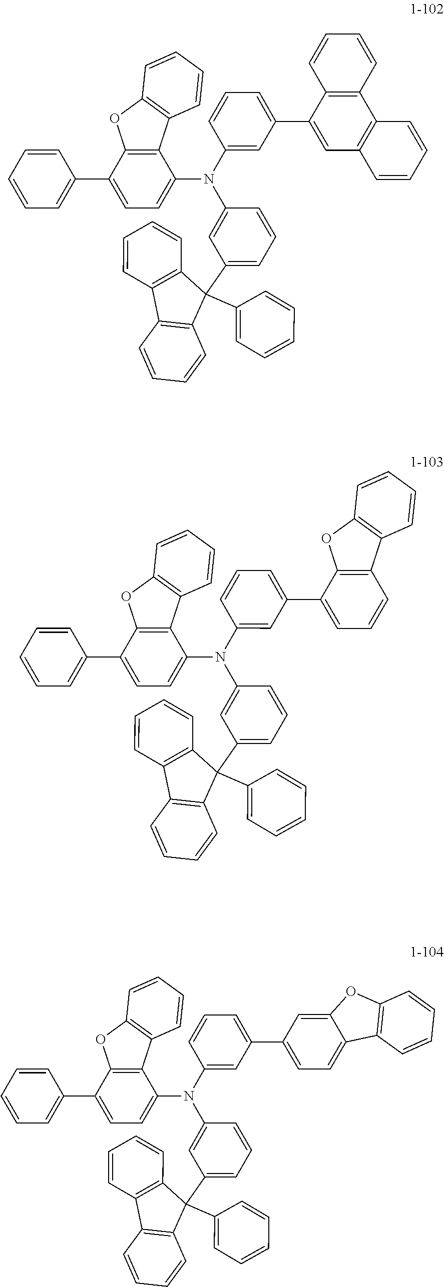 [SMILES: c1ccc(-c2ccc(N(c3cccc(-c4cc5ccccc5c5ccccc45)c3)c3cccc(C4(c5ccccc5)c5ccccc5-c5ccccc54)c3)c3c2oc2ccccc23)cc1 ; c1ccc(-c2ccc(N(c3cccc(-c4cccc5c4oc4ccccc45)c3)c3cccc(C4(c5ccccc5)c5ccccc5-c5ccccc54)c3)c3c2oc2ccccc23)cc1 ; c1ccc(-c2ccc(N(c3cccc(-c4ccc5c(c4)oc4ccccc45)c3)c3cccc(C4(c5ccccc5)c5ccccc5-c5ccccc54)c3)c3c2oc2ccccc23)cc1]

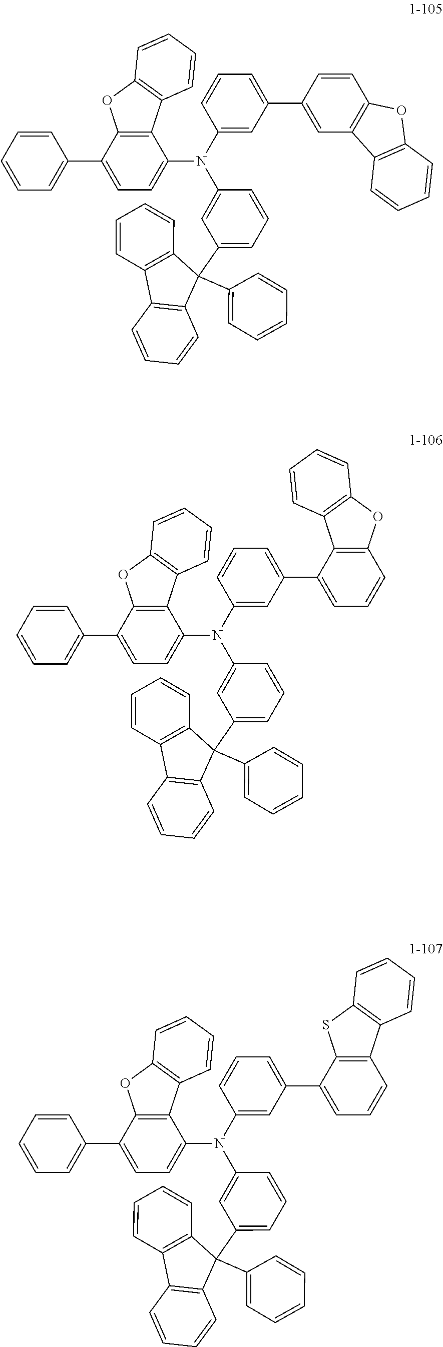 [SMILES: c1ccc(-c2ccc(N(c3cccc(-c4ccc5oc6ccccc6c5c4)c3)c3cccc(C4(c5ccccc5)c5ccccc5-c5ccccc54)c3)c3c2oc2ccccc23)cc1 ; c1ccc(-c2ccc(N(c3cccc(-c4cccc5oc6ccccc6c45)c3)c3cccc(C4(c5ccccc5)c5ccccc5-c5ccccc54)c3)c3c2oc2ccccc23)cc1 ; c1ccc(-c2ccc(N(c3cccc(-c4cccc5c4sc4ccccc45)c3)c3cccc(C4(c5ccccc5)c5ccccc5-c5ccccc54)c3)c3c2oc2ccccc23)cc1]

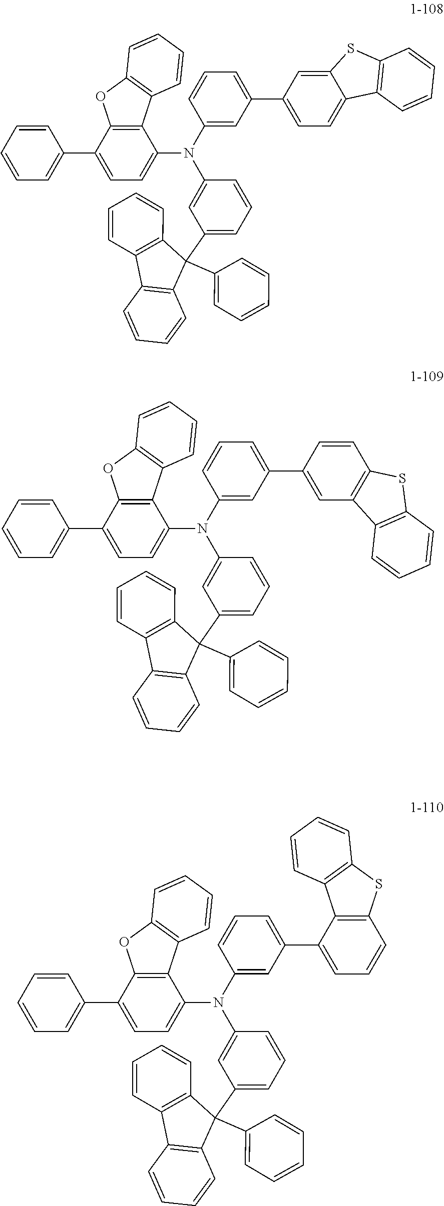 [SMILES: c1ccc(-c2ccc(N(c3cccc(-c4ccc5c(c4)sc4ccccc45)c3)c3cccc(C4(c5ccccc5)c5ccccc5-c5ccccc54)c3)c3c2oc2ccccc23)cc1 ; c1ccc(-c2ccc(N(c3cccc(-c4ccc5sc6ccccc6c5c4)c3)c3cccc(C4(c5ccccc5)c5ccccc5-c5ccccc54)c3)c3c2oc2ccccc23)cc1 ; c1ccc(-c2ccc(N(c3cccc(-c4cccc5sc6ccccc6c45)c3)c3cccc(C4(c5ccccc5)c5ccccc5-c5ccccc54)c3)c3c2oc2ccccc23)cc1]

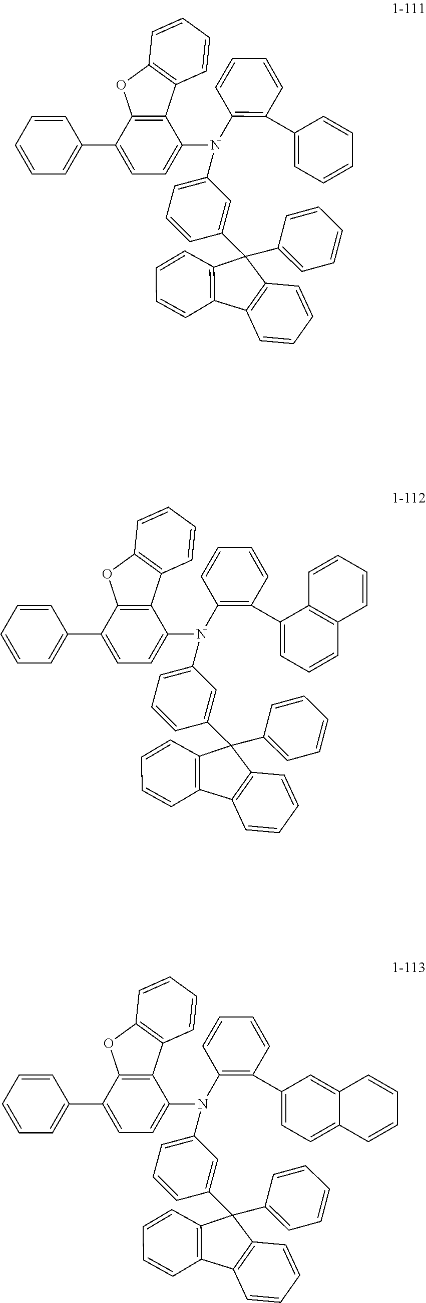 [SMILES: c1ccc(-c2ccccc2N(c2cccc(C3(c4ccccc4)c4ccccc4-c4ccccc43)c2)c2ccc(-c3ccccc3)c3oc4ccccc4c23)cc1 ; c1ccc(-c2ccc(N(c3cccc(C4(c5ccccc5)c5ccccc5-c5ccccc54)c3)c3ccccc3-c3cccc4ccccc34)c3c2oc2ccccc23)cc1 ; c1ccc(-c2ccc(N(c3cccc(C4(c5ccccc5)c5ccccc5-c5ccccc54)c3)c3ccccc3-c3ccc4ccccc4c3)c3c2oc2ccccc23)cc1]

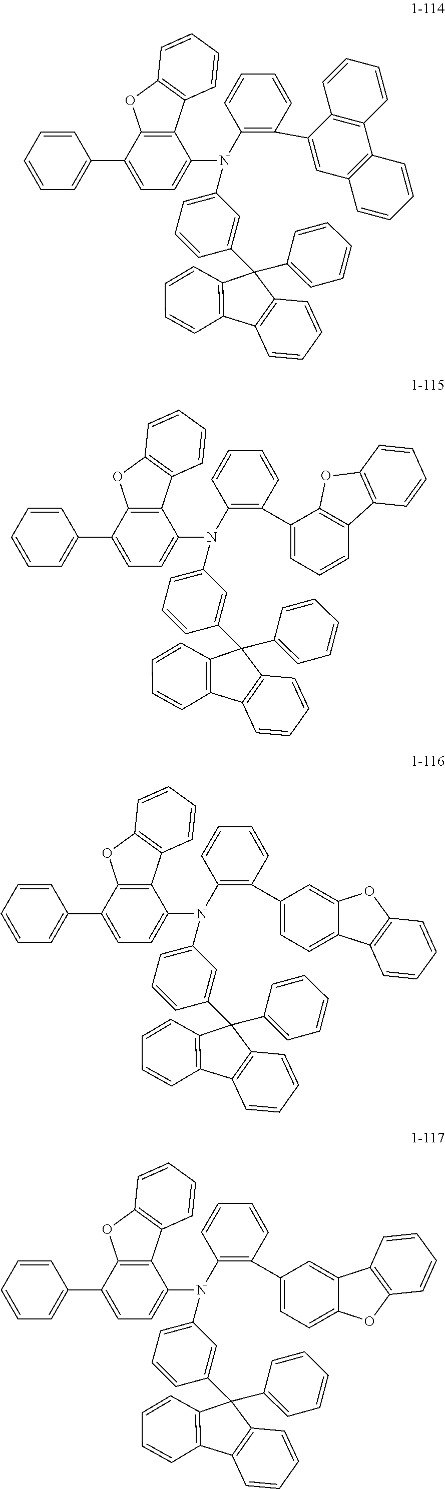 [SMILES: c1ccc(-c2ccc(N(c3cccc(C4(c5ccccc5)c5ccccc5-c5ccccc54)c3)c3ccccc3-c3cc4ccccc4c4ccccc34)c3c2oc2ccccc23)cc1 ; c1ccc(-c2ccc(N(c3cccc(C4(c5ccccc5)c5ccccc5-c5ccccc54)c3)c3ccccc3-c3cccc4c3oc3ccccc34)c3c2oc2ccccc23)cc1 ; c1ccc(-c2ccc(N(c3cccc(C4(c5ccccc5)c5ccccc5-c5ccccc54)c3)c3ccccc3-c3ccc4c(c3)oc3ccccc34)c3c2oc2ccccc23)cc1 ; c1ccc(-c2ccc(N(c3cccc(C4(c5ccccc5)c5ccccc5-c5ccccc54)c3)c3ccccc3-c3ccc4oc5ccccc5c4c3)c3c2oc2ccccc23)cc1]

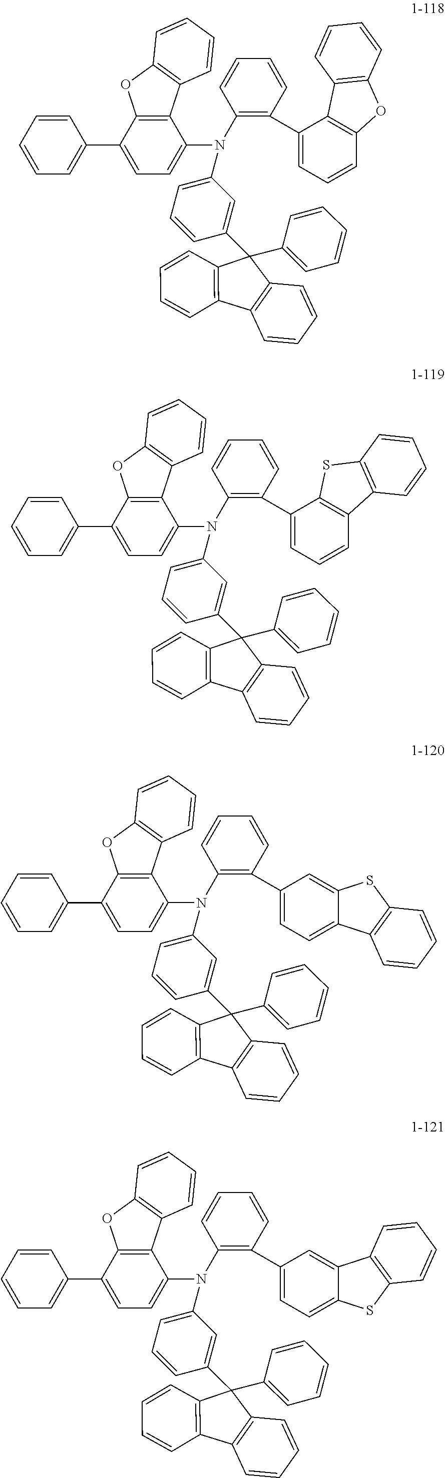 [SMILES: c1ccc(-c2ccc(N(c3cccc(C4(c5ccccc5)c5ccccc5-c5ccccc54)c3)c3ccccc3-c3cccc4oc5ccccc5c34)c3c2oc2ccccc23)cc1 ; c1ccc(-c2ccc(N(c3cccc(C4(c5ccccc5)c5ccccc5-c5ccccc54)c3)c3ccccc3-c3cccc4c3sc3ccccc34)c3c2oc2ccccc23)cc1 ; c1ccc(-c2ccc(N(c3cccc(C4(c5ccccc5)c5ccccc5-c5ccccc54)c3)c3ccccc3-c3ccc4c(c3)sc3ccccc34)c3c2oc2ccccc23)cc1 ; c1ccc(-c2ccc(N(c3cccc(C4(c5ccccc5)c5ccccc5-c5ccccc54)c3)c3ccccc3-c3ccc4sc5ccccc5c4c3)c3c2oc2ccccc23)cc1]

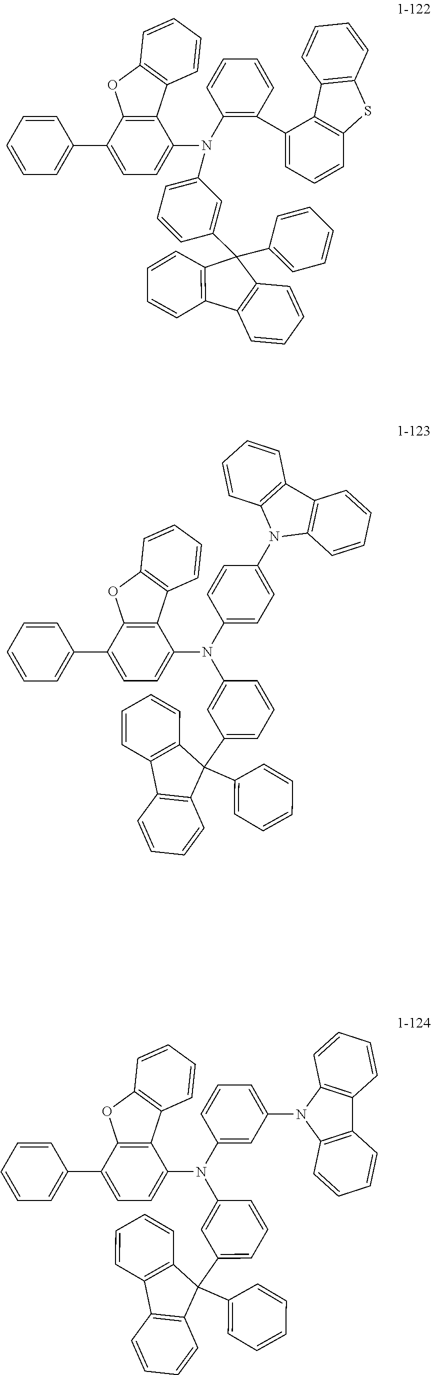 [SMILES: c1ccc(-c2ccc(N(c3cccc(C4(c5ccccc5)c5ccccc5-c5ccccc54)c3)c3ccccc3-c3cccc4sc5ccccc5c34)c3c2oc2ccccc23)cc1 ; c1ccc(-c2ccc(N(c3ccc(-n4c5ccccc5c5ccccc54)cc3)c3cccc(C4(c5ccccc5)c5ccccc5-c5ccccc54)c3)c3c2oc2ccccc23)cc1 ; c1ccc(-c2ccc(N(c3cccc(-n4c5ccccc5c5ccccc54)c3)c3cccc(C4(c5ccccc5)c5ccccc5-c5ccccc54)c3)c3c2oc2ccccc23)cc1]

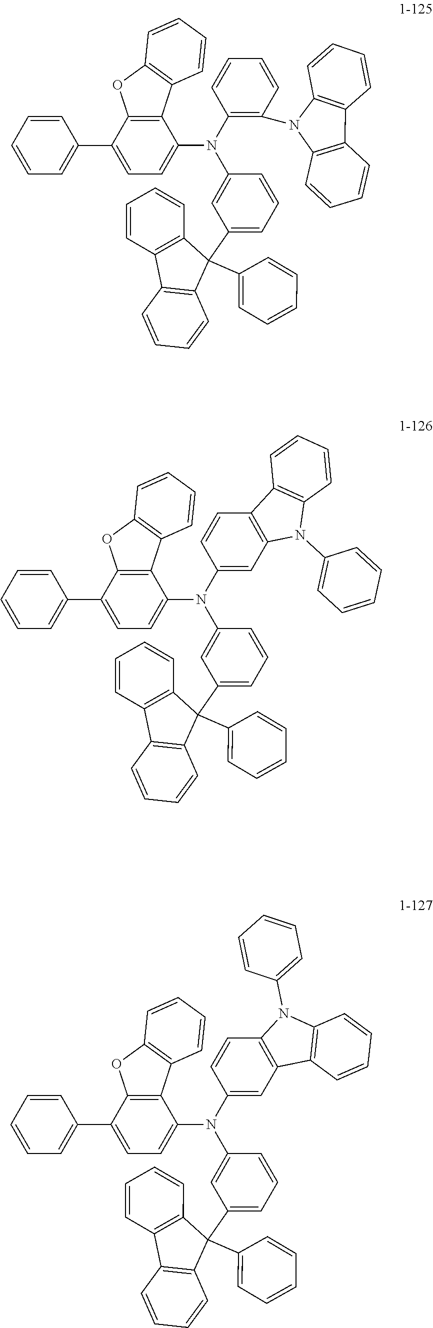 [SMILES: c1ccc(-c2ccc(N(c3cccc(C4(c5ccccc5)c5ccccc5-c5ccccc54)c3)c3ccccc3-n3c4ccccc4c4ccccc43)c3c2oc2ccccc23)cc1 ; c1ccc(-c2ccc(N(c3cccc(C4(c5ccccc5)c5ccccc5-c5ccccc54)c3)c3ccc4c5ccccc5n(-c5ccccc5)c4c3)c3c2oc2ccccc23)cc1 ; c1ccc(-c2ccc(N(c3cccc(C4(c5ccccc5)c5ccccc5-c5ccccc54)c3)c3ccc4c(c3)c3ccccc3n4-c3ccccc3)c3c2oc2ccccc23)cc1]

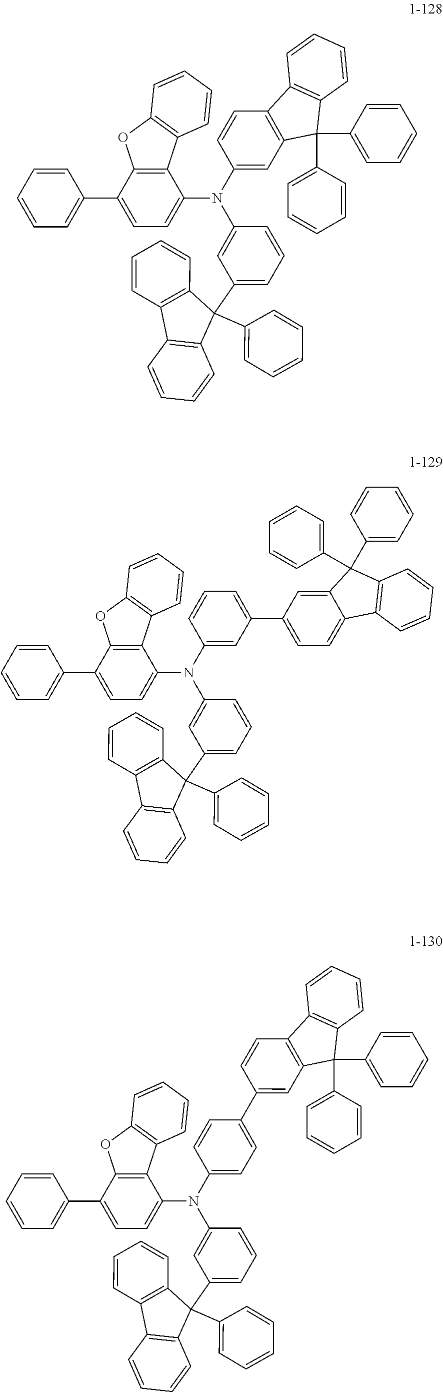 [SMILES: c1ccc(-c2ccc(N(c3cccc(C4(c5ccccc5)c5ccccc5-c5ccccc54)c3)c3ccc4c(c3)C(c3ccccc3)(c3ccccc3)c3ccccc3-4)c3c2oc2ccccc23)cc1 ; c1ccc(-c2ccc(N(c3cccc(-c4ccc5c(c4)C(c4ccccc4)(c4ccccc4)c4ccccc4-5)c3)c3cccc(C4(c5ccccc5)c5ccccc5-c5ccccc54)c3)c3c2oc2ccccc23)cc1 ; c1ccc(-c2ccc(N(c3ccc(-c4ccc5c(c4)C(c4ccccc4)(c4ccccc4)c4ccccc4-5)cc3)c3cccc(C4(c5ccccc5)c5ccccc5-c5ccccc54)c3)c3c2oc2ccccc23)cc1]

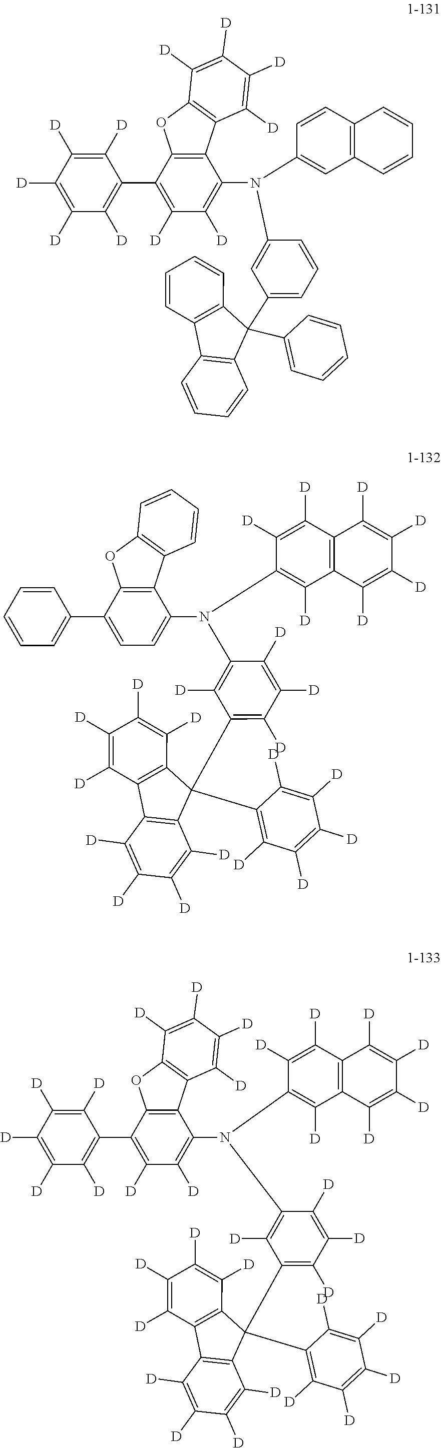

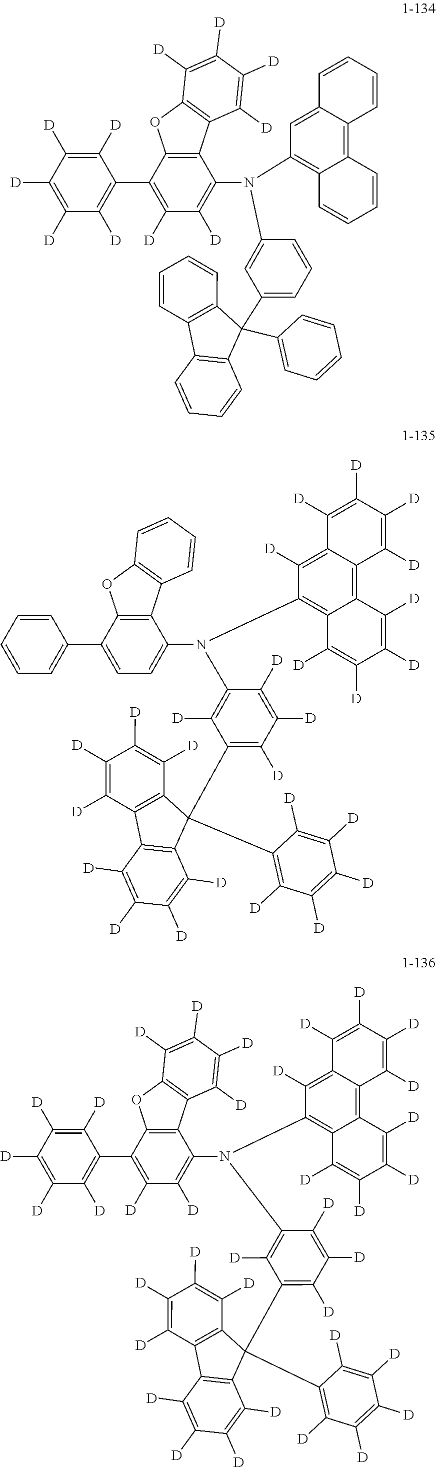

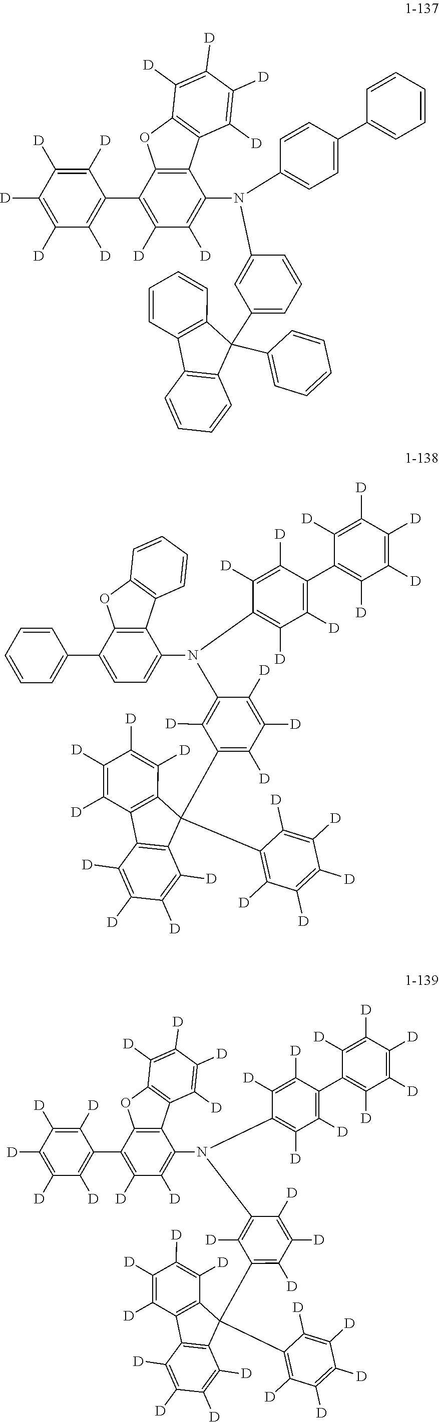

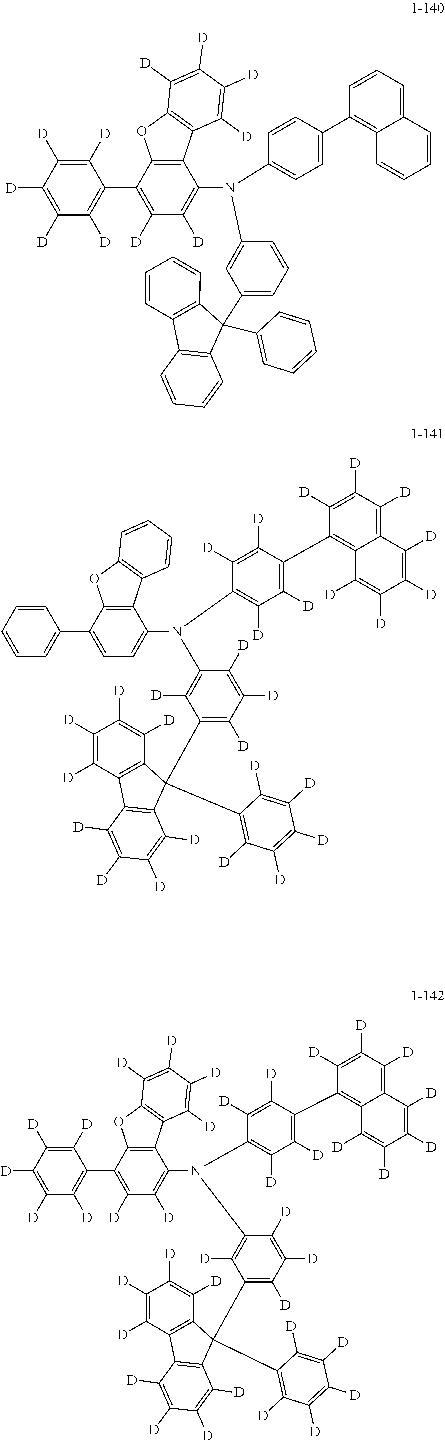

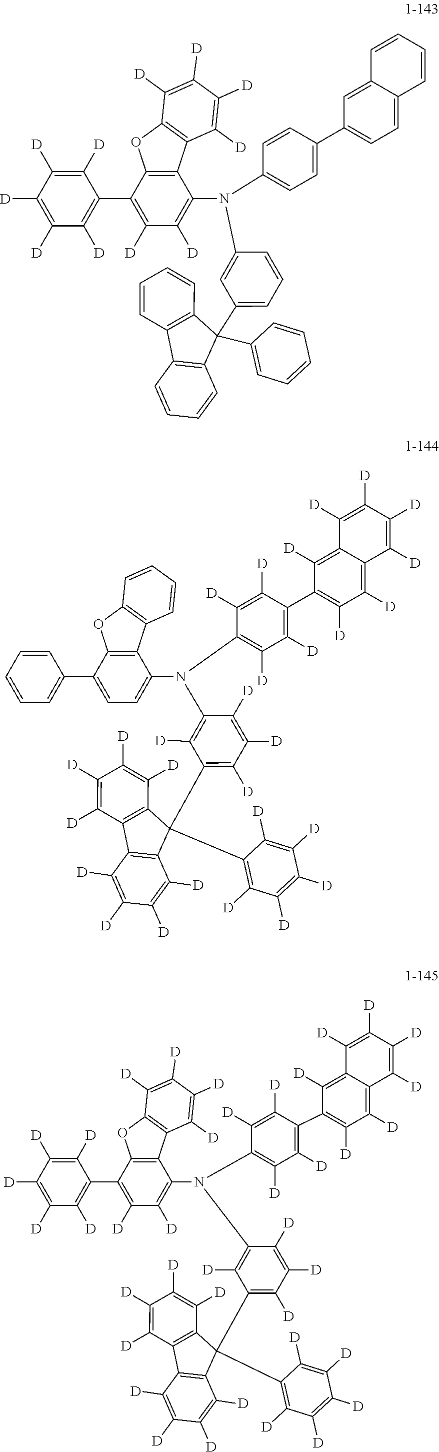

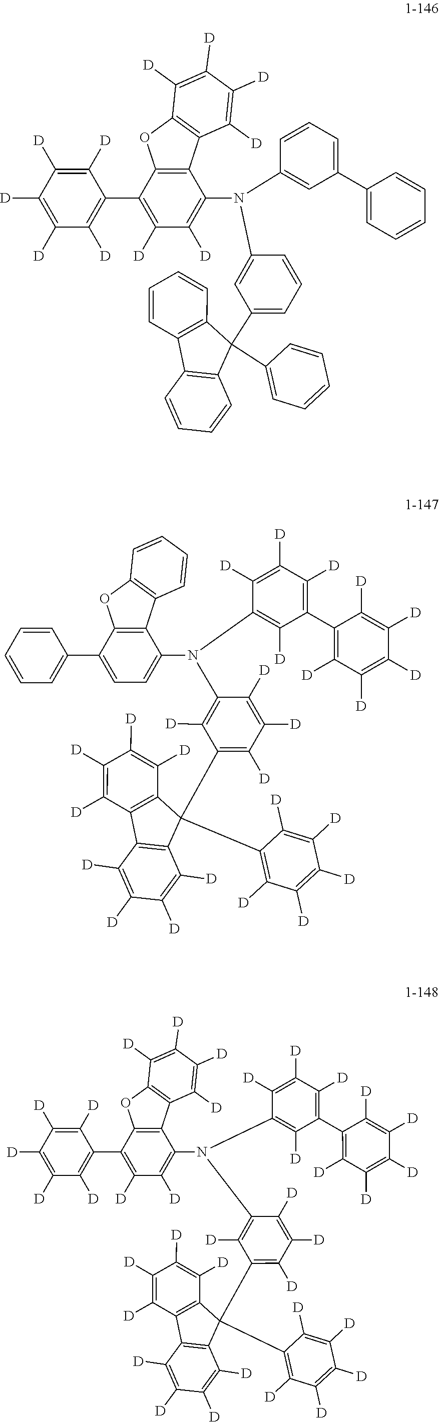

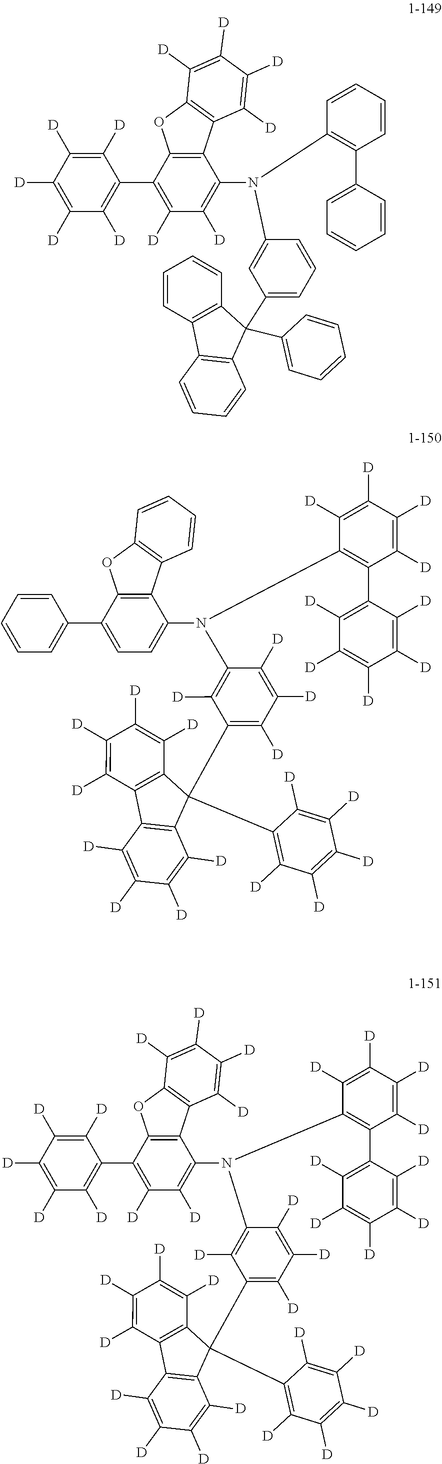

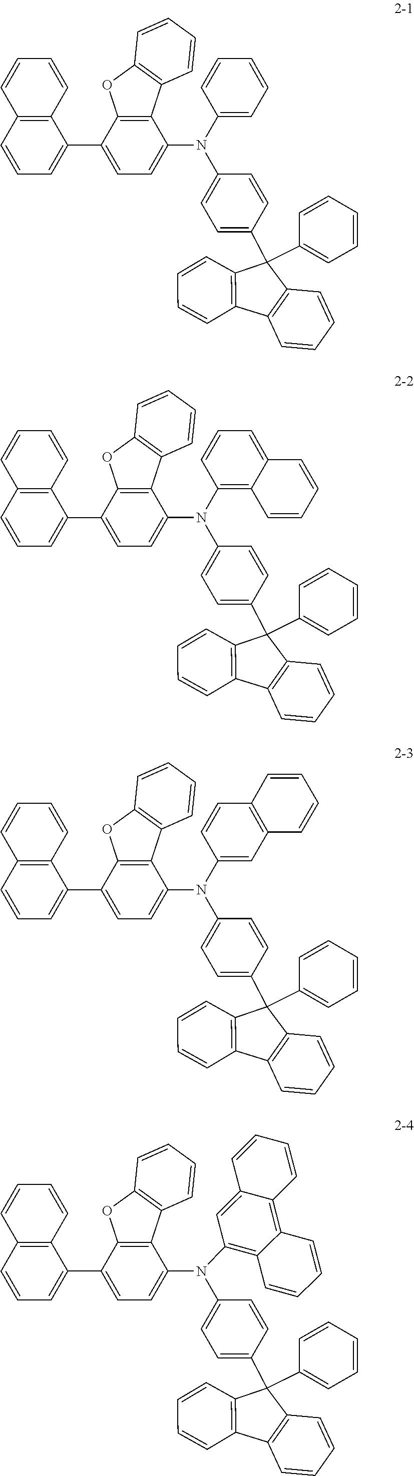 [SMILES: c1ccc(N(c2ccc(C3(c4ccccc4)c4ccccc4-c4ccccc43)cc2)c2ccc(-c3cccc4ccccc34)c3oc4ccccc4c23)cc1 ; c1ccc(C2(c3ccc(N(c4cccc5ccccc45)c4ccc(-c5cccc6ccccc56)c5oc6ccccc6c45)cc3)c3ccccc3-c3ccccc32)cc1 ; c1ccc(C2(c3ccc(N(c4ccc5ccccc5c4)c4ccc(-c5cccc6ccccc56)c5oc6ccccc6c45)cc3)c3ccccc3-c3ccccc32)cc1 ; c1ccc(C2(c3ccc(N(c4cc5ccccc5c5ccccc45)c4ccc(-c5cccc6ccccc56)c5oc6ccccc6c45)cc3)c3ccccc3-c3ccccc32)cc1]

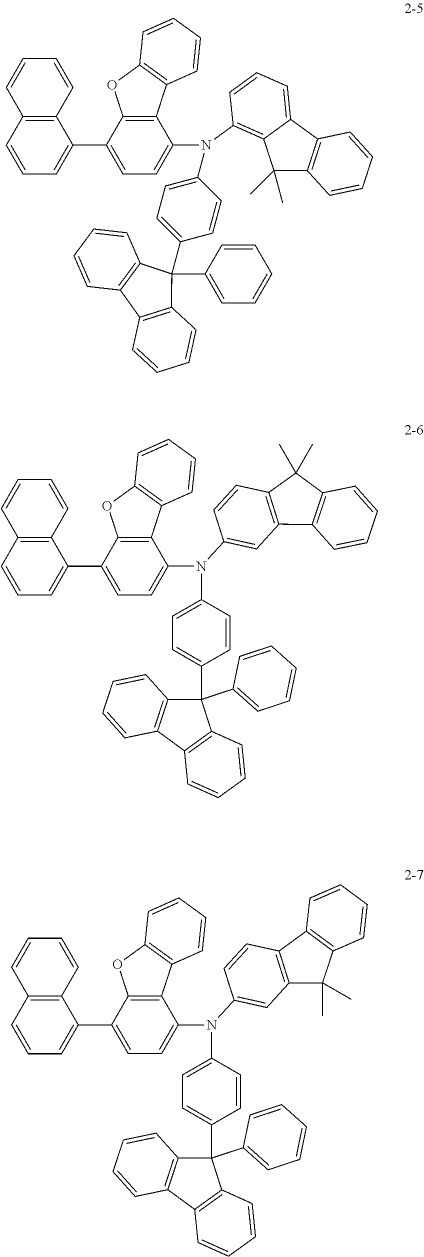 [SMILES: CC1(C)c2ccccc2-c2cccc(N(c3ccc(C4(c5ccccc5)c5ccccc5-c5ccccc54)cc3)c3ccc(-c4cccc5ccccc45)c4oc5ccccc5c34)c21 ; CC1(C)c2ccccc2-c2cc(N(c3ccc(C4(c5ccccc5)c5ccccc5-c5ccccc54)cc3)c3ccc(-c4cccc5ccccc45)c4oc5ccccc5c34)ccc21 ; CC1(C)c2ccccc2-c2ccc(N(c3ccc(C4(c5ccccc5)c5ccccc5-c5ccccc54)cc3)c3ccc(-c4cccc5ccccc45)c4oc5ccccc5c34)cc21]

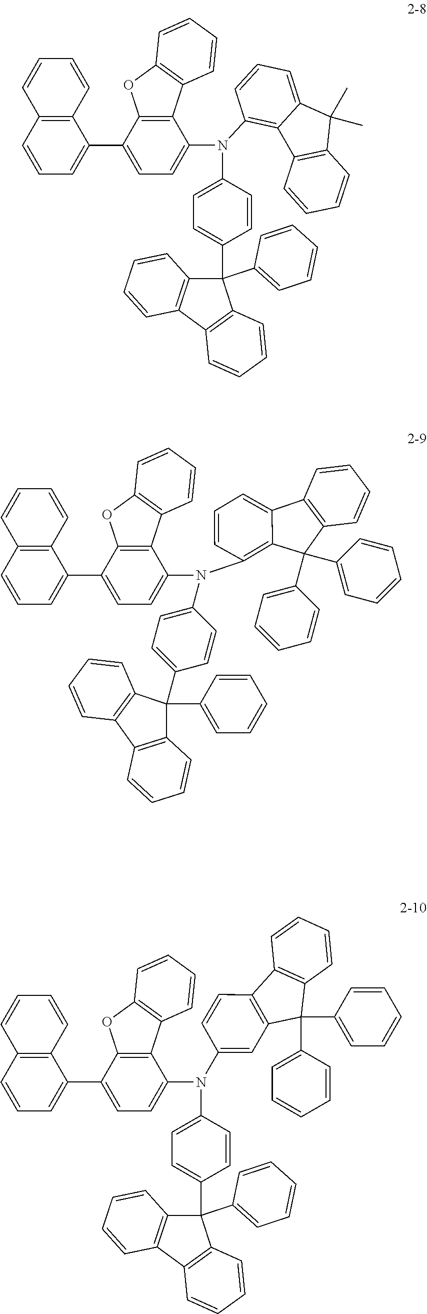 [SMILES: CC1(C)c2ccccc2-c2c(N(c3ccc(C4(c5ccccc5)c5ccccc5-c5ccccc54)cc3)c3ccc(-c4cccc5ccccc45)c4oc5ccccc5c34)cccc21 ; c1ccc(C2(c3ccc(N(c4cccc5c4C(c4ccccc4)(c4ccccc4)c4ccccc4-5)c4ccc(-c5cccc6ccccc56)c5oc6ccccc6c45)cc3)c3ccccc3-c3ccccc32)cc1 ; c1ccc(C2(c3ccc(N(c4ccc5c(c4)C(c4ccccc4)(c4ccccc4)c4ccccc4-5)c4ccc(-c5cccc6ccccc56)c5oc6ccccc6c45)cc3)c3ccccc3-c3ccccc32)cc1]

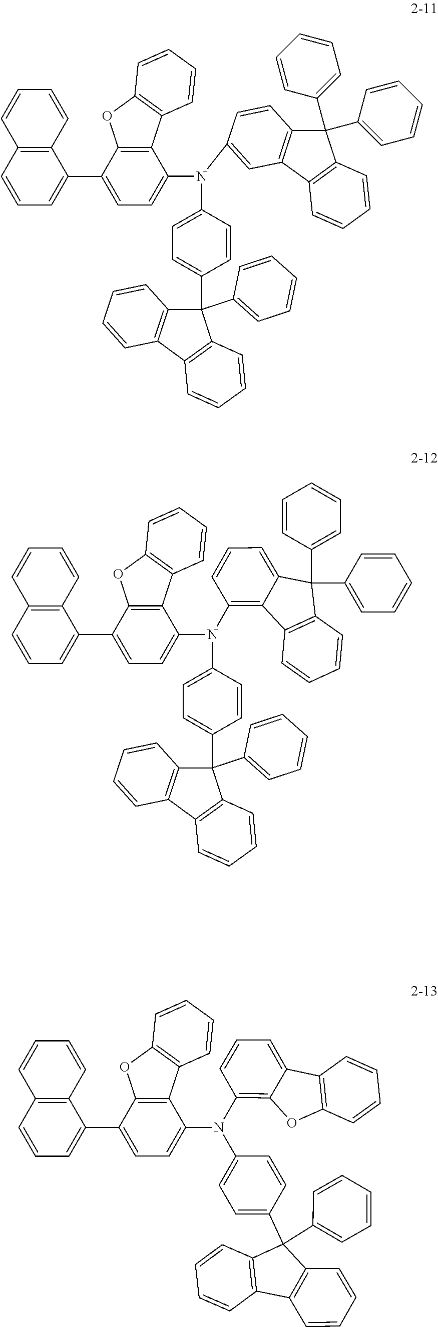 [SMILES: c1ccc(C2(c3ccc(N(c4ccc5c(c4)-c4ccccc4C5(c4ccccc4)c4ccccc4)c4ccc(-c5cccc6ccccc56)c5oc6ccccc6c45)cc3)c3ccccc3-c3ccccc32)cc1 ; c1ccc(C2(c3ccc(N(c4cccc5c4-c4ccccc4C5(c4ccccc4)c4ccccc4)c4ccc(-c5cccc6ccccc56)c5oc6ccccc6c45)cc3)c3ccccc3-c3ccccc32)cc1 ; c1ccc(C2(c3ccc(N(c4cccc5c4oc4ccccc45)c4ccc(-c5cccc6ccccc56)c5oc6ccccc6c45)cc3)c3ccccc3-c3ccccc32)cc1]

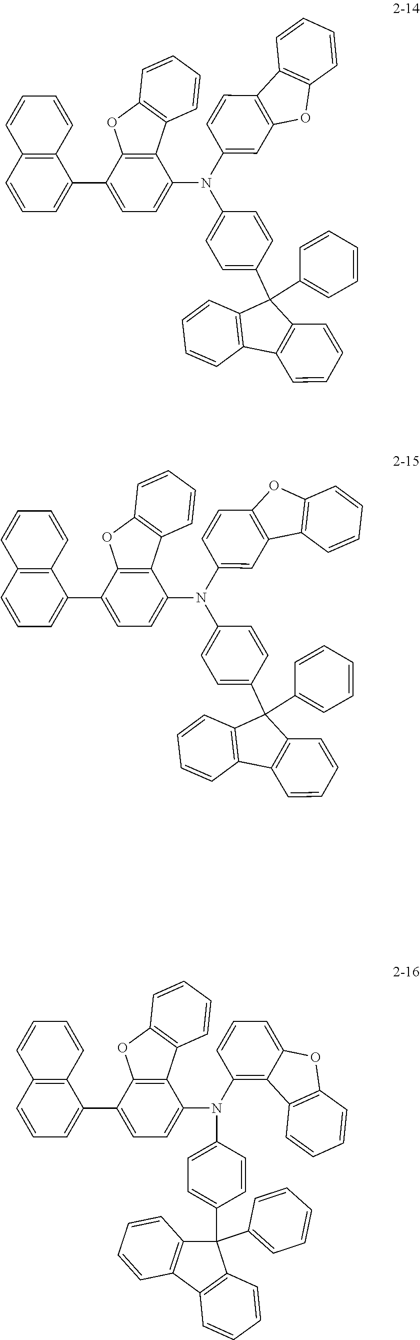 [SMILES: c1ccc(C2(c3ccc(N(c4ccc5c(c4)oc4ccccc45)c4ccc(-c5cccc6ccccc56)c5oc6ccccc6c45)cc3)c3ccccc3-c3ccccc32)cc1 ; c1ccc(C2(c3ccc(N(c4ccc5oc6ccccc6c5c4)c4ccc(-c5cccc6ccccc56)c5oc6ccccc6c45)cc3)c3ccccc3-c3ccccc32)cc1 ; c1ccc(C2(c3ccc(N(c4cccc5oc6ccccc6c45)c4ccc(-c5cccc6ccccc56)c5oc6ccccc6c45)cc3)c3ccccc3-c3ccccc32)cc1]

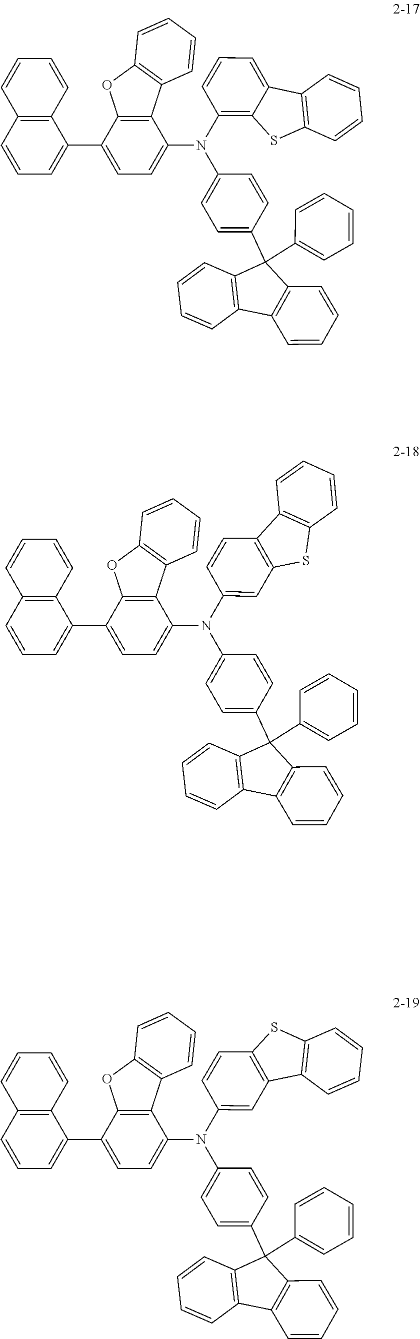 [SMILES: c1ccc(C2(c3ccc(N(c4cccc5c4sc4ccccc45)c4ccc(-c5cccc6ccccc56)c5oc6ccccc6c45)cc3)c3ccccc3-c3ccccc32)cc1 ; c1ccc(C2(c3ccc(N(c4ccc5c(c4)sc4ccccc45)c4ccc(-c5cccc6ccccc56)c5oc6ccccc6c45)cc3)c3ccccc3-c3ccccc32)cc1 ; c1ccc(C2(c3ccc(N(c4ccc5sc6ccccc6c5c4)c4ccc(-c5cccc6ccccc56)c5oc6ccccc6c45)cc3)c3ccccc3-c3ccccc32)cc1]

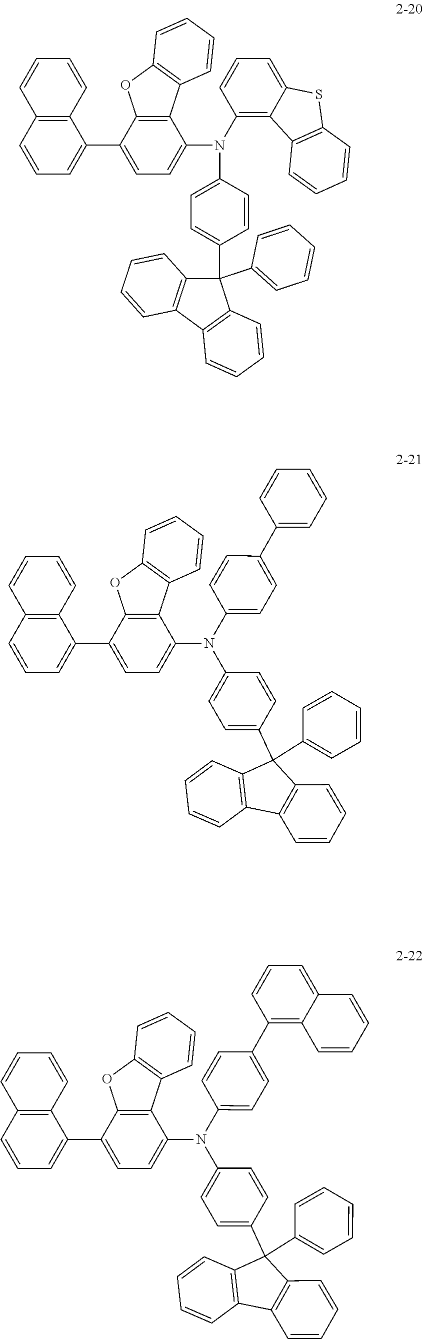 [SMILES: c1ccc(C2(c3ccc(N(c4cccc5sc6ccccc6c45)c4ccc(-c5cccc6ccccc56)c5oc6ccccc6c45)cc3)c3ccccc3-c3ccccc32)cc1 ; c1ccc(-c2ccc(N(c3ccc(C4(c5ccccc5)c5ccccc5-c5ccccc54)cc3)c3ccc(-c4cccc5ccccc45)c4oc5ccccc5c34)cc2)cc1 ; c1ccc(C2(c3ccc(N(c4ccc(-c5cccc6ccccc56)cc4)c4ccc(-c5cccc6ccccc56)c5oc6ccccc6c45)cc3)c3ccccc3-c3ccccc32)cc1]

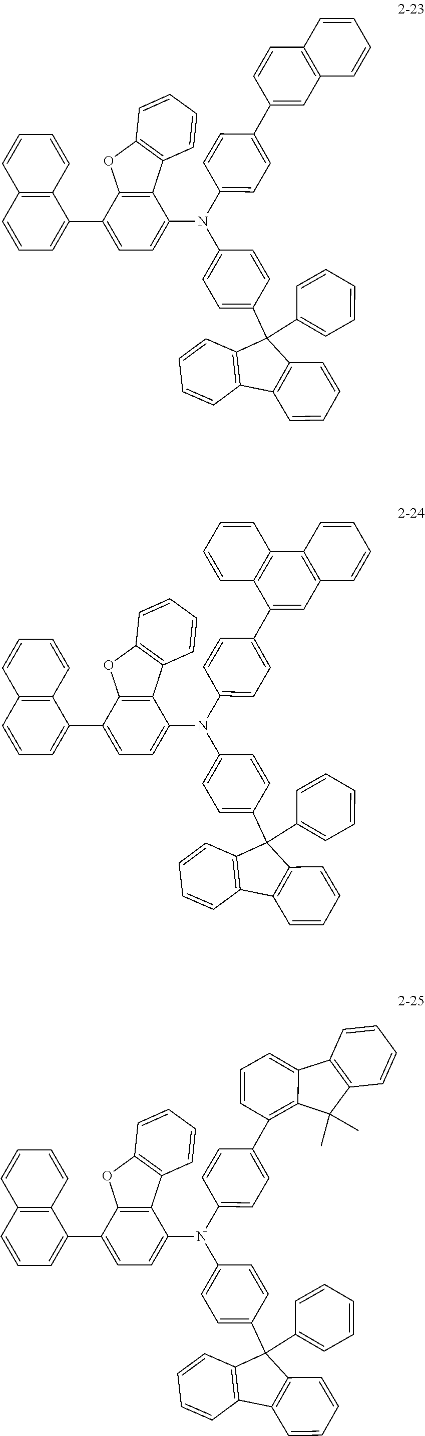 [SMILES: c1ccc(C2(c3ccc(N(c4ccc(-c5ccc6ccccc6c5)cc4)c4ccc(-c5cccc6ccccc56)c5oc6ccccc6c45)cc3)c3ccccc3-c3ccccc32)cc1 ; c1ccc(C2(c3ccc(N(c4ccc(-c5cc6ccccc6c6ccccc56)cc4)c4ccc(-c5cccc6ccccc56)c5oc6ccccc6c45)cc3)c3ccccc3-c3ccccc32)cc1 ; CC1(C)c2ccccc2-c2cccc(-c3ccc(N(c4ccc(C5(c6ccccc6)c6ccccc6-c6ccccc65)cc4)c4ccc(-c5cccc6ccccc56)c5oc6ccccc6c45)cc3)c21]

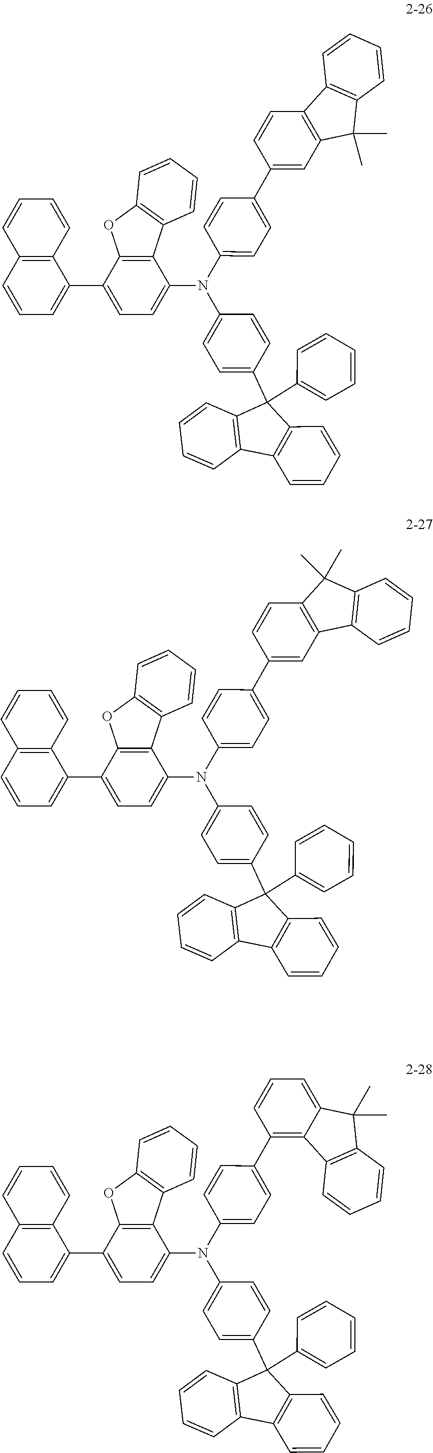 [SMILES: CC1(C)c2ccccc2-c2ccc(-c3ccc(N(c4ccc(C5(c6ccccc6)c6ccccc6-c6ccccc65)cc4)c4ccc(-c5cccc6ccccc56)c5oc6ccccc6c45)cc3)cc21 ; CC1(C)c2ccccc2-c2cc(-c3ccc(N(c4ccc(C5(c6ccccc6)c6ccccc6-c6ccccc65)cc4)c4ccc(-c5cccc6ccccc56)c5oc6ccccc6c45)cc3)ccc21 ; CC1(C)c2ccccc2-c2c(-c3ccc(N(c4ccc(C5(c6ccccc6)c6ccccc6-c6ccccc65)cc4)c4ccc(-c5cccc6ccccc56)c5oc6ccccc6c45)cc3)cccc21]

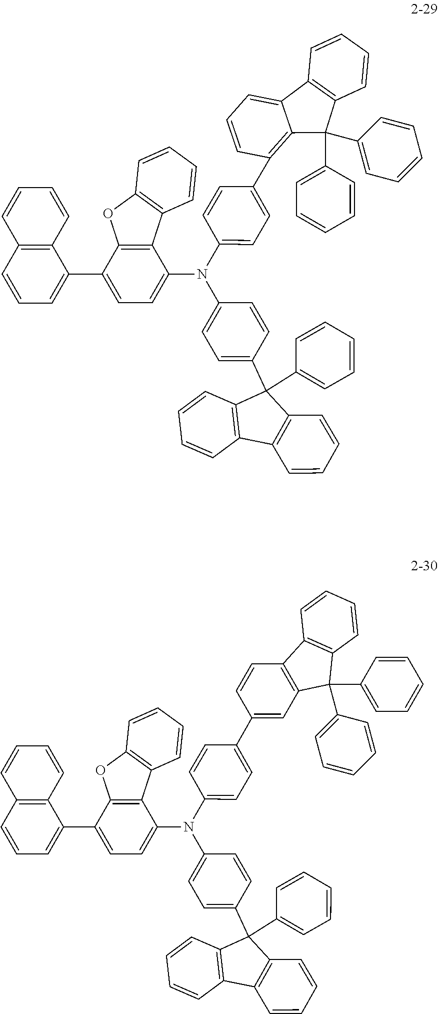 [SMILES: c1ccc(C2(c3ccc(N(c4ccc(-c5cccc6c5C(c5ccccc5)(c5ccccc5)c5ccccc5-6)cc4)c4ccc(-c5cccc6ccccc56)c5oc6ccccc6c45)cc3)c3ccccc3-c3ccccc32)cc1 ; c1ccc(C2(c3ccc(N(c4ccc(-c5ccc6c(c5)C(c5ccccc5)(c5ccccc5)c5ccccc5-6)cc4)c4ccc(-c5cccc6ccccc56)c5oc6ccccc6c45)cc3)c3ccccc3-c3ccccc32)cc1]

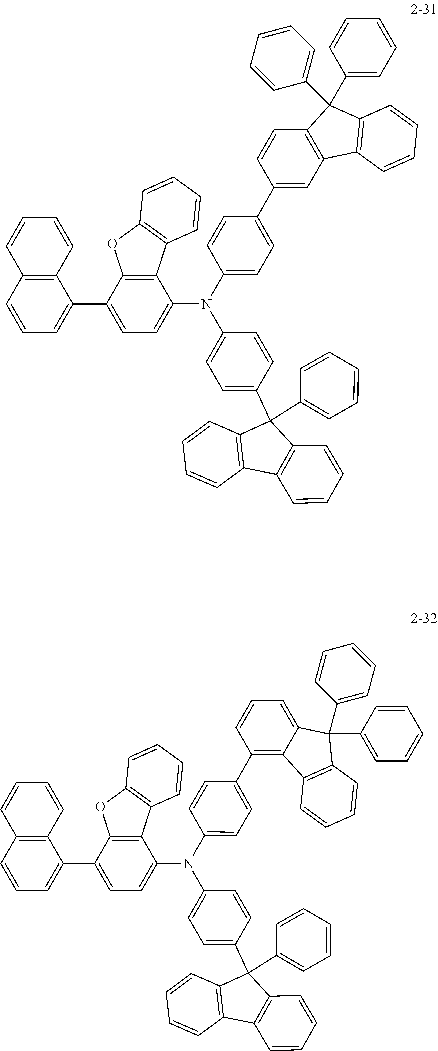 [SMILES: c1ccc(C2(c3ccc(N(c4ccc(-c5ccc6c(c5)-c5ccccc5C6(c5ccccc5)c5ccccc5)cc4)c4ccc(-c5cccc6ccccc56)c5oc6ccccc6c45)cc3)c3ccccc3-c3ccccc32)cc1 ; c1ccc(C2(c3ccc(N(c4ccc(-c5cccc6c5-c5ccccc5C6(c5ccccc5)c5ccccc5)cc4)c4ccc(-c5cccc6ccccc56)c5oc6ccccc6c45)cc3)c3ccccc3-c3ccccc32)cc1]

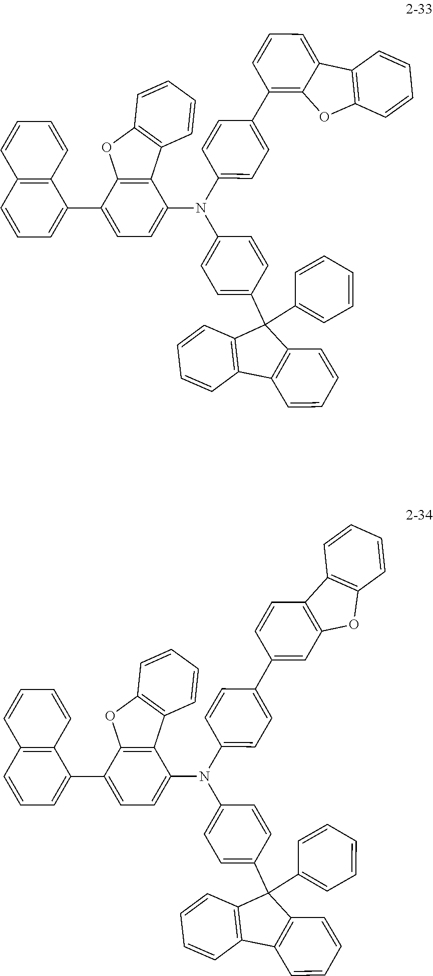 [SMILES: c1ccc(C2(c3ccc(N(c4ccc(-c5cccc6c5oc5ccccc56)cc4)c4ccc(-c5cccc6ccccc56)c5oc6ccccc6c45)cc3)c3ccccc3-c3ccccc32)cc1 ; c1ccc(C2(c3ccc(N(c4ccc(-c5ccc6c(c5)oc5ccccc56)cc4)c4ccc(-c5cccc6ccccc56)c5oc6ccccc6c45)cc3)c3ccccc3-c3ccccc32)cc1]

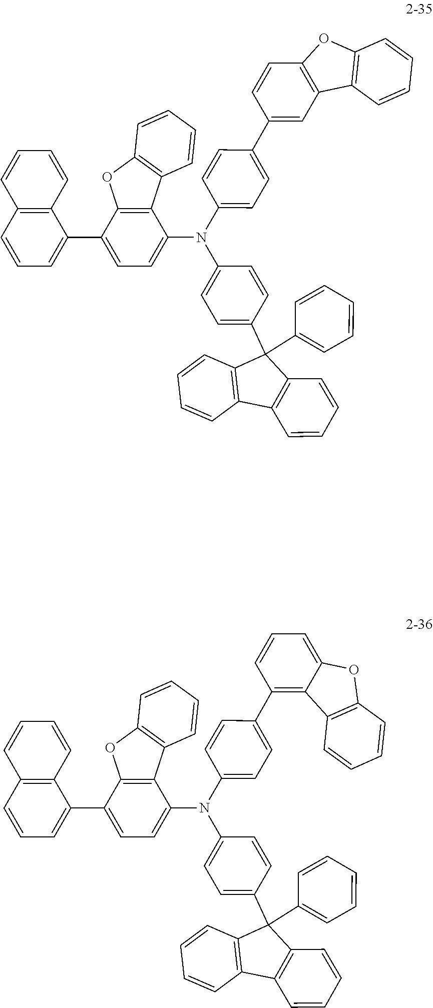 [SMILES: c1ccc(C2(c3ccc(N(c4ccc(-c5ccc6oc7ccccc7c6c5)cc4)c4ccc(-c5cccc6ccccc56)c5oc6ccccc6c45)cc3)c3ccccc3-c3ccccc32)cc1 ; c1ccc(C2(c3ccc(N(c4ccc(-c5cccc6oc7ccccc7c56)cc4)c4ccc(-c5cccc6ccccc56)c5oc6ccccc6c45)cc3)c3ccccc3-c3ccccc32)cc1]

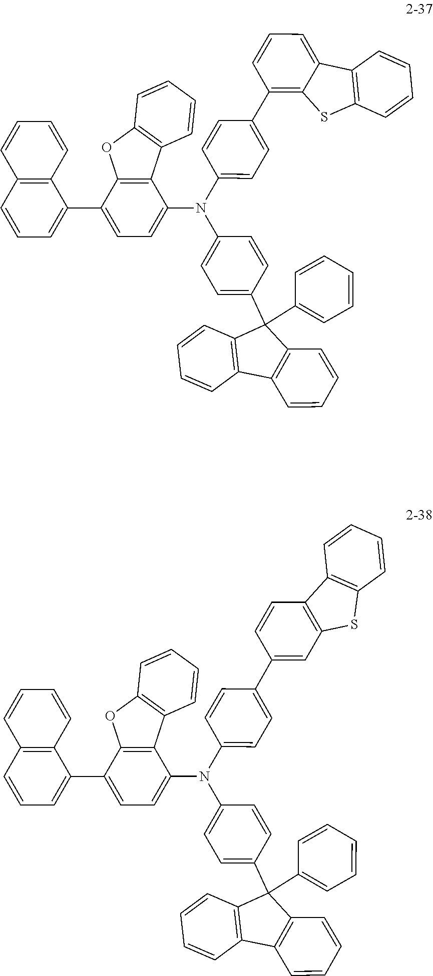 [SMILES: c1ccc(C2(c3ccc(N(c4ccc(-c5cccc6c5sc5ccccc56)cc4)c4ccc(-c5cccc6ccccc56)c5oc6ccccc6c45)cc3)c3ccccc3-c3ccccc32)cc1 ; c1ccc(C2(c3ccc(N(c4ccc(-c5ccc6c(c5)sc5ccccc56)cc4)c4ccc(-c5cccc6ccccc56)c5oc6ccccc6c45)cc3)c3ccccc3-c3ccccc32)cc1]

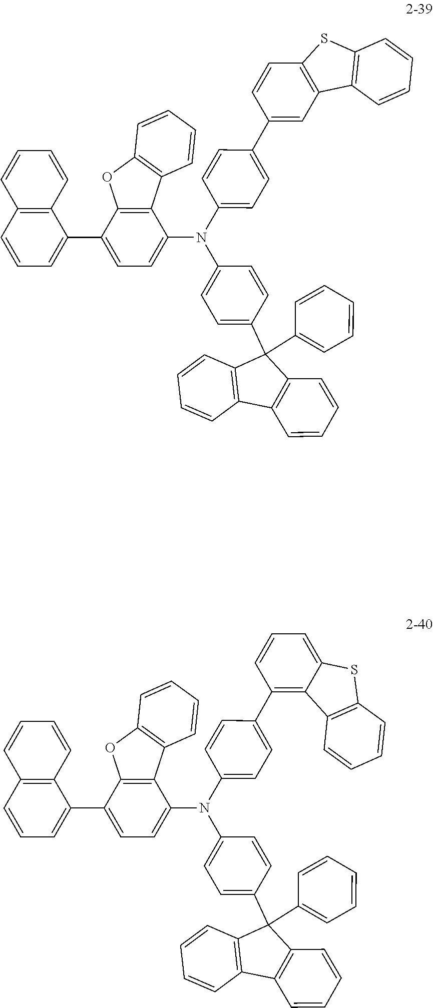 [SMILES: c1ccc(C2(c3ccc(N(c4ccc(-c5ccc6sc7ccccc7c6c5)cc4)c4ccc(-c5cccc6ccccc56)c5oc6ccccc6c45)cc3)c3ccccc3-c3ccccc32)cc1 ; c1ccc(C2(c3ccc(N(c4ccc(-c5cccc6sc7ccccc7c56)cc4)c4ccc(-c5cccc6ccccc56)c5oc6ccccc6c45)cc3)c3ccccc3-c3ccccc32)cc1]

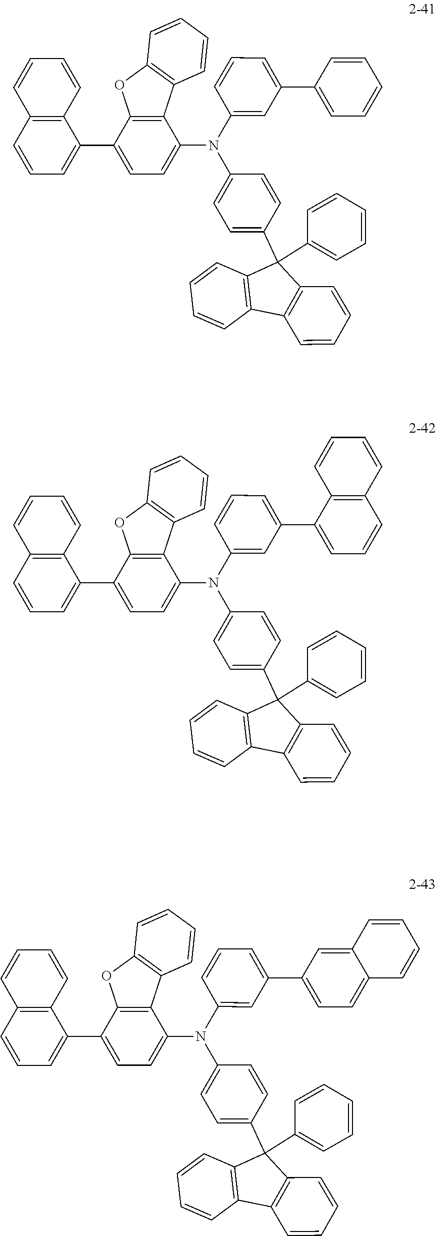 [SMILES: c1ccc(-c2cccc(N(c3ccc(C4(c5ccccc5)c5ccccc5-c5ccccc54)cc3)c3ccc(-c4cccc5ccccc45)c4oc5ccccc5c34)c2)cc1 ; c1ccc(C2(c3ccc(N(c4cccc(-c5cccc6ccccc56)c4)c4ccc(-c5cccc6ccccc56)c5oc6ccccc6c45)cc3)c3ccccc3-c3ccccc32)cc1 ; c1ccc(C2(c3ccc(N(c4cccc(-c5ccc6ccccc6c5)c4)c4ccc(-c5cccc6ccccc56)c5oc6ccccc6c45)cc3)c3ccccc3-c3ccccc32)cc1]

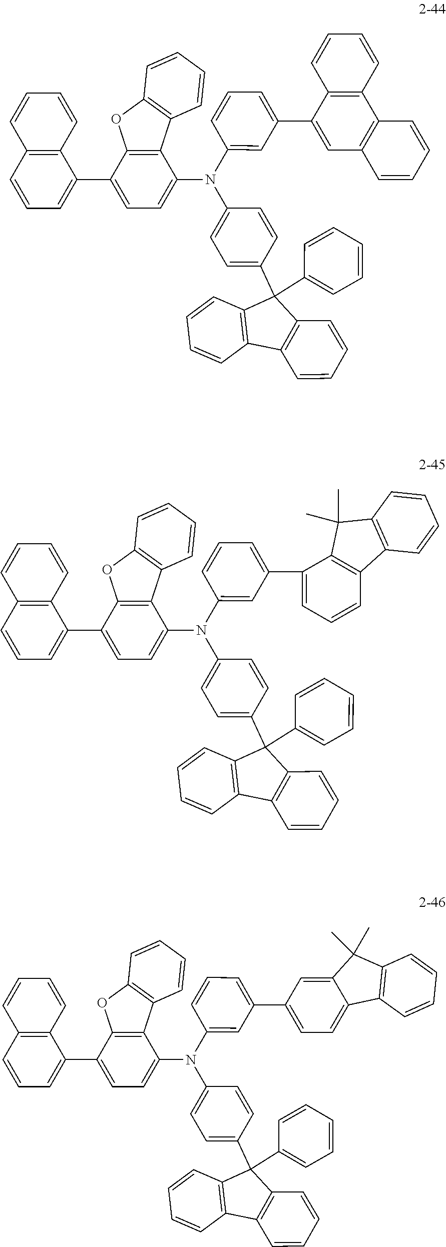 [SMILES: c1ccc(C2(c3ccc(N(c4cccc(-c5cc6ccccc6c6ccccc56)c4)c4ccc(-c5cccc6ccccc56)c5oc6ccccc6c45)cc3)c3ccccc3-c3ccccc32)cc1 ; CC1(C)c2ccccc2-c2cccc(-c3cccc(N(c4ccc(C5(c6ccccc6)c6ccccc6-c6ccccc65)cc4)c4ccc(-c5cccc6ccccc56)c5oc6ccccc6c45)c3)c21 ; CC1(C)c2ccccc2-c2ccc(-c3cccc(N(c4ccc(C5(c6ccccc6)c6ccccc6-c6ccccc65)cc4)c4ccc(-c5cccc6ccccc56)c5oc6ccccc6c45)c3)cc21]

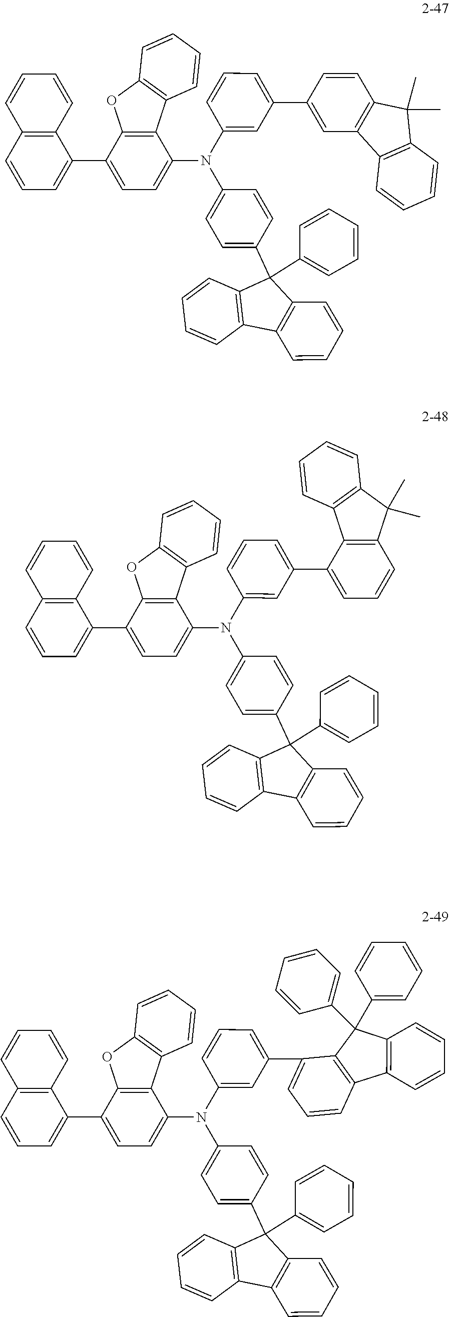 [SMILES: CC1(C)c2ccccc2-c2cc(-c3cccc(N(c4ccc(C5(c6ccccc6)c6ccccc6-c6ccccc65)cc4)c4ccc(-c5cccc6ccccc56)c5oc6ccccc6c45)c3)ccc21 ; CC1(C)c2ccccc2-c2c(-c3cccc(N(c4ccc(C5(c6ccccc6)c6ccccc6-c6ccccc65)cc4)c4ccc(-c5cccc6ccccc56)c5oc6ccccc6c45)c3)cccc21 ; c1ccc(C2(c3ccc(N(c4cccc(-c5cccc6c5C(c5ccccc5)(c5ccccc5)c5ccccc5-6)c4)c4ccc(-c5cccc6ccccc56)c5oc6ccccc6c45)cc3)c3ccccc3-c3ccccc32)cc1]

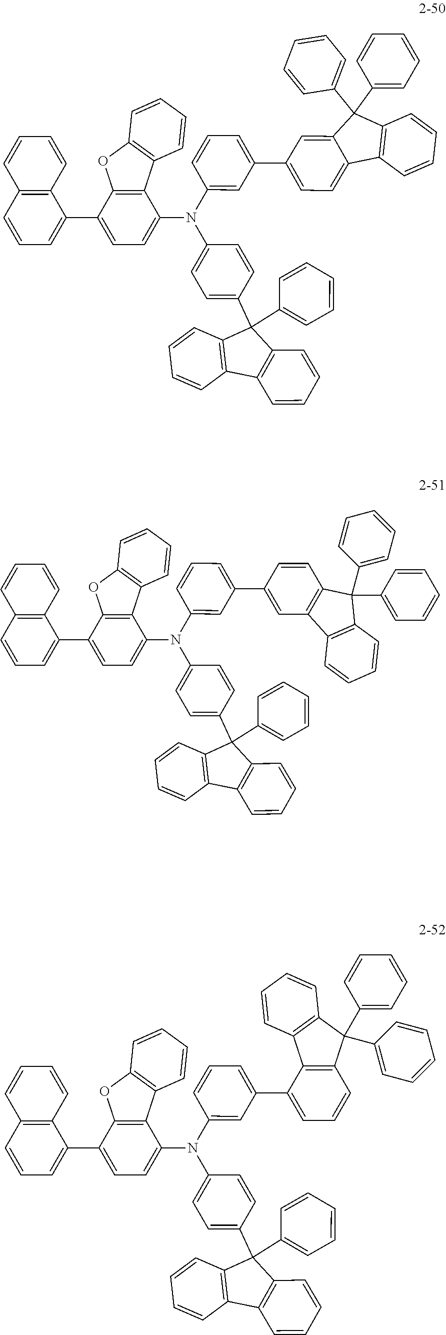 [SMILES: c1ccc(C2(c3ccc(N(c4cccc(-c5ccc6c(c5)C(c5ccccc5)(c5ccccc5)c5ccccc5-6)c4)c4ccc(-c5cccc6ccccc56)c5oc6ccccc6c45)cc3)c3ccccc3-c3ccccc32)cc1 ; c1ccc(C2(c3ccc(N(c4cccc(-c5ccc6c(c5)-c5ccccc5C6(c5ccccc5)c5ccccc5)c4)c4ccc(-c5cccc6ccccc56)c5oc6ccccc6c45)cc3)c3ccccc3-c3ccccc32)cc1 ; c1ccc(C2(c3ccc(N(c4cccc(-c5cccc6c5-c5ccccc5C6(c5ccccc5)c5ccccc5)c4)c4ccc(-c5cccc6ccccc56)c5oc6ccccc6c45)cc3)c3ccccc3-c3ccccc32)cc1]

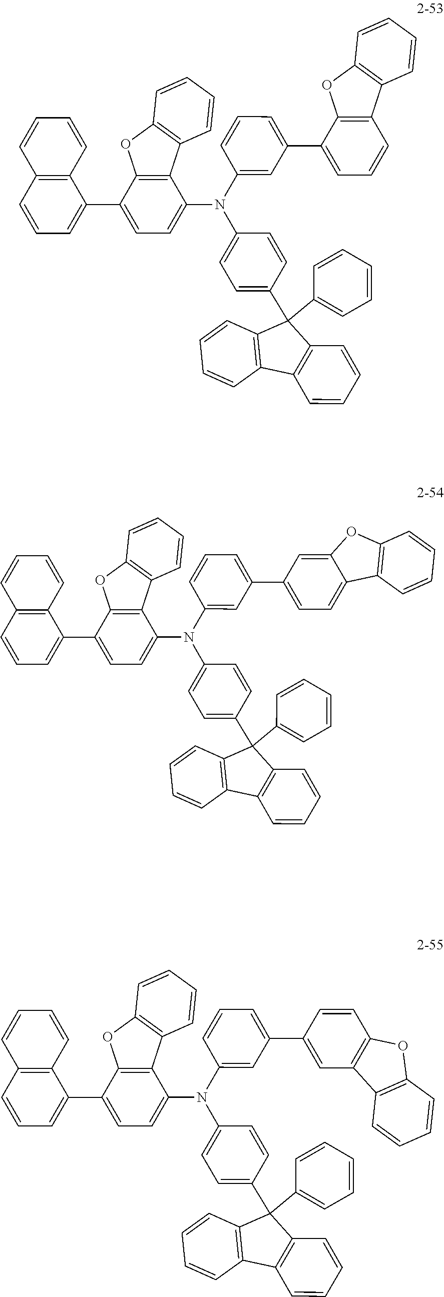 [SMILES: c1ccc(C2(c3ccc(N(c4cccc(-c5cccc6c5oc5ccccc56)c4)c4ccc(-c5cccc6ccccc56)c5oc6ccccc6c45)cc3)c3ccccc3-c3ccccc32)cc1 ; c1ccc(C2(c3ccc(N(c4cccc(-c5ccc6c(c5)oc5ccccc56)c4)c4ccc(-c5cccc6ccccc56)c5oc6ccccc6c45)cc3)c3ccccc3-c3ccccc32)cc1 ; c1ccc(C2(c3ccc(N(c4cccc(-c5ccc6oc7ccccc7c6c5)c4)c4ccc(-c5cccc6ccccc56)c5oc6ccccc6c45)cc3)c3ccccc3-c3ccccc32)cc1]

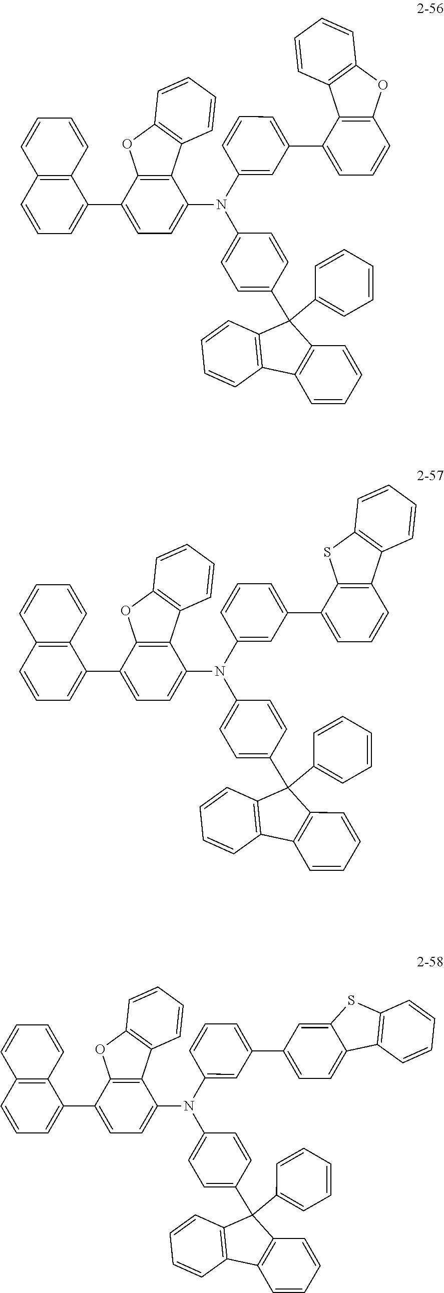 [SMILES: c1ccc(C2(c3ccc(N(c4cccc(-c5cccc6oc7ccccc7c56)c4)c4ccc(-c5cccc6ccccc56)c5oc6ccccc6c45)cc3)c3ccccc3-c3ccccc32)cc1 ; c1ccc(C2(c3ccc(N(c4cccc(-c5cccc6c5sc5ccccc56)c4)c4ccc(-c5cccc6ccccc56)c5oc6ccccc6c45)cc3)c3ccccc3-c3ccccc32)cc1 ; c1ccc(C2(c3ccc(N(c4cccc(-c5ccc6c(c5)sc5ccccc56)c4)c4ccc(-c5cccc6ccccc56)c5oc6ccccc6c45)cc3)c3ccccc3-c3ccccc32)cc1]

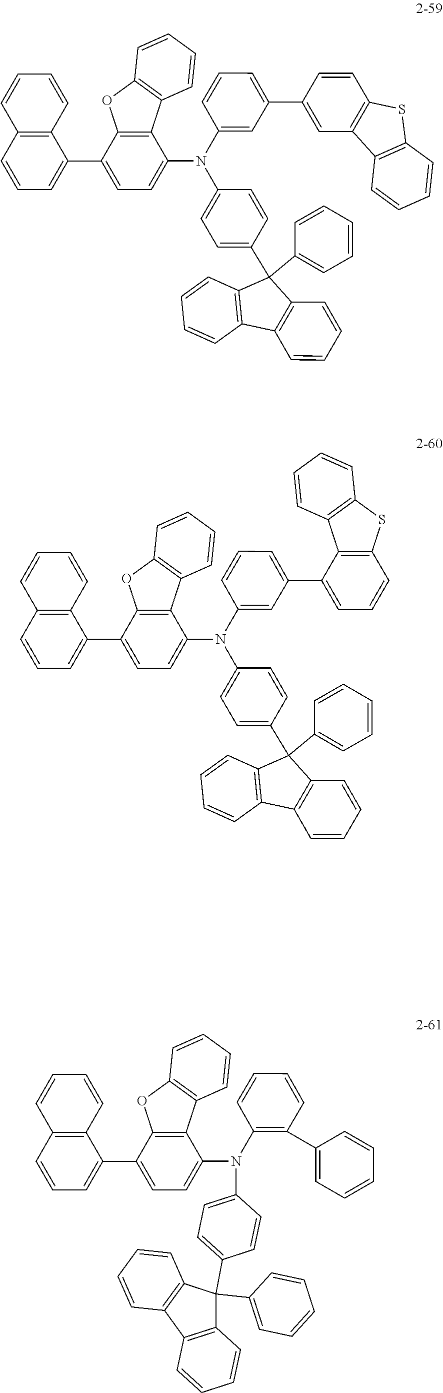 [SMILES: c1ccc(C2(c3ccc(N(c4cccc(-c5ccc6sc7ccccc7c6c5)c4)c4ccc(-c5cccc6ccccc56)c5oc6ccccc6c45)cc3)c3ccccc3-c3ccccc32)cc1 ; c1ccc(C2(c3ccc(N(c4cccc(-c5cccc6sc7ccccc7c56)c4)c4ccc(-c5cccc6ccccc56)c5oc6ccccc6c45)cc3)c3ccccc3-c3ccccc32)cc1 ; c1ccc(-c2ccccc2N(c2ccc(C3(c4ccccc4)c4ccccc4-c4ccccc43)cc2)c2ccc(-c3cccc4ccccc34)c3oc4ccccc4c23)cc1]

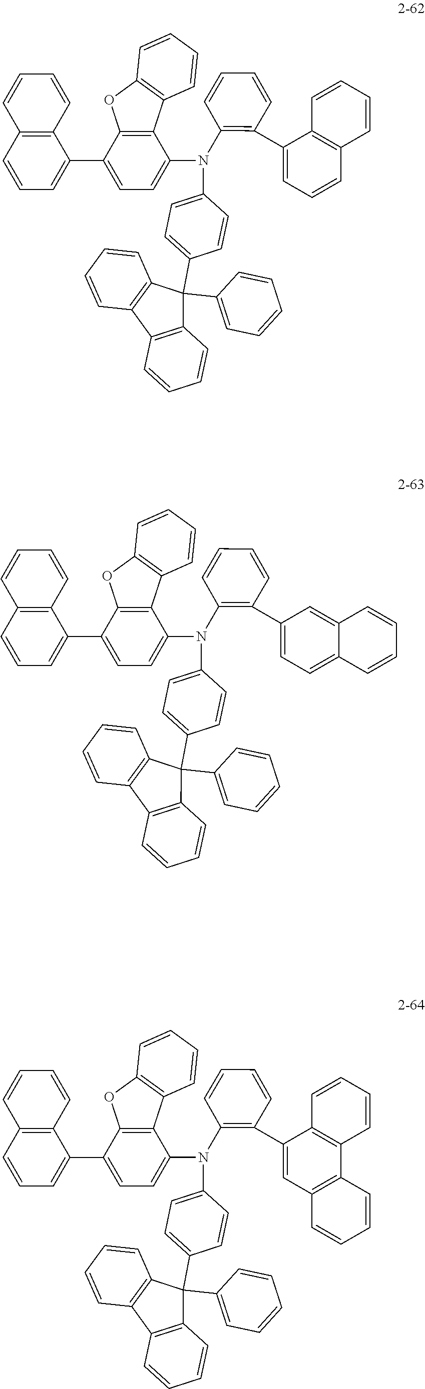 [SMILES: c1ccc(C2(c3ccc(N(c4ccccc4-c4cccc5ccccc45)c4ccc(-c5cccc6ccccc56)c5oc6ccccc6c45)cc3)c3ccccc3-c3ccccc32)cc1 ; c1ccc(C2(c3ccc(N(c4ccccc4-c4ccc5ccccc5c4)c4ccc(-c5cccc6ccccc56)c5oc6ccccc6c45)cc3)c3ccccc3-c3ccccc32)cc1 ; c1ccc(C2(c3ccc(N(c4ccccc4-c4cc5ccccc5c5ccccc45)c4ccc(-c5cccc6ccccc56)c5oc6ccccc6c45)cc3)c3ccccc3-c3ccccc32)cc1]

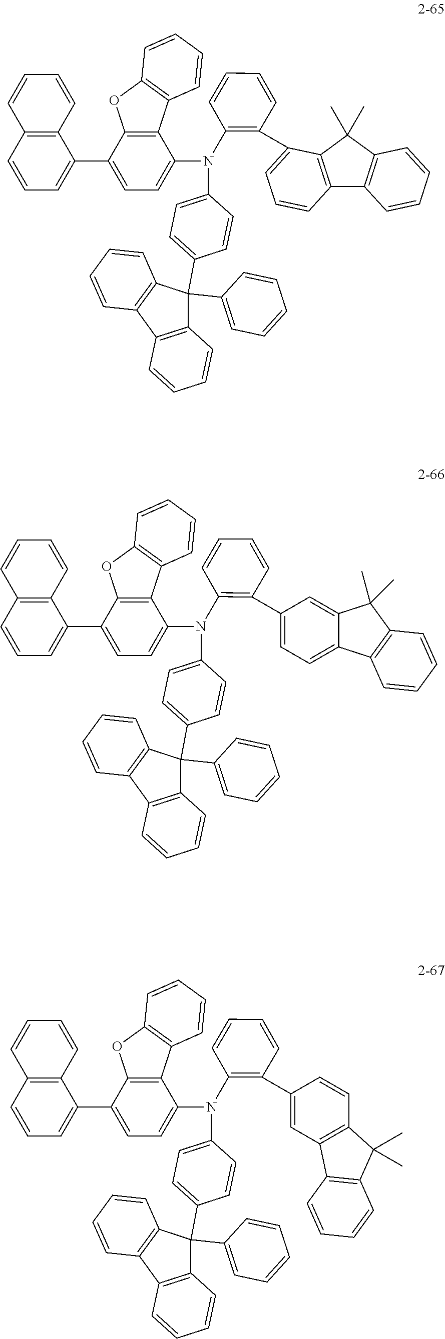 [SMILES: CC1(C)c2ccccc2-c2cccc(-c3ccccc3N(c3ccc(C4(c5ccccc5)c5ccccc5-c5ccccc54)cc3)c3ccc(-c4cccc5ccccc45)c4oc5ccccc5c34)c21 ; CC1(C)c2ccccc2-c2ccc(-c3ccccc3N(c3ccc(C4(c5ccccc5)c5ccccc5-c5ccccc54)cc3)c3ccc(-c4cccc5ccccc45)c4oc5ccccc5c34)cc21 ; CC1(C)c2ccccc2-c2cc(-c3ccccc3N(c3ccc(C4(c5ccccc5)c5ccccc5-c5ccccc54)cc3)c3ccc(-c4cccc5ccccc45)c4oc5ccccc5c34)ccc21]

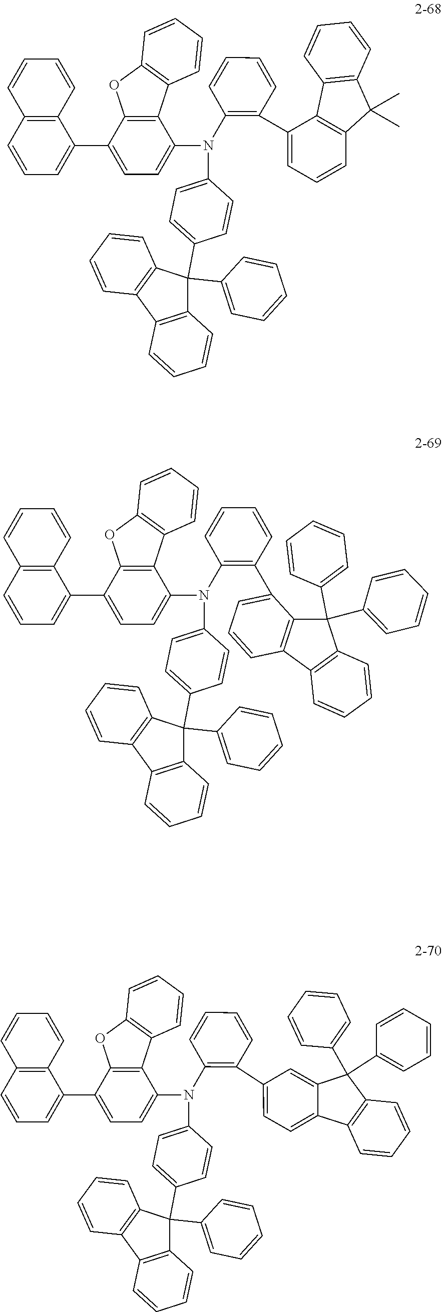 [SMILES: CC1(C)c2ccccc2-c2c(-c3ccccc3N(c3ccc(C4(c5ccccc5)c5ccccc5-c5ccccc54)cc3)c3ccc(-c4cccc5ccccc45)c4oc5ccccc5c34)cccc21 ; c1ccc(C2(c3ccc(N(c4ccccc4-c4cccc5c4C(c4ccccc4)(c4ccccc4)c4ccccc4-5)c4ccc(-c5cccc6ccccc56)c5oc6ccccc6c45)cc3)c3ccccc3-c3ccccc32)cc1 ; c1ccc(C2(c3ccc(N(c4ccccc4-c4ccc5c(c4)C(c4ccccc4)(c4ccccc4)c4ccccc4-5)c4ccc(-c5cccc6ccccc56)c5oc6ccccc6c45)cc3)c3ccccc3-c3ccccc32)cc1]

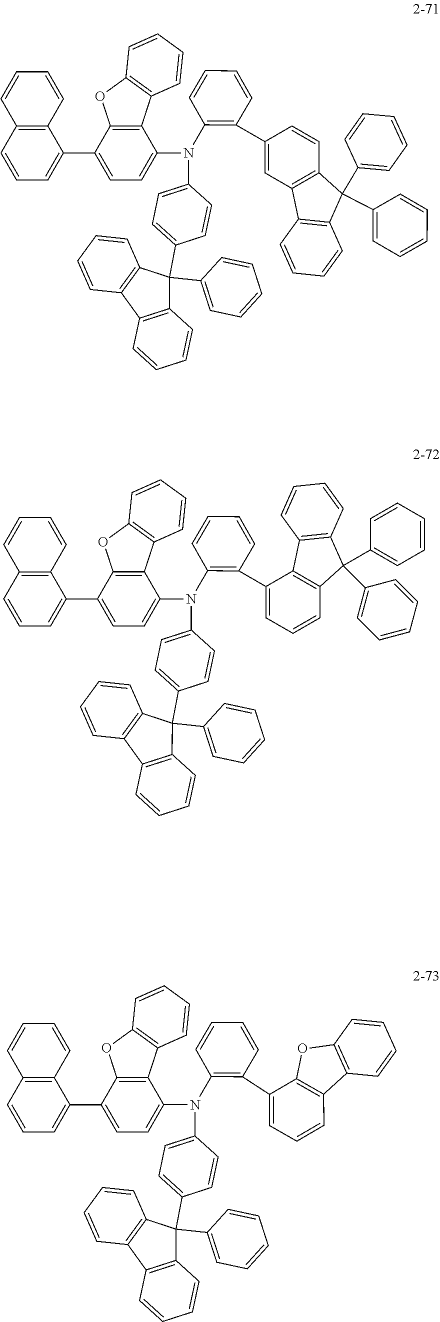 [SMILES: c1ccc(C2(c3ccc(N(c4ccccc4-c4ccc5c(c4)-c4ccccc4C5(c4ccccc4)c4ccccc4)c4ccc(-c5cccc6ccccc56)c5oc6ccccc6c45)cc3)c3ccccc3-c3ccccc32)cc1 ; c1ccc(C2(c3ccc(N(c4ccccc4-c4cccc5c4-c4ccccc4C5(c4ccccc4)c4ccccc4)c4ccc(-c5cccc6ccccc56)c5oc6ccccc6c45)cc3)c3ccccc3-c3ccccc32)cc1 ; c1ccc(C2(c3ccc(N(c4ccccc4-c4cccc5c4oc4ccccc45)c4ccc(-c5cccc6ccccc56)c5oc6ccccc6c45)cc3)c3ccccc3-c3ccccc32)cc1]

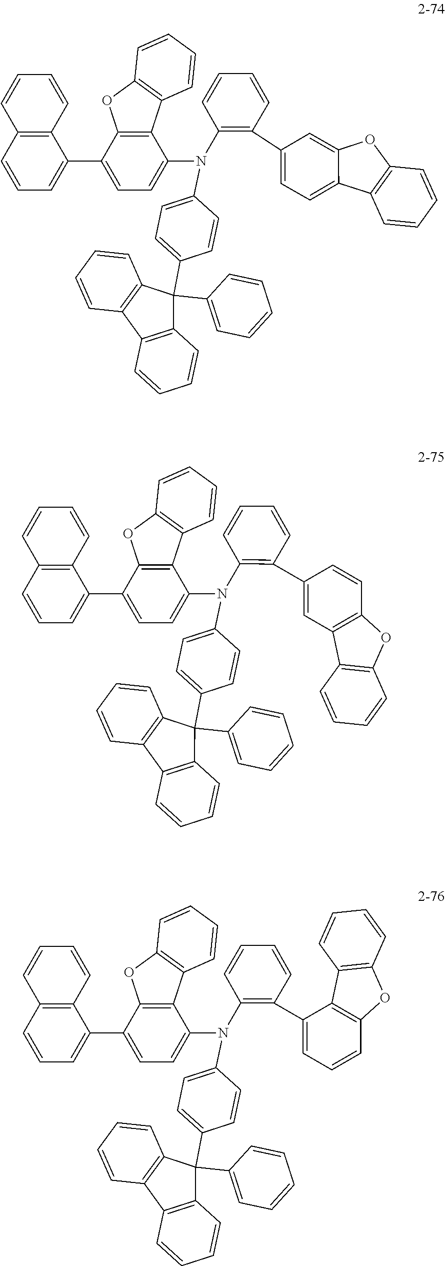 [SMILES: c1ccc(C2(c3ccc(N(c4ccccc4-c4ccc5c(c4)oc4ccccc45)c4ccc(-c5cccc6ccccc56)c5oc6ccccc6c45)cc3)c3ccccc3-c3ccccc32)cc1 ; c1ccc(C2(c3ccc(N(c4ccccc4-c4ccc5oc6ccccc6c5c4)c4ccc(-c5cccc6ccccc56)c5oc6ccccc6c45)cc3)c3ccccc3-c3ccccc32)cc1 ; c1ccc(C2(c3ccc(N(c4ccccc4-c4cccc5oc6ccccc6c45)c4ccc(-c5cccc6ccccc56)c5oc6ccccc6c45)cc3)c3ccccc3-c3ccccc32)cc1]

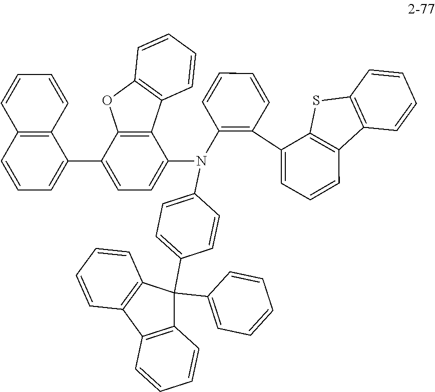 [SMILES: c1ccc(C2(c3ccc(N(c4ccccc4-c4cccc5c4sc4ccccc45)c4ccc(-c5cccc6ccccc56)c5oc6ccccc6c45)cc3)c3ccccc3-c3ccccc32)cc1]

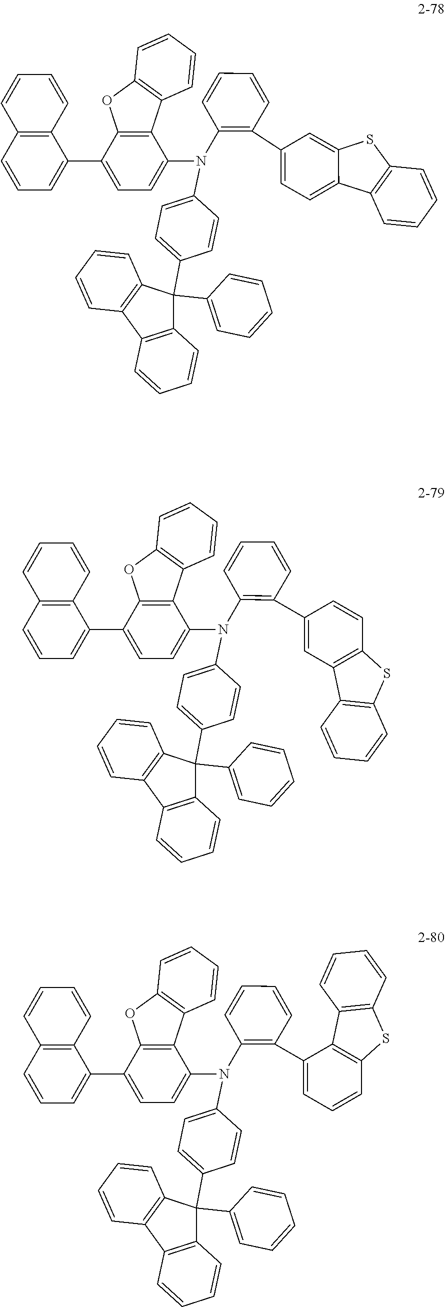 [SMILES: c1ccc(C2(c3ccc(N(c4ccccc4-c4ccc5c(c4)sc4ccccc45)c4ccc(-c5cccc6ccccc56)c5oc6ccccc6c45)cc3)c3ccccc3-c3ccccc32)cc1 ; c1ccc(C2(c3ccc(N(c4ccccc4-c4ccc5sc6ccccc6c5c4)c4ccc(-c5cccc6ccccc56)c5oc6ccccc6c45)cc3)c3ccccc3-c3ccccc32)cc1 ; c1ccc(C2(c3ccc(N(c4ccccc4-c4cccc5sc6ccccc6c45)c4ccc(-c5cccc6ccccc56)c5oc6ccccc6c45)cc3)c3ccccc3-c3ccccc32)cc1]

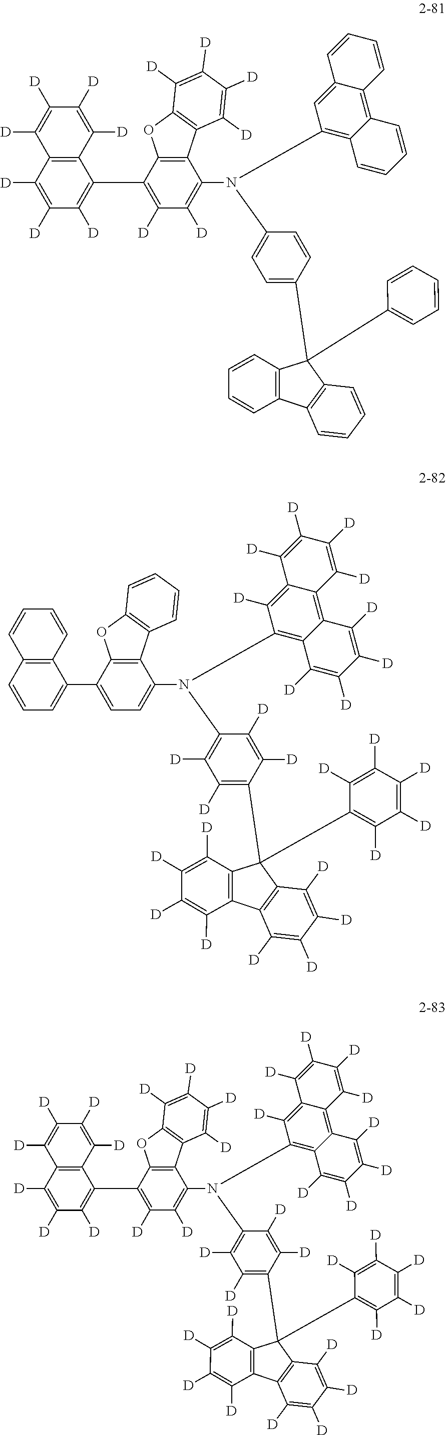

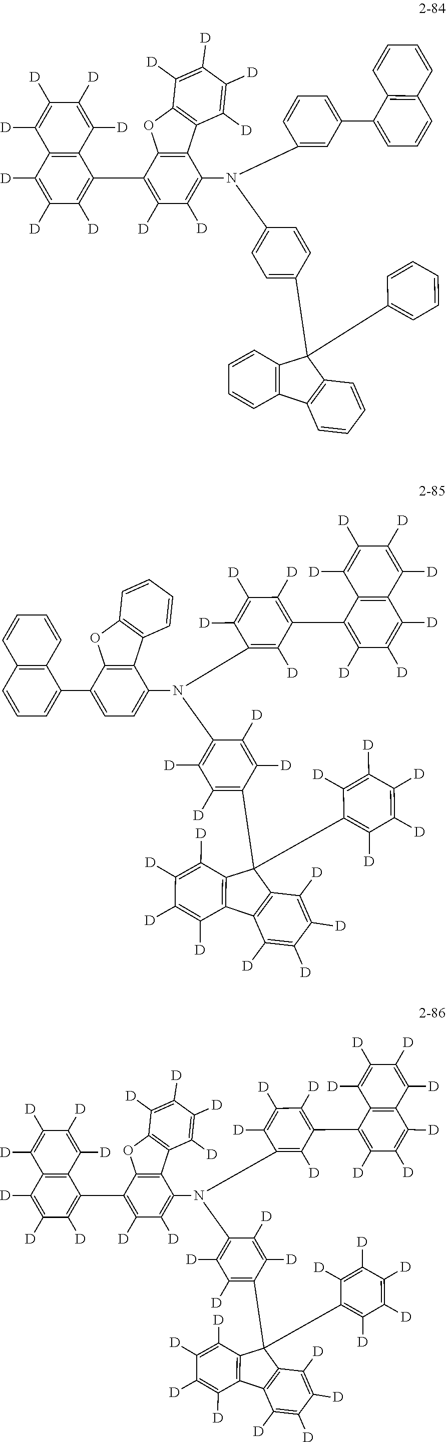

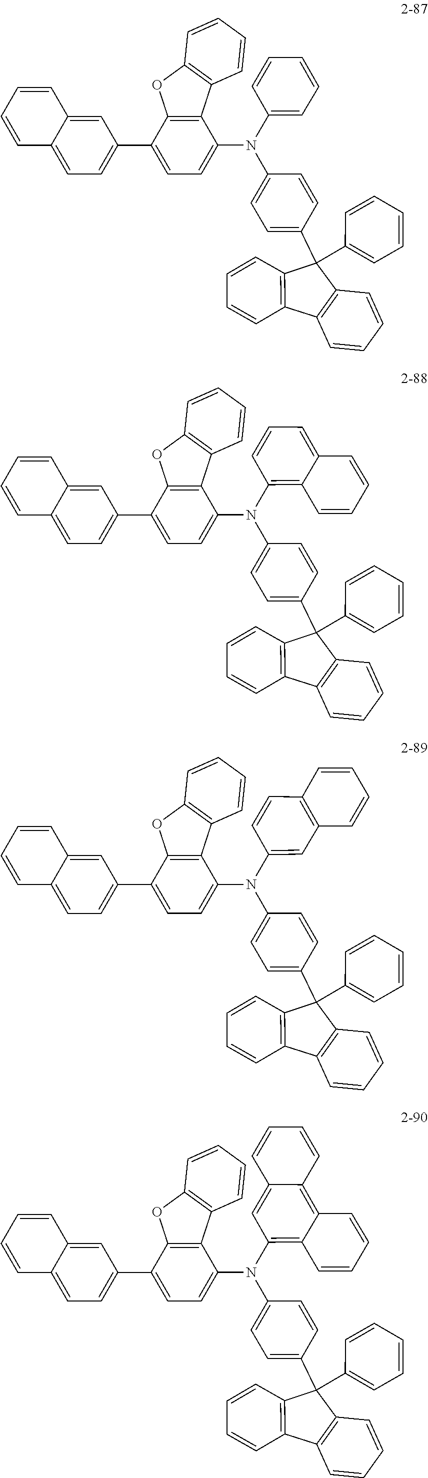 [SMILES: c1ccc(N(c2ccc(C3(c4ccccc4)c4ccccc4-c4ccccc43)cc2)c2ccc(-c3ccc4ccccc4c3)c3oc4ccccc4c23)cc1 ; c1ccc(C2(c3ccc(N(c4cccc5ccccc45)c4ccc(-c5ccc6ccccc6c5)c5oc6ccccc6c45)cc3)c3ccccc3-c3ccccc32)cc1 ; c1ccc(C2(c3ccc(N(c4ccc5ccccc5c4)c4ccc(-c5ccc6ccccc6c5)c5oc6ccccc6c45)cc3)c3ccccc3-c3ccccc32)cc1 ; c1ccc(C2(c3ccc(N(c4cc5ccccc5c5ccccc45)c4ccc(-c5ccc6ccccc6c5)c5oc6ccccc6c45)cc3)c3ccccc3-c3ccccc32)cc1]

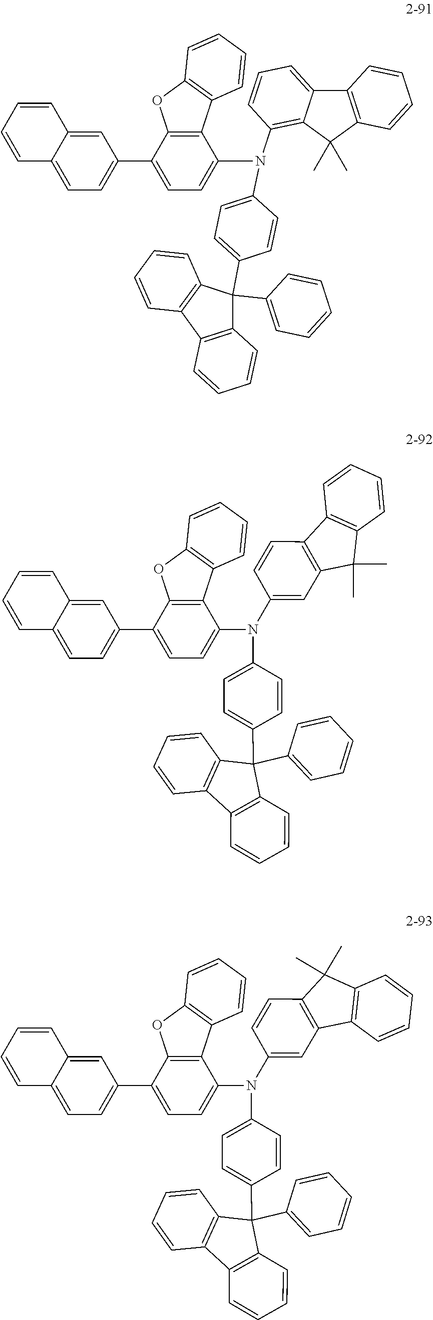 [SMILES: CC1(C)c2ccccc2-c2cccc(N(c3ccc(C4(c5ccccc5)c5ccccc5-c5ccccc54)cc3)c3ccc(-c4ccc5ccccc5c4)c4oc5ccccc5c34)c21 ; CC1(C)c2ccccc2-c2ccc(N(c3ccc(C4(c5ccccc5)c5ccccc5-c5ccccc54)cc3)c3ccc(-c4ccc5ccccc5c4)c4oc5ccccc5c34)cc21 ; CC1(C)c2ccccc2-c2cc(N(c3ccc(C4(c5ccccc5)c5ccccc5-c5ccccc54)cc3)c3ccc(-c4ccc5ccccc5c4)c4oc5ccccc5c34)ccc21]

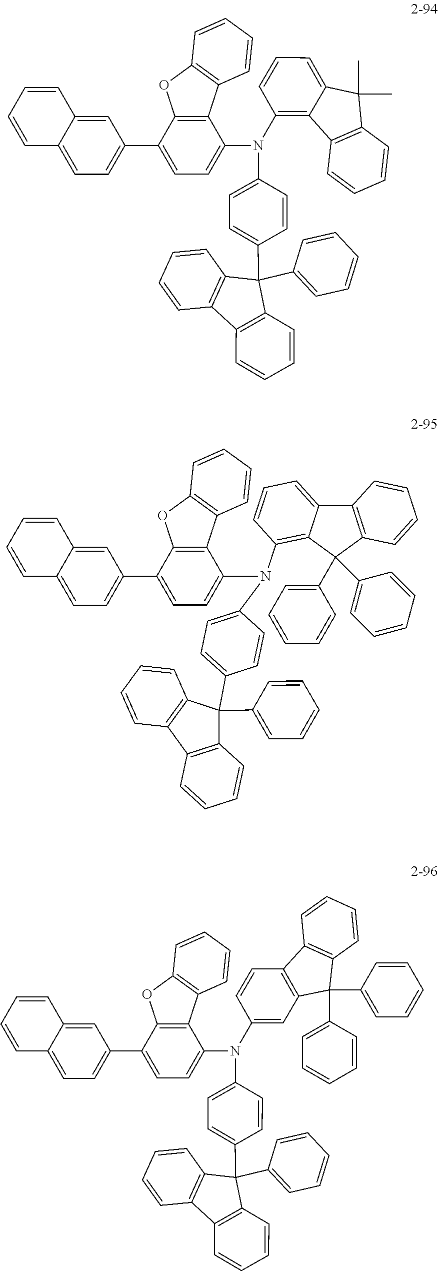 [SMILES: CC1(C)c2ccccc2-c2c(N(c3ccc(C4(c5ccccc5)c5ccccc5-c5ccccc54)cc3)c3ccc(-c4ccc5ccccc5c4)c4oc5ccccc5c34)cccc21 ; c1ccc(C2(c3ccc(N(c4cccc5c4C(c4ccccc4)(c4ccccc4)c4ccccc4-5)c4ccc(-c5ccc6ccccc6c5)c5oc6ccccc6c45)cc3)c3ccccc3-c3ccccc32)cc1 ; c1ccc(C2(c3ccc(N(c4ccc5c(c4)C(c4ccccc4)(c4ccccc4)c4ccccc4-5)c4ccc(-c5ccc6ccccc6c5)c5oc6ccccc6c45)cc3)c3ccccc3-c3ccccc32)cc1]

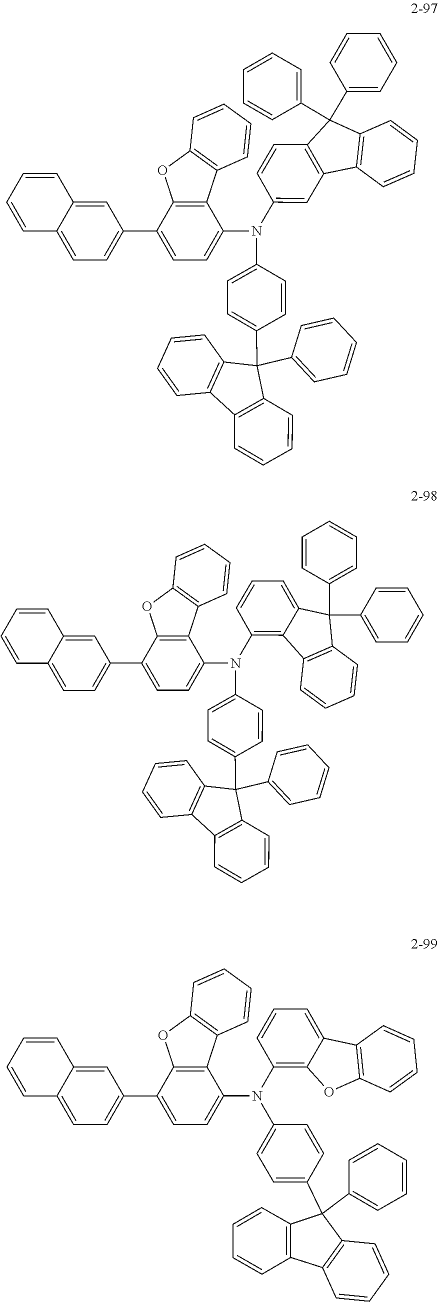 [SMILES: c1ccc(C2(c3ccc(N(c4ccc5c(c4)-c4ccccc4C5(c4ccccc4)c4ccccc4)c4ccc(-c5ccc6ccccc6c5)c5oc6ccccc6c45)cc3)c3ccccc3-c3ccccc32)cc1 ; c1ccc(C2(c3ccc(N(c4cccc5c4-c4ccccc4C5(c4ccccc4)c4ccccc4)c4ccc(-c5ccc6ccccc6c5)c5oc6ccccc6c45)cc3)c3ccccc3-c3ccccc32)cc1 ; c1ccc(C2(c3ccc(N(c4cccc5c4oc4ccccc45)c4ccc(-c5ccc6ccccc6c5)c5oc6ccccc6c45)cc3)c3ccccc3-c3ccccc32)cc1]

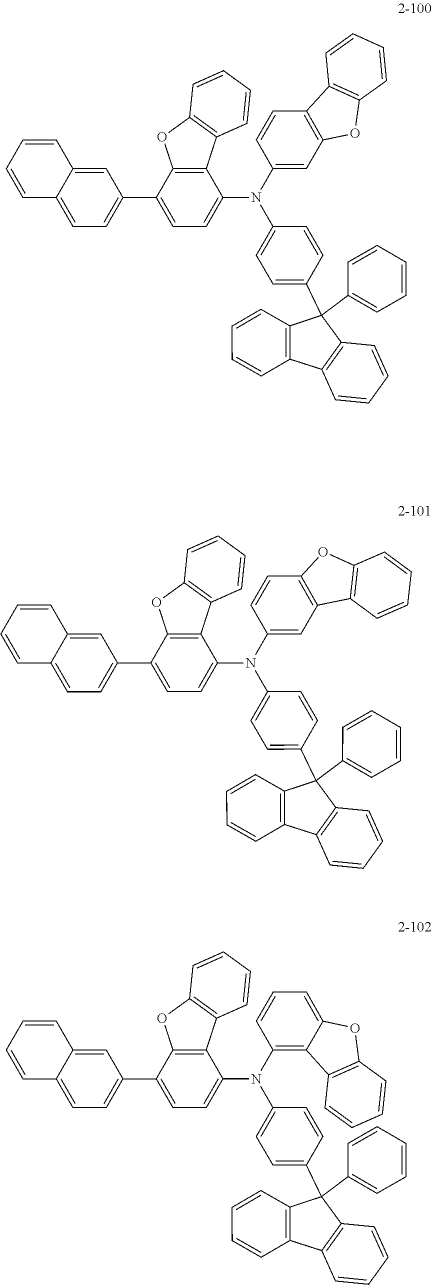 [SMILES: c1ccc(C2(c3ccc(N(c4ccc5c(c4)oc4ccccc45)c4ccc(-c5ccc6ccccc6c5)c5oc6ccccc6c45)cc3)c3ccccc3-c3ccccc32)cc1 ; c1ccc(C2(c3ccc(N(c4ccc5oc6ccccc6c5c4)c4ccc(-c5ccc6ccccc6c5)c5oc6ccccc6c45)cc3)c3ccccc3-c3ccccc32)cc1 ; c1ccc(C2(c3ccc(N(c4cccc5oc6ccccc6c45)c4ccc(-c5ccc6ccccc6c5)c5oc6ccccc6c45)cc3)c3ccccc3-c3ccccc32)cc1]

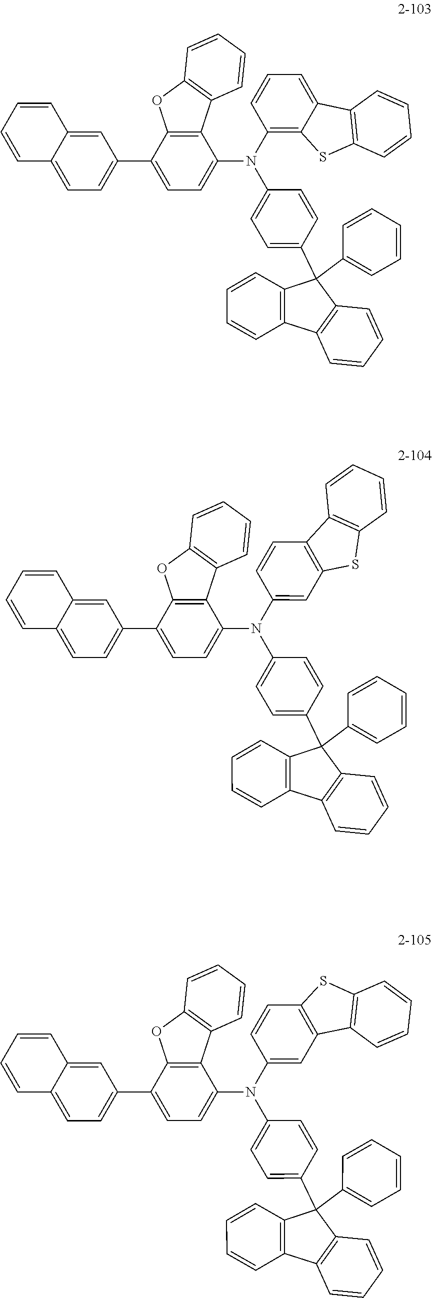 [SMILES: c1ccc(C2(c3ccc(N(c4cccc5c4sc4ccccc45)c4ccc(-c5ccc6ccccc6c5)c5oc6ccccc6c45)cc3)c3ccccc3-c3ccccc32)cc1 ; c1ccc(C2(c3ccc(N(c4ccc5c(c4)sc4ccccc45)c4ccc(-c5ccc6ccccc6c5)c5oc6ccccc6c45)cc3)c3ccccc3-c3ccccc32)cc1 ; c1ccc(C2(c3ccc(N(c4ccc5sc6ccccc6c5c4)c4ccc(-c5ccc6ccccc6c5)c5oc6ccccc6c45)cc3)c3ccccc3-c3ccccc32)cc1]

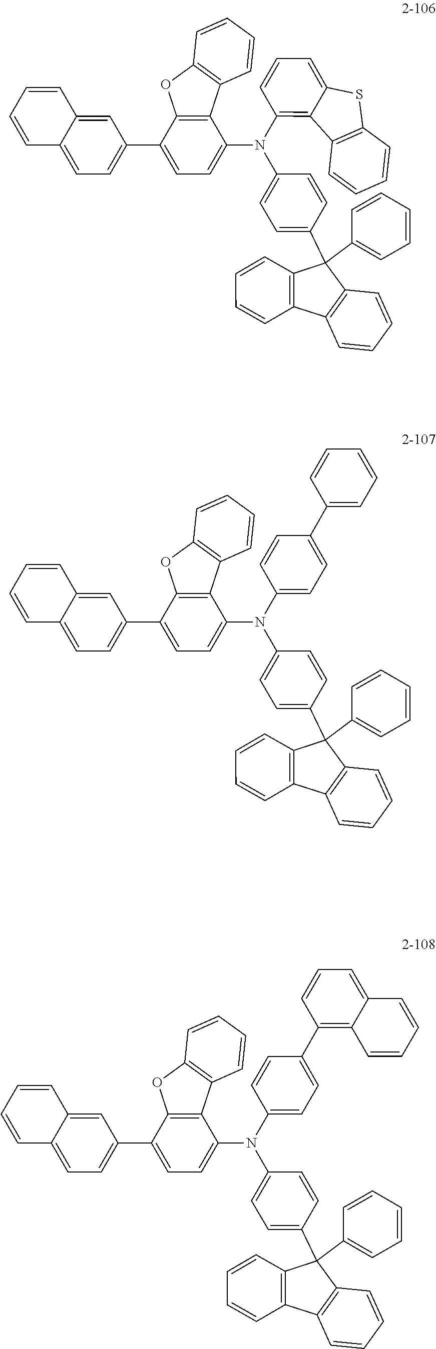 [SMILES: c1ccc(C2(c3ccc(N(c4cccc5sc6ccccc6c45)c4ccc(-c5ccc6ccccc6c5)c5oc6ccccc6c45)cc3)c3ccccc3-c3ccccc32)cc1 ; c1ccc(-c2ccc(N(c3ccc(C4(c5ccccc5)c5ccccc5-c5ccccc54)cc3)c3ccc(-c4ccc5ccccc5c4)c4oc5ccccc5c34)cc2)cc1 ; c1ccc(C2(c3ccc(N(c4ccc(-c5cccc6ccccc56)cc4)c4ccc(-c5ccc6ccccc6c5)c5oc6ccccc6c45)cc3)c3ccccc3-c3ccccc32)cc1]

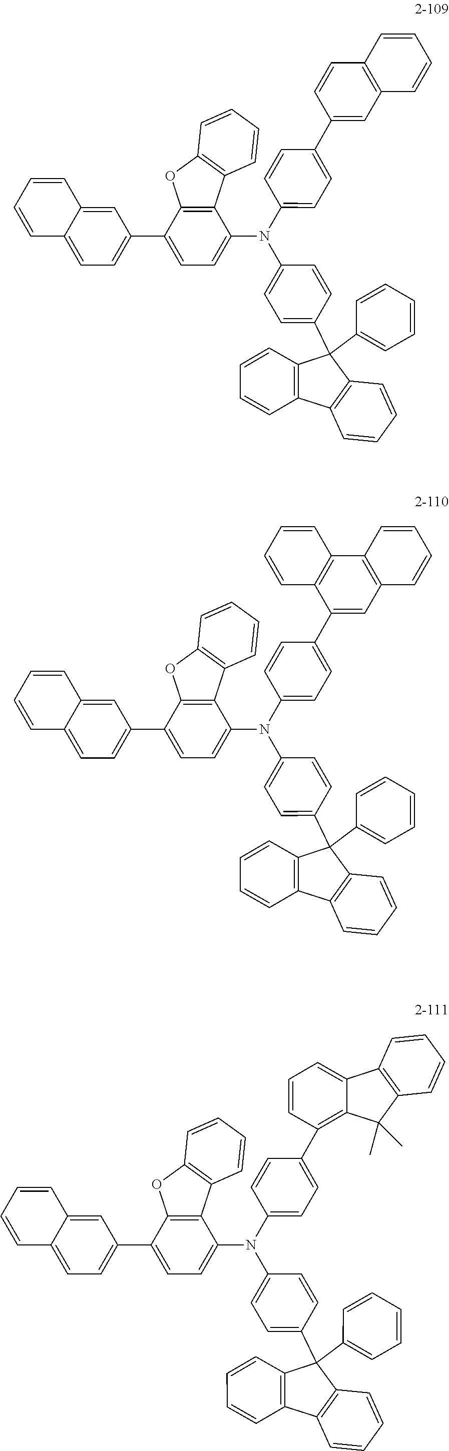 [SMILES: c1ccc(C2(c3ccc(N(c4ccc(-c5ccc6ccccc6c5)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5cc6ccccc6c6ccccc56)cc4)c4ccc(-c5ccc6ccccc6c5)c5oc6ccccc6c45)cc3)c3ccccc3-c3ccccc32)cc1 ; CC1(C)c2ccccc2-c2cccc(-c3ccc(N(c4ccc(C5(c6ccccc6)c6ccccc6-c6ccccc65)cc4)c4ccc(-c5ccc6ccccc6c5)c5oc6ccccc6c45)cc3)c21]

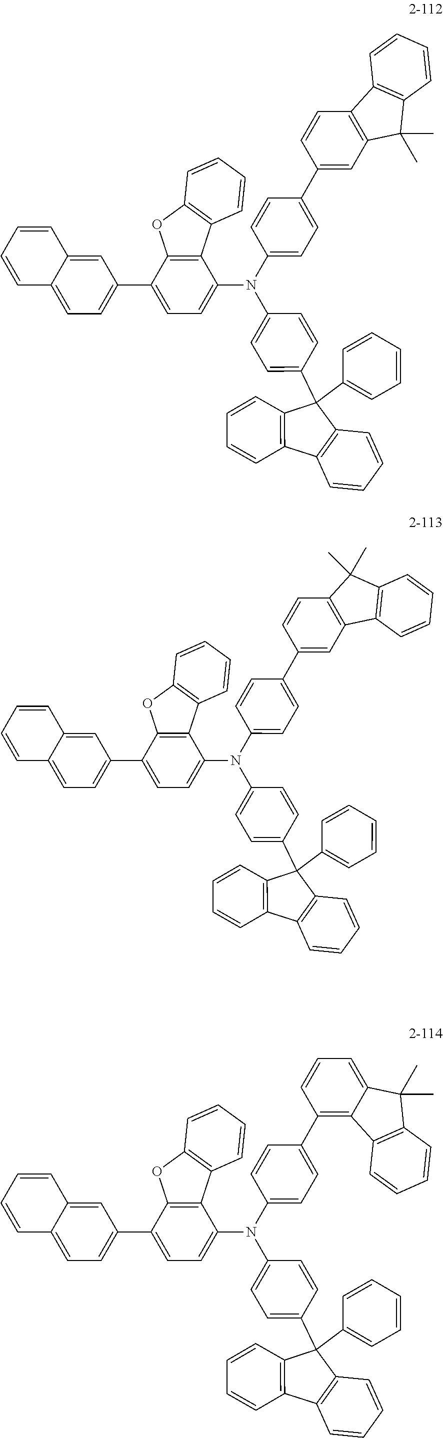 [SMILES: CC1(C)c2ccccc2-c2ccc(-c3ccc(N(c4ccc(C5(c6ccccc6)c6ccccc6-c6ccccc65)cc4)c4ccc(-c5ccc6ccccc6c5)c5oc6ccccc6c45)cc3)cc21 ; CC1(C)c2ccccc2-c2cc(-c3ccc(N(c4ccc(C5(c6ccccc6)c6ccccc6-c6ccccc65)cc4)c4ccc(-c5ccc6ccccc6c5)c5oc6ccccc6c45)cc3)ccc21 ; CC1(C)c2ccccc2-c2c(-c3ccc(N(c4ccc(C5(c6ccccc6)c6ccccc6-c6ccccc65)cc4)c4ccc(-c5ccc6ccccc6c5)c5oc6ccccc6c45)cc3)cccc21]

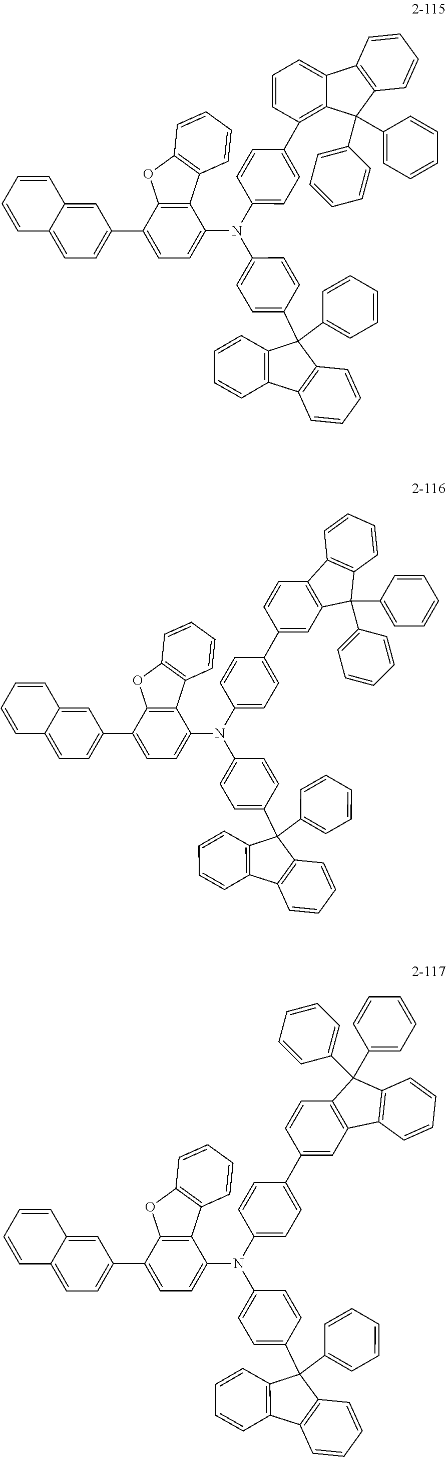 [SMILES: c1ccc(C2(c3ccc(N(c4ccc(-c5cccc6c5C(c5ccccc5)(c5ccccc5)c5ccccc5-6)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5ccc6c(c5)C(c5ccccc5)(c5ccccc5)c5ccccc5-6)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5ccc6c(c5)-c5ccccc5C6(c5ccccc5)c5ccccc5)cc4)c4ccc(-c5ccc6ccccc6c5)c5oc6ccccc6c45)cc3)c3ccccc3-c3ccccc32)cc1]

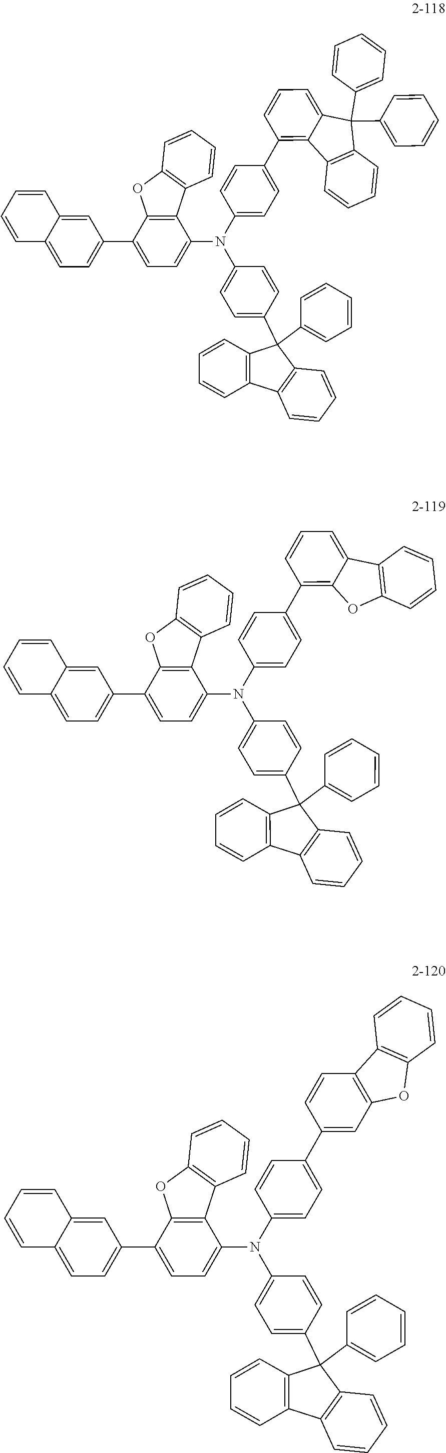 [SMILES: c1ccc(C2(c3ccc(N(c4ccc(-c5cccc6c5-c5ccccc5C6(c5ccccc5)c5ccccc5)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5cccc6c5oc5ccccc56)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5ccc6c(c5)oc5ccccc56)cc4)c4ccc(-c5ccc6ccccc6c5)c5oc6ccccc6c45)cc3)c3ccccc3-c3ccccc32)cc1]

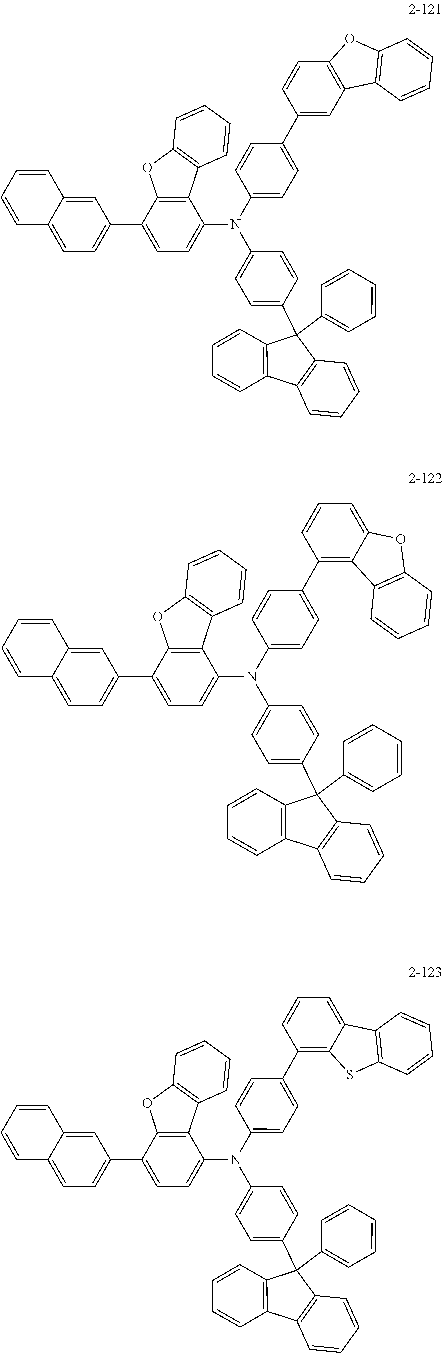 [SMILES: c1ccc(C2(c3ccc(N(c4ccc(-c5ccc6oc7ccccc7c6c5)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5cccc6oc7ccccc7c56)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5cccc6c5sc5ccccc56)cc4)c4ccc(-c5ccc6ccccc6c5)c5oc6ccccc6c45)cc3)c3ccccc3-c3ccccc32)cc1]

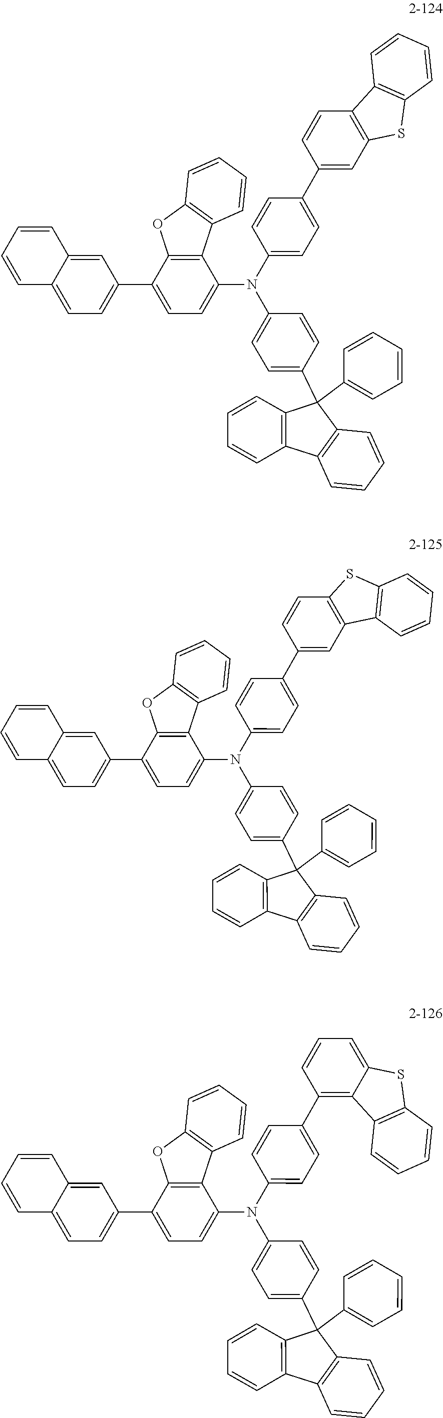 [SMILES: c1ccc(C2(c3ccc(N(c4ccc(-c5ccc6c(c5)sc5ccccc56)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5ccc6sc7ccccc7c6c5)cc4)c4ccc(-c5ccc6ccccc6c5)c5oc6ccccc6c45)cc3)c3ccccc3-c3ccccc32)cc1 ; c1ccc(C2(c3ccc(N(c4ccc(-c5cccc6sc7ccccc7c56)cc4)c4ccc(-c5ccc6ccccc6c5)c5oc6ccccc6c45)cc3)c3ccccc3-c3ccccc32)cc1]

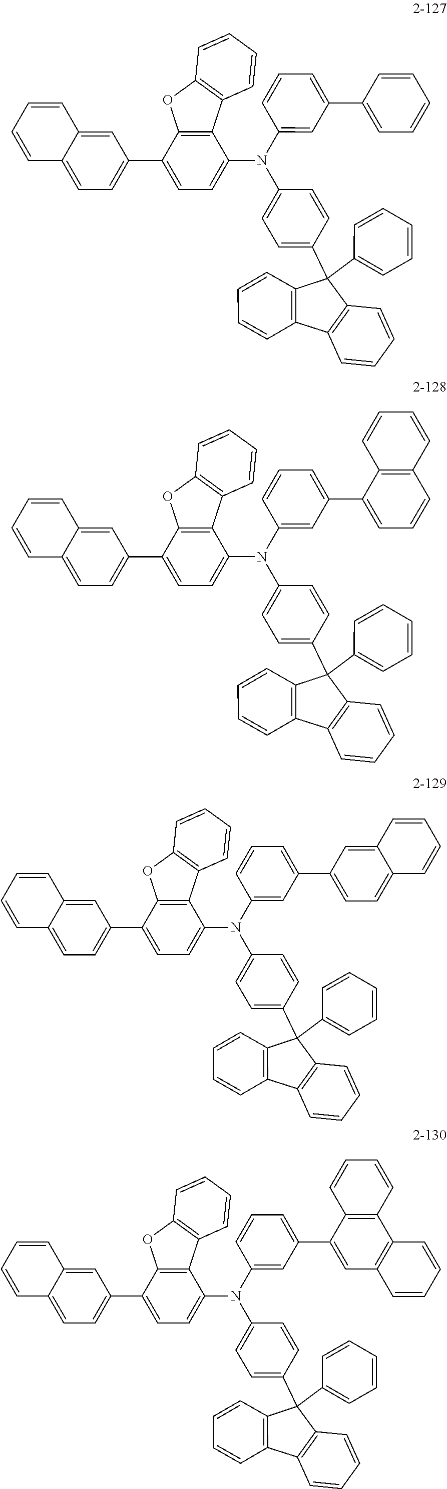

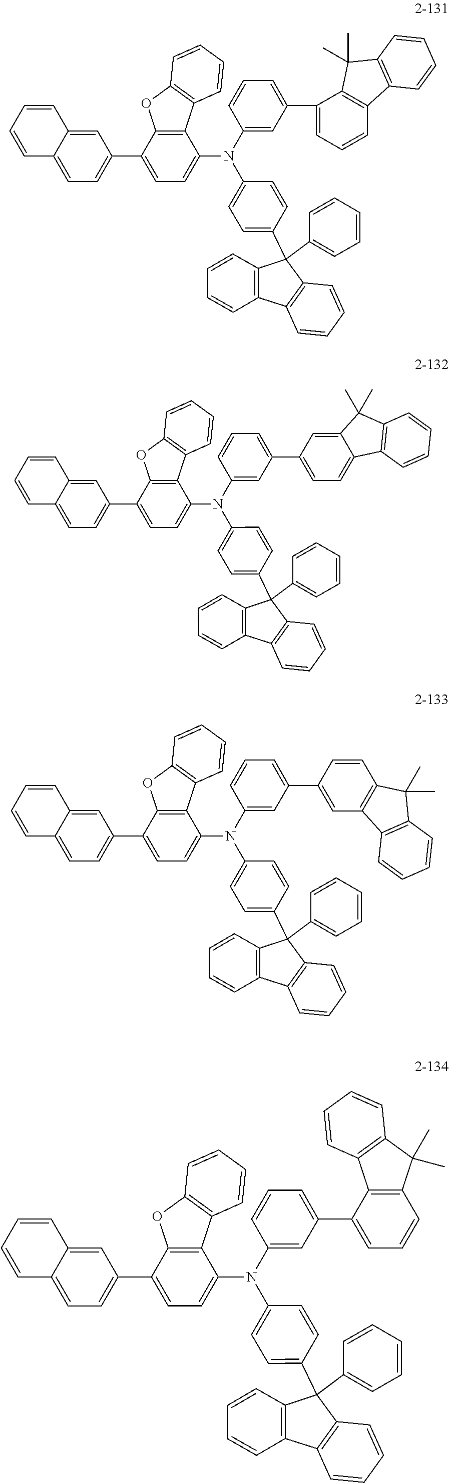 [SMILES: CC1(C)c2ccccc2-c2cccc(-c3cccc(N(c4ccc(C5(c6ccccc6)c6ccccc6-c6ccccc65)cc4)c4ccc(-c5ccc6ccccc6c5)c5oc6ccccc6c45)c3)c21 ; CC1(C)c2ccccc2-c2ccc(-c3cccc(N(c4ccc(C5(c6ccccc6)c6ccccc6-c6ccccc65)cc4)c4ccc(-c5ccc6ccccc6c5)c5oc6ccccc6c45)c3)cc21 ; CC1(C)c2ccccc2-c2cc(-c3cccc(N(c4ccc(C5(c6ccccc6)c6ccccc6-c6ccccc65)cc4)c4ccc(-c5ccc6ccccc6c5)c5oc6ccccc6c45)c3)ccc21 ; CC1(C)c2ccccc2-c2c(-c3cccc(N(c4ccc(C5(c6ccccc6)c6ccccc6-c6ccccc65)cc4)c4ccc(-c5ccc6ccccc6c5)c5oc6ccccc6c45)c3)cccc21]

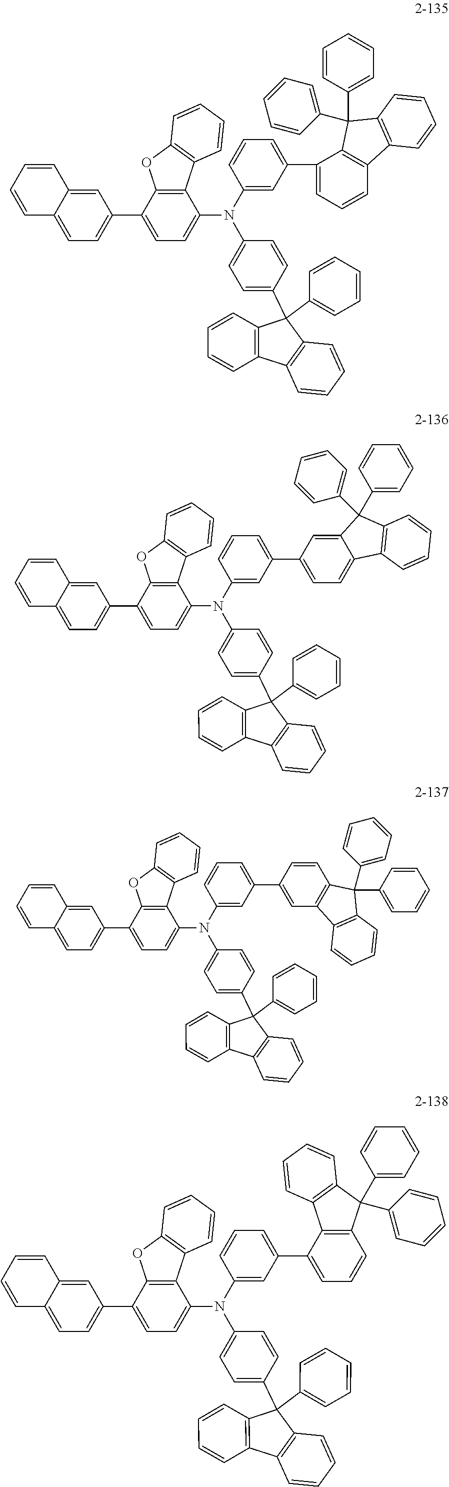

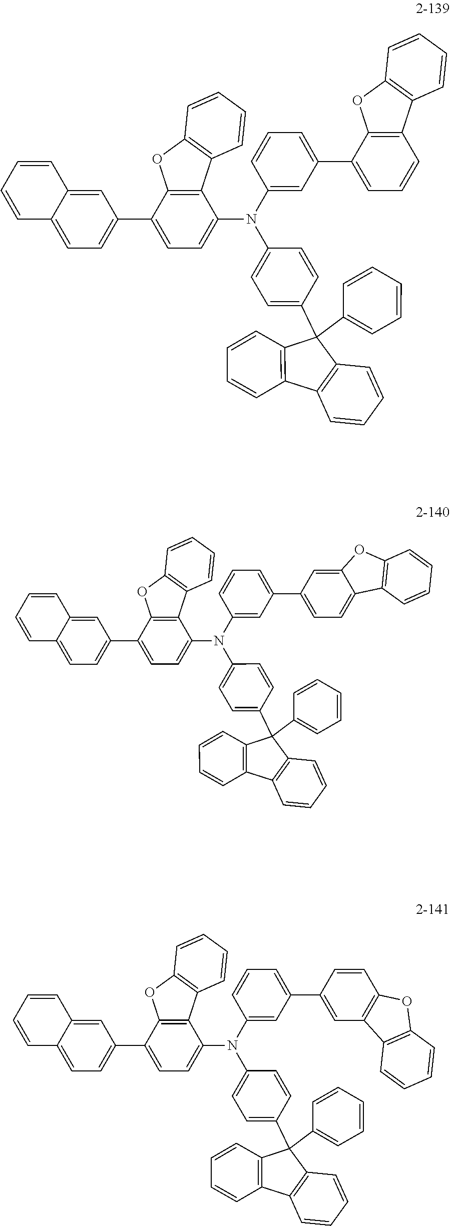 [SMILES: c1ccc(C2(c3ccc(N(c4cccc(-c5cccc6c5oc5ccccc56)c4)c4ccc(-c5ccc6ccccc6c5)c5oc6ccccc6c45)cc3)c3ccccc3-c3ccccc32)cc1 ; c1ccc(C2(c3ccc(N(c4cccc(-c5ccc6c(c5)oc5ccccc56)c4)c4ccc(-c5ccc6ccccc6c5)c5oc6ccccc6c45)cc3)c3ccccc3-c3ccccc32)cc1 ; c1ccc(C2(c3ccc(N(c4cccc(-c5ccc6oc7ccccc7c6c5)c4)c4ccc(-c5ccc6ccccc6c5)c5oc6ccccc6c45)cc3)c3ccccc3-c3ccccc32)cc1]

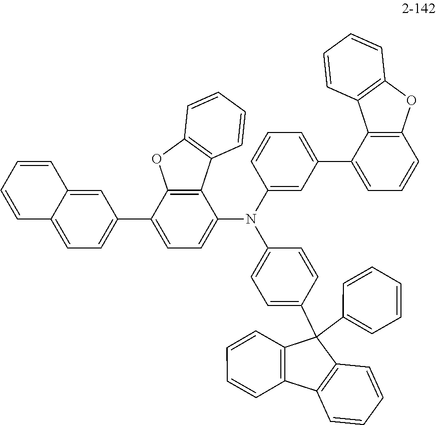 [SMILES: c1ccc(C2(c3ccc(N(c4cccc(-c5cccc6oc7ccccc7c56)c4)c4ccc(-c5ccc6ccccc6c5)c5oc6ccccc6c45)cc3)c3ccccc3-c3ccccc32)cc1]

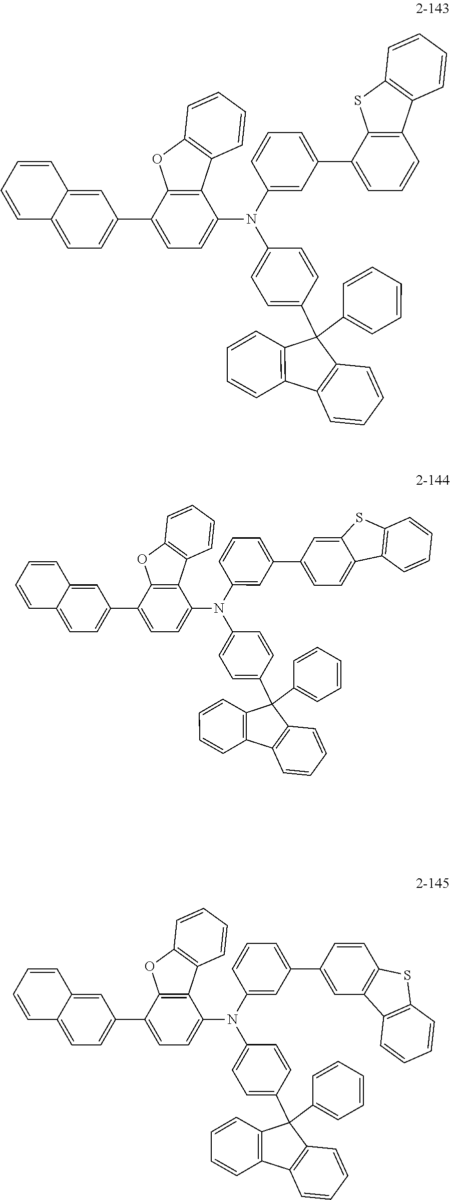 [SMILES: c1ccc(C2(c3ccc(N(c4cccc(-c5cccc6c5sc5ccccc56)c4)c4ccc(-c5ccc6ccccc6c5)c5oc6ccccc6c45)cc3)c3ccccc3-c3ccccc32)cc1 ; c1ccc(C2(c3ccc(N(c4cccc(-c5ccc6c(c5)sc5ccccc56)c4)c4ccc(-c5ccc6ccccc6c5)c5oc6ccccc6c45)cc3)c3ccccc3-c3ccccc32)cc1 ; c1ccc(C2(c3ccc(N(c4cccc(-c5ccc6sc7ccccc7c6c5)c4)c4ccc(-c5ccc6ccccc6c5)c5oc6ccccc6c45)cc3)c3ccccc3-c3ccccc32)cc1]

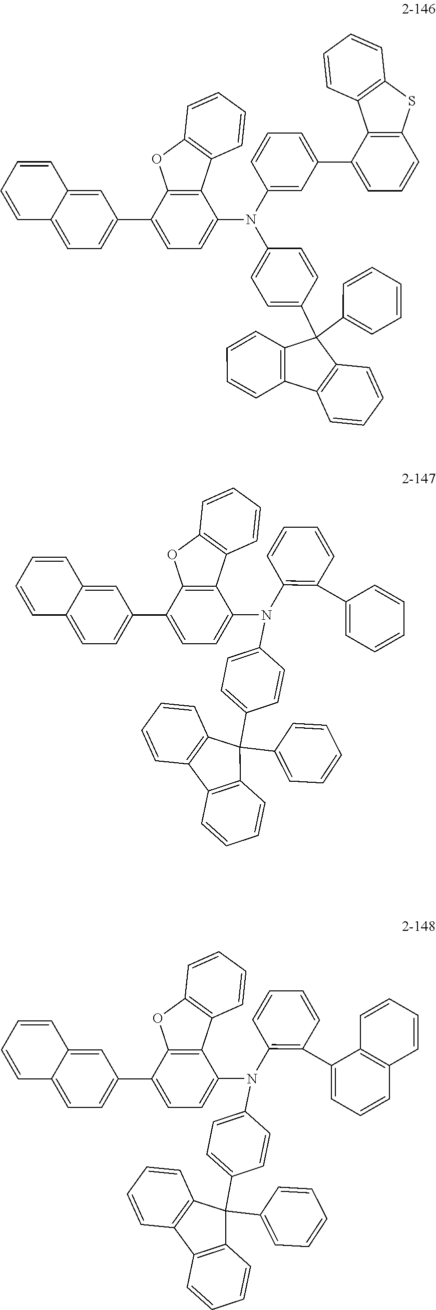 [SMILES: c1ccc(C2(c3ccc(N(c4cccc(-c5cccc6sc7ccccc7c56)c4)c4ccc(-c5ccc6ccccc6c5)c5oc6ccccc6c45)cc3)c3ccccc3-c3ccccc32)cc1 ; c1ccc(-c2ccccc2N(c2ccc(C3(c4ccccc4)c4ccccc4-c4ccccc43)cc2)c2ccc(-c3ccc4ccccc4c3)c3oc4ccccc4c23)cc1 ; c1ccc(C2(c3ccc(N(c4ccccc4-c4cccc5ccccc45)c4ccc(-c5ccc6ccccc6c5)c5oc6ccccc6c45)cc3)c3ccccc3-c3ccccc32)cc1]

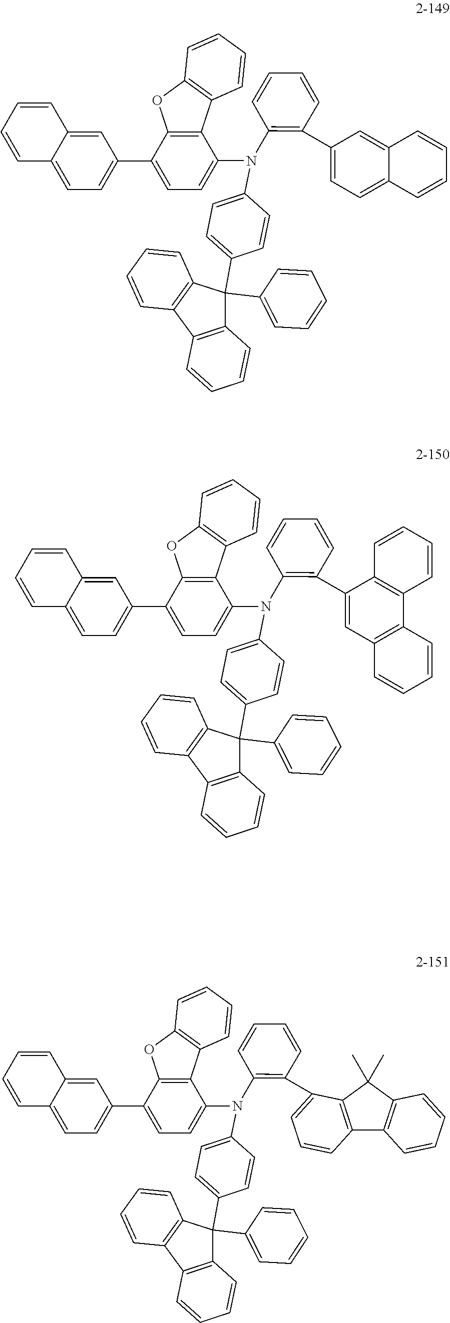 [SMILES: c1ccc(C2(c3ccc(N(c4ccccc4-c4ccc5ccccc5c4)c4ccc(-c5ccc6ccccc6c5)c5oc6ccccc6c45)cc3)c3ccccc3-c3ccccc32)cc1 ; c1ccc(C2(c3ccc(N(c4ccccc4-c4cc5ccccc5c5ccccc45)c4ccc(-c5ccc6ccccc6c5)c5oc6ccccc6c45)cc3)c3ccccc3-c3ccccc32)cc1 ; CC1(C)c2ccccc2-c2cccc(-c3ccccc3N(c3ccc(C4(c5ccccc5)c5ccccc5-c5ccccc54)cc3)c3ccc(-c4ccc5ccccc5c4)c4oc5ccccc5c34)c21]

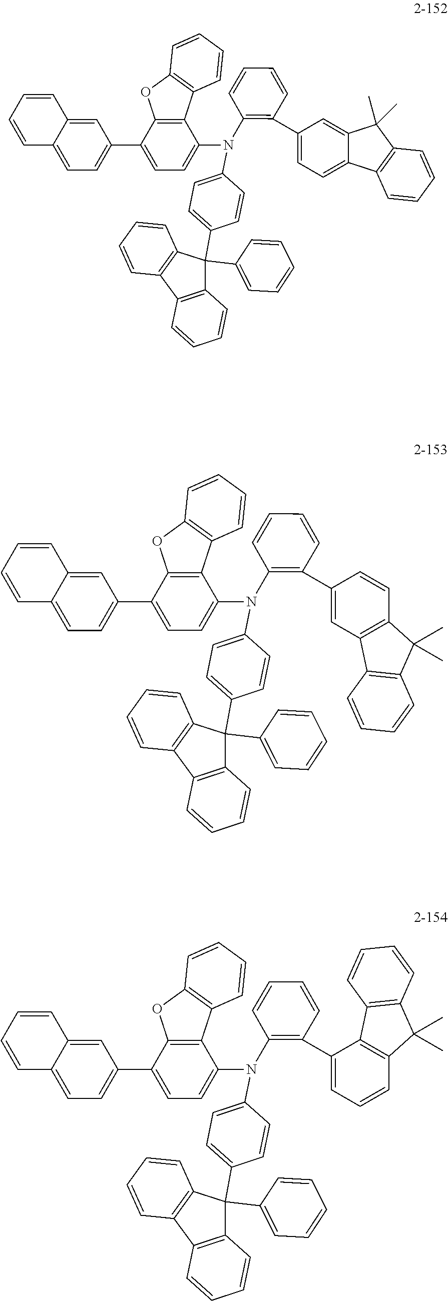 [SMILES: CC1(C)c2ccccc2-c2ccc(-c3ccccc3N(c3ccc(C4(c5ccccc5)c5ccccc5-c5ccccc54)cc3)c3ccc(-c4ccc5ccccc5c4)c4oc5ccccc5c34)cc21 ; CC1(C)c2ccccc2-c2cc(-c3ccccc3N(c3ccc(C4(c5ccccc5)c5ccccc5-c5ccccc54)cc3)c3ccc(-c4ccc5ccccc5c4)c4oc5ccccc5c34)ccc21 ; CC1(C)c2ccccc2-c2c(-c3ccccc3N(c3ccc(C4(c5ccccc5)c5ccccc5-c5ccccc54)cc3)c3ccc(-c4ccc5ccccc5c4)c4oc5ccccc5c34)cccc21]

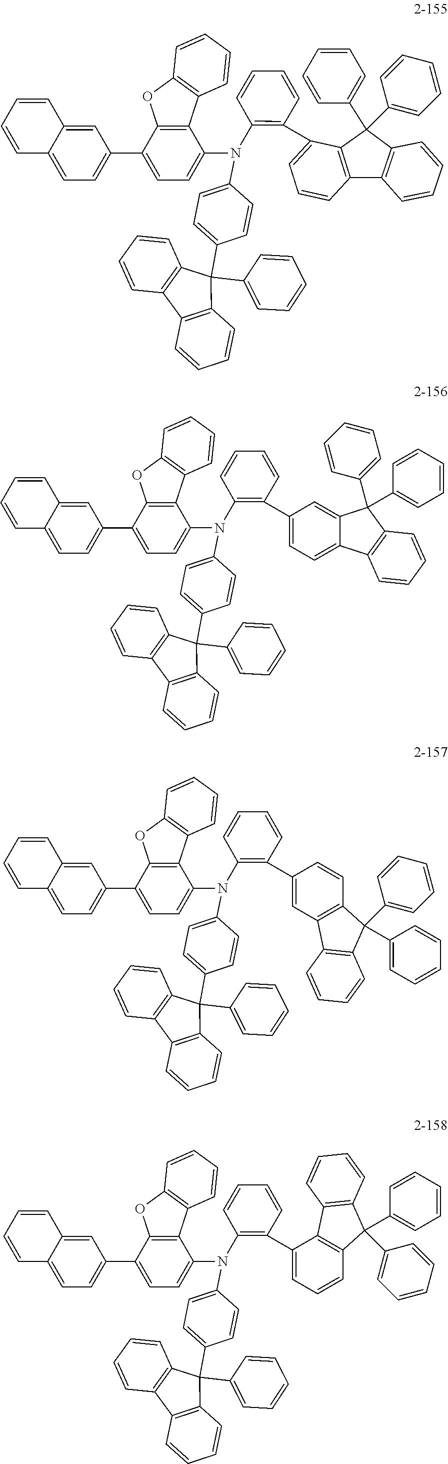

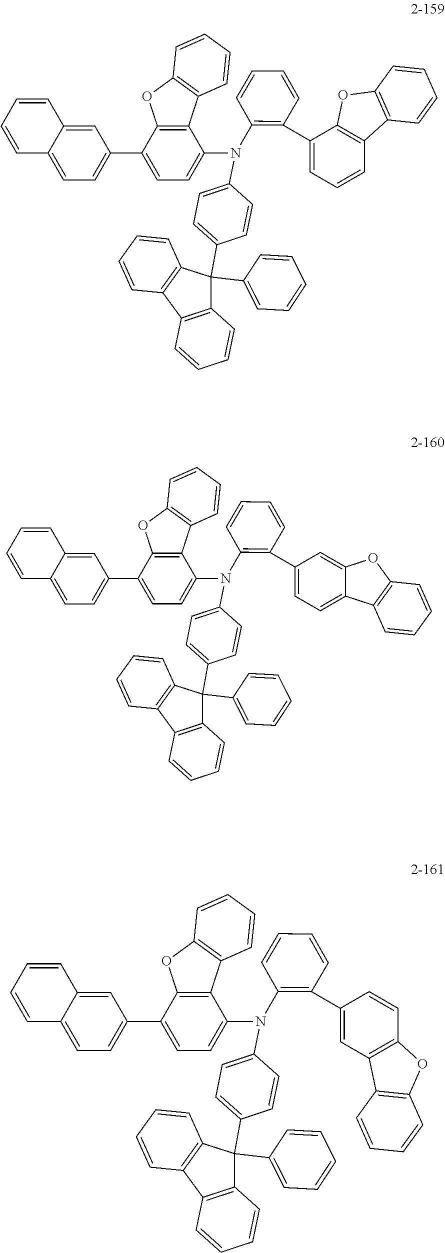 [SMILES: c1ccc(C2(c3ccc(N(c4ccccc4-c4cccc5c4oc4ccccc45)c4ccc(-c5ccc6ccccc6c5)c5oc6ccccc6c45)cc3)c3ccccc3-c3ccccc32)cc1 ; c1ccc(C2(c3ccc(N(c4ccccc4-c4ccc5c(c4)oc4ccccc45)c4ccc(-c5ccc6ccccc6c5)c5oc6ccccc6c45)cc3)c3ccccc3-c3ccccc32)cc1 ; c1ccc(C2(c3ccc(N(c4ccccc4-c4ccc5oc6ccccc6c5c4)c4ccc(-c5ccc6ccccc6c5)c5oc6ccccc6c45)cc3)c3ccccc3-c3ccccc32)cc1]

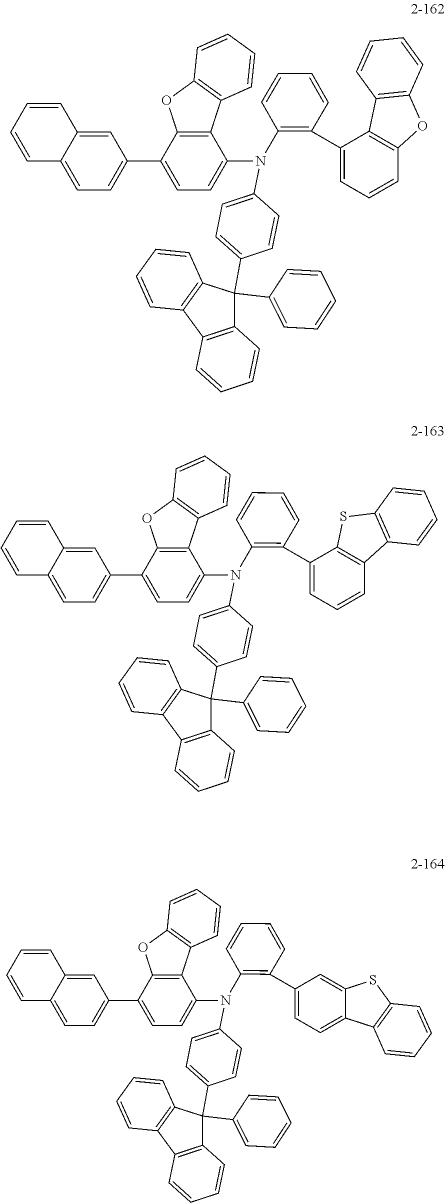 [SMILES: c1ccc(C2(c3ccc(N(c4ccccc4-c4cccc5oc6ccccc6c45)c4ccc(-c5ccc6ccccc6c5)c5oc6ccccc6c45)cc3)c3ccccc3-c3ccccc32)cc1 ; c1ccc(C2(c3ccc(N(c4ccccc4-c4cccc5c4sc4ccccc45)c4ccc(-c5ccc6ccccc6c5)c5oc6ccccc6c45)cc3)c3ccccc3-c3ccccc32)cc1 ; c1ccc(C2(c3ccc(N(c4ccccc4-c4ccc5c(c4)sc4ccccc45)c4ccc(-c5ccc6ccccc6c5)c5oc6ccccc6c45)cc3)c3ccccc3-c3ccccc32)cc1]

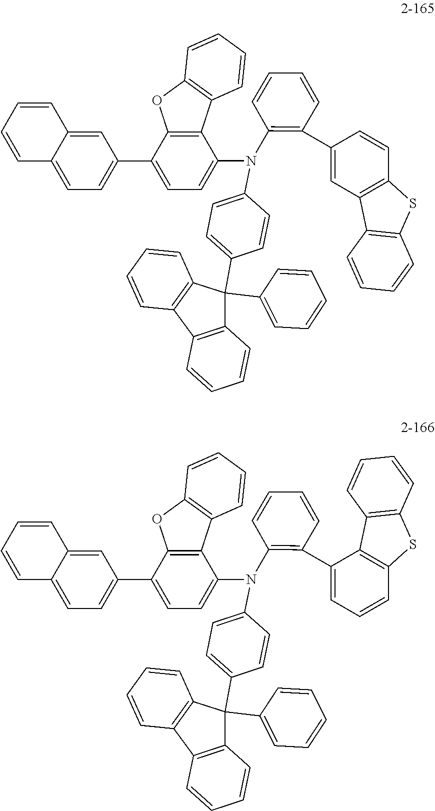 [SMILES: c1ccc(C2(c3ccc(N(c4ccccc4-c4ccc5sc6ccccc6c5c4)c4ccc(-c5ccc6ccccc6c5)c5oc6ccccc6c45)cc3)c3ccccc3-c3ccccc32)cc1 ; c1ccc(C2(c3ccc(N(c4ccccc4-c4cccc5sc6ccccc6c45)c4ccc(-c5ccc6ccccc6c5)c5oc6ccccc6c45)cc3)c3ccccc3-c3ccccc32)cc1]

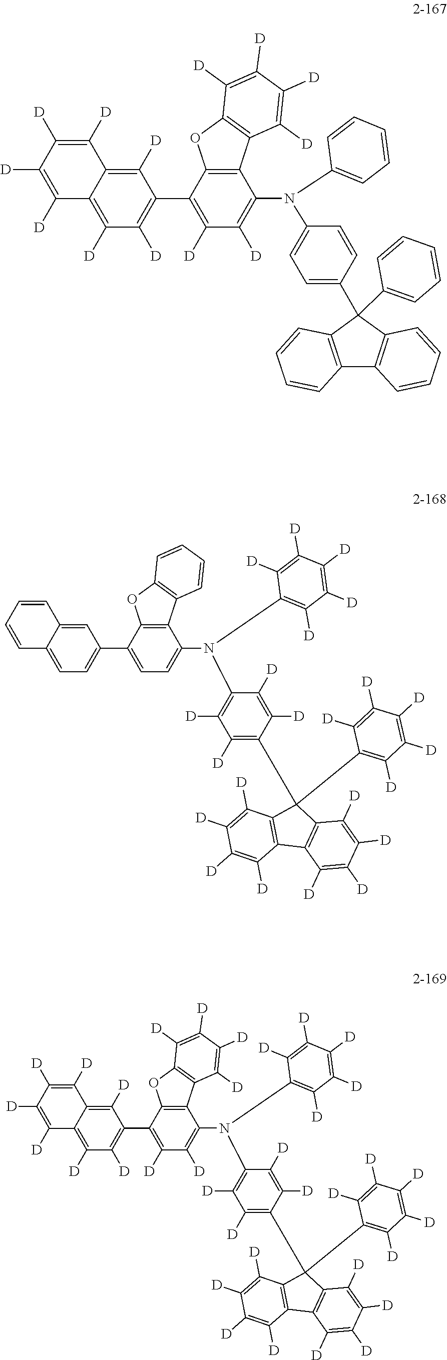

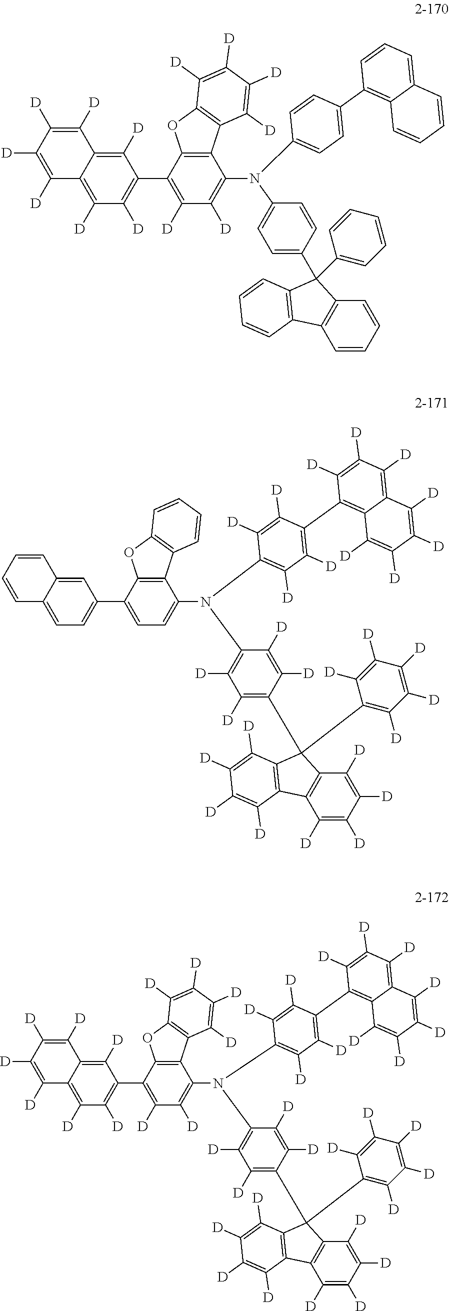

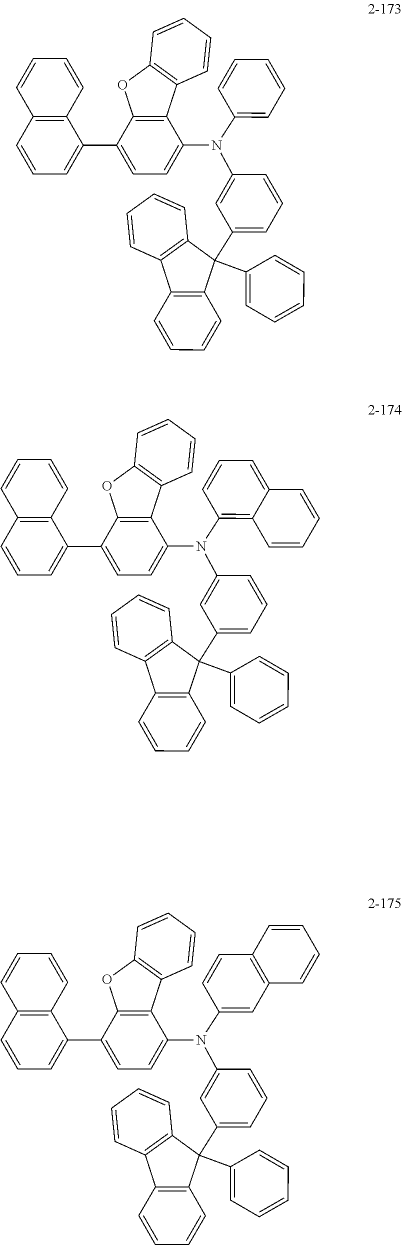 [SMILES: c1ccc(N(c2cccc(C3(c4ccccc4)c4ccccc4-c4ccccc43)c2)c2ccc(-c3cccc4ccccc34)c3oc4ccccc4c23)cc1 ; c1ccc(C2(c3cccc(N(c4cccc5ccccc45)c4ccc(-c5cccc6ccccc56)c5oc6ccccc6c45)c3)c3ccccc3-c3ccccc32)cc1 ; c1ccc(C2(c3cccc(N(c4ccc5ccccc5c4)c4ccc(-c5cccc6ccccc56)c5oc6ccccc6c45)c3)c3ccccc3-c3ccccc32)cc1]

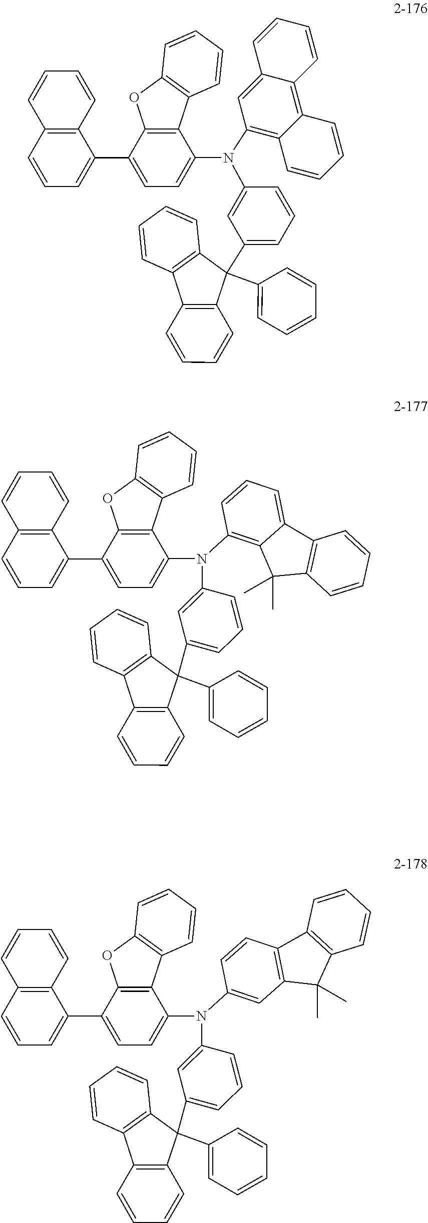 [SMILES: c1ccc(C2(c3cccc(N(c4cc5ccccc5c5ccccc45)c4ccc(-c5cccc6ccccc56)c5oc6ccccc6c45)c3)c3ccccc3-c3ccccc32)cc1 ; CC1(C)c2ccccc2-c2cccc(N(c3cccc(C4(c5ccccc5)c5ccccc5-c5ccccc54)c3)c3ccc(-c4cccc5ccccc45)c4oc5ccccc5c34)c21 ; CC1(C)c2ccccc2-c2ccc(N(c3cccc(C4(c5ccccc5)c5ccccc5-c5ccccc54)c3)c3ccc(-c4cccc5ccccc45)c4oc5ccccc5c34)cc21]

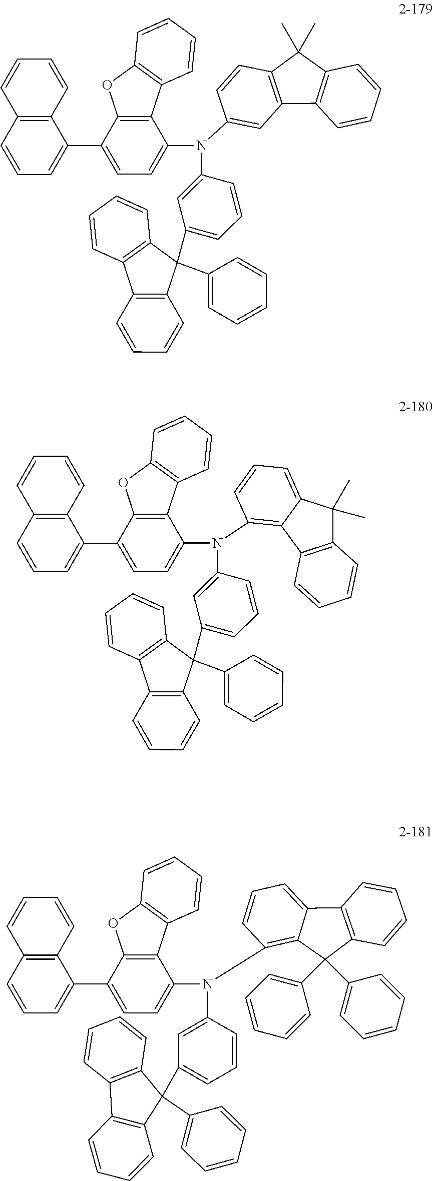 [SMILES: CC1(C)c2ccccc2-c2cc(N(c3cccc(C4(c5ccccc5)c5ccccc5-c5ccccc54)c3)c3ccc(-c4cccc5ccccc45)c4oc5ccccc5c34)ccc21 ; CC1(C)c2ccccc2-c2c(N(c3cccc(C4(c5ccccc5)c5ccccc5-c5ccccc54)c3)c3ccc(-c4cccc5ccccc45)c4oc5ccccc5c34)cccc21 ; c1ccc(C2(c3cccc(N(c4cccc5c4C(c4ccccc4)(c4ccccc4)c4ccccc4-5)c4ccc(-c5cccc6ccccc56)c5oc6ccccc6c45)c3)c3ccccc3-c3ccccc32)cc1]

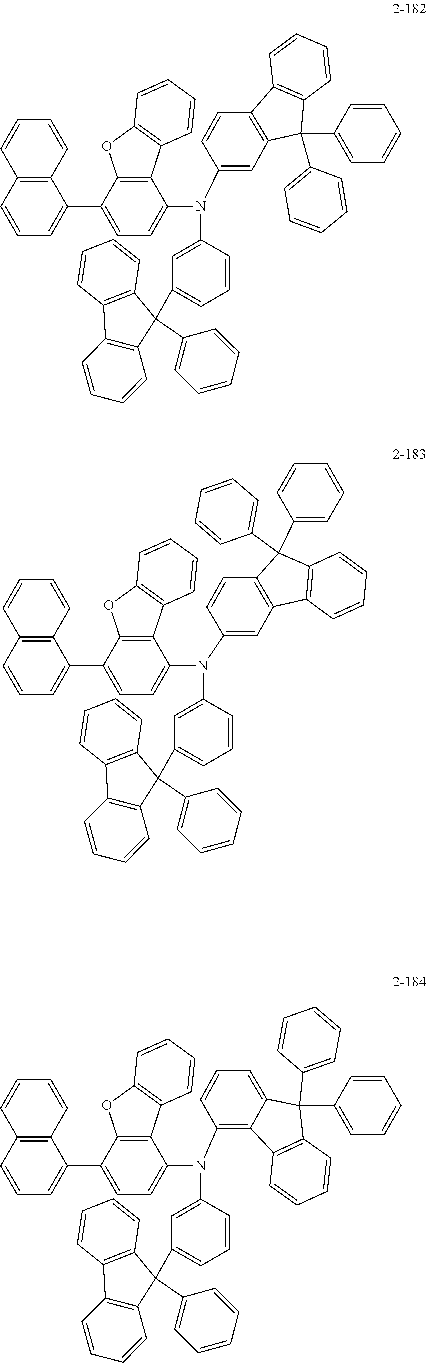 [SMILES: c1ccc(C2(c3cccc(N(c4ccc5c(c4)C(c4ccccc4)(c4ccccc4)c4ccccc4-5)c4ccc(-c5cccc6ccccc56)c5oc6ccccc6c45)c3)c3ccccc3-c3ccccc32)cc1 ; c1ccc(C2(c3cccc(N(c4ccc5c(c4)-c4ccccc4C5(c4ccccc4)c4ccccc4)c4ccc(-c5cccc6ccccc56)c5oc6ccccc6c45)c3)c3ccccc3-c3ccccc32)cc1 ; c1ccc(C2(c3cccc(N(c4cccc5c4-c4ccccc4C5(c4ccccc4)c4ccccc4)c4ccc(-c5cccc6ccccc56)c5oc6ccccc6c45)c3)c3ccccc3-c3ccccc32)cc1]

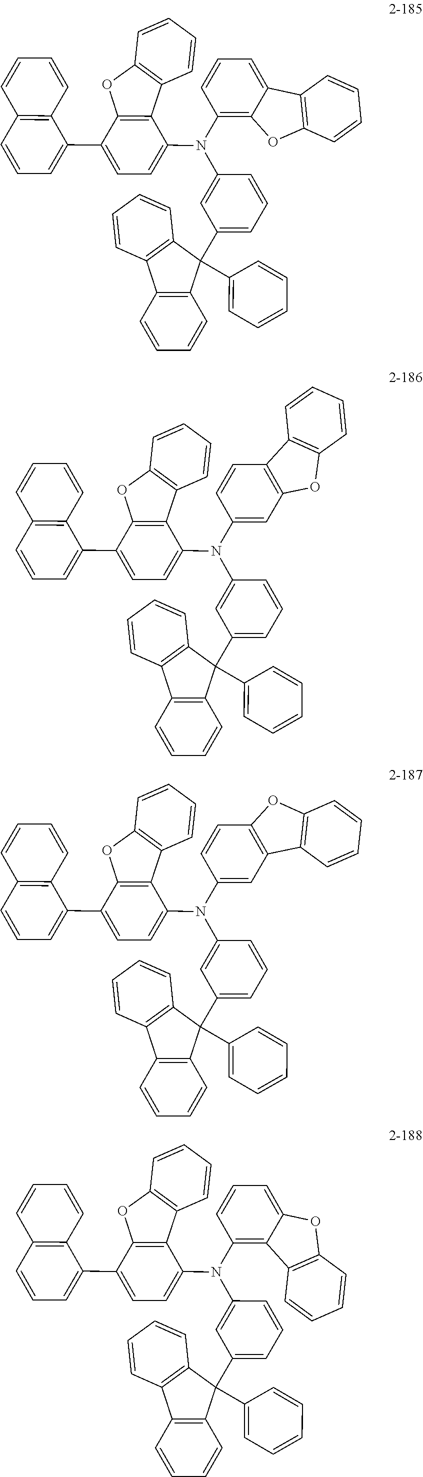

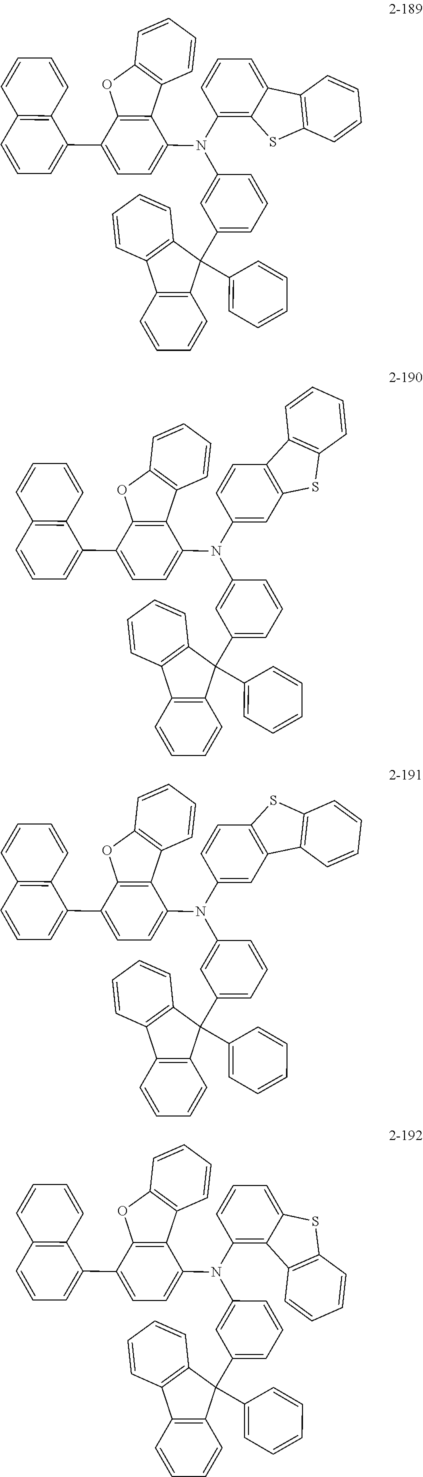

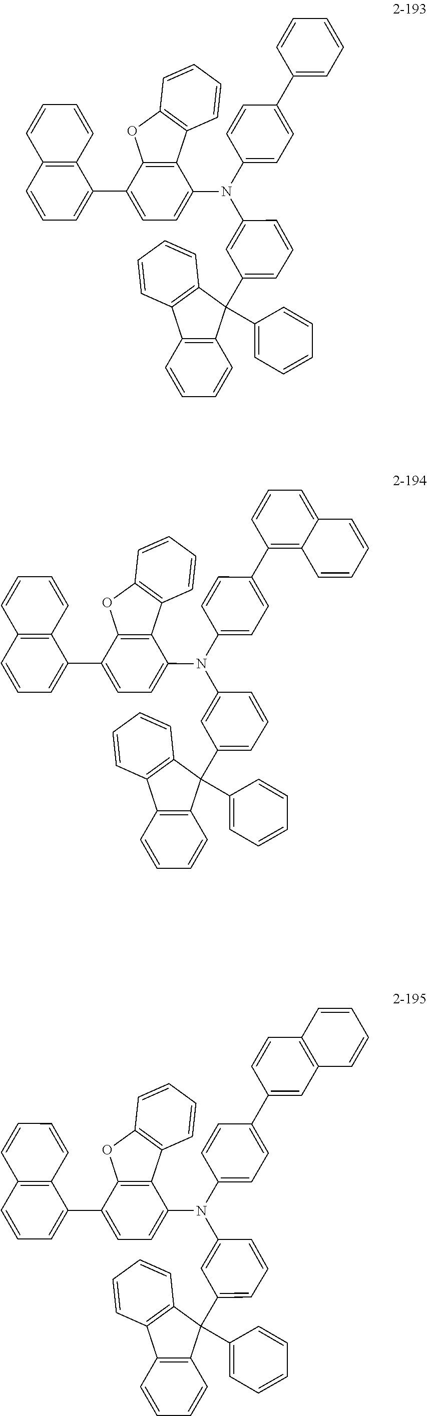

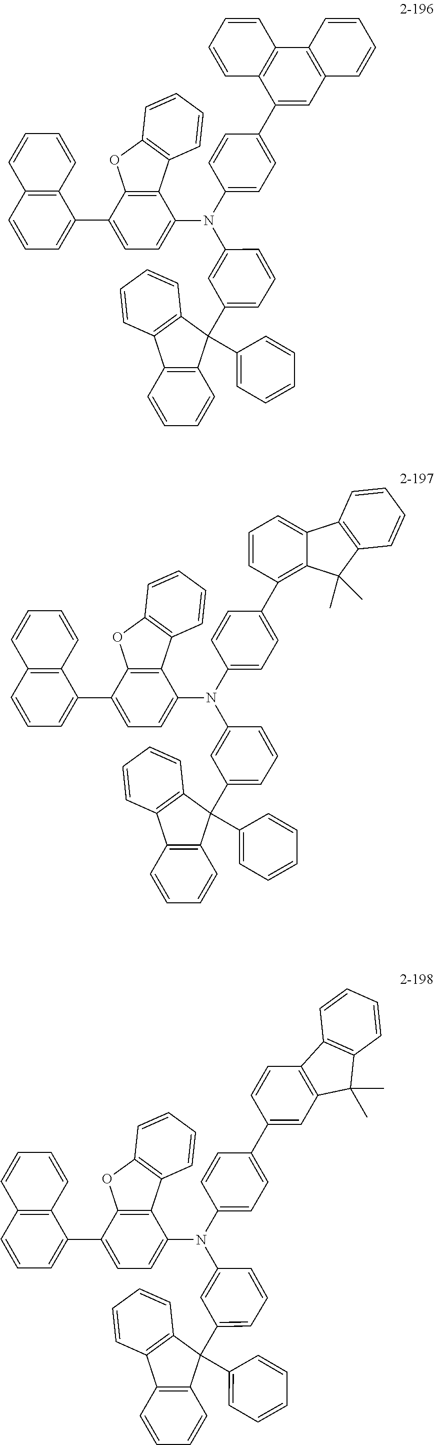

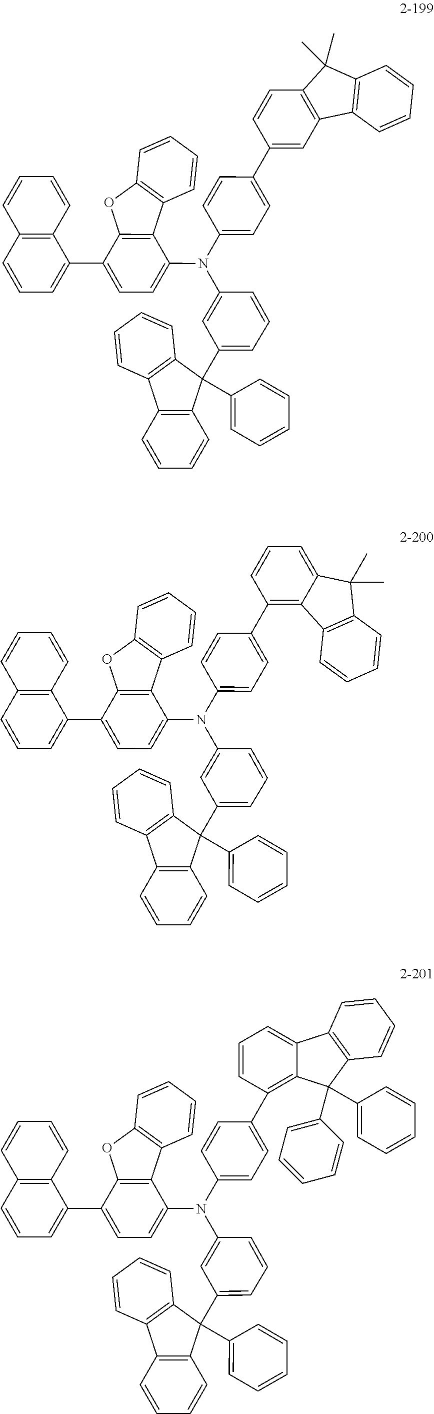

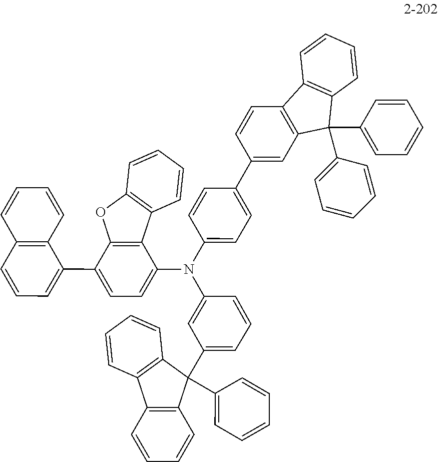

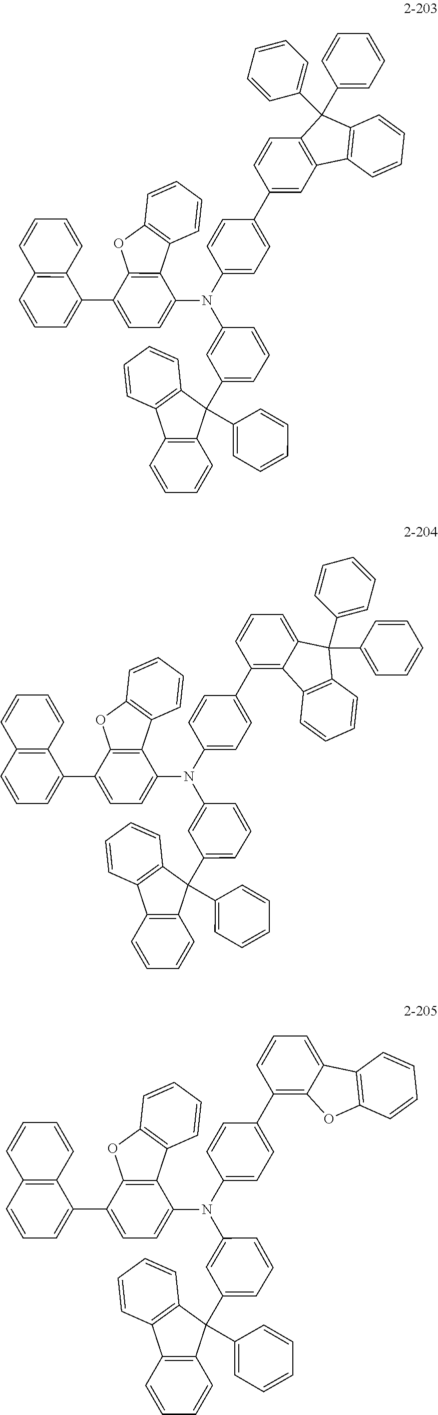

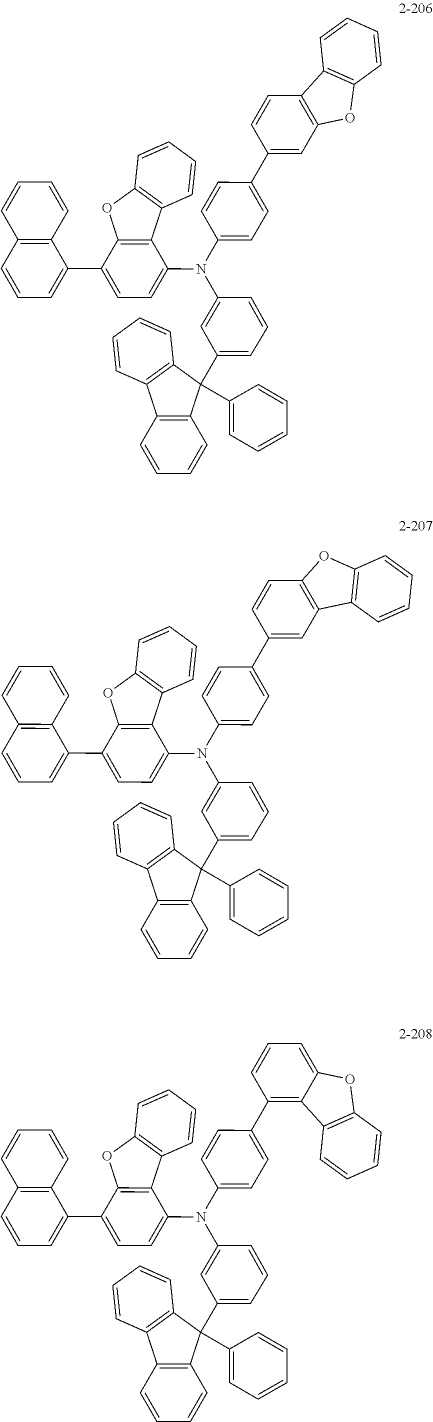

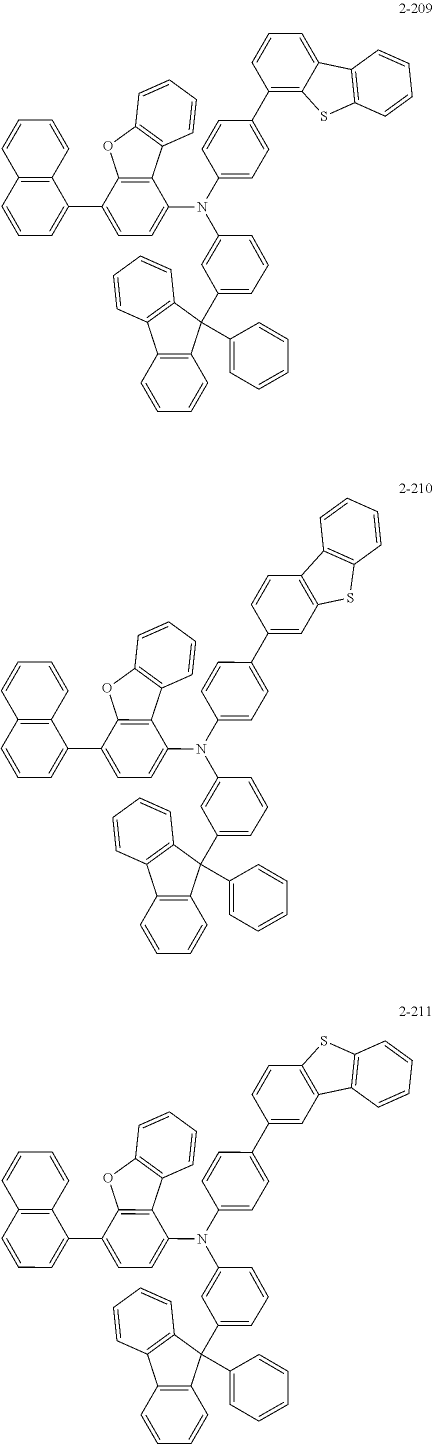

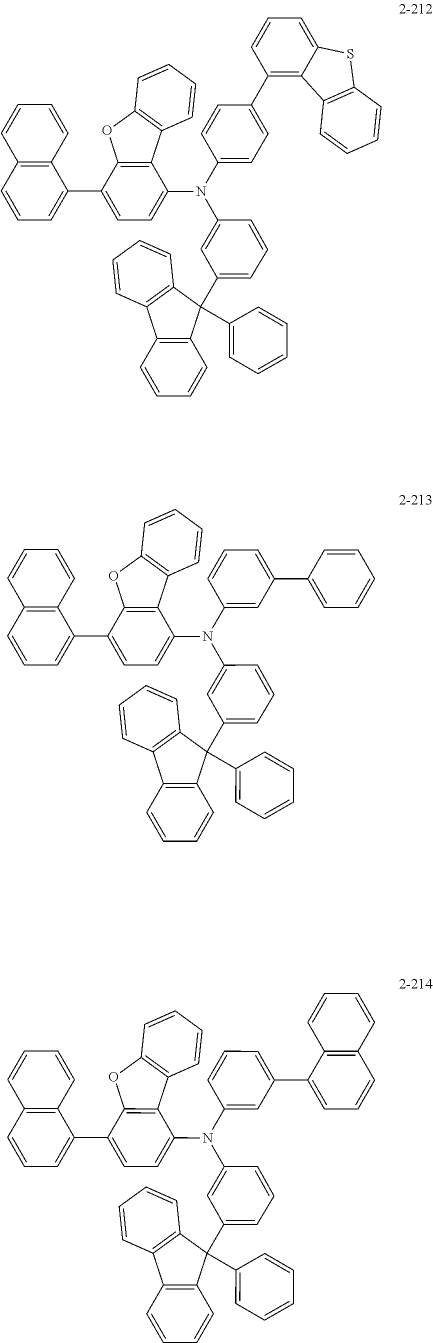 [SMILES: c1ccc(C2(c3cccc(N(c4ccc(-c5cccc6sc7ccccc7c56)cc4)c4ccc(-c5cccc6ccccc56)c5oc6ccccc6c45)c3)c3ccccc3-c3ccccc32)cc1 ; c1ccc(-c2cccc(N(c3cccc(C4(c5ccccc5)c5ccccc5-c5ccccc54)c3)c3ccc(-c4cccc5ccccc45)c4oc5ccccc5c34)c2)cc1 ; c1ccc(C2(c3cccc(N(c4cccc(-c5cccc6ccccc56)c4)c4ccc(-c5cccc6ccccc56)c5oc6ccccc6c45)c3)c3ccccc3-c3ccccc32)cc1]

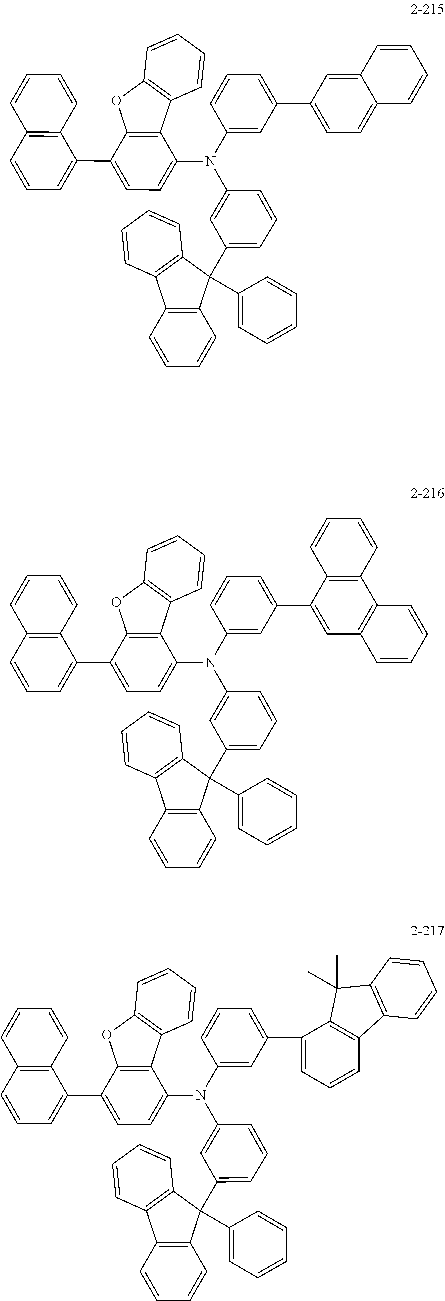 [SMILES: c1ccc(C2(c3cccc(N(c4cccc(-c5ccc6ccccc6c5)c4)c4ccc(-c5cccc6ccccc56)c5oc6ccccc6c45)c3)c3ccccc3-c3ccccc32)cc1 ; c1ccc(C2(c3cccc(N(c4cccc(-c5cc6ccccc6c6ccccc56)c4)c4ccc(-c5cccc6ccccc56)c5oc6ccccc6c45)c3)c3ccccc3-c3ccccc32)cc1 ; CC1(C)c2ccccc2-c2cccc(-c3cccc(N(c4cccc(C5(c6ccccc6)c6ccccc6-c6ccccc65)c4)c4ccc(-c5cccc6ccccc56)c5oc6ccccc6c45)c3)c21]

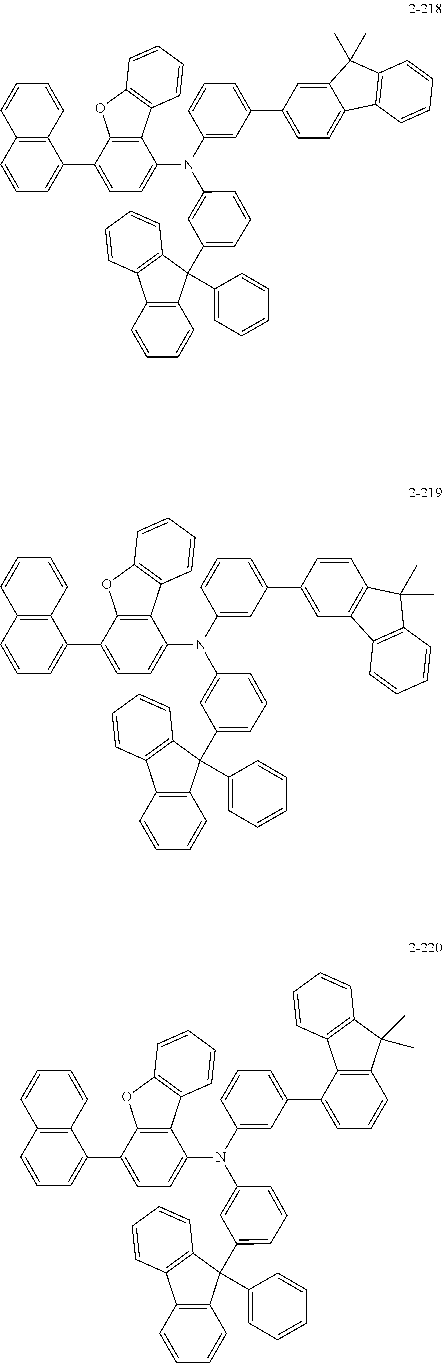 [SMILES: CC1(C)c2ccccc2-c2ccc(-c3cccc(N(c4cccc(C5(c6ccccc6)c6ccccc6-c6ccccc65)c4)c4ccc(-c5cccc6ccccc56)c5oc6ccccc6c45)c3)cc21 ; CC1(C)c2ccccc2-c2cc(-c3cccc(N(c4cccc(C5(c6ccccc6)c6ccccc6-c6ccccc65)c4)c4ccc(-c5cccc6ccccc56)c5oc6ccccc6c45)c3)ccc21 ; CC1(C)c2ccccc2-c2c(-c3cccc(N(c4cccc(C5(c6ccccc6)c6ccccc6-c6ccccc65)c4)c4ccc(-c5cccc6ccccc56)c5oc6ccccc6c45)c3)cccc21]

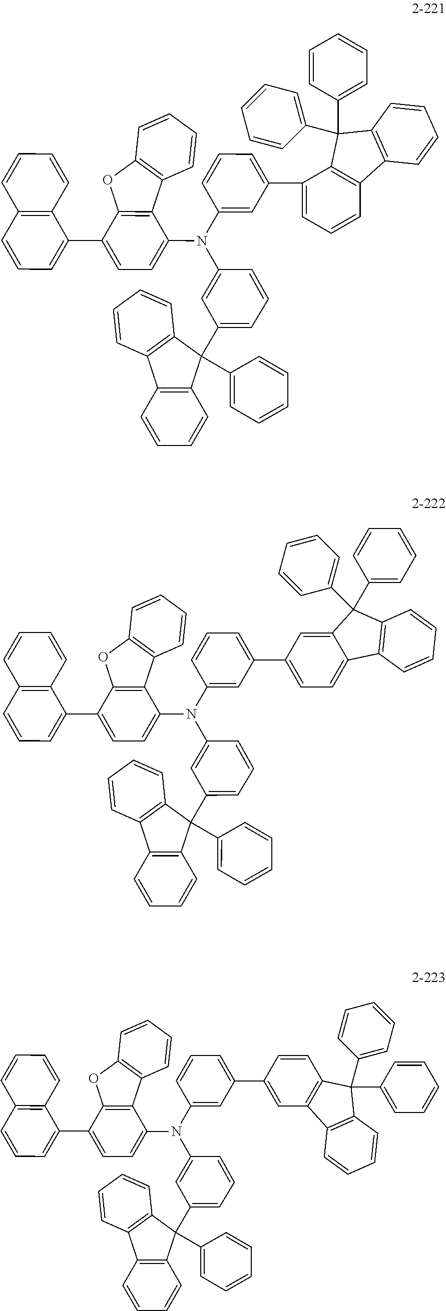 [SMILES: c1ccc(C2(c3cccc(N(c4cccc(-c5cccc6c5C(c5ccccc5)(c5ccccc5)c5ccccc5-6)c4)c4ccc(-c5cccc6ccccc56)c5oc6ccccc6c45)c3)c3ccccc3-c3ccccc32)cc1 ; c1ccc(C2(c3cccc(N(c4cccc(-c5ccc6c(c5)C(c5ccccc5)(c5ccccc5)c5ccccc5-6)c4)c4ccc(-c5cccc6ccccc56)c5oc6ccccc6c45)c3)c3ccccc3-c3ccccc32)cc1 ; c1ccc(C2(c3cccc(N(c4cccc(-c5ccc6c(c5)-c5ccccc5C6(c5ccccc5)c5ccccc5)c4)c4ccc(-c5cccc6ccccc56)c5oc6ccccc6c45)c3)c3ccccc3-c3ccccc32)cc1]

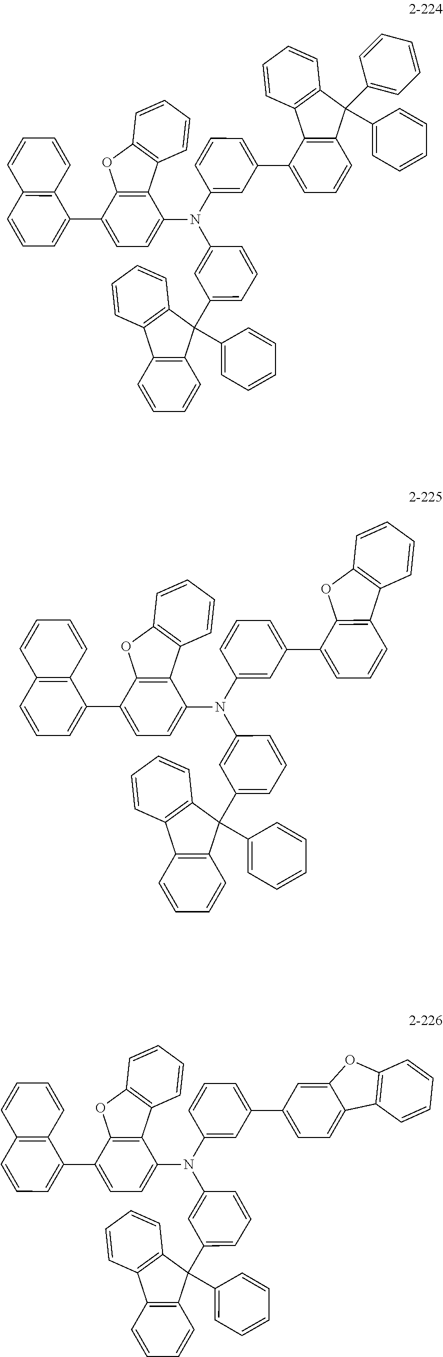 [SMILES: c1ccc(C2(c3cccc(N(c4cccc(-c5cccc6c5-c5ccccc5C6(c5ccccc5)c5ccccc5)c4)c4ccc(-c5cccc6ccccc56)c5oc6ccccc6c45)c3)c3ccccc3-c3ccccc32)cc1 ; c1ccc(C2(c3cccc(N(c4cccc(-c5cccc6c5oc5ccccc56)c4)c4ccc(-c5cccc6ccccc56)c5oc6ccccc6c45)c3)c3ccccc3-c3ccccc32)cc1 ; c1ccc(C2(c3cccc(N(c4cccc(-c5ccc6c(c5)oc5ccccc56)c4)c4ccc(-c5cccc6ccccc56)c5oc6ccccc6c45)c3)c3ccccc3-c3ccccc32)cc1]

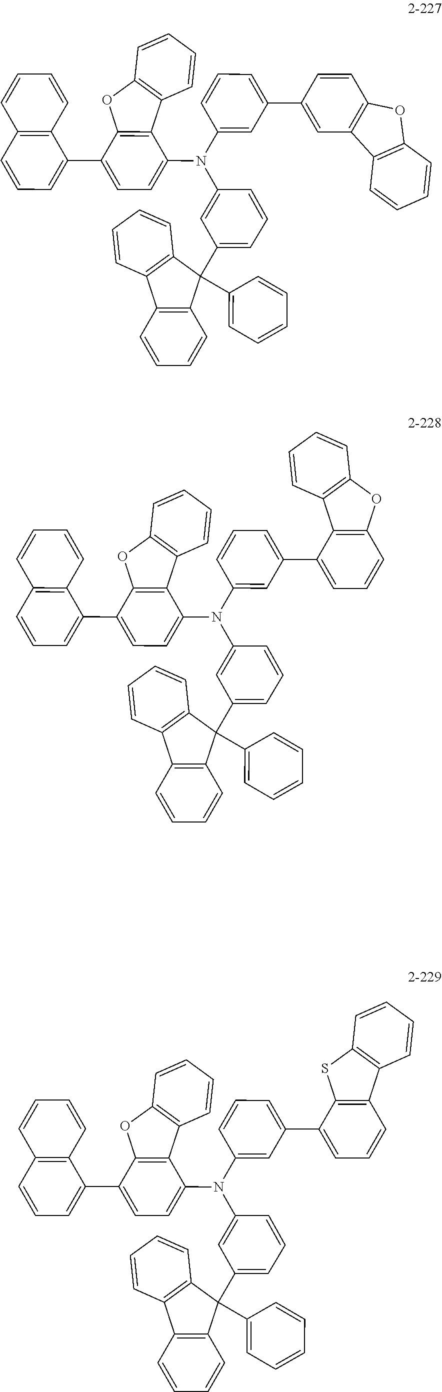 [SMILES: c1ccc(C2(c3cccc(N(c4cccc(-c5ccc6oc7ccccc7c6c5)c4)c4ccc(-c5cccc6ccccc56)c5oc6ccccc6c45)c3)c3ccccc3-c3ccccc32)cc1 ; c1ccc(C2(c3cccc(N(c4cccc(-c5cccc6oc7ccccc7c56)c4)c4ccc(-c5cccc6ccccc56)c5oc6ccccc6c45)c3)c3ccccc3-c3ccccc32)cc1 ; c1ccc(C2(c3cccc(N(c4cccc(-c5cccc6c5sc5ccccc56)c4)c4ccc(-c5cccc6ccccc56)c5oc6ccccc6c45)c3)c3ccccc3-c3ccccc32)cc1]

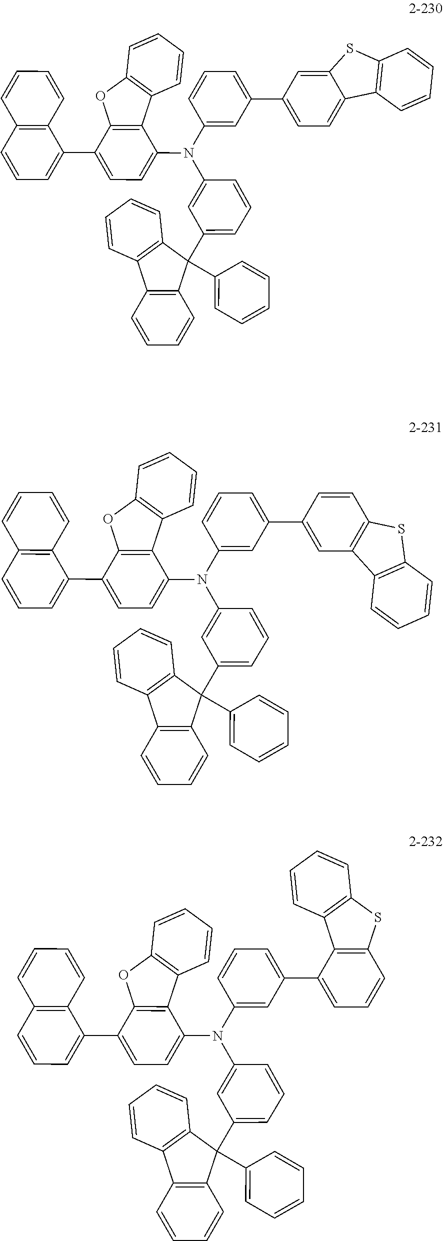 [SMILES: c1ccc(C2(c3cccc(N(c4cccc(-c5ccc6c(c5)sc5ccccc56)c4)c4ccc(-c5cccc6ccccc56)c5oc6ccccc6c45)c3)c3ccccc3-c3ccccc32)cc1 ; c1ccc(C2(c3cccc(N(c4cccc(-c5ccc6sc7ccccc7c6c5)c4)c4ccc(-c5cccc6ccccc56)c5oc6ccccc6c45)c3)c3ccccc3-c3ccccc32)cc1 ; c1ccc(C2(c3cccc(N(c4cccc(-c5cccc6sc7ccccc7c56)c4)c4ccc(-c5cccc6ccccc56)c5oc6ccccc6c45)c3)c3ccccc3-c3ccccc32)cc1]

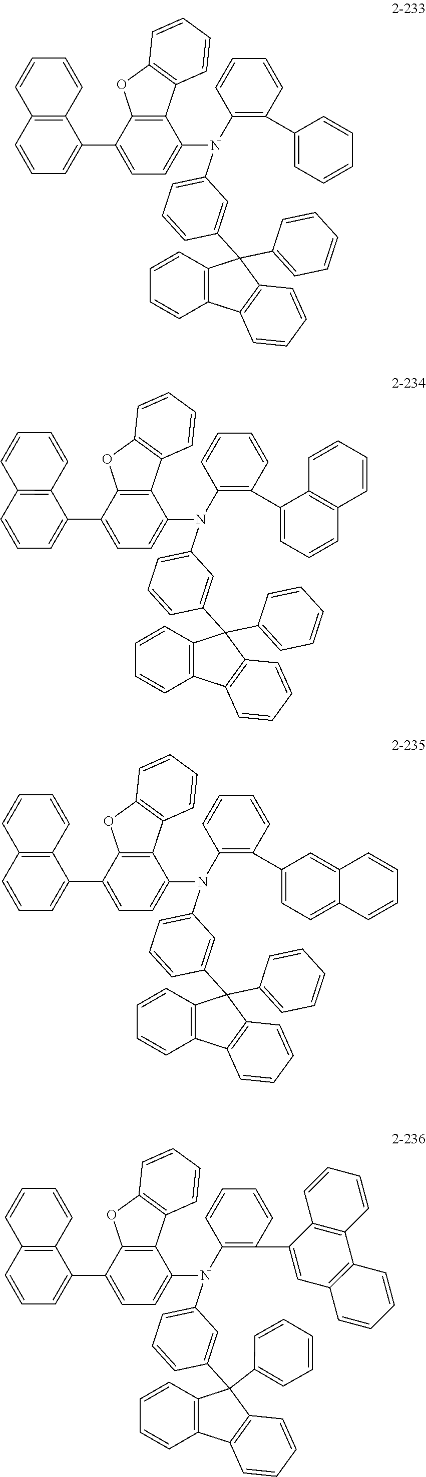 [SMILES: c1ccc(-c2ccccc2N(c2cccc(C3(c4ccccc4)c4ccccc4-c4ccccc43)c2)c2ccc(-c3cccc4ccccc34)c3oc4ccccc4c23)cc1 ; c1ccc(C2(c3cccc(N(c4ccccc4-c4cccc5ccccc45)c4ccc(-c5cccc6ccccc56)c5oc6ccccc6c45)c3)c3ccccc3-c3ccccc32)cc1 ; c1ccc(C2(c3cccc(N(c4ccccc4-c4ccc5ccccc5c4)c4ccc(-c5cccc6ccccc56)c5oc6ccccc6c45)c3)c3ccccc3-c3ccccc32)cc1 ; c1ccc(C2(c3cccc(N(c4ccccc4-c4cc5ccccc5c5ccccc45)c4ccc(-c5cccc6ccccc56)c5oc6ccccc6c45)c3)c3ccccc3-c3ccccc32)cc1]

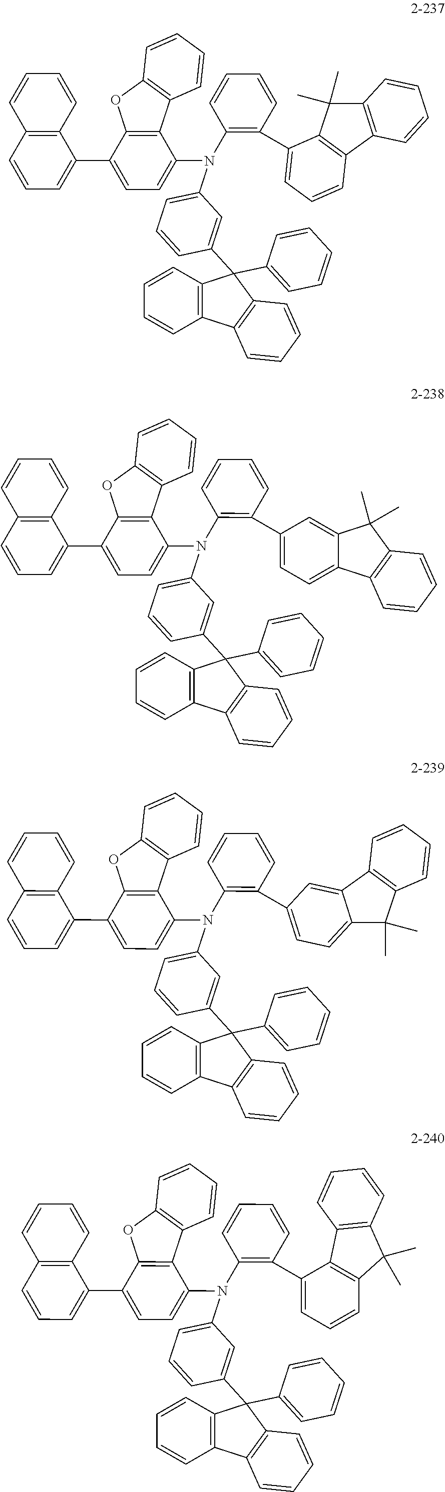 [SMILES: CC1(C)c2ccccc2-c2cccc(-c3ccccc3N(c3cccc(C4(c5ccccc5)c5ccccc5-c5ccccc54)c3)c3ccc(-c4cccc5ccccc45)c4oc5ccccc5c34)c21 ; CC1(C)c2ccccc2-c2ccc(-c3ccccc3N(c3cccc(C4(c5ccccc5)c5ccccc5-c5ccccc54)c3)c3ccc(-c4cccc5ccccc45)c4oc5ccccc5c34)cc21 ; CC1(C)c2ccccc2-c2cc(-c3ccccc3N(c3cccc(C4(c5ccccc5)c5ccccc5-c5ccccc54)c3)c3ccc(-c4cccc5ccccc45)c4oc5ccccc5c34)ccc21 ; CC1(C)c2ccccc2-c2c(-c3ccccc3N(c3cccc(C4(c5ccccc5)c5ccccc5-c5ccccc54)c3)c3ccc(-c4cccc5ccccc45)c4oc5ccccc5c34)cccc21]

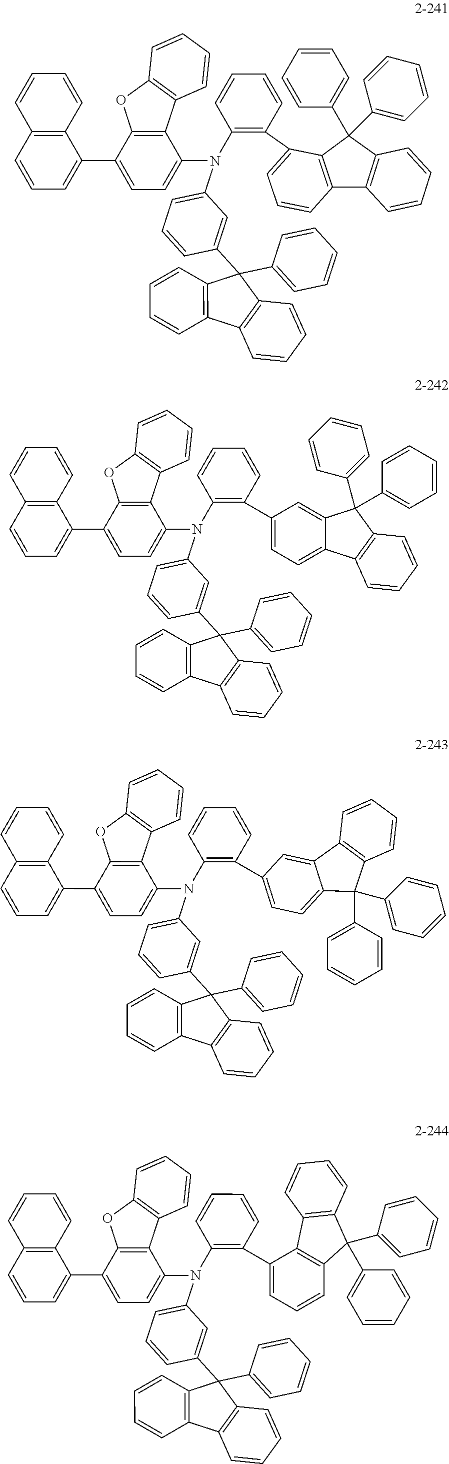

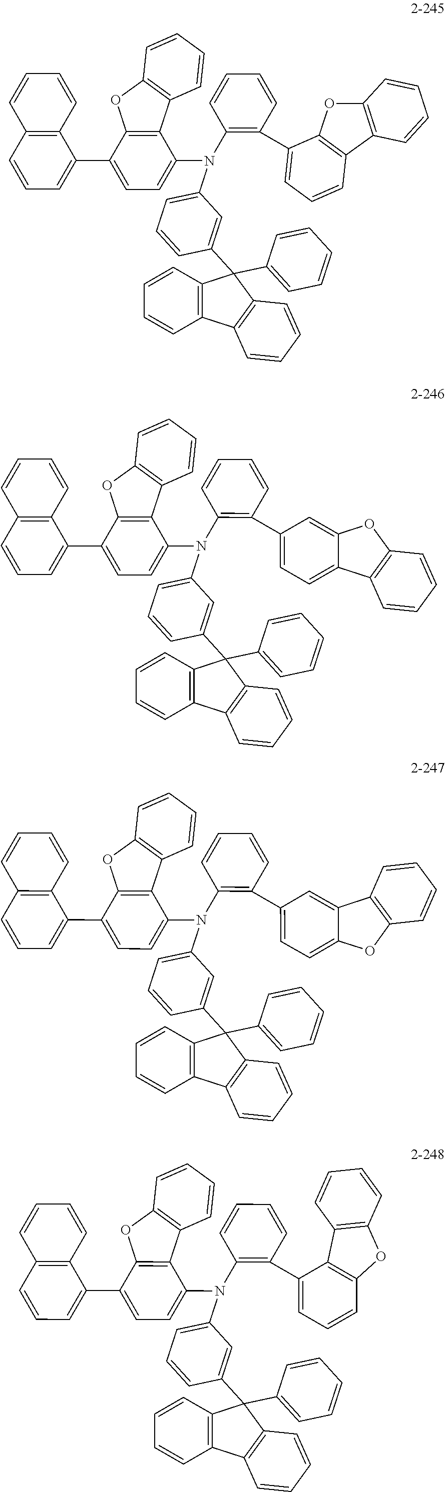 [SMILES: c1ccc(C2(c3cccc(N(c4ccccc4-c4cccc5c4oc4ccccc45)c4ccc(-c5cccc6ccccc56)c5oc6ccccc6c45)c3)c3ccccc3-c3ccccc32)cc1 ; c1ccc(C2(c3cccc(N(c4ccccc4-c4ccc5c(c4)oc4ccccc45)c4ccc(-c5cccc6ccccc56)c5oc6ccccc6c45)c3)c3ccccc3-c3ccccc32)cc1 ; c1ccc(C2(c3cccc(N(c4ccccc4-c4ccc5oc6ccccc6c5c4)c4ccc(-c5cccc6ccccc56)c5oc6ccccc6c45)c3)c3ccccc3-c3ccccc32)cc1 ; c1ccc(C2(c3cccc(N(c4ccccc4-c4cccc5oc6ccccc6c45)c4ccc(-c5cccc6ccccc56)c5oc6ccccc6c45)c3)c3ccccc3-c3ccccc32)cc1]

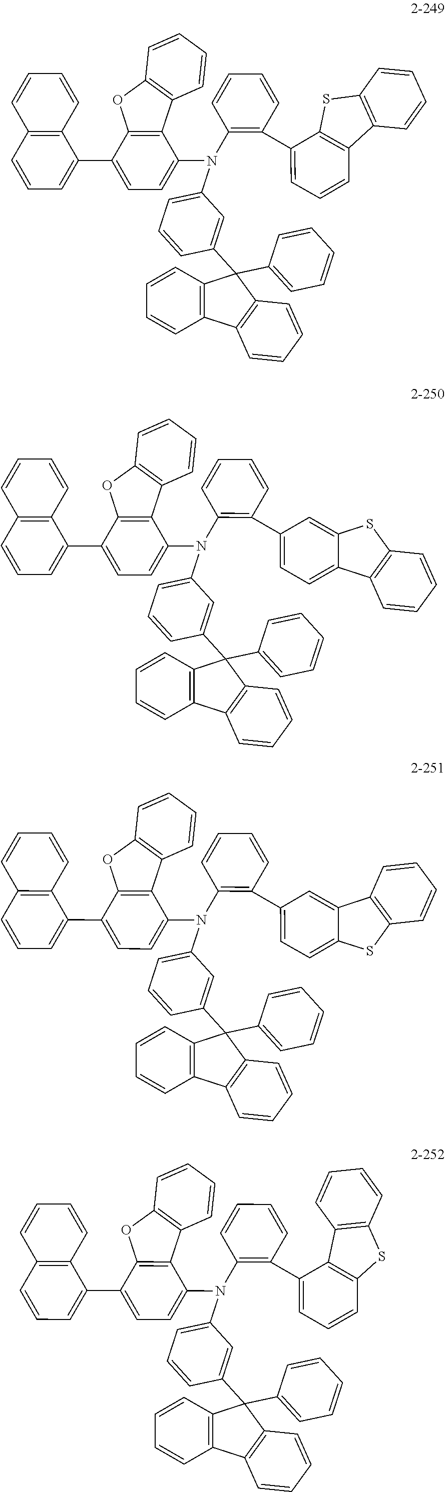 [SMILES: c1ccc(C2(c3cccc(N(c4ccccc4-c4cccc5c4sc4ccccc45)c4ccc(-c5cccc6ccccc56)c5oc6ccccc6c45)c3)c3ccccc3-c3ccccc32)cc1 ; c1ccc(C2(c3cccc(N(c4ccccc4-c4ccc5c(c4)sc4ccccc45)c4ccc(-c5cccc6ccccc56)c5oc6ccccc6c45)c3)c3ccccc3-c3ccccc32)cc1 ; c1ccc(C2(c3cccc(N(c4ccccc4-c4ccc5sc6ccccc6c5c4)c4ccc(-c5cccc6ccccc56)c5oc6ccccc6c45)c3)c3ccccc3-c3ccccc32)cc1 ; c1ccc(C2(c3cccc(N(c4ccccc4-c4cccc5sc6ccccc6c45)c4ccc(-c5cccc6ccccc56)c5oc6ccccc6c45)c3)c3ccccc3-c3ccccc32)cc1]

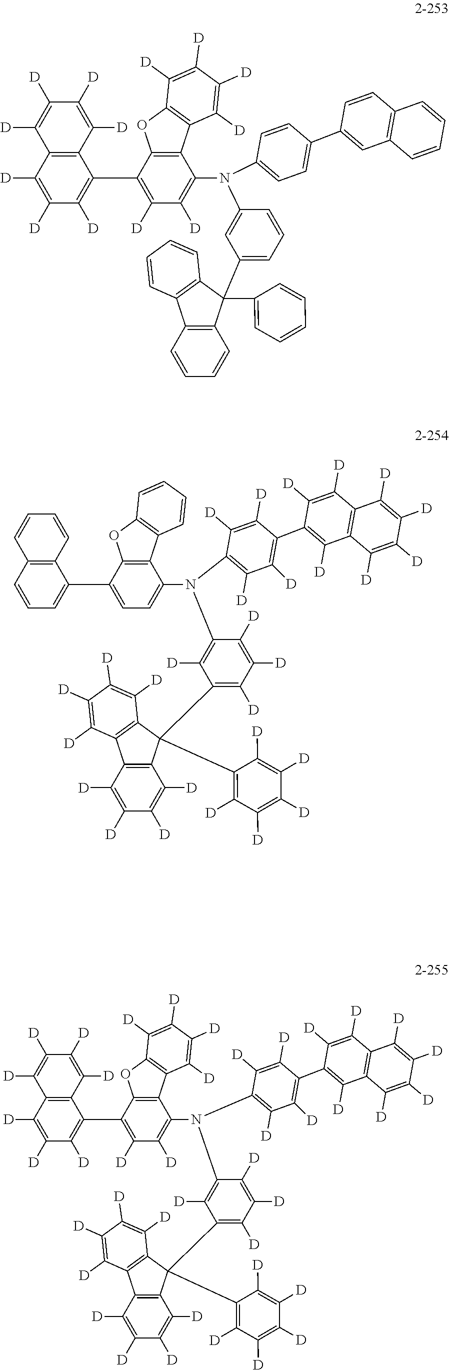

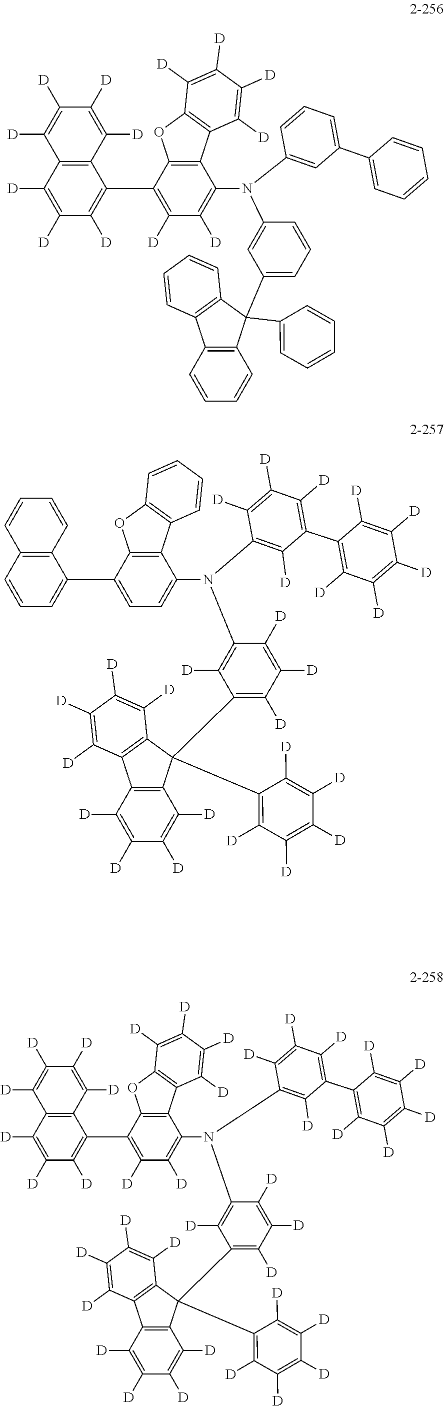

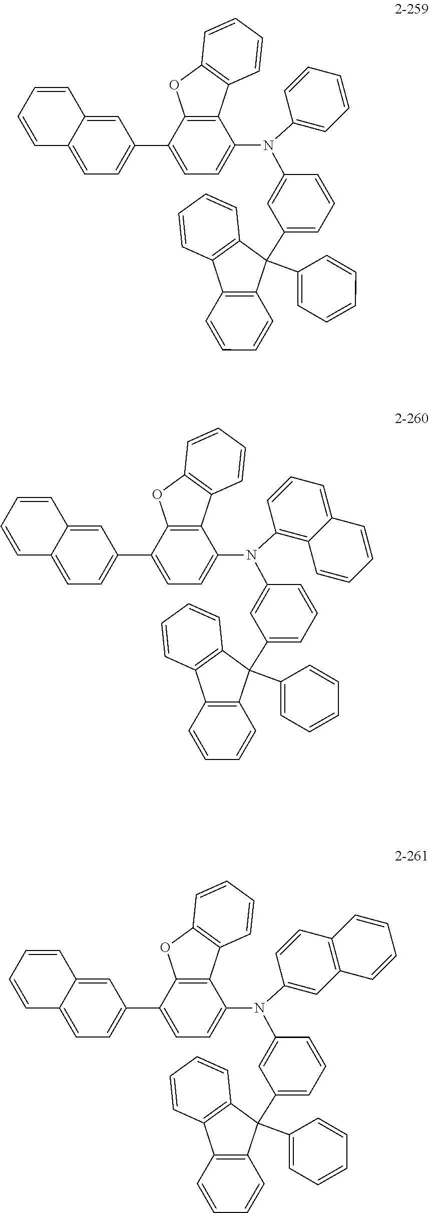 [SMILES: c1ccc(N(c2cccc(C3(c4ccccc4)c4ccccc4-c4ccccc43)c2)c2ccc(-c3ccc4ccccc4c3)c3oc4ccccc4c23)cc1 ; c1ccc(C2(c3cccc(N(c4cccc5ccccc45)c4ccc(-c5ccc6ccccc6c5)c5oc6ccccc6c45)c3)c3ccccc3-c3ccccc32)cc1 ; c1ccc(C2(c3cccc(N(c4ccc5ccccc5c4)c4ccc(-c5ccc6ccccc6c5)c5oc6ccccc6c45)c3)c3ccccc3-c3ccccc32)cc1]

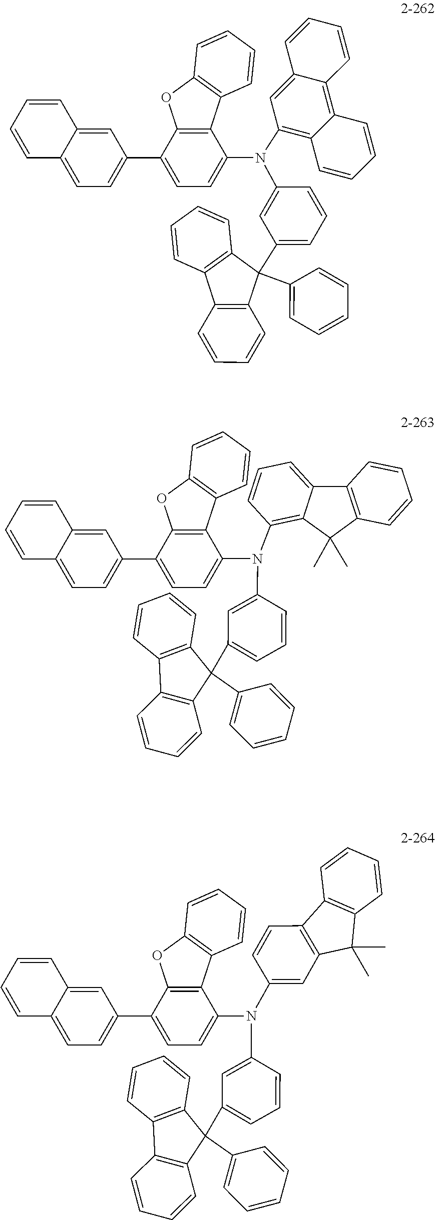 [SMILES: c1ccc(C2(c3cccc(N(c4cc5ccccc5c5ccccc45)c4ccc(-c5ccc6ccccc6c5)c5oc6ccccc6c45)c3)c3ccccc3-c3ccccc32)cc1 ; CC1(C)c2ccccc2-c2cccc(N(c3cccc(C4(c5ccccc5)c5ccccc5-c5ccccc54)c3)c3ccc(-c4ccc5ccccc5c4)c4oc5ccccc5c34)c21 ; CC1(C)c2ccccc2-c2ccc(N(c3cccc(C4(c5ccccc5)c5ccccc5-c5ccccc54)c3)c3ccc(-c4ccc5ccccc5c4)c4oc5ccccc5c34)cc21]

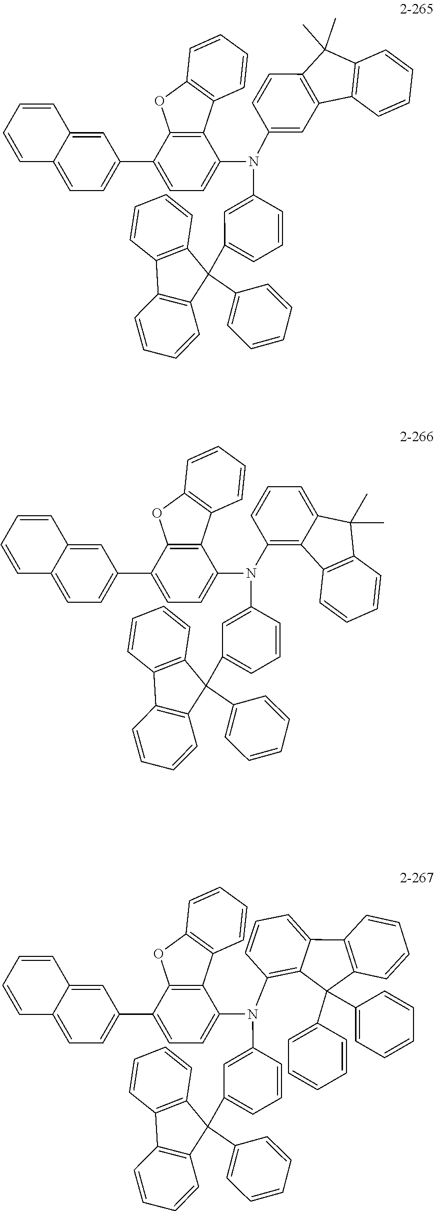 [SMILES: CC1(C)c2ccccc2-c2cc(N(c3cccc(C4(c5ccccc5)c5ccccc5-c5ccccc54)c3)c3ccc(-c4ccc5ccccc5c4)c4oc5ccccc5c34)ccc21 ; CC1(C)c2ccccc2-c2c(N(c3cccc(C4(c5ccccc5)c5ccccc5-c5ccccc54)c3)c3ccc(-c4ccc5ccccc5c4)c4oc5ccccc5c34)cccc21 ; c1ccc(C2(c3cccc(N(c4cccc5c4C(c4ccccc4)(c4ccccc4)c4ccccc4-5)c4ccc(-c5ccc6ccccc6c5)c5oc6ccccc6c45)c3)c3ccccc3-c3ccccc32)cc1]

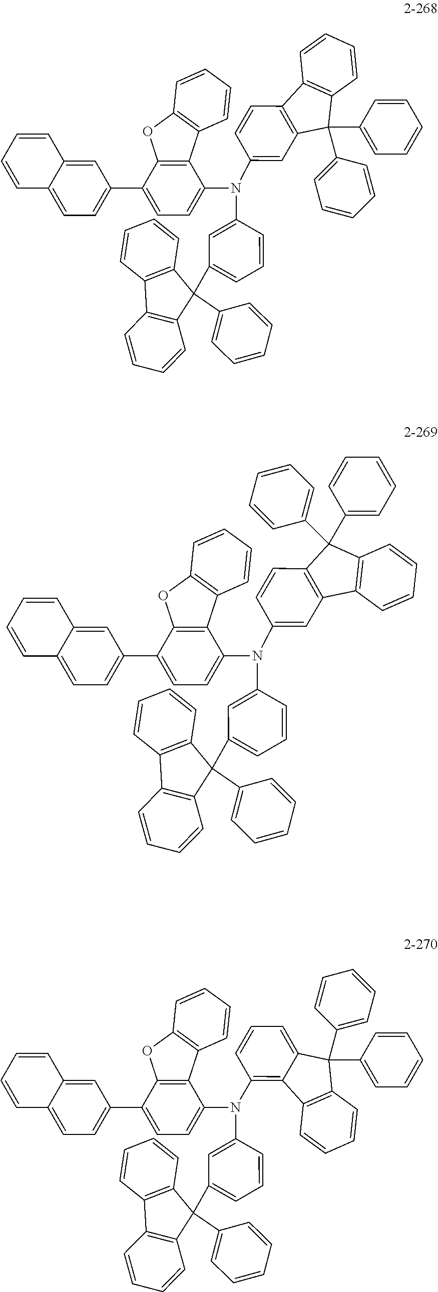 [SMILES: c1ccc(C2(c3cccc(N(c4ccc5c(c4)C(c4ccccc4)(c4ccccc4)c4ccccc4-5)c4ccc(-c5ccc6ccccc6c5)c5oc6ccccc6c45)c3)c3ccccc3-c3ccccc32)cc1 ; c1ccc(C2(c3cccc(N(c4ccc5c(c4)-c4ccccc4C5(c4ccccc4)c4ccccc4)c4ccc(-c5ccc6ccccc6c5)c5oc6ccccc6c45)c3)c3ccccc3-c3ccccc32)cc1 ; c1ccc(C2(c3cccc(N(c4cccc5c4-c4ccccc4C5(c4ccccc4)c4ccccc4)c4ccc(-c5ccc6ccccc6c5)c5oc6ccccc6c45)c3)c3ccccc3-c3ccccc32)cc1]

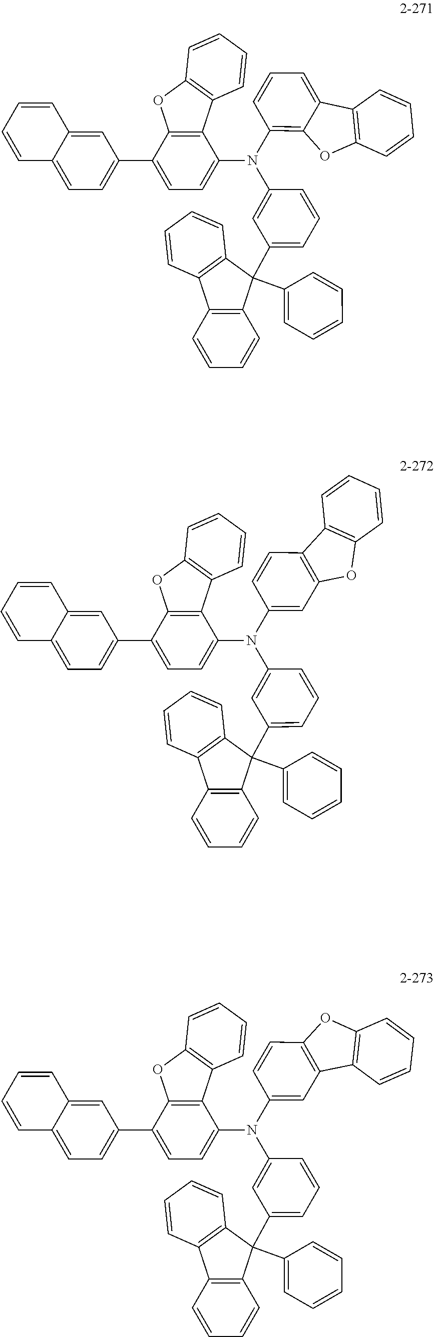 [SMILES: c1ccc(C2(c3cccc(N(c4cccc5c4oc4ccccc45)c4ccc(-c5ccc6ccccc6c5)c5oc6ccccc6c45)c3)c3ccccc3-c3ccccc32)cc1 ; c1ccc(C2(c3cccc(N(c4ccc5c(c4)oc4ccccc45)c4ccc(-c5ccc6ccccc6c5)c5oc6ccccc6c45)c3)c3ccccc3-c3ccccc32)cc1 ; c1ccc(C2(c3cccc(N(c4ccc5oc6ccccc6c5c4)c4ccc(-c5ccc6ccccc6c5)c5oc6ccccc6c45)c3)c3ccccc3-c3ccccc32)cc1]

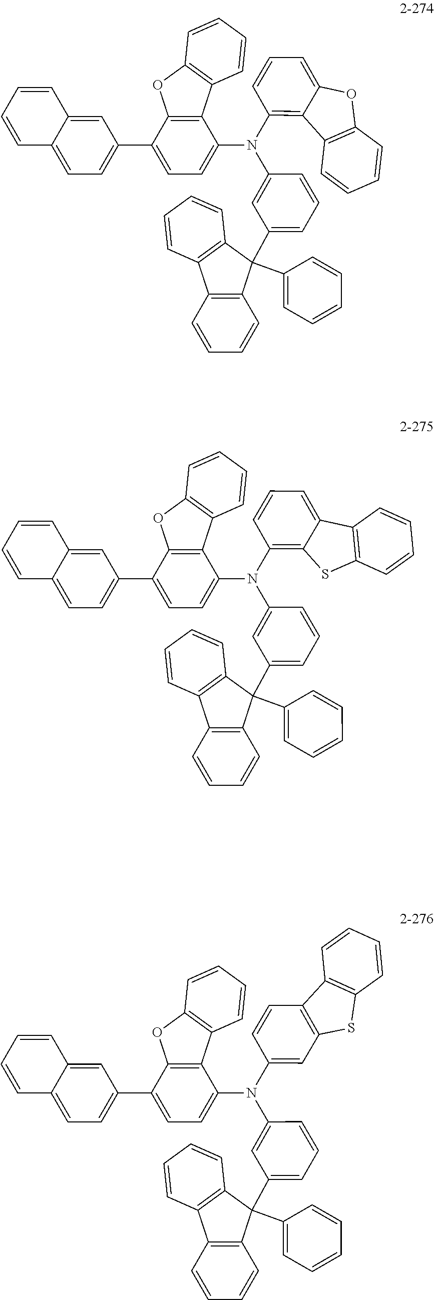 [SMILES: c1ccc(C2(c3cccc(N(c4cccc5oc6ccccc6c45)c4ccc(-c5ccc6ccccc6c5)c5oc6ccccc6c45)c3)c3ccccc3-c3ccccc32)cc1 ; c1ccc(C2(c3cccc(N(c4cccc5c4sc4ccccc45)c4ccc(-c5ccc6ccccc6c5)c5oc6ccccc6c45)c3)c3ccccc3-c3ccccc32)cc1 ; c1ccc(C2(c3cccc(N(c4ccc5c(c4)sc4ccccc45)c4ccc(-c5ccc6ccccc6c5)c5oc6ccccc6c45)c3)c3ccccc3-c3ccccc32)cc1]

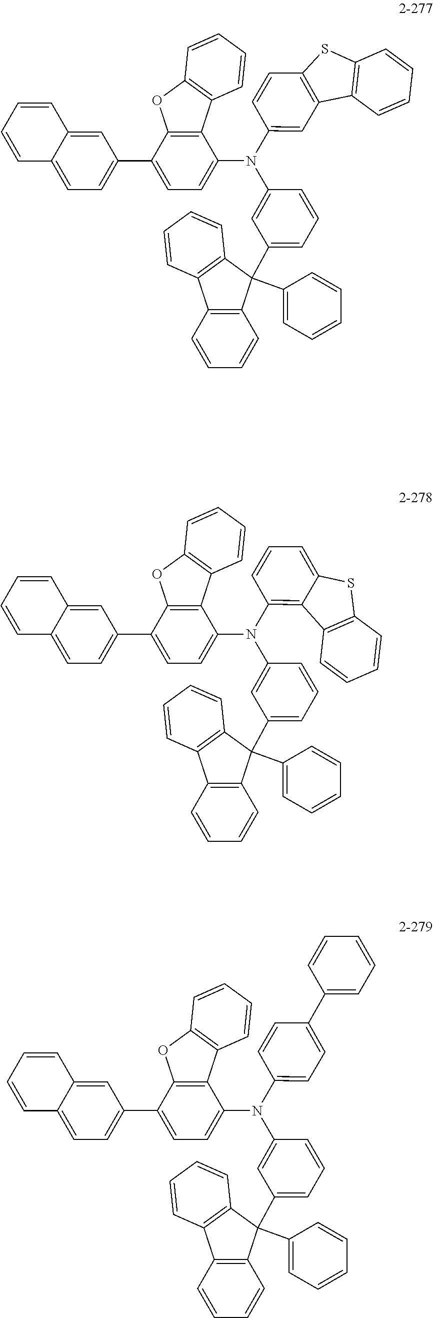 [SMILES: c1ccc(C2(c3cccc(N(c4ccc5sc6ccccc6c5c4)c4ccc(-c5ccc6ccccc6c5)c5oc6ccccc6c45)c3)c3ccccc3-c3ccccc32)cc1 ; c1ccc(C2(c3cccc(N(c4cccc5sc6ccccc6c45)c4ccc(-c5ccc6ccccc6c5)c5oc6ccccc6c45)c3)c3ccccc3-c3ccccc32)cc1 ; c1ccc(-c2ccc(N(c3cccc(C4(c5ccccc5)c5ccccc5-c5ccccc54)c3)c3ccc(-c4ccc5ccccc5c4)c4oc5ccccc5c34)cc2)cc1]

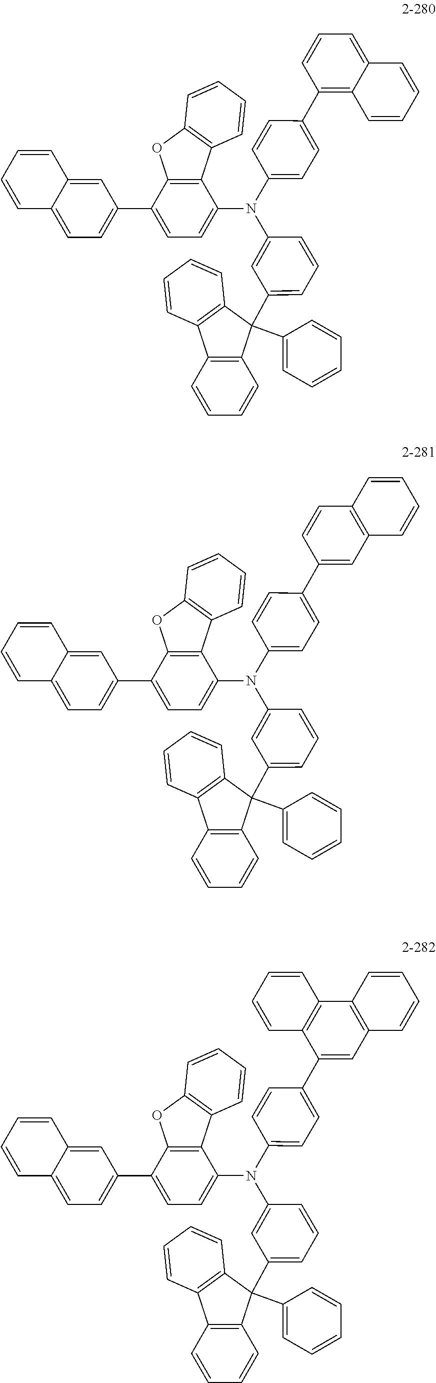 [SMILES: c1ccc(C2(c3cccc(N(c4ccc(-c5cccc6ccccc56)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5ccc6ccccc6c5)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5cc6ccccc6c6ccccc56)cc4)c4ccc(-c5ccc6ccccc6c5)c5oc6ccccc6c45)c3)c3ccccc3-c3ccccc32)cc1]

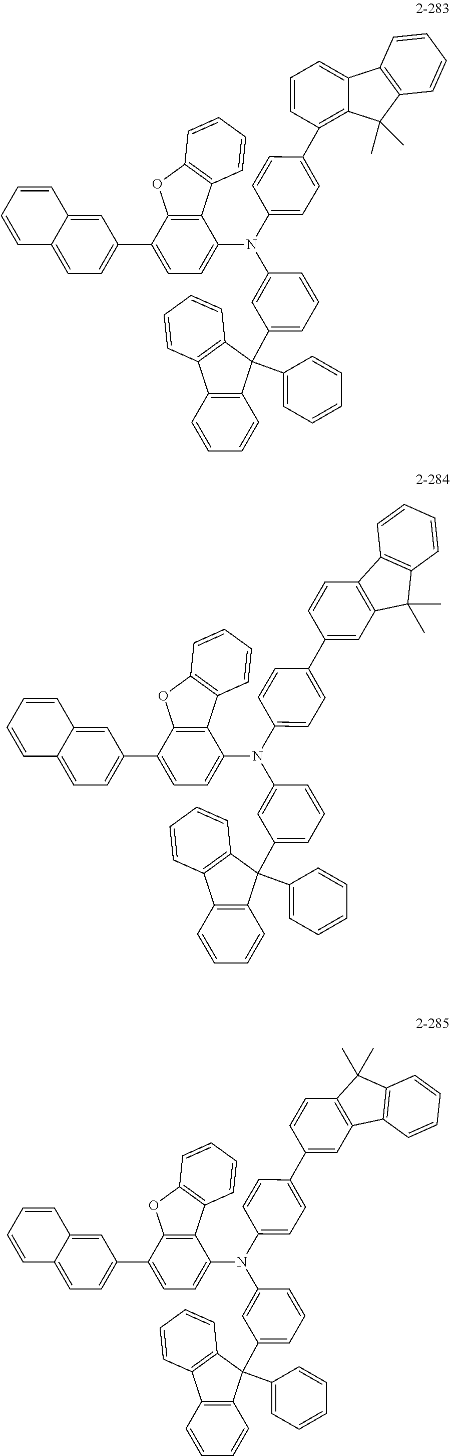 [SMILES: CC1(C)c2ccccc2-c2cccc(-c3ccc(N(c4cccc(C5(c6ccccc6)c6ccccc6-c6ccccc65)c4)c4ccc(-c5ccc6ccccc6c5)c5oc6ccccc6c45)cc3)c21 ; CC1(C)c2ccccc2-c2ccc(-c3ccc(N(c4cccc(C5(c6ccccc6)c6ccccc6-c6ccccc65)c4)c4ccc(-c5ccc6ccccc6c5)c5oc6ccccc6c45)cc3)cc21 ; CC1(C)c2ccccc2-c2cc(-c3ccc(N(c4cccc(C5(c6ccccc6)c6ccccc6-c6ccccc65)c4)c4ccc(-c5ccc6ccccc6c5)c5oc6ccccc6c45)cc3)ccc21]

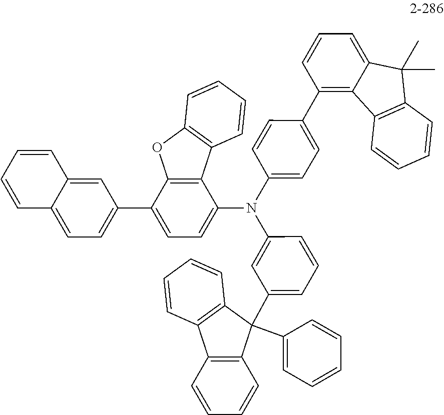 [SMILES: CC1(C)c2ccccc2-c2c(-c3ccc(N(c4cccc(C5(c6ccccc6)c6ccccc6-c6ccccc65)c4)c4ccc(-c5ccc6ccccc6c5)c5oc6ccccc6c45)cc3)cccc21]

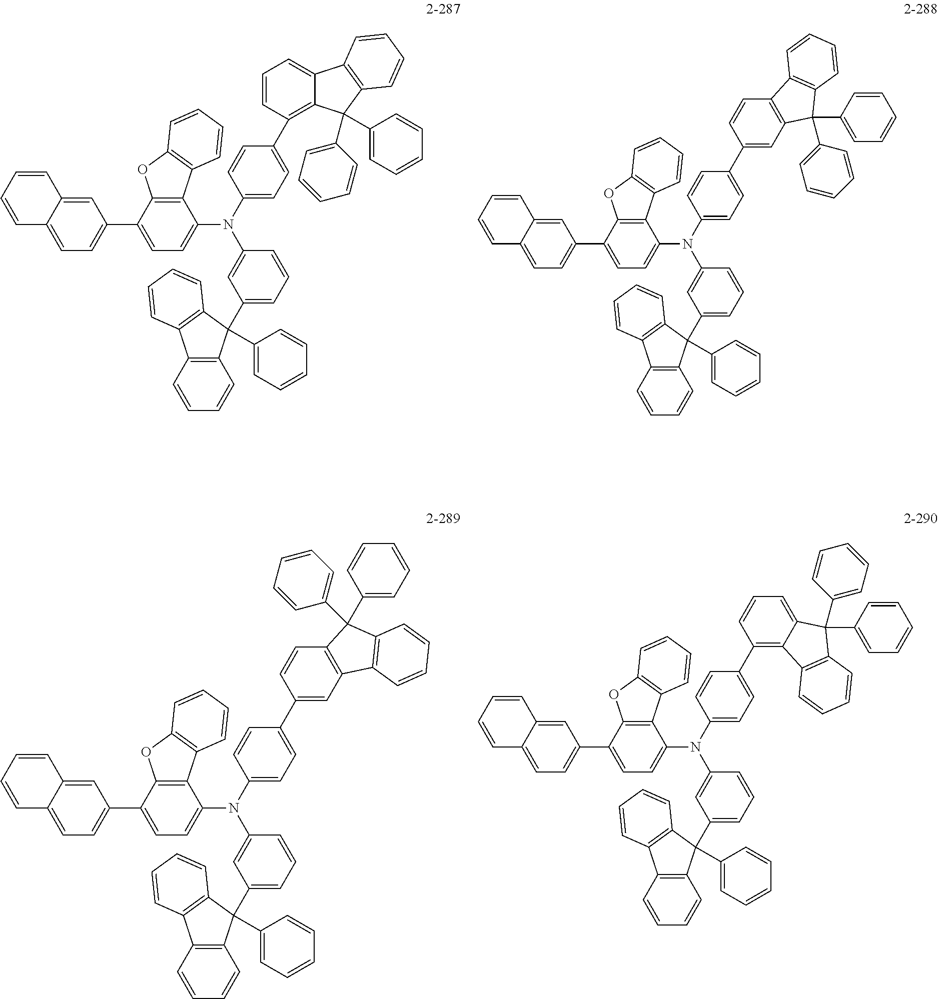

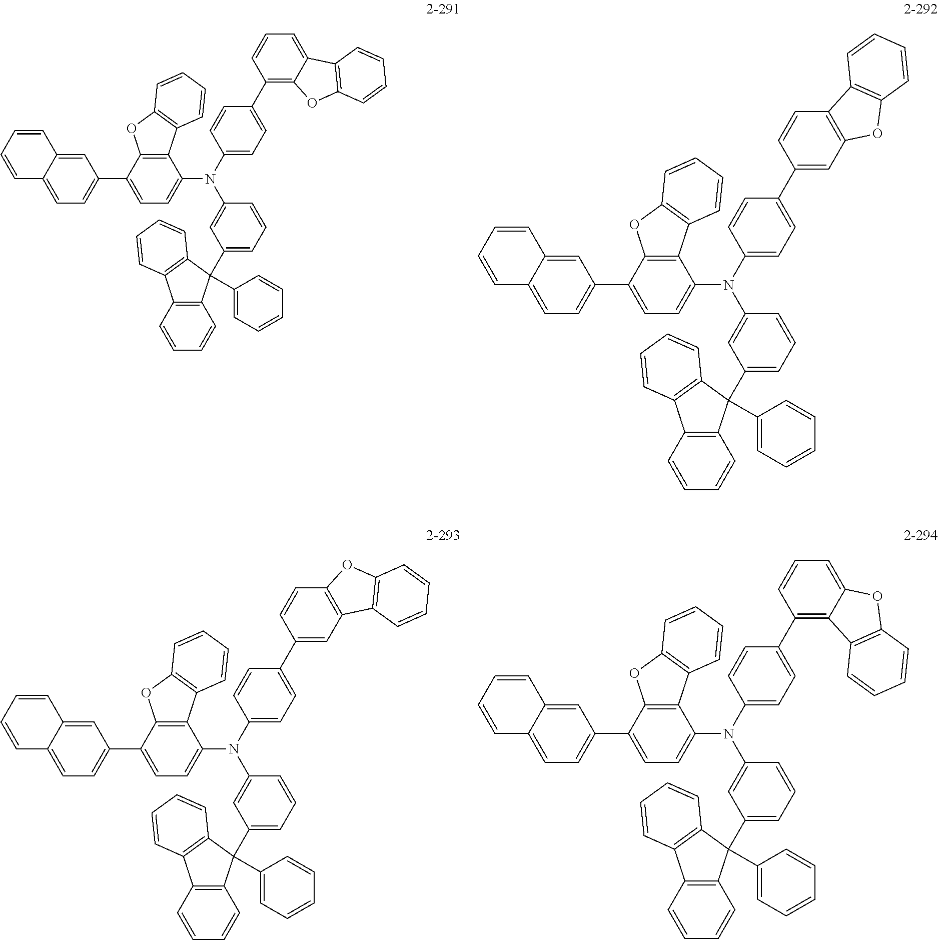 [SMILES: c1ccc(C2(c3cccc(N(c4ccc(-c5cccc6c5oc5ccccc56)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5ccc6c(c5)oc5ccccc56)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5ccc6oc7ccccc7c6c5)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5cccc6oc7ccccc7c56)cc4)c4ccc(-c5ccc6ccccc6c5)c5oc6ccccc6c45)c3)c3ccccc3-c3ccccc32)cc1]

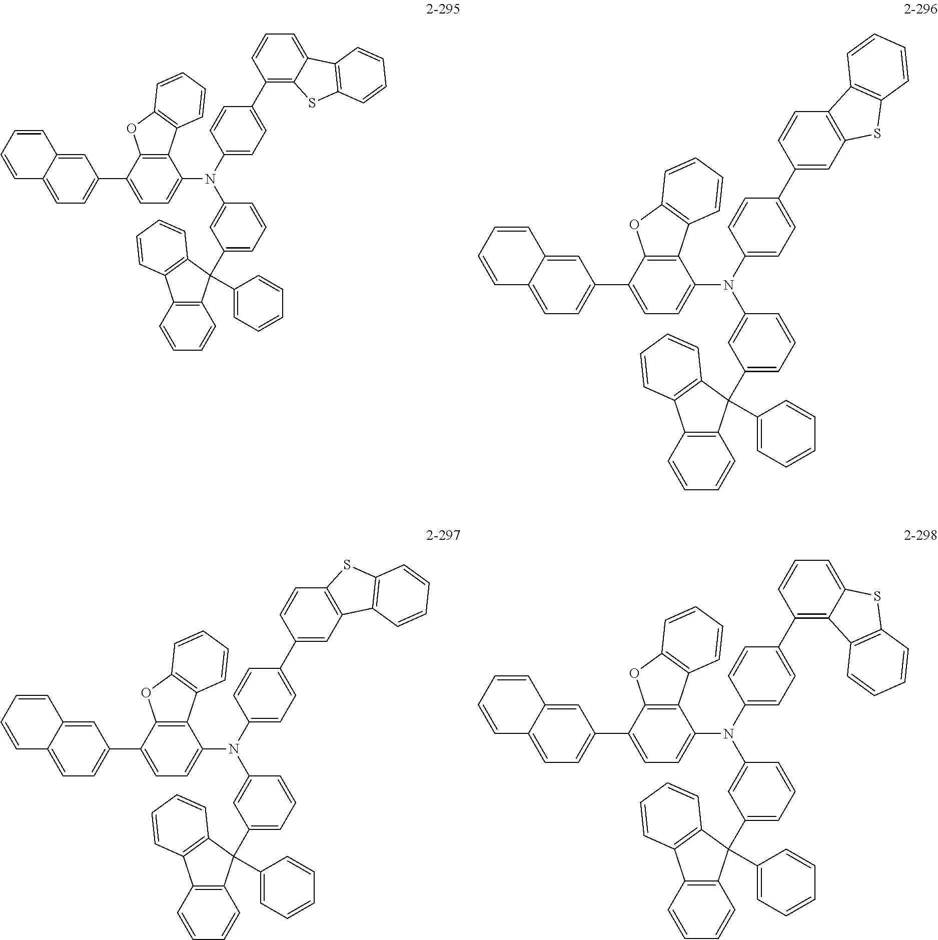 [SMILES: c1ccc(C2(c3cccc(N(c4ccc(-c5cccc6c5sc5ccccc56)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5ccc6c(c5)sc5ccccc56)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5ccc6sc7ccccc7c6c5)cc4)c4ccc(-c5ccc6ccccc6c5)c5oc6ccccc6c45)c3)c3ccccc3-c3ccccc32)cc1 ; c1ccc(C2(c3cccc(N(c4ccc(-c5cccc6sc7ccccc7c56)cc4)c4ccc(-c5ccc6ccccc6c5)c5oc6ccccc6c45)c3)c3ccccc3-c3ccccc32)cc1]

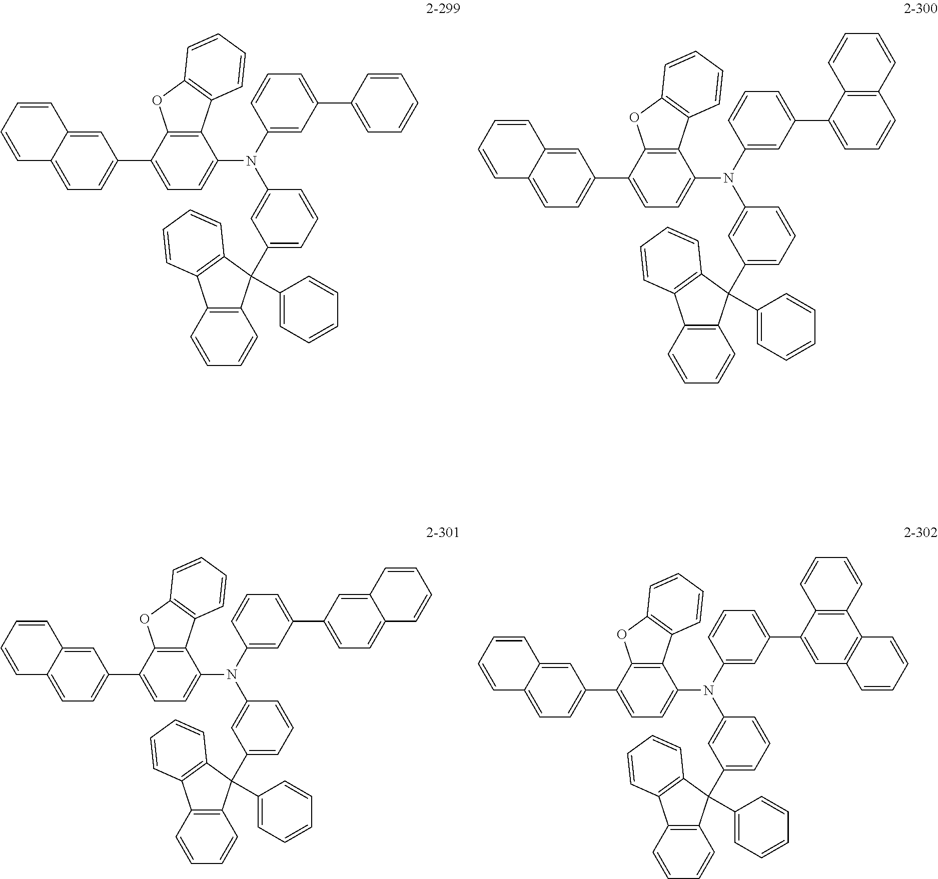 [SMILES: c1ccc(-c2cccc(N(c3cccc(C4(c5ccccc5)c5ccccc5-c5ccccc54)c3)c3ccc(-c4ccc5ccccc5c4)c4oc5ccccc5c34)c2)cc1 ; c1ccc(C2(c3cccc(N(c4cccc(-c5cccc6ccccc56)c4)c4ccc(-c5ccc6ccccc6c5)c5oc6ccccc6c45)c3)c3ccccc3-c3ccccc32)cc1 ; c1ccc(C2(c3cccc(N(c4cccc(-c5ccc6ccccc6c5)c4)c4ccc(-c5ccc6ccccc6c5)c5oc6ccccc6c45)c3)c3ccccc3-c3ccccc32)cc1 ; c1ccc(C2(c3cccc(N(c4cccc(-c5cc6ccccc6c6ccccc56)c4)c4ccc(-c5ccc6ccccc6c5)c5oc6ccccc6c45)c3)c3ccccc3-c3ccccc32)cc1]

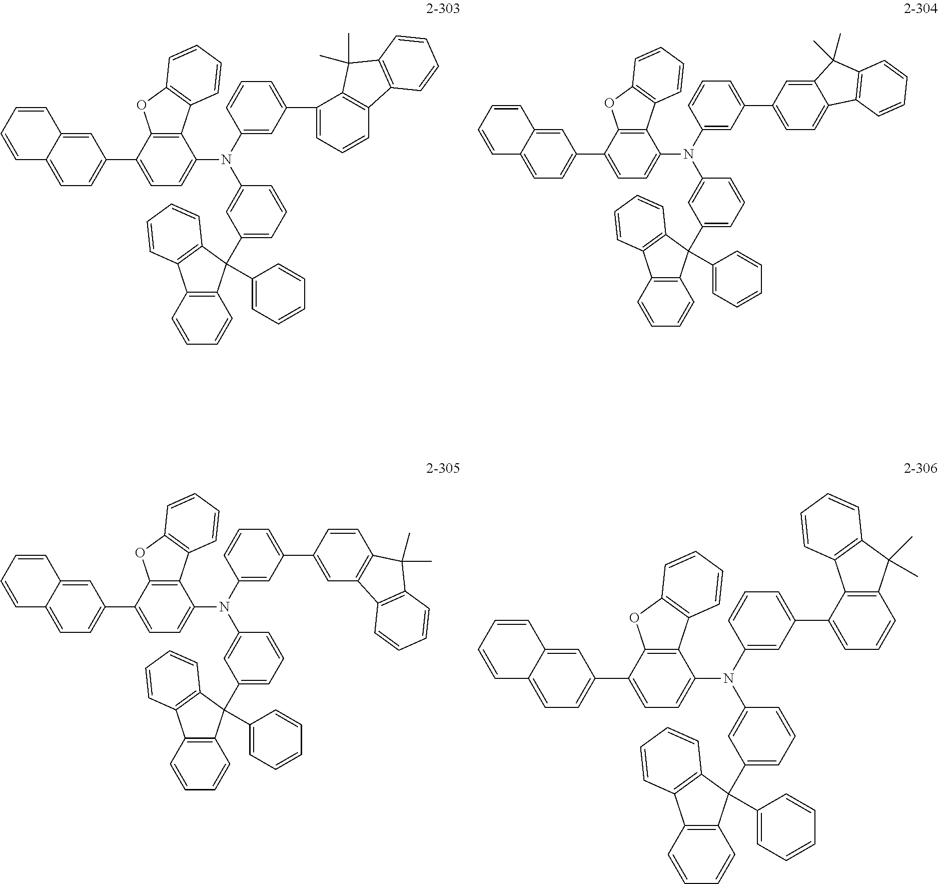 [SMILES: CC1(C)c2ccccc2-c2cccc(-c3cccc(N(c4cccc(C5(c6ccccc6)c6ccccc6-c6ccccc65)c4)c4ccc(-c5ccc6ccccc6c5)c5oc6ccccc6c45)c3)c21 ; CC1(C)c2ccccc2-c2ccc(-c3cccc(N(c4cccc(C5(c6ccccc6)c6ccccc6-c6ccccc65)c4)c4ccc(-c5ccc6ccccc6c5)c5oc6ccccc6c45)c3)cc21 ; CC1(C)c2ccccc2-c2cc(-c3cccc(N(c4cccc(C5(c6ccccc6)c6ccccc6-c6ccccc65)c4)c4ccc(-c5ccc6ccccc6c5)c5oc6ccccc6c45)c3)ccc21 ; CC1(C)c2ccccc2-c2c(-c3cccc(N(c4cccc(C5(c6ccccc6)c6ccccc6-c6ccccc65)c4)c4ccc(-c5ccc6ccccc6c5)c5oc6ccccc6c45)c3)cccc21]

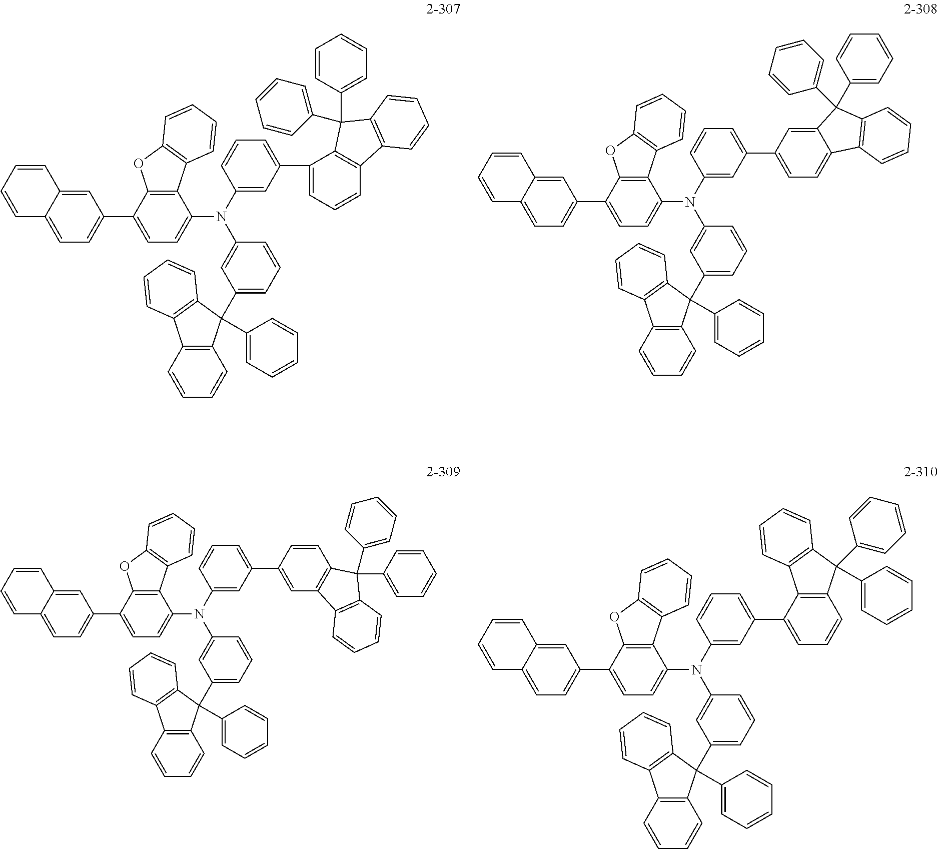

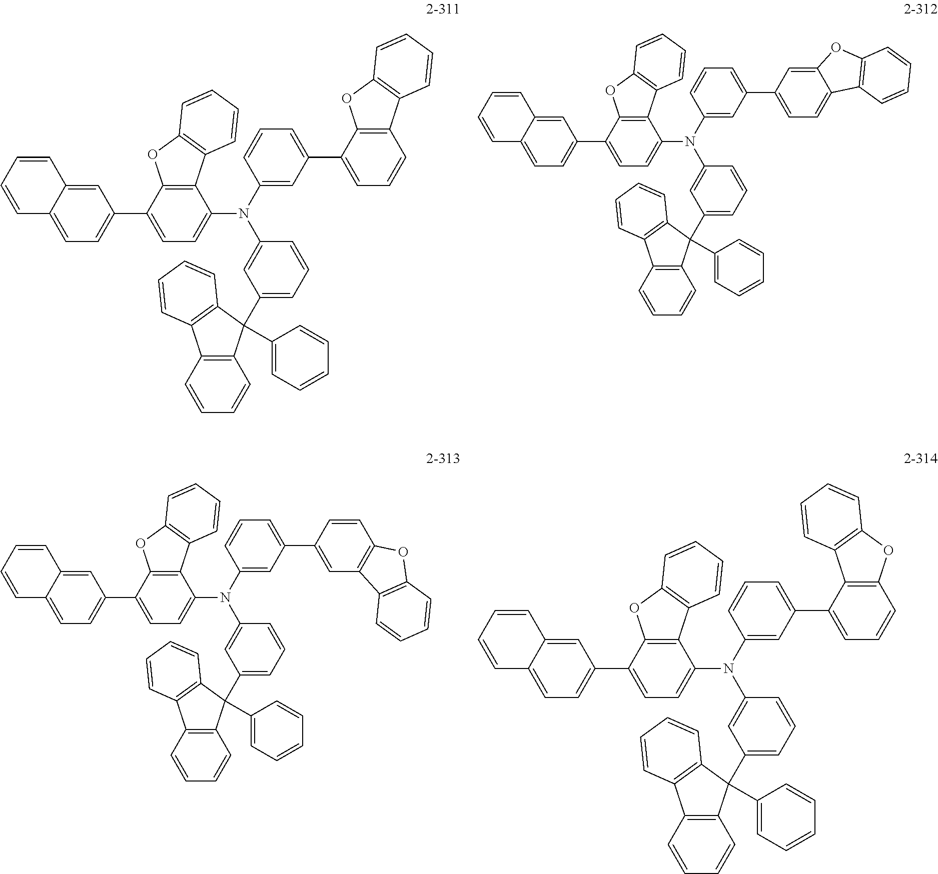 [SMILES: c1ccc(C2(c3cccc(N(c4cccc(-c5cccc6c5oc5ccccc56)c4)c4ccc(-c5ccc6ccccc6c5)c5oc6ccccc6c45)c3)c3ccccc3-c3ccccc32)cc1 ; c1ccc(C2(c3cccc(N(c4cccc(-c5ccc6c(c5)oc5ccccc56)c4)c4ccc(-c5ccc6ccccc6c5)c5oc6ccccc6c45)c3)c3ccccc3-c3ccccc32)cc1 ; c1ccc(C2(c3cccc(N(c4cccc(-c5ccc6oc7ccccc7c6c5)c4)c4ccc(-c5ccc6ccccc6c5)c5oc6ccccc6c45)c3)c3ccccc3-c3ccccc32)cc1 ; c1ccc(C2(c3cccc(N(c4cccc(-c5cccc6oc7ccccc7c56)c4)c4ccc(-c5ccc6ccccc6c5)c5oc6ccccc6c45)c3)c3ccccc3-c3ccccc32)cc1]

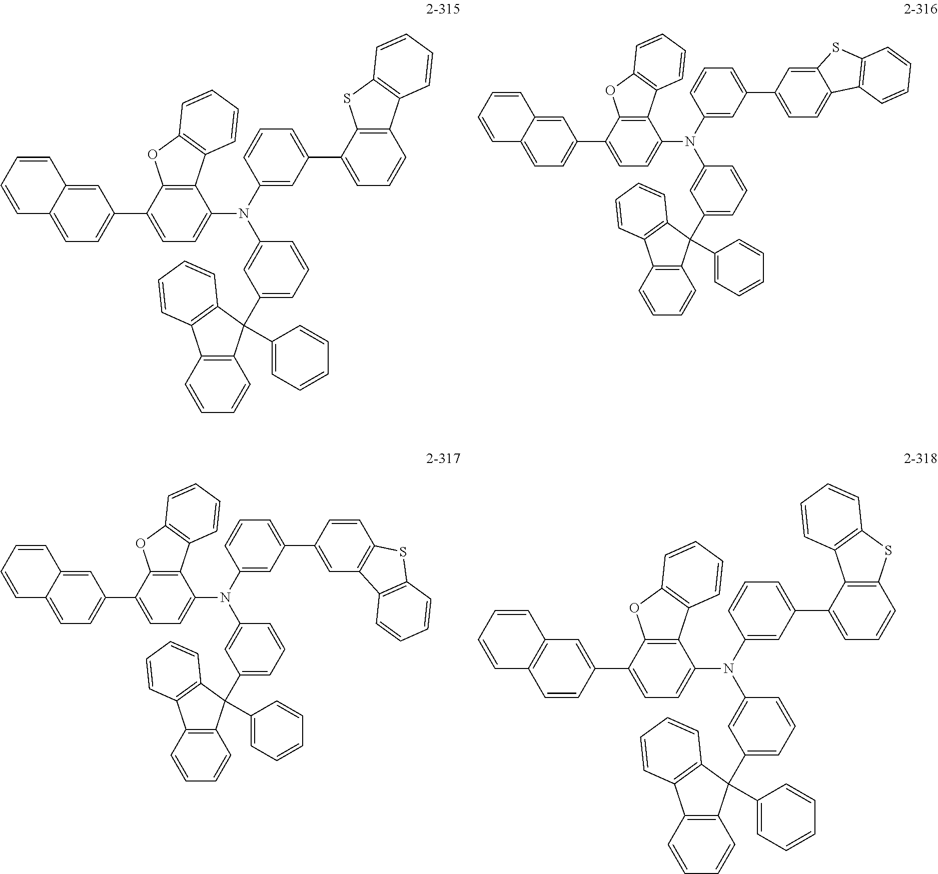 [SMILES: c1ccc(C2(c3cccc(N(c4cccc(-c5cccc6c5sc5ccccc56)c4)c4ccc(-c5ccc6ccccc6c5)c5oc6ccccc6c45)c3)c3ccccc3-c3ccccc32)cc1 ; c1ccc(C2(c3cccc(N(c4cccc(-c5ccc6c(c5)sc5ccccc56)c4)c4ccc(-c5ccc6ccccc6c5)c5oc6ccccc6c45)c3)c3ccccc3-c3ccccc32)cc1 ; c1ccc(C2(c3cccc(N(c4cccc(-c5ccc6sc7ccccc7c6c5)c4)c4ccc(-c5ccc6ccccc6c5)c5oc6ccccc6c45)c3)c3ccccc3-c3ccccc32)cc1 ; c1ccc(C2(c3cccc(N(c4cccc(-c5cccc6sc7ccccc7c56)c4)c4ccc(-c5ccc6ccccc6c5)c5oc6ccccc6c45)c3)c3ccccc3-c3ccccc32)cc1]

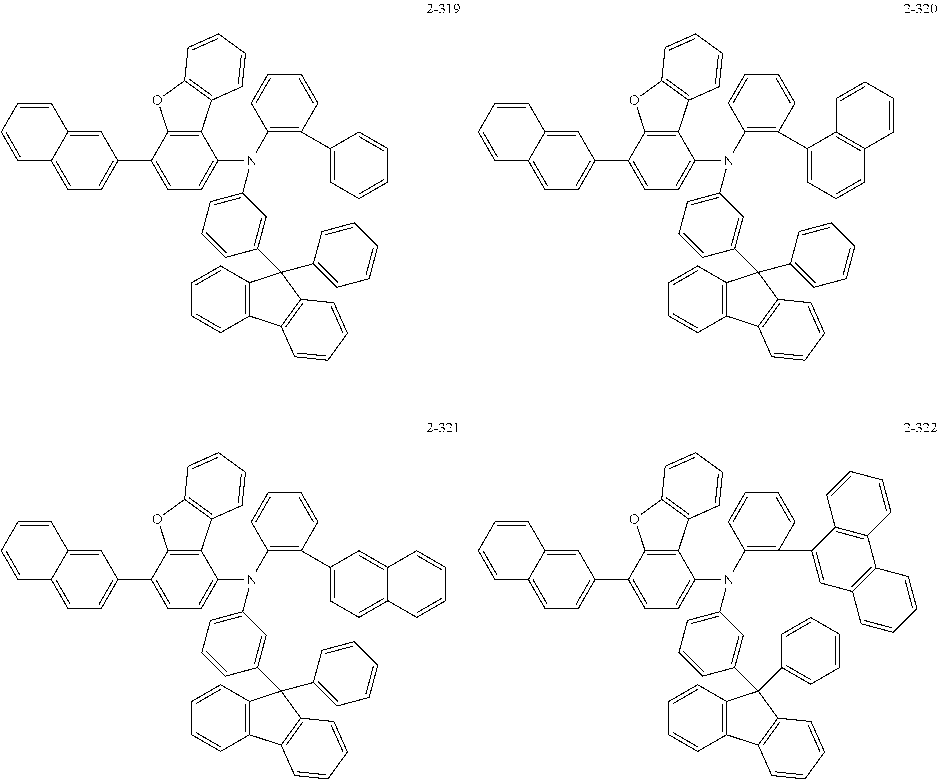

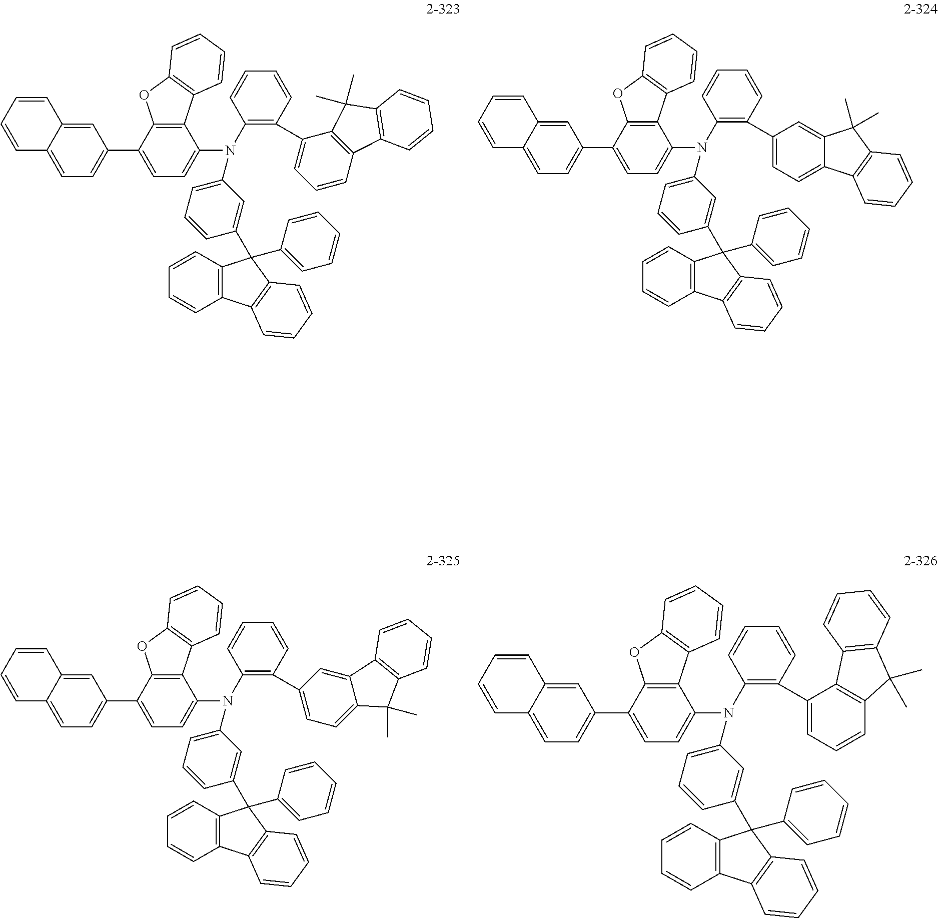 [SMILES: CC1(C)c2ccccc2-c2cccc(-c3ccccc3N(c3cccc(C4(c5ccccc5)c5ccccc5-c5ccccc54)c3)c3ccc(-c4ccc5ccccc5c4)c4oc5ccccc5c34)c21 ; CC1(C)c2ccccc2-c2ccc(-c3ccccc3N(c3cccc(C4(c5ccccc5)c5ccccc5-c5ccccc54)c3)c3ccc(-c4ccc5ccccc5c4)c4oc5ccccc5c34)cc21 ; CC1(C)c2ccccc2-c2cc(-c3ccccc3N(c3cccc(C4(c5ccccc5)c5ccccc5-c5ccccc54)c3)c3ccc(-c4ccc5ccccc5c4)c4oc5ccccc5c34)ccc21 ; CC1(C)c2ccccc2-c2c(-c3ccccc3N(c3cccc(C4(c5ccccc5)c5ccccc5-c5ccccc54)c3)c3ccc(-c4ccc5ccccc5c4)c4oc5ccccc5c34)cccc21]

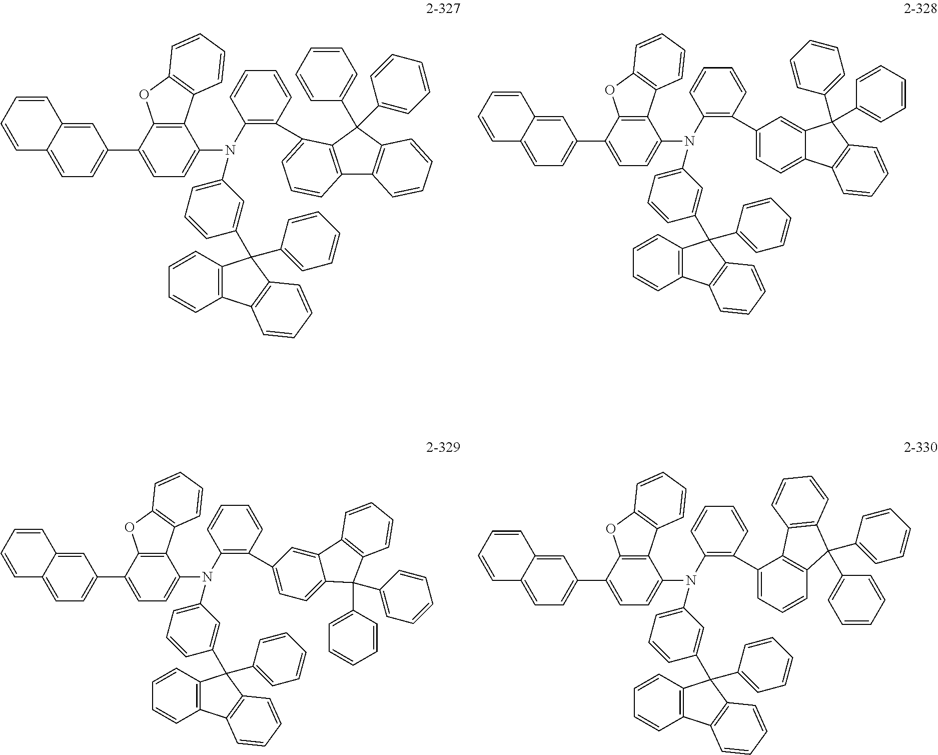

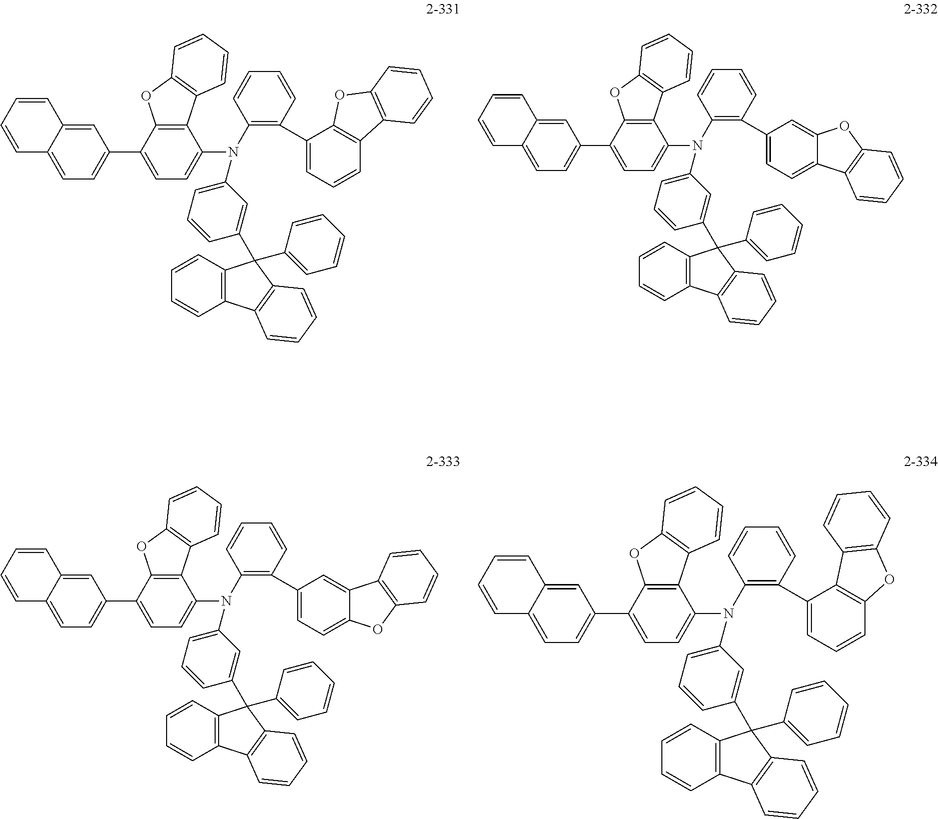

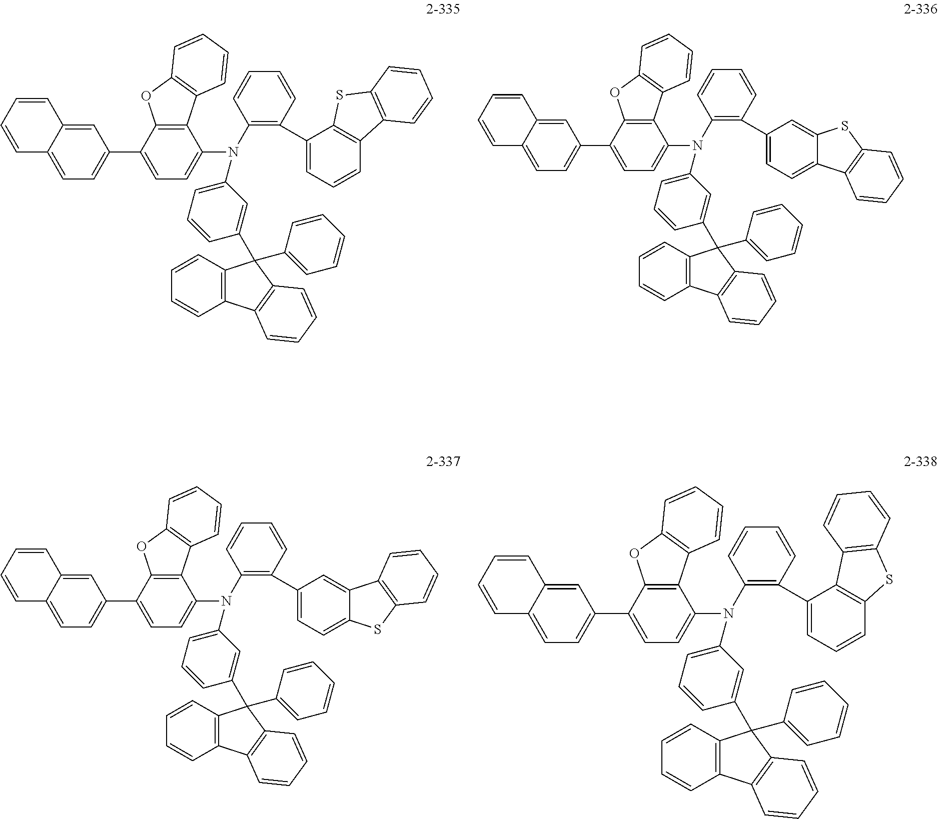

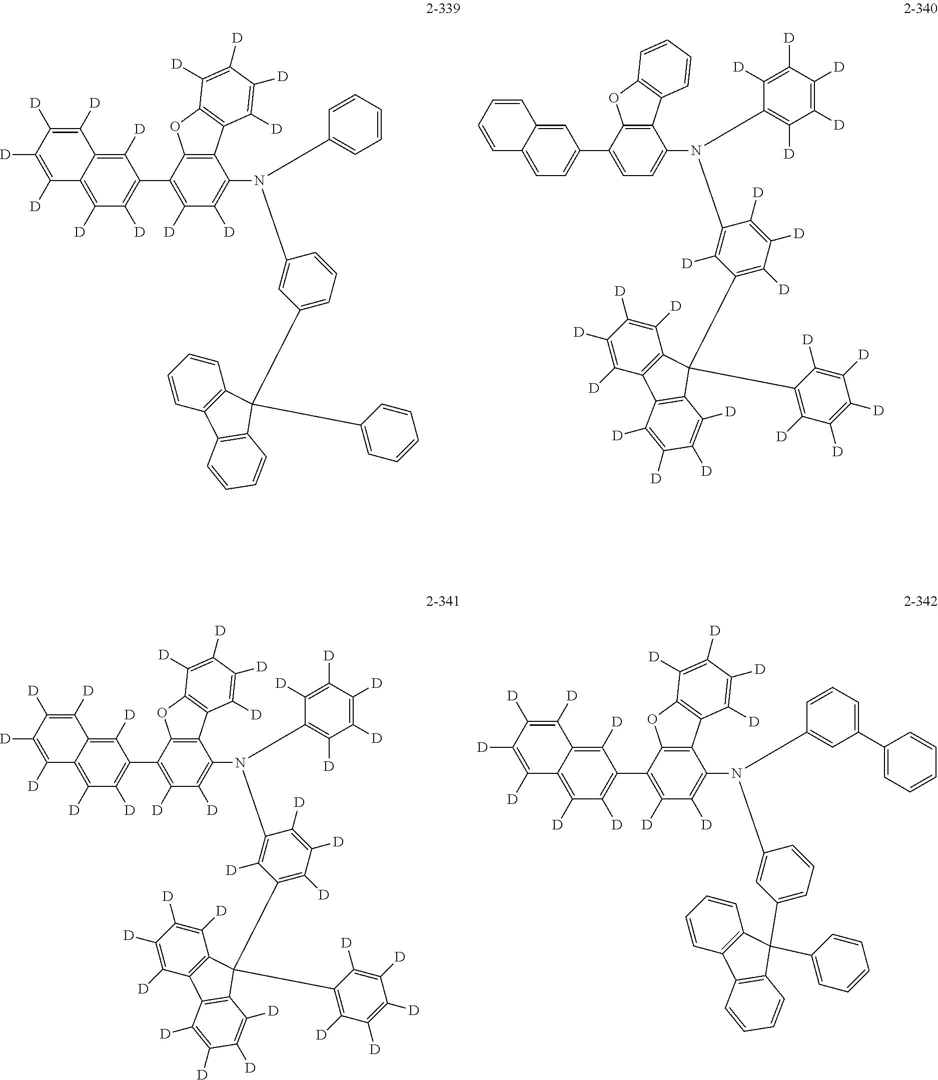

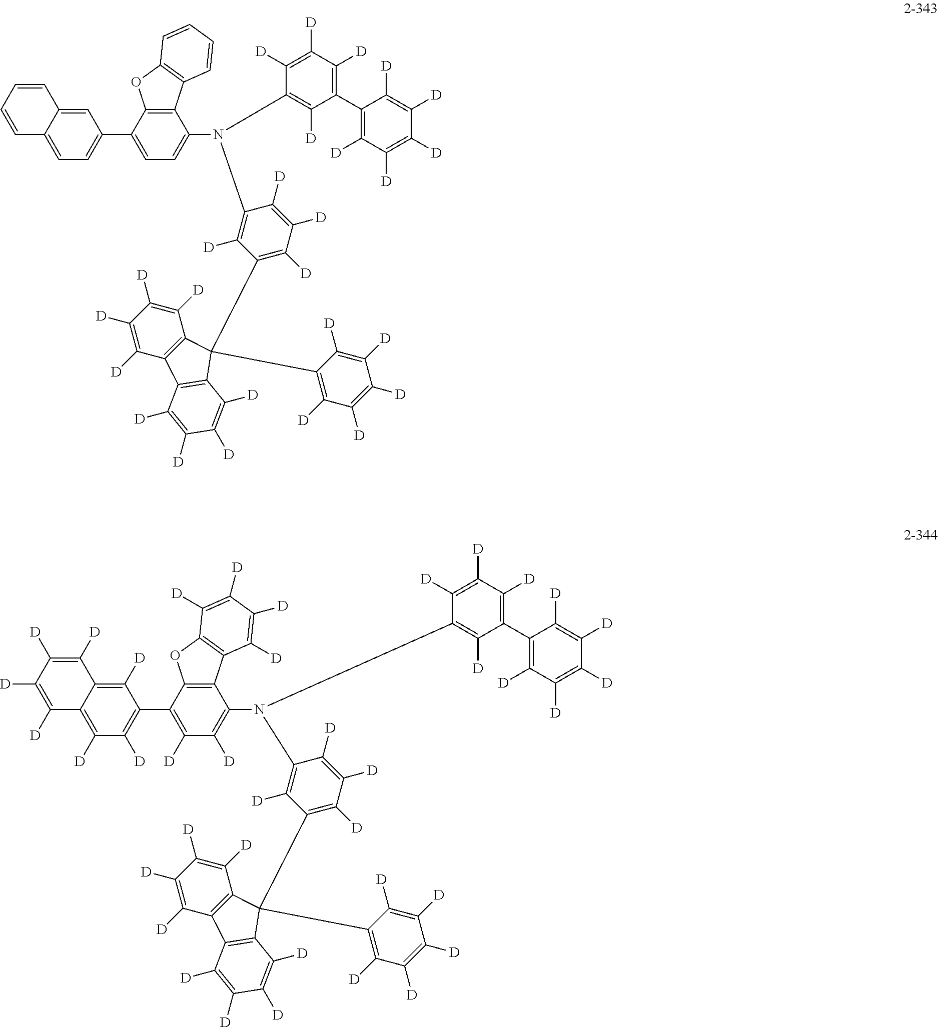

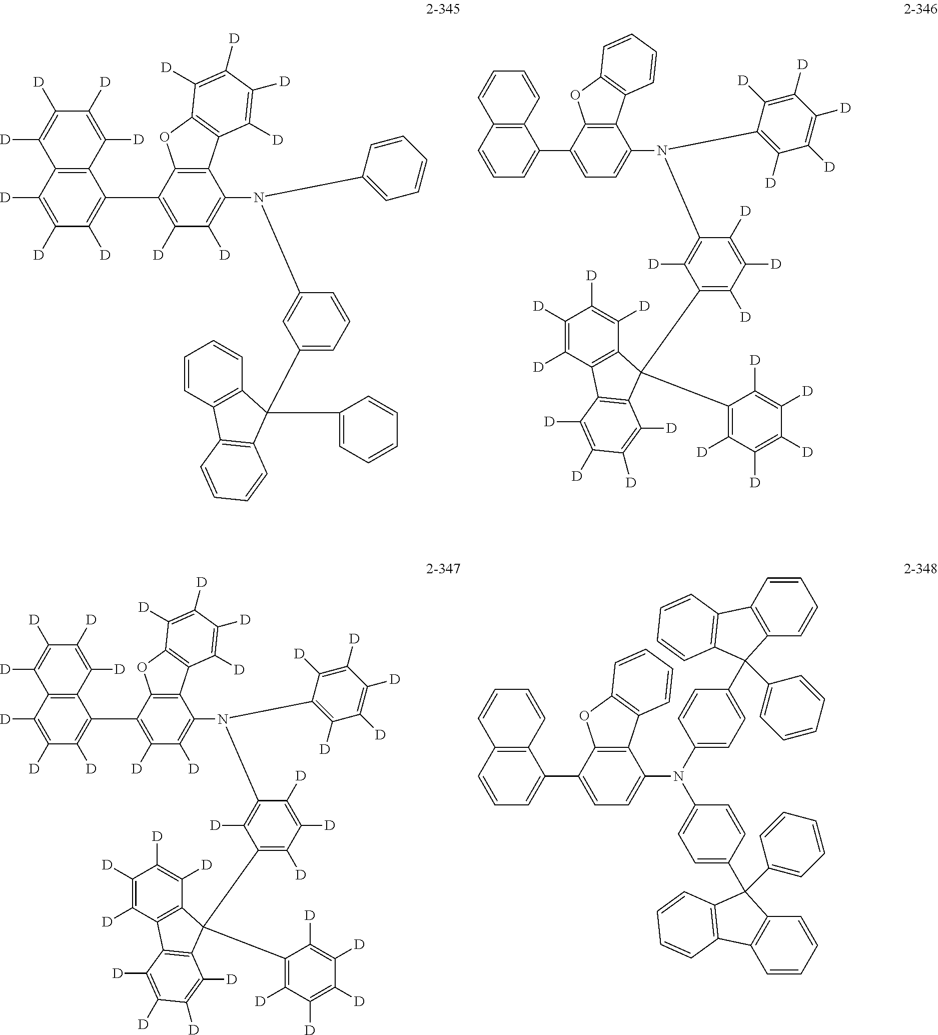

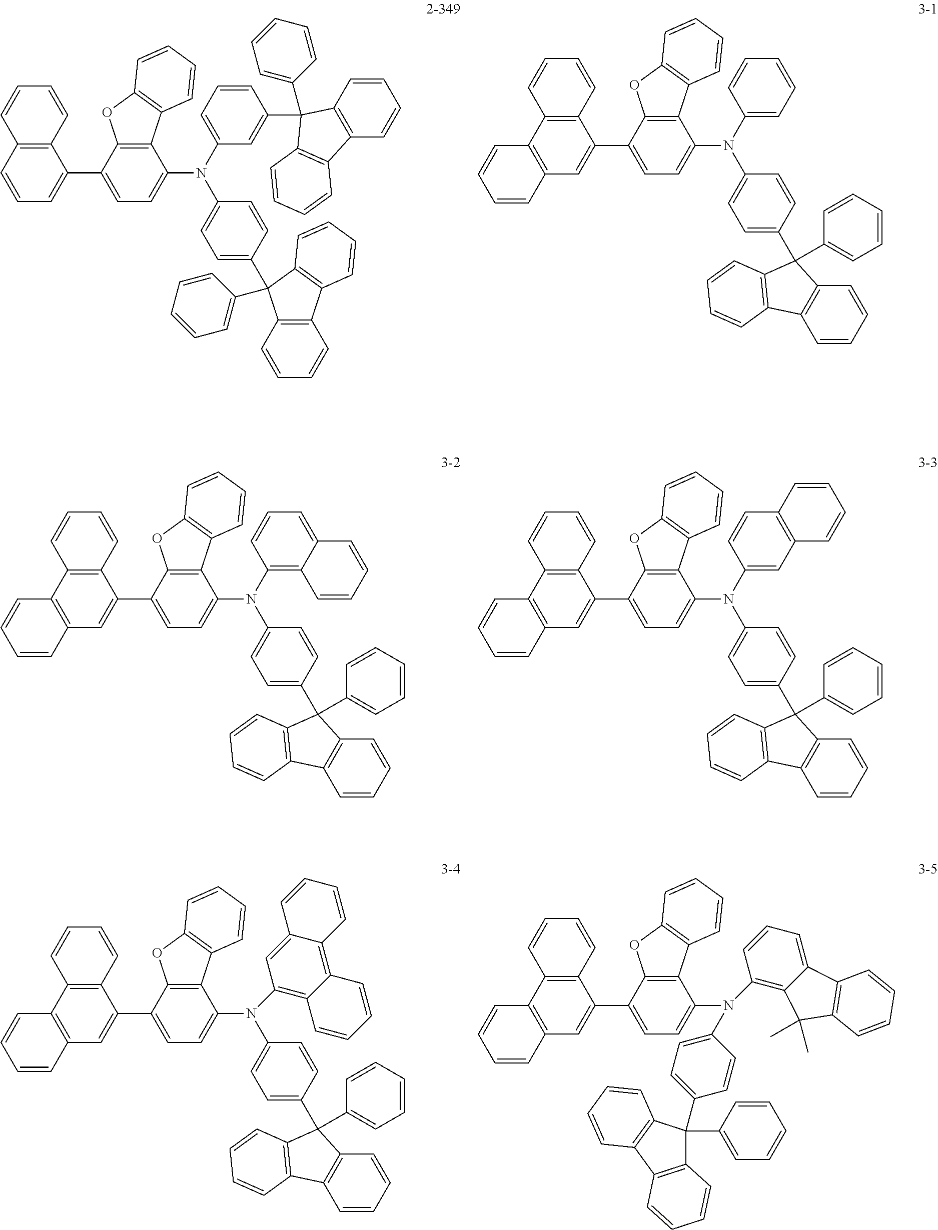

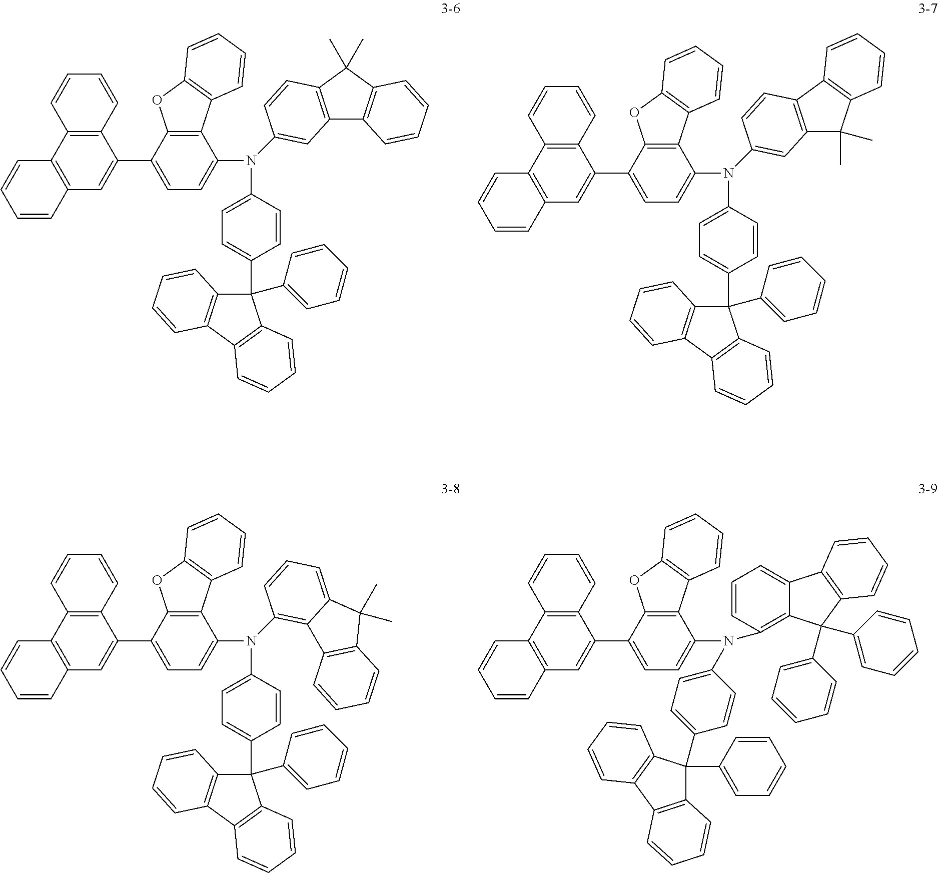 [SMILES: CC1(C)c2ccccc2-c2cc(N(c3ccc(C4(c5ccccc5)c5ccccc5-c5ccccc54)cc3)c3ccc(-c4cc5ccccc5c5ccccc45)c4oc5ccccc5c34)ccc21 ; CC1(C)c2ccccc2-c2ccc(N(c3ccc(C4(c5ccccc5)c5ccccc5-c5ccccc54)cc3)c3ccc(-c4cc5ccccc5c5ccccc45)c4oc5ccccc5c34)cc21 ; CC1(C)c2ccccc2-c2c(N(c3ccc(C4(c5ccccc5)c5ccccc5-c5ccccc54)cc3)c3ccc(-c4cc5ccccc5c5ccccc45)c4oc5ccccc5c34)cccc21 ; c1ccc(C2(c3ccc(N(c4cccc5c4C(c4ccccc4)(c4ccccc4)c4ccccc4-5)c4ccc(-c5cc6ccccc6c6ccccc56)c5oc6ccccc6c45)cc3)c3ccccc3-c3ccccc32)cc1]

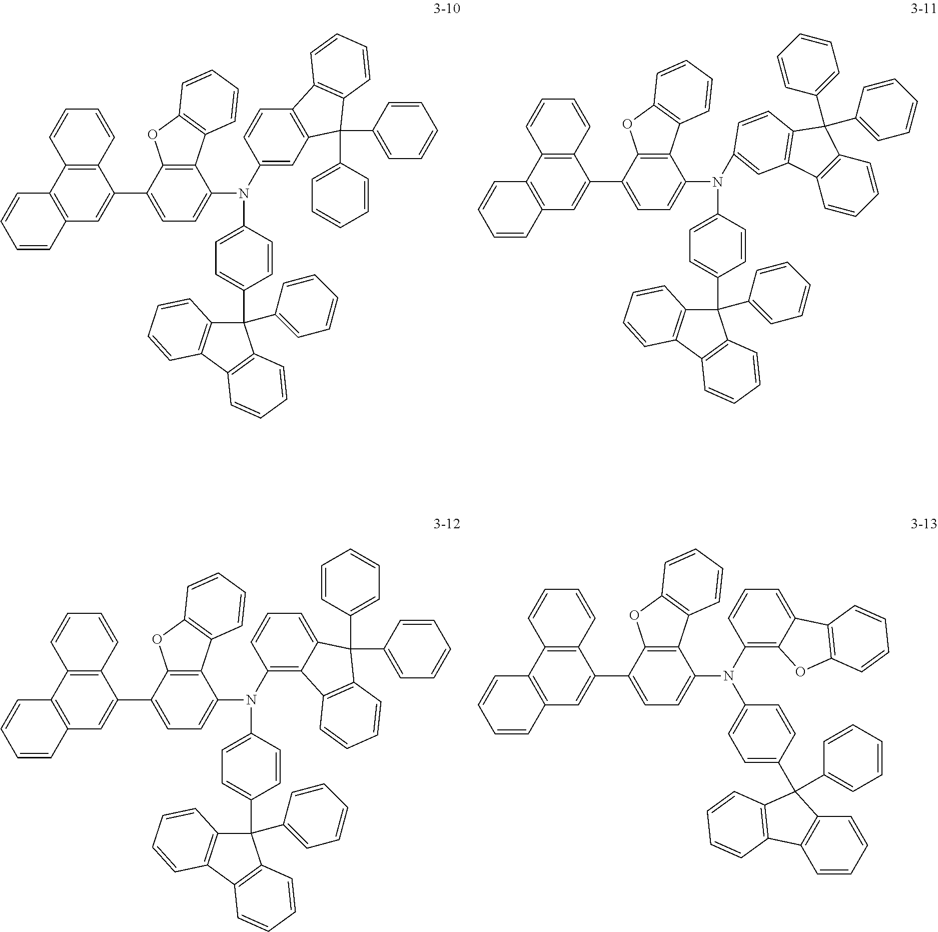 [SMILES: c1ccc(C2(c3ccc(N(c4ccc5c(c4)C(c4ccccc4)(c4ccccc4)c4ccccc4-5)c4ccc(-c5cc6ccccc6c6ccccc56)c5oc6ccccc6c45)cc3)c3ccccc3-c3ccccc32)cc1 ; c1ccc(C2(c3ccc(N(c4ccc5c(c4)-c4ccccc4C5(c4ccccc4)c4ccccc4)c4ccc(-c5cc6ccccc6c6ccccc56)c5oc6ccccc6c45)cc3)c3ccccc3-c3ccccc32)cc1 ; c1ccc(C2(c3ccc(N(c4cccc5c4-c4ccccc4C5(c4ccccc4)c4ccccc4)c4ccc(-c5cc6ccccc6c6ccccc56)c5oc6ccccc6c45)cc3)c3ccccc3-c3ccccc32)cc1 ; c1ccc(C2(c3ccc(N(c4cccc5c4oc4ccccc45)c4ccc(-c5cc6ccccc6c6ccccc56)c5oc6ccccc6c45)cc3)c3ccccc3-c3ccccc32)cc1]

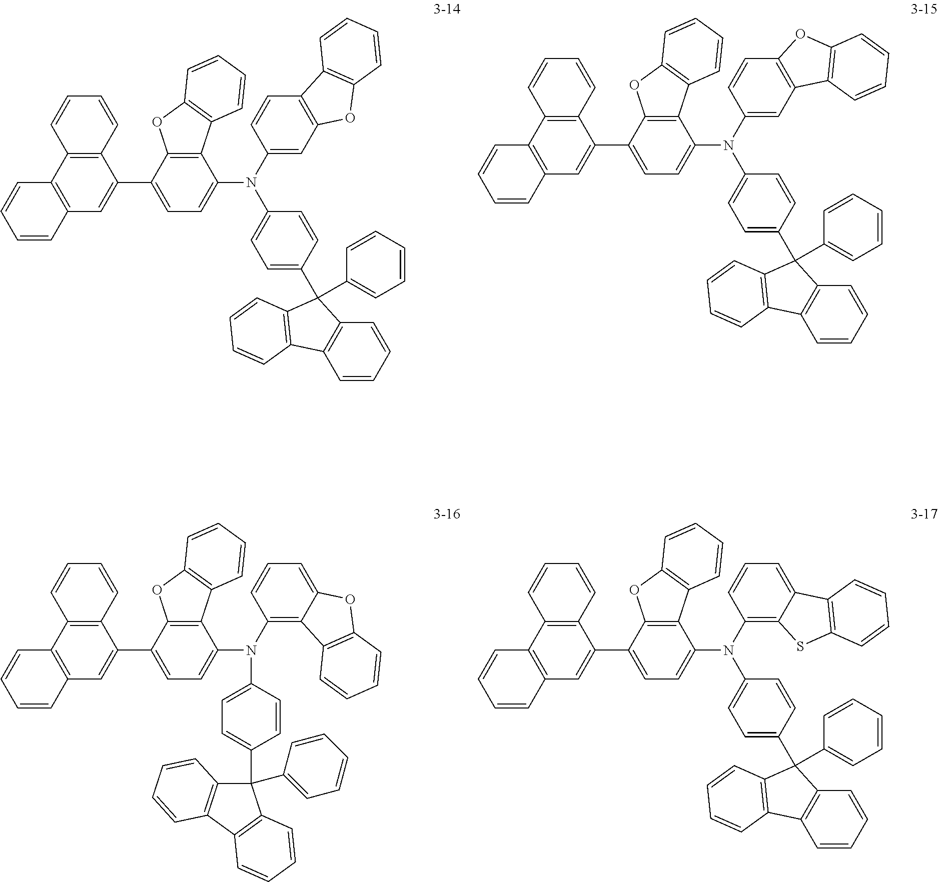 [SMILES: c1ccc(C2(c3ccc(N(c4ccc5c(c4)oc4ccccc45)c4ccc(-c5cc6ccccc6c6ccccc56)c5oc6ccccc6c45)cc3)c3ccccc3-c3ccccc32)cc1 ; c1ccc(C2(c3ccc(N(c4ccc5oc6ccccc6c5c4)c4ccc(-c5cc6ccccc6c6ccccc56)c5oc6ccccc6c45)cc3)c3ccccc3-c3ccccc32)cc1 ; c1ccc(C2(c3ccc(N(c4cccc5oc6ccccc6c45)c4ccc(-c5cc6ccccc6c6ccccc56)c5oc6ccccc6c45)cc3)c3ccccc3-c3ccccc32)cc1 ; c1ccc(C2(c3ccc(N(c4cccc5c4sc4ccccc45)c4ccc(-c5cc6ccccc6c6ccccc56)c5oc6ccccc6c45)cc3)c3ccccc3-c3ccccc32)cc1]

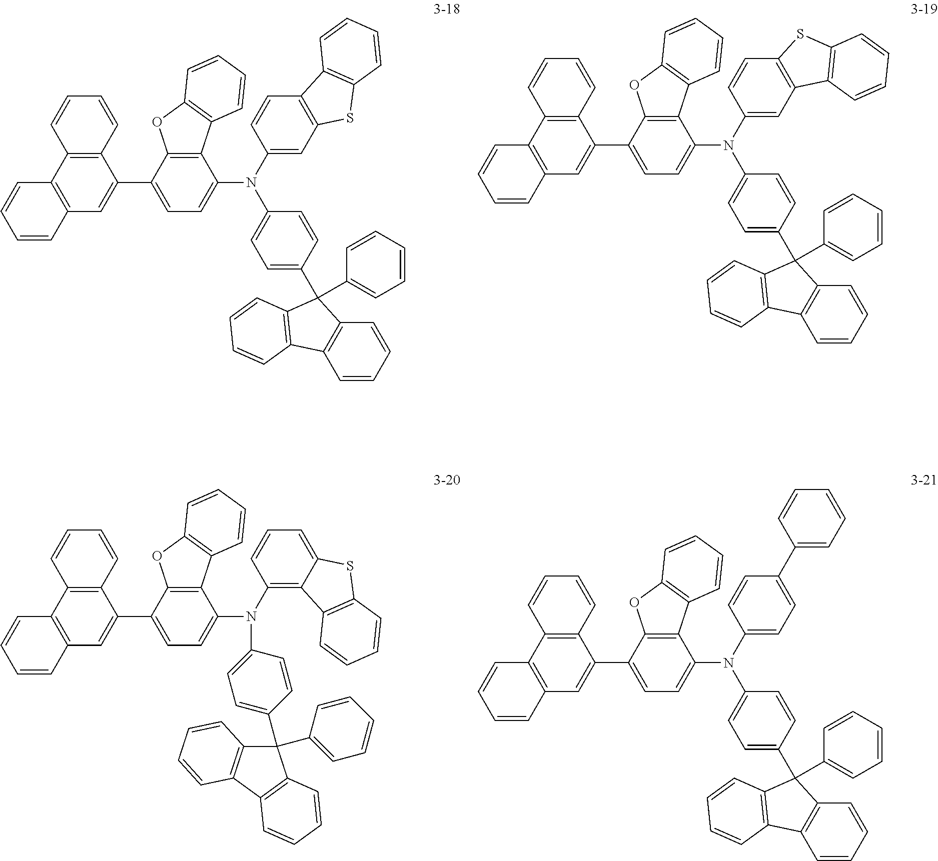 [SMILES: c1ccc(C2(c3ccc(N(c4ccc5c(c4)sc4ccccc45)c4ccc(-c5cc6ccccc6c6ccccc56)c5oc6ccccc6c45)cc3)c3ccccc3-c3ccccc32)cc1 ; c1ccc(C2(c3ccc(N(c4ccc5sc6ccccc6c5c4)c4ccc(-c5cc6ccccc6c6ccccc56)c5oc6ccccc6c45)cc3)c3ccccc3-c3ccccc32)cc1 ; c1ccc(C2(c3ccc(N(c4cccc5sc6ccccc6c45)c4ccc(-c5cc6ccccc6c6ccccc56)c5oc6ccccc6c45)cc3)c3ccccc3-c3ccccc32)cc1 ; c1ccc(-c2ccc(N(c3ccc(C4(c5ccccc5)c5ccccc5-c5ccccc54)cc3)c3ccc(-c4cc5ccccc5c5ccccc45)c4oc5ccccc5c34)cc2)cc1]

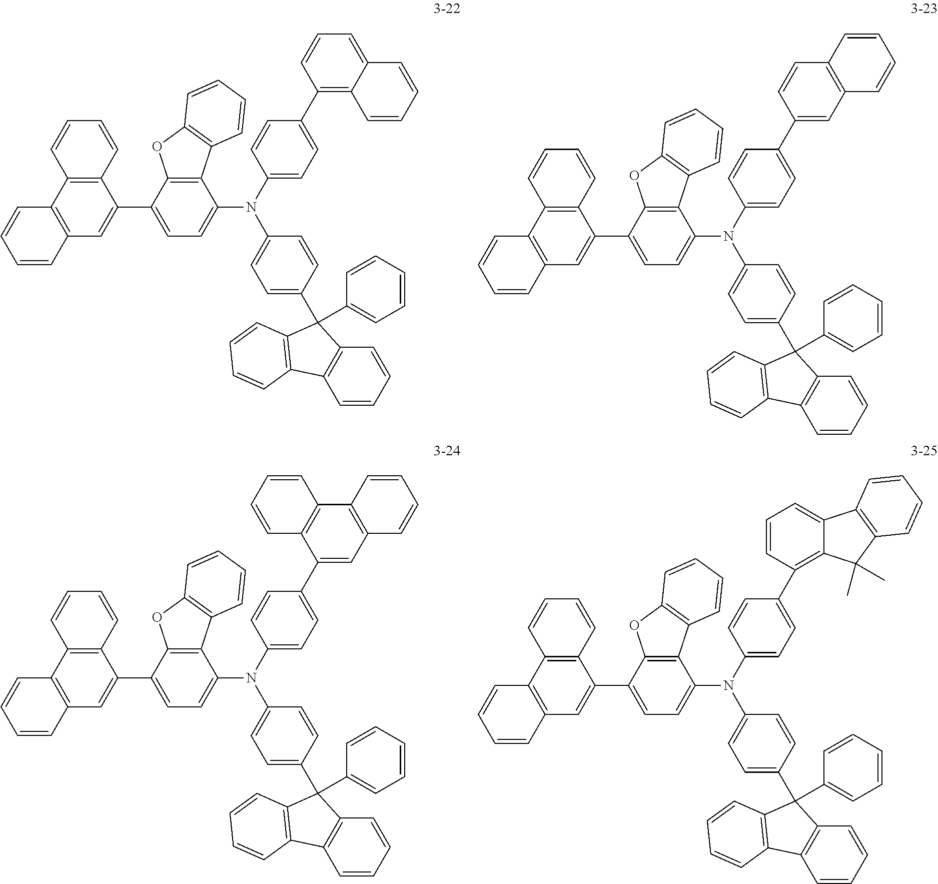 [SMILES: c1ccc(C2(c3ccc(N(c4ccc(-c5cccc6ccccc56)cc4)c4ccc(-c5cc6ccccc6c6ccccc56)c5oc6ccccc6c45)cc3)c3ccccc3-c3ccccc32)cc1 ; c1ccc(C2(c3ccc(N(c4ccc(-c5ccc6ccccc6c5)cc4)c4ccc(-c5cc6ccccc6c6ccccc56)c5oc6ccccc6c45)cc3)c3ccccc3-c3ccccc32)cc1 ; c1ccc(C2(c3ccc(N(c4ccc(-c5cc6ccccc6c6ccccc56)cc4)c4ccc(-c5cc6ccccc6c6ccccc56)c5oc6ccccc6c45)cc3)c3ccccc3-c3ccccc32)cc1 ; CC1(C)c2ccccc2-c2cccc(-c3ccc(N(c4ccc(C5(c6ccccc6)c6ccccc6-c6ccccc65)cc4)c4ccc(-c5cc6ccccc6c6ccccc56)c5oc6ccccc6c45)cc3)c21]

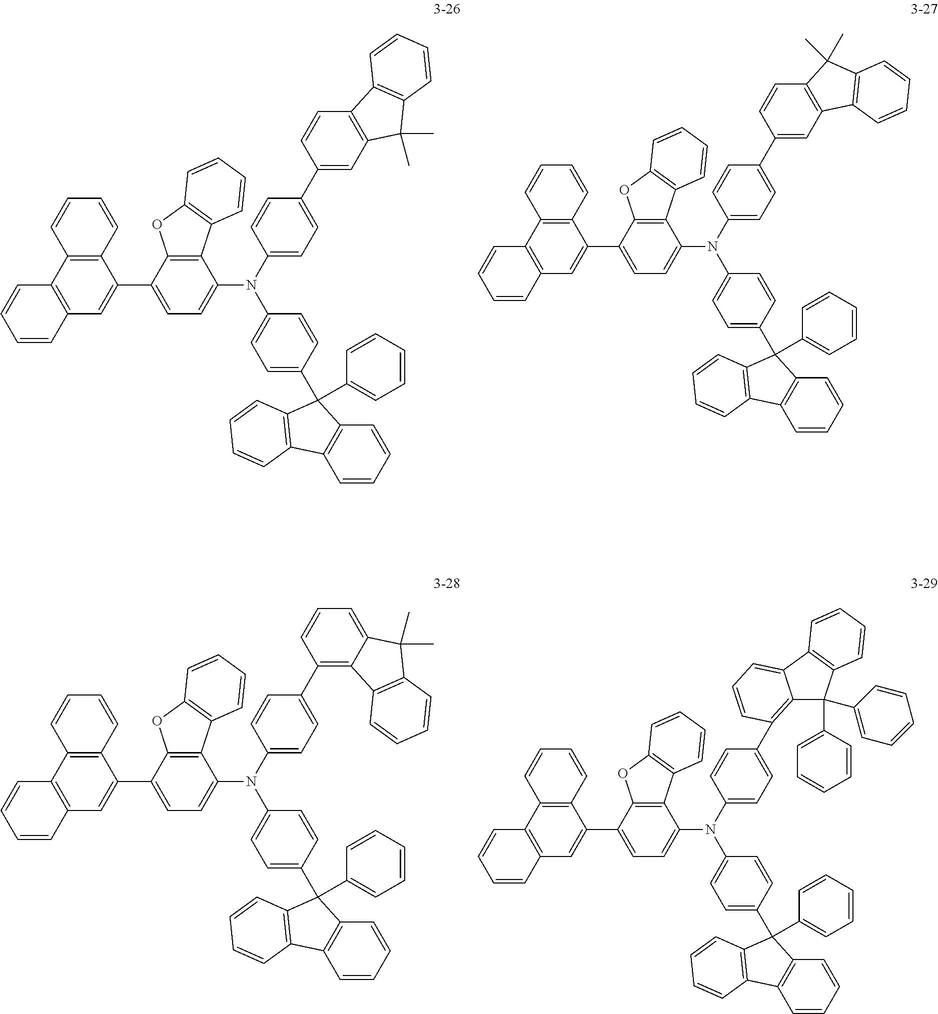

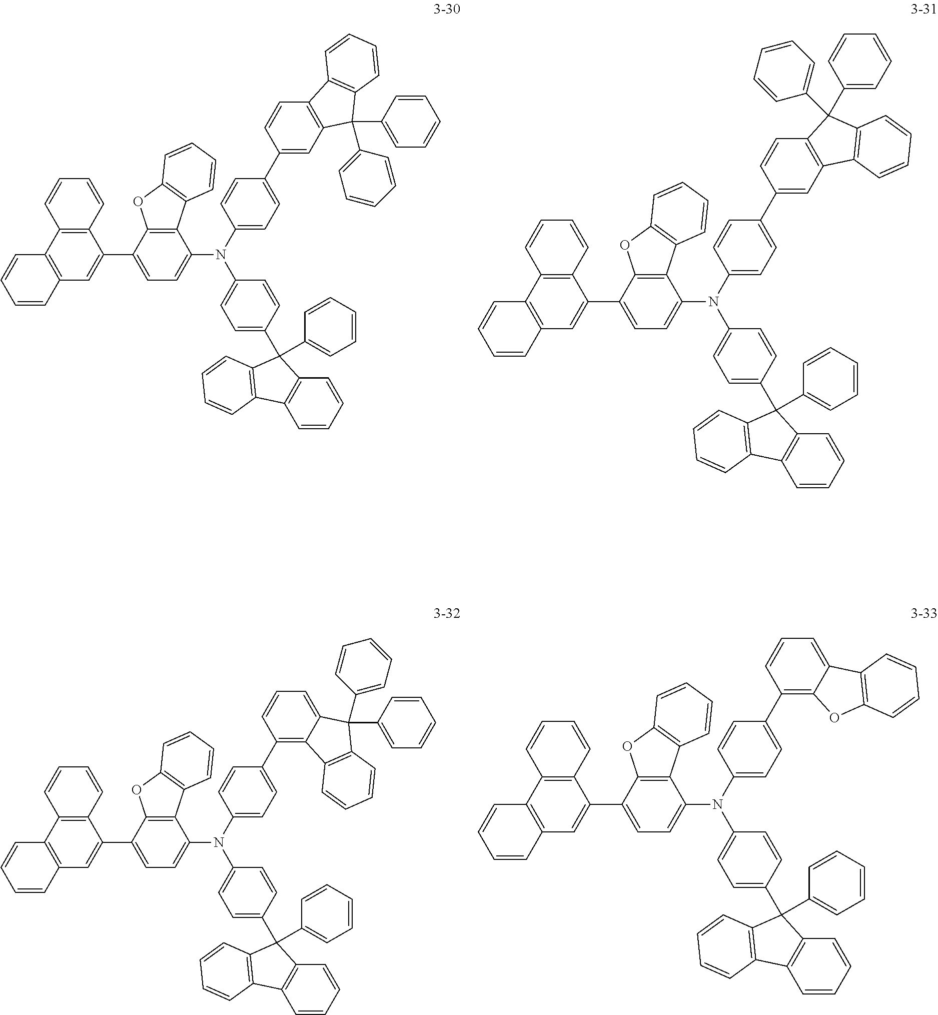

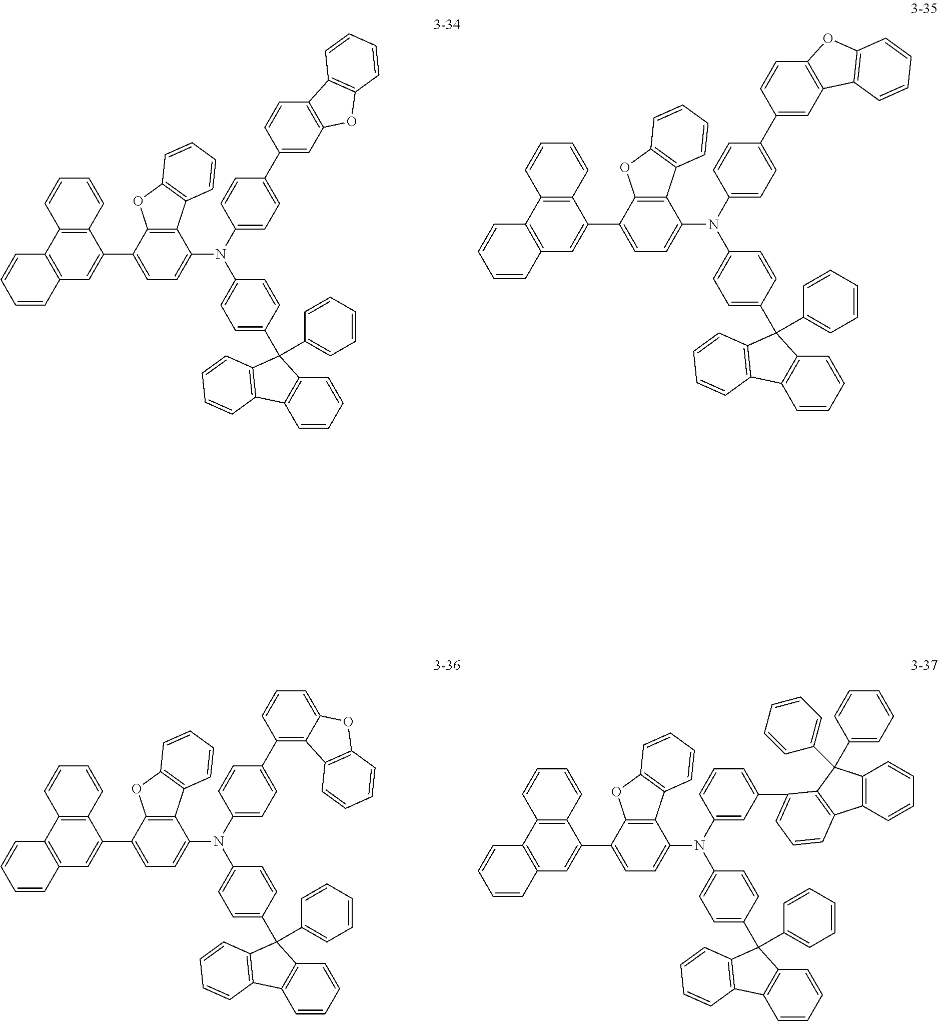 [SMILES: c1ccc(C2(c3ccc(N(c4ccc(-c5ccc6c(c5)oc5ccccc56)cc4)c4ccc(-c5cc6ccccc6c6ccccc56)c5oc6ccccc6c45)cc3)c3ccccc3-c3ccccc32)cc1 ; c1ccc(C2(c3ccc(N(c4ccc(-c5ccc6oc7ccccc7c6c5)cc4)c4ccc(-c5cc6ccccc6c6ccccc56)c5oc6ccccc6c45)cc3)c3ccccc3-c3ccccc32)cc1 ; c1ccc(C2(c3ccc(N(c4ccc(-c5cccc6oc7ccccc7c56)cc4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5cccc6c5C(c5ccccc5)(c5ccccc5)c5ccccc5-6)c4)c4ccc(-c5cc6ccccc6c6ccccc56)c5oc6ccccc6c45)cc3)c3ccccc3-c3ccccc32)cc1]

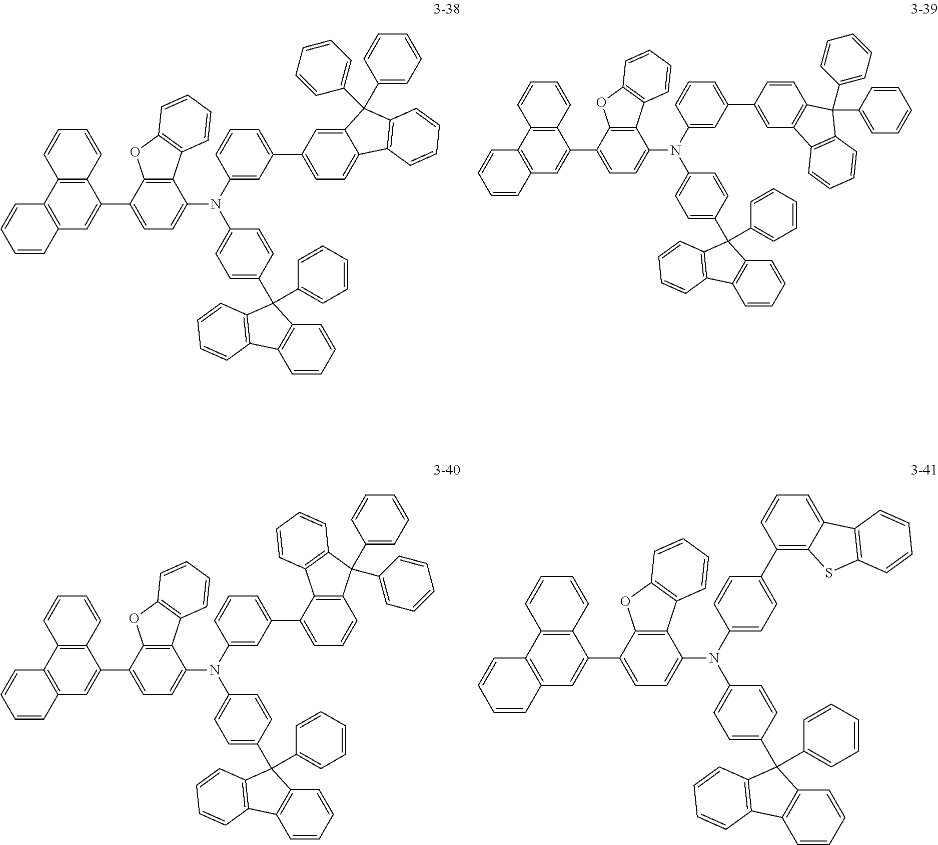

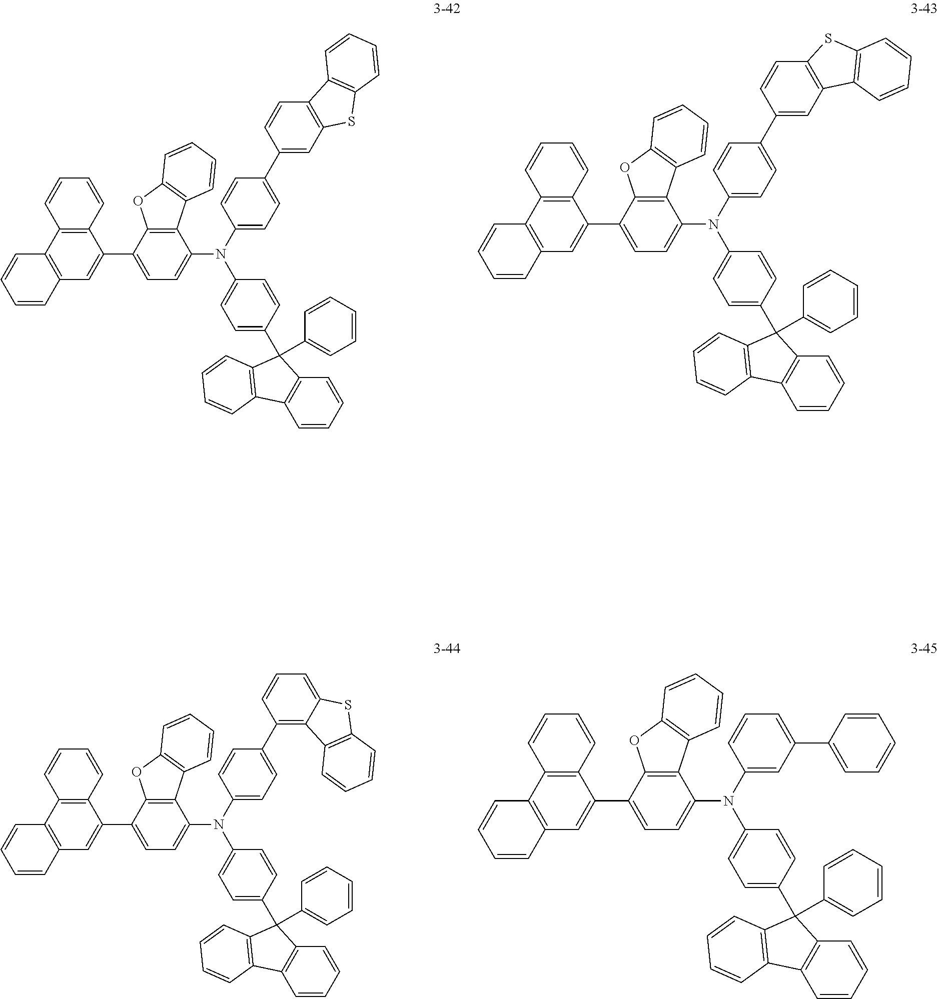 [SMILES: c1ccc(C2(c3ccc(N(c4ccc(-c5ccc6c(c5)sc5ccccc56)cc4)c4ccc(-c5cc6ccccc6c6ccccc56)c5oc6ccccc6c45)cc3)c3ccccc3-c3ccccc32)cc1 ; c1ccc(C2(c3ccc(N(c4ccc(-c5ccc6sc7ccccc7c6c5)cc4)c4ccc(-c5cc6ccccc6c6ccccc56)c5oc6ccccc6c45)cc3)c3ccccc3-c3ccccc32)cc1 ; c1ccc(C2(c3ccc(N(c4ccc(-c5cccc6sc7ccccc7c56)cc4)c4ccc(-c5cc6ccccc6c6ccccc56)c5oc6ccccc6c45)cc3)c3ccccc3-c3ccccc32)cc1 ; c1ccc(-c2cccc(N(c3ccc(C4(c5ccccc5)c5ccccc5-c5ccccc54)cc3)c3ccc(-c4cc5ccccc5c5ccccc45)c4oc5ccccc5c34)c2)cc1]

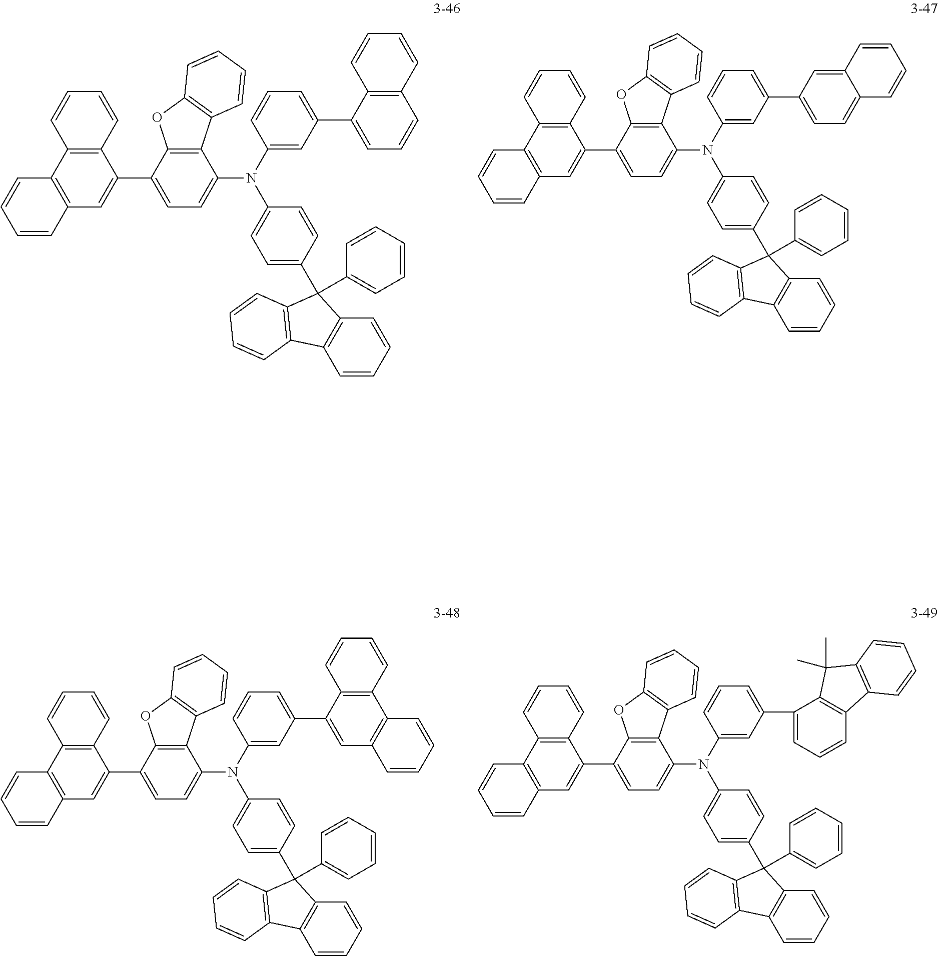 [SMILES: c1ccc(C2(c3ccc(N(c4cccc(-c5cccc6ccccc56)c4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5ccc6ccccc6c5)c4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5cc6ccccc6c6ccccc56)c4)c4ccc(-c5cc6ccccc6c6ccccc56)c5oc6ccccc6c45)cc3)c3ccccc3-c3ccccc32)cc1 ; CC1(C)c2ccccc2-c2cccc(-c3cccc(N(c4ccc(C5(c6ccccc6)c6ccccc6-c6ccccc65)cc4)c4ccc(-c5cc6ccccc6c6ccccc56)c5oc6ccccc6c45)c3)c21]

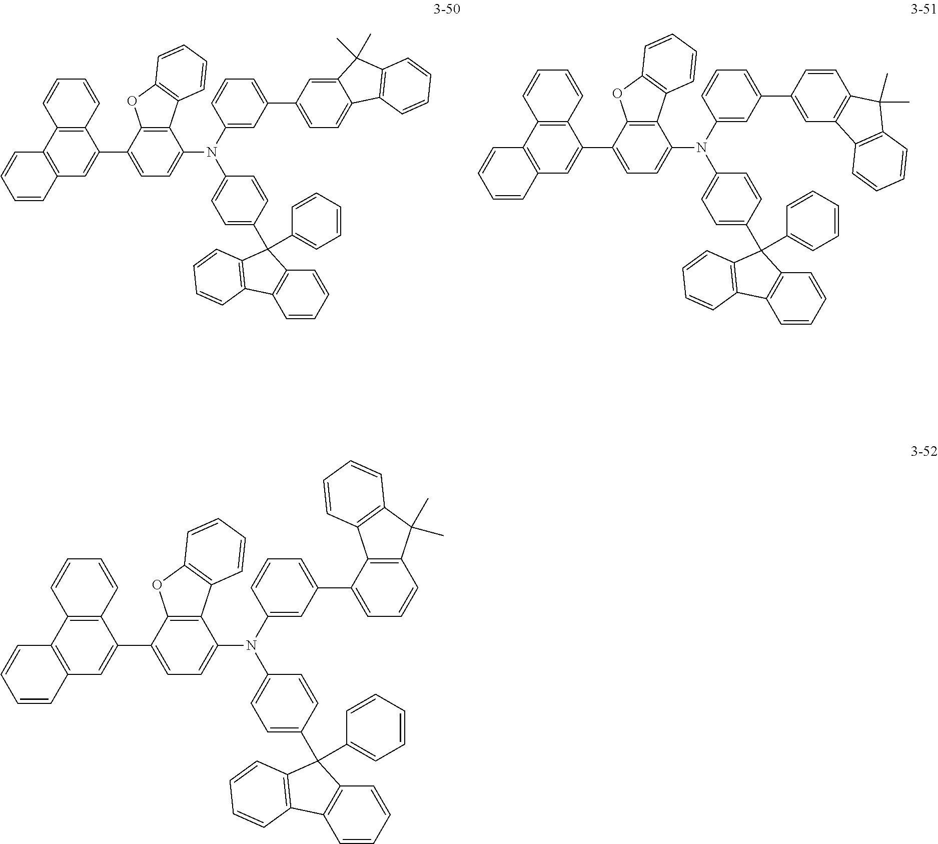 [SMILES: CC1(C)c2ccccc2-c2ccc(-c3cccc(N(c4ccc(C5(c6ccccc6)c6ccccc6-c6ccccc65)cc4)c4ccc(-c5cc6ccccc6c6ccccc56)c5oc6ccccc6c45)c3)cc21 ; CC1(C)c2ccccc2-c2cc(-c3cccc(N(c4ccc(C5(c6ccccc6)c6ccccc6-c6ccccc65)cc4)c4ccc(-c5cc6ccccc6c6ccccc56)c5oc6ccccc6c45)c3)ccc21 ; CC1(C)c2ccccc2-c2c(-c3cccc(N(c4ccc(C5(c6ccccc6)c6ccccc6-c6ccccc65)cc4)c4ccc(-c5cc6ccccc6c6ccccc56)c5oc6ccccc6c45)c3)cccc21]

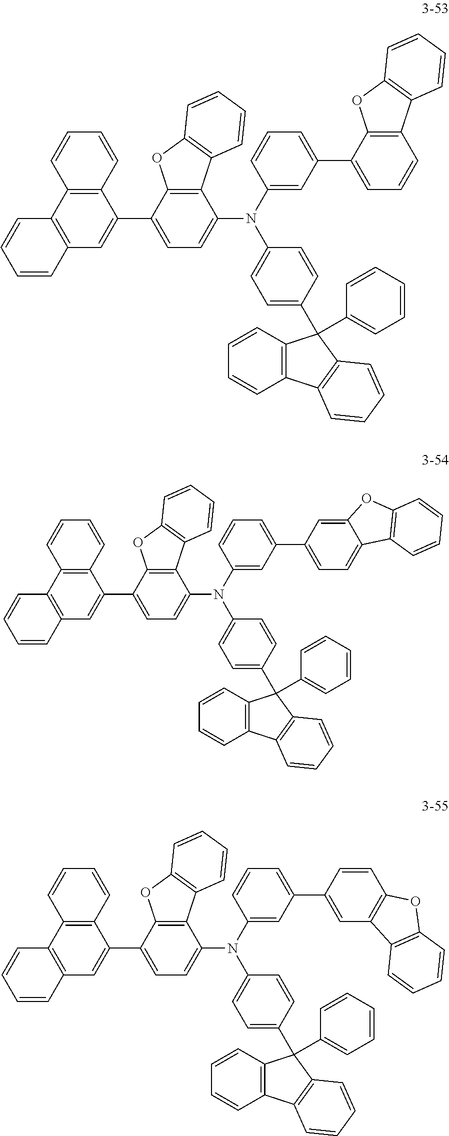 [SMILES: c1ccc(C2(c3ccc(N(c4cccc(-c5cccc6c5oc5ccccc56)c4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5ccc6c(c5)oc5ccccc56)c4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5ccc6oc7ccccc7c6c5)c4)c4ccc(-c5cc6ccccc6c6ccccc56)c5oc6ccccc6c45)cc3)c3ccccc3-c3ccccc32)cc1]

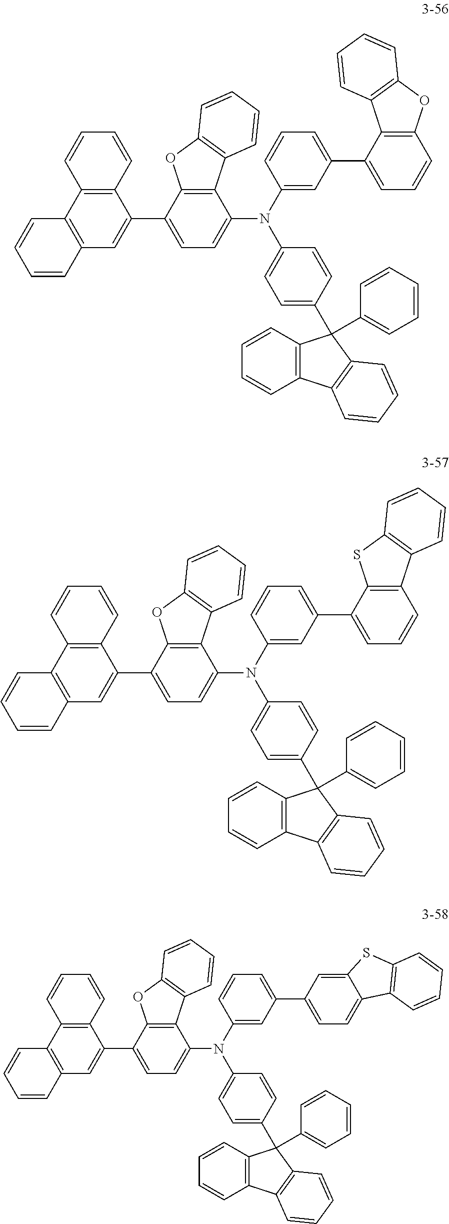 [SMILES: c1ccc(C2(c3ccc(N(c4cccc(-c5cccc6oc7ccccc7c56)c4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5cccc6c5sc5ccccc56)c4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5ccc6c(c5)sc5ccccc56)c4)c4ccc(-c5cc6ccccc6c6ccccc56)c5oc6ccccc6c45)cc3)c3ccccc3-c3ccccc32)cc1]

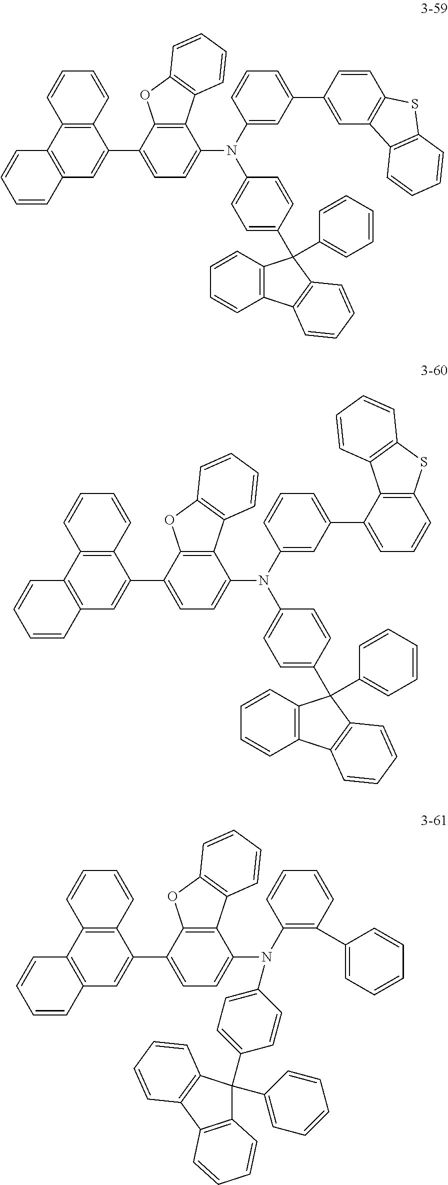 [SMILES: c1ccc(C2(c3ccc(N(c4cccc(-c5ccc6sc7ccccc7c6c5)c4)c4ccc(-c5cc6ccccc6c6ccccc56)c5oc6ccccc6c45)cc3)c3ccccc3-c3ccccc32)cc1 ; c1ccc(C2(c3ccc(N(c4cccc(-c5cccc6sc7ccccc7c56)c4)c4ccc(-c5cc6ccccc6c6ccccc56)c5oc6ccccc6c45)cc3)c3ccccc3-c3ccccc32)cc1 ; c1ccc(-c2ccccc2N(c2ccc(C3(c4ccccc4)c4ccccc4-c4ccccc43)cc2)c2ccc(-c3cc4ccccc4c4ccccc34)c3oc4ccccc4c23)cc1]

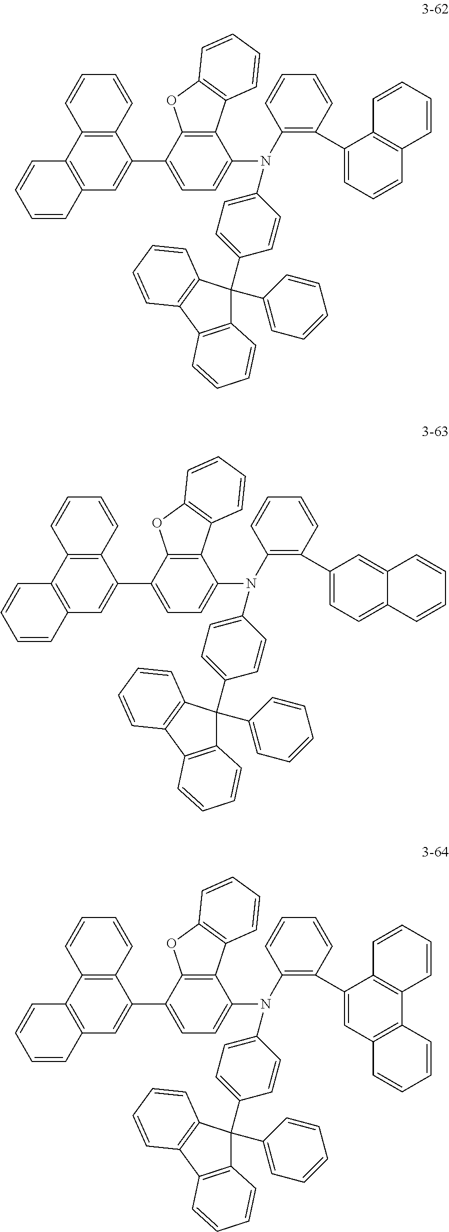 [SMILES: c1ccc(C2(c3ccc(N(c4ccccc4-c4cccc5ccccc45)c4ccc(-c5cc6ccccc6c6ccccc56)c5oc6ccccc6c45)cc3)c3ccccc3-c3ccccc32)cc1 ; c1ccc(C2(c3ccc(N(c4ccccc4-c4ccc5ccccc5c4)c4ccc(-c5cc6ccccc6c6ccccc56)c5oc6ccccc6c45)cc3)c3ccccc3-c3ccccc32)cc1 ; c1ccc(C2(c3ccc(N(c4ccccc4-c4cc5ccccc5c5ccccc45)c4ccc(-c5cc6ccccc6c6ccccc56)c5oc6ccccc6c45)cc3)c3ccccc3-c3ccccc32)cc1]

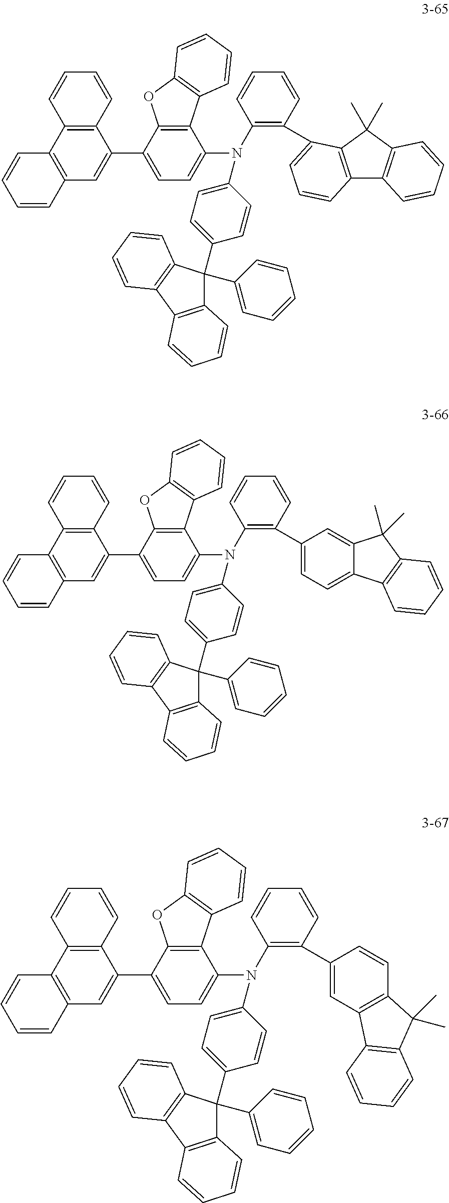 [SMILES: CC1(C)c2ccccc2-c2cccc(-c3ccccc3N(c3ccc(C4(c5ccccc5)c5ccccc5-c5ccccc54)cc3)c3ccc(-c4cc5ccccc5c5ccccc45)c4oc5ccccc5c34)c21 ; CC1(C)c2ccccc2-c2ccc(-c3ccccc3N(c3ccc(C4(c5ccccc5)c5ccccc5-c5ccccc54)cc3)c3ccc(-c4cc5ccccc5c5ccccc45)c4oc5ccccc5c34)cc21 ; CC1(C)c2ccccc2-c2cc(-c3ccccc3N(c3ccc(C4(c5ccccc5)c5ccccc5-c5ccccc54)cc3)c3ccc(-c4cc5ccccc5c5ccccc45)c4oc5ccccc5c34)ccc21]

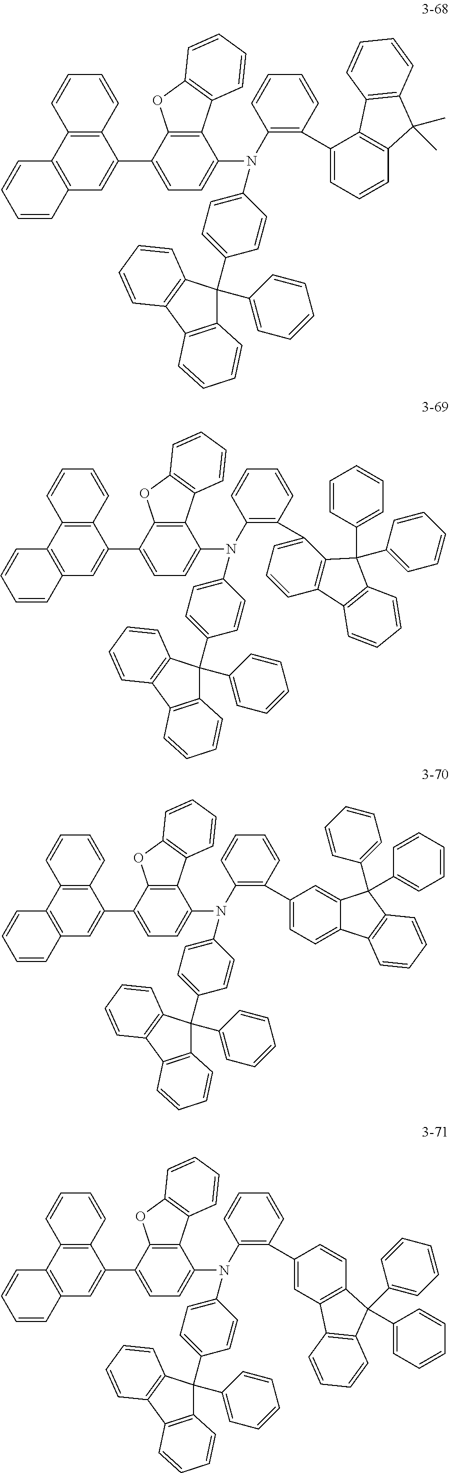

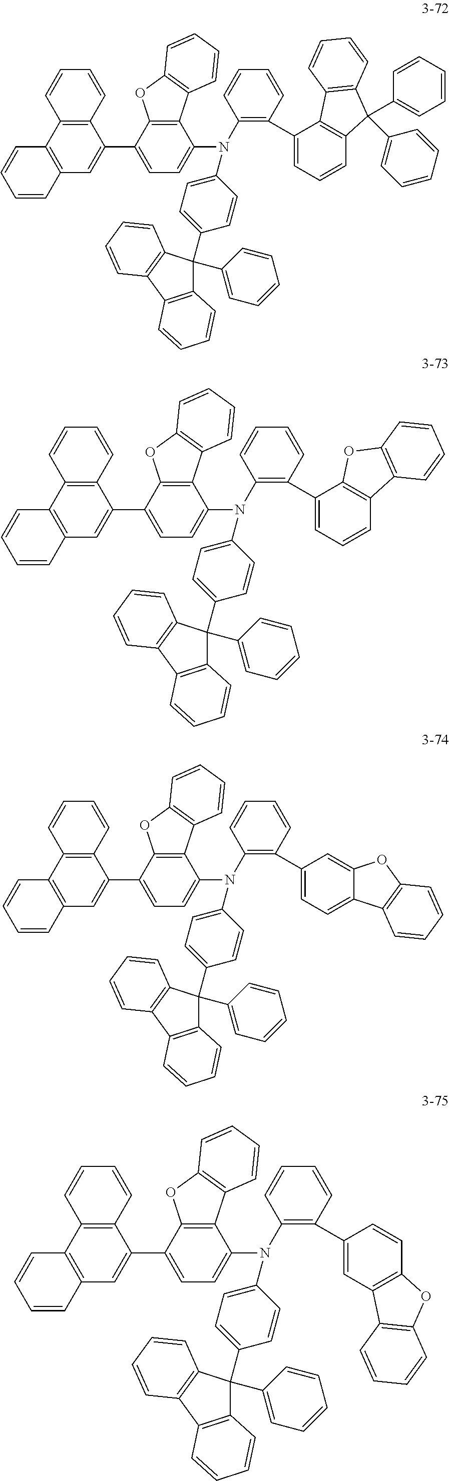

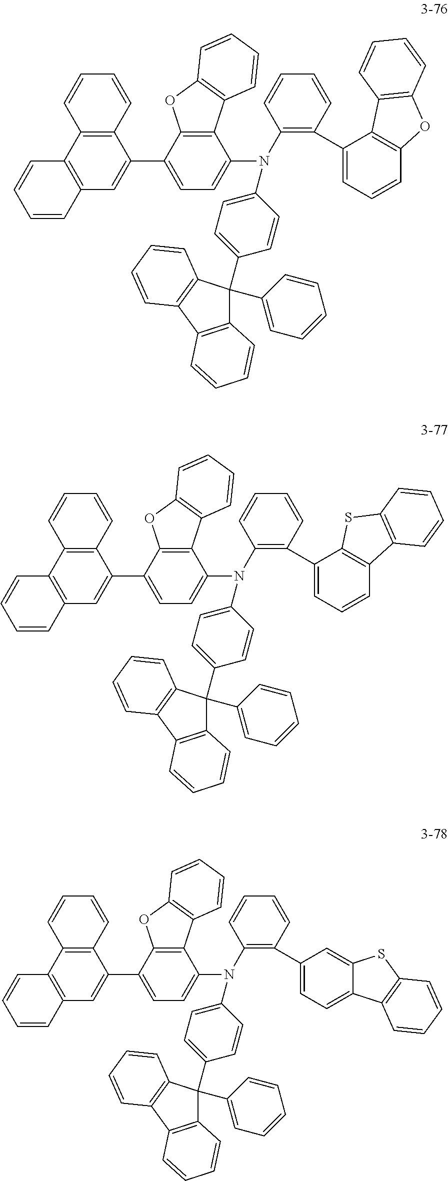 [SMILES: c1ccc(C2(c3ccc(N(c4ccccc4-c4cccc5oc6ccccc6c45)c4ccc(-c5cc6ccccc6c6ccccc56)c5oc6ccccc6c45)cc3)c3ccccc3-c3ccccc32)cc1 ; c1ccc(C2(c3ccc(N(c4ccccc4-c4cccc5c4sc4ccccc45)c4ccc(-c5cc6ccccc6c6ccccc56)c5oc6ccccc6c45)cc3)c3ccccc3-c3ccccc32)cc1 ; c1ccc(C2(c3ccc(N(c4ccccc4-c4ccc5c(c4)sc4ccccc45)c4ccc(-c5cc6ccccc6c6ccccc56)c5oc6ccccc6c45)cc3)c3ccccc3-c3ccccc32)cc1]

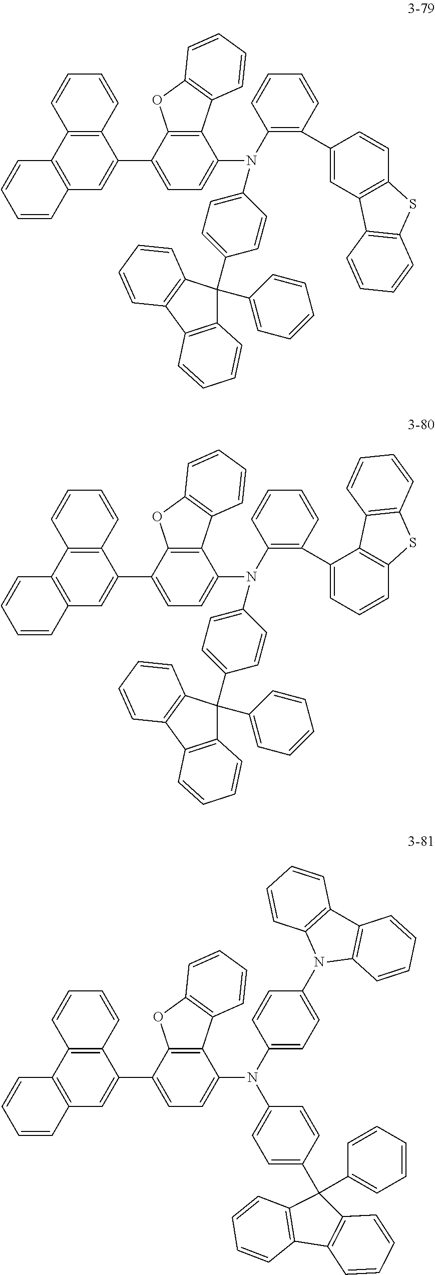 [SMILES: c1ccc(C2(c3ccc(N(c4ccccc4-c4ccc5sc6ccccc6c5c4)c4ccc(-c5cc6ccccc6c6ccccc56)c5oc6ccccc6c45)cc3)c3ccccc3-c3ccccc32)cc1 ; c1ccc(C2(c3ccc(N(c4ccccc4-c4cccc5sc6ccccc6c45)c4ccc(-c5cc6ccccc6c6ccccc56)c5oc6ccccc6c45)cc3)c3ccccc3-c3ccccc32)cc1 ; c1ccc(C2(c3ccc(N(c4ccc(-n5c6ccccc6c6ccccc65)cc4)c4ccc(-c5cc6ccccc6c6ccccc56)c5oc6ccccc6c45)cc3)c3ccccc3-c3ccccc32)cc1]

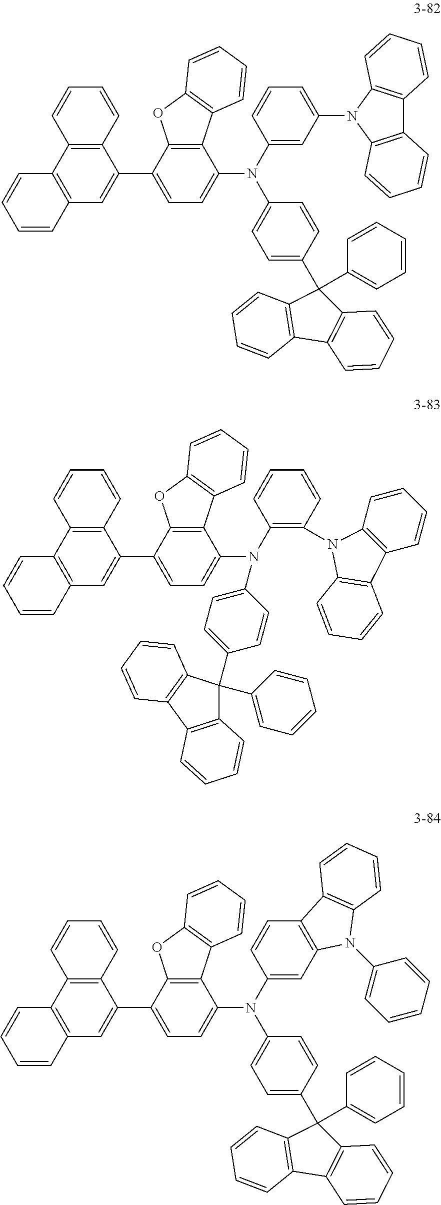 [SMILES: c1ccc(C2(c3ccc(N(c4cccc(-n5c6ccccc6c6ccccc65)c4)c4ccc(-c5cc6ccccc6c6ccccc56)c5oc6ccccc6c45)cc3)c3ccccc3-c3ccccc32)cc1 ; c1ccc(C2(c3ccc(N(c4ccccc4-n4c5ccccc5c5ccccc54)c4ccc(-c5cc6ccccc6c6ccccc56)c5oc6ccccc6c45)cc3)c3ccccc3-c3ccccc32)cc1 ; c1ccc(-n2c3ccccc3c3ccc(N(c4ccc(C5(c6ccccc6)c6ccccc6-c6ccccc65)cc4)c4ccc(-c5cc6ccccc6c6ccccc56)c5oc6ccccc6c45)cc32)cc1]

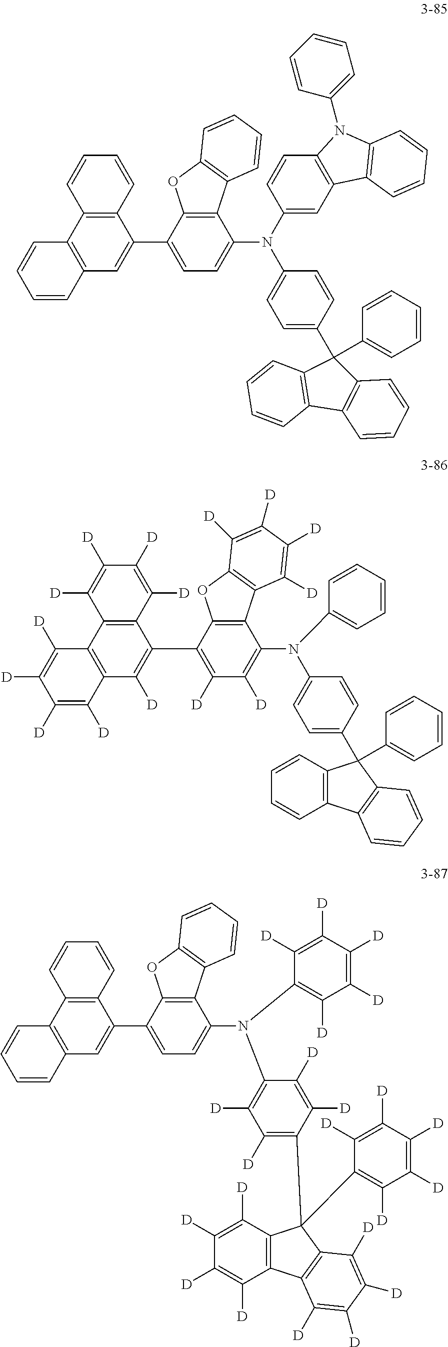

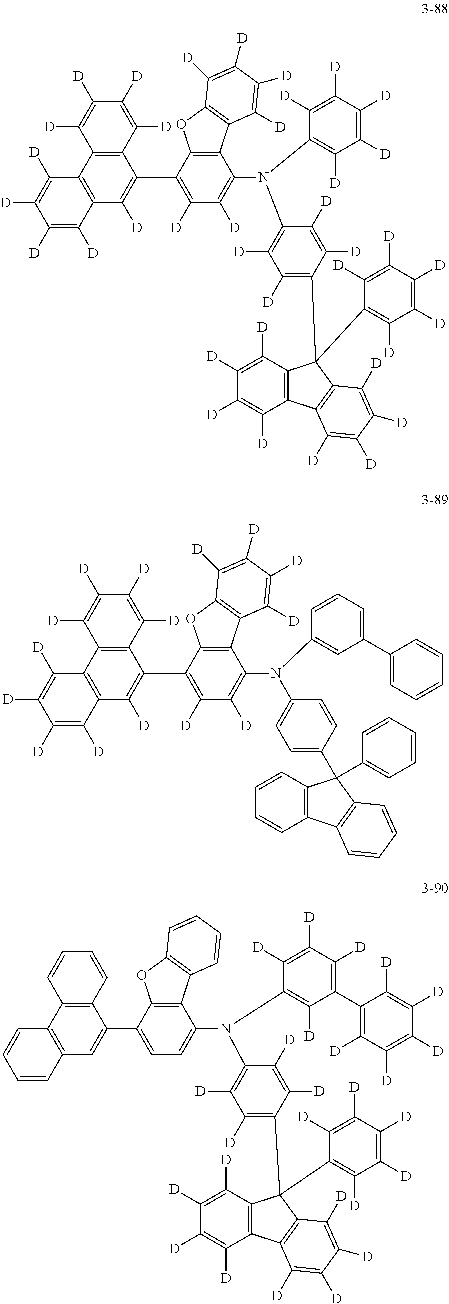

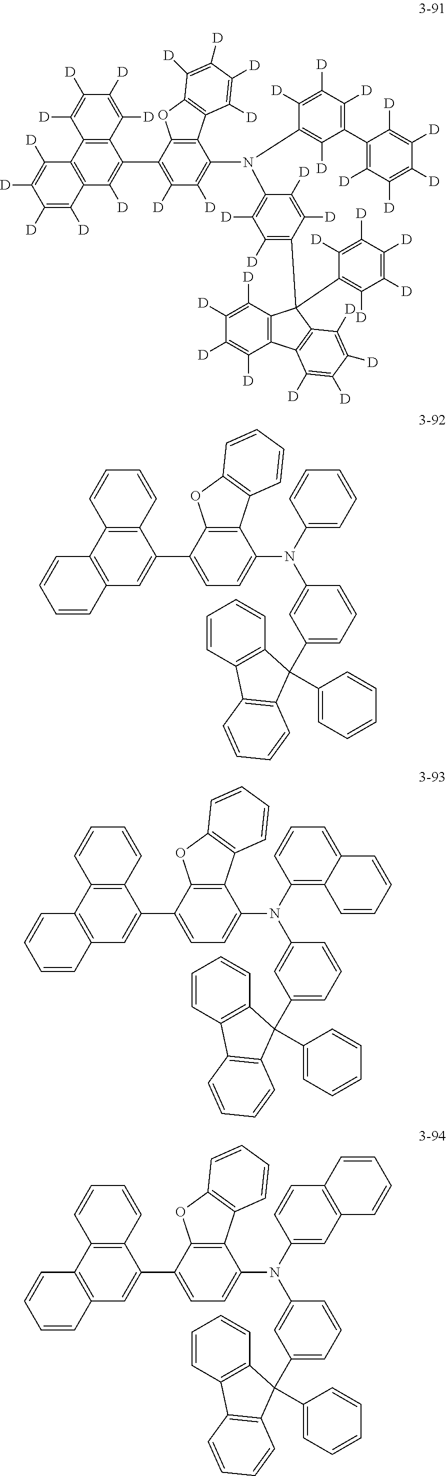

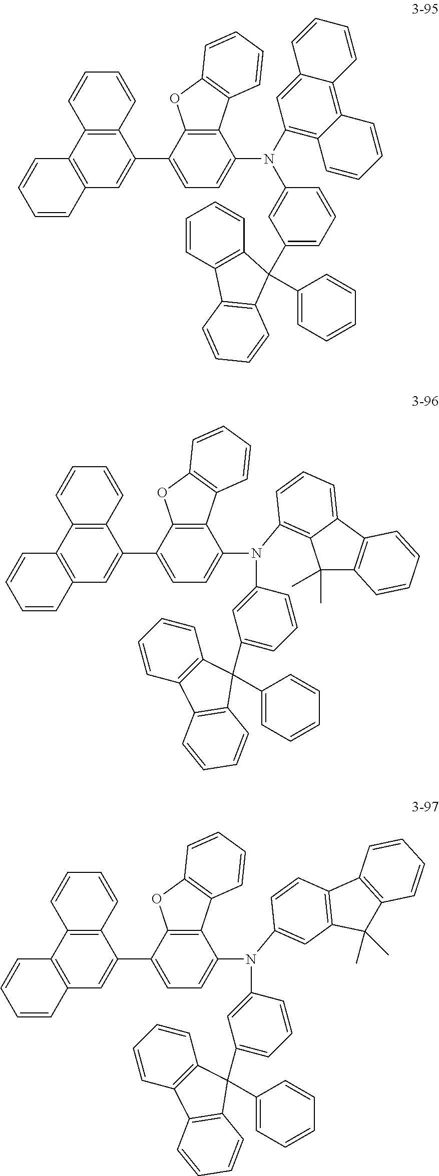 [SMILES: c1ccc(C2(c3cccc(N(c4cc5ccccc5c5ccccc45)c4ccc(-c5cc6ccccc6c6ccccc56)c5oc6ccccc6c45)c3)c3ccccc3-c3ccccc32)cc1 ; CC1(C)c2ccccc2-c2cccc(N(c3cccc(C4(c5ccccc5)c5ccccc5-c5ccccc54)c3)c3ccc(-c4cc5ccccc5c5ccccc45)c4oc5ccccc5c34)c21 ; CC1(C)c2ccccc2-c2ccc(N(c3cccc(C4(c5ccccc5)c5ccccc5-c5ccccc54)c3)c3ccc(-c4cc5ccccc5c5ccccc45)c4oc5ccccc5c34)cc21]

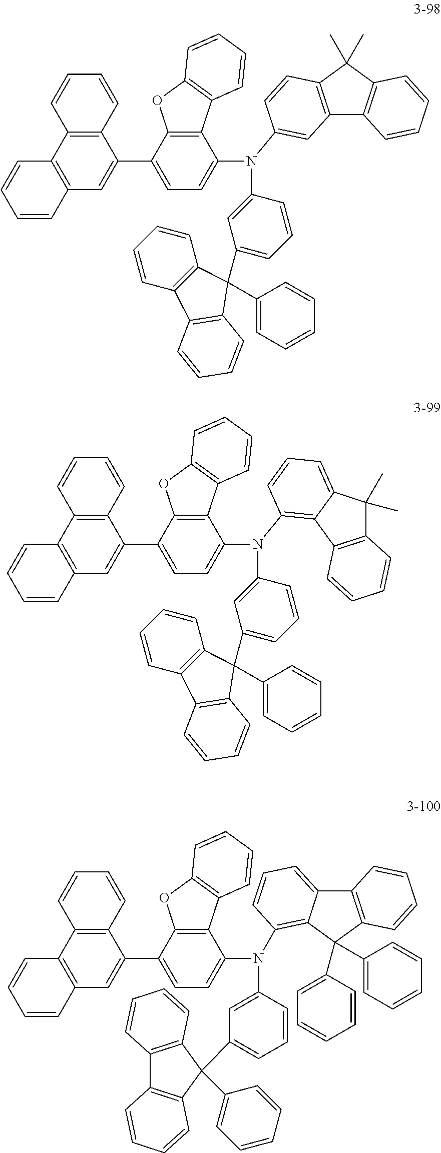

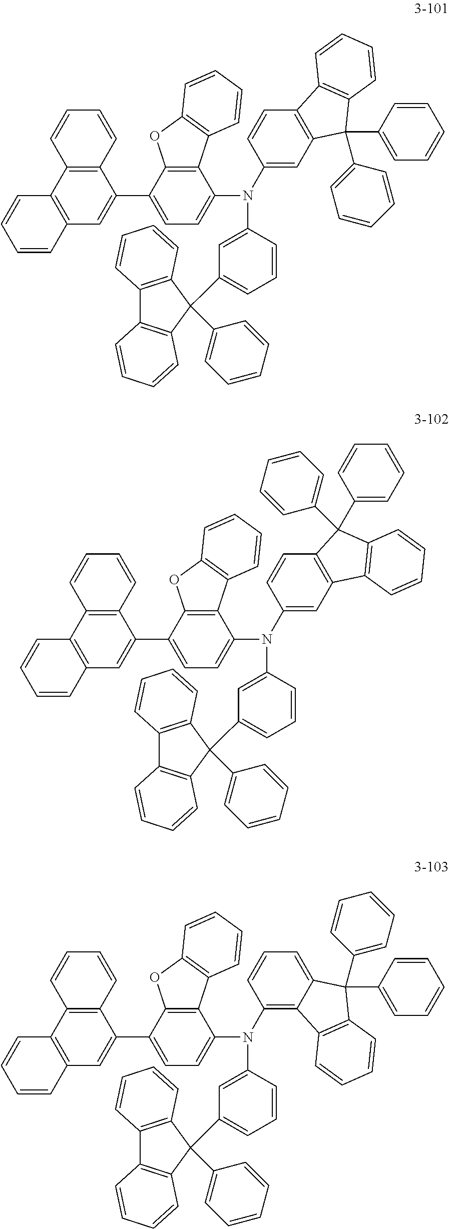 [SMILES: c1ccc(C2(c3cccc(N(c4ccc5c(c4)C(c4ccccc4)(c4ccccc4)c4ccccc4-5)c4ccc(-c5cc6ccccc6c6ccccc56)c5oc6ccccc6c45)c3)c3ccccc3-c3ccccc32)cc1 ; c1ccc(C2(c3cccc(N(c4ccc5c(c4)-c4ccccc4C5(c4ccccc4)c4ccccc4)c4ccc(-c5cc6ccccc6c6ccccc56)c5oc6ccccc6c45)c3)c3ccccc3-c3ccccc32)cc1 ; c1ccc(C2(c3cccc(N(c4cccc5c4-c4ccccc4C5(c4ccccc4)c4ccccc4)c4ccc(-c5cc6ccccc6c6ccccc56)c5oc6ccccc6c45)c3)c3ccccc3-c3ccccc32)cc1]

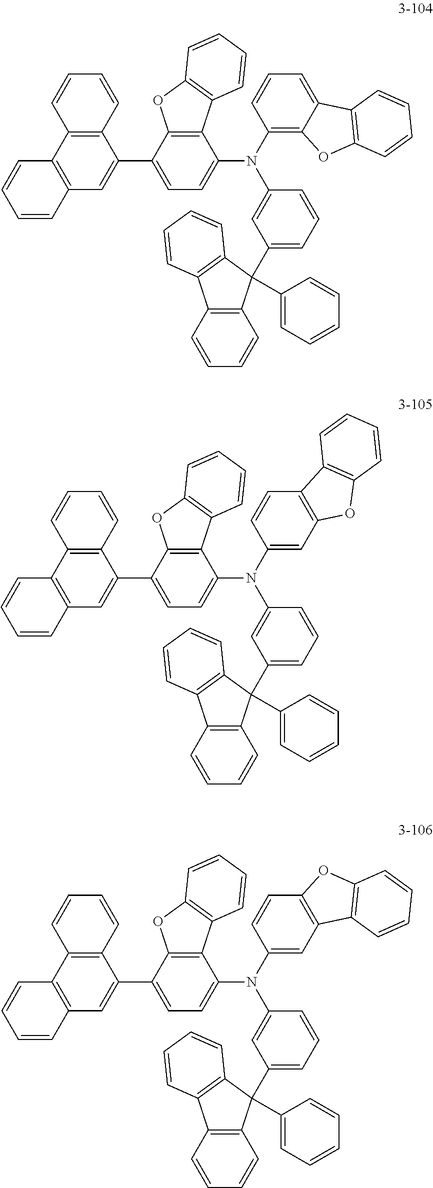 [SMILES: c1ccc(C2(c3cccc(N(c4cccc5c4oc4ccccc45)c4ccc(-c5cc6ccccc6c6ccccc56)c5oc6ccccc6c45)c3)c3ccccc3-c3ccccc32)cc1 ; c1ccc(C2(c3cccc(N(c4ccc5c(c4)oc4ccccc45)c4ccc(-c5cc6ccccc6c6ccccc56)c5oc6ccccc6c45)c3)c3ccccc3-c3ccccc32)cc1 ; c1ccc(C2(c3cccc(N(c4ccc5oc6ccccc6c5c4)c4ccc(-c5cc6ccccc6c6ccccc56)c5oc6ccccc6c45)c3)c3ccccc3-c3ccccc32)cc1]

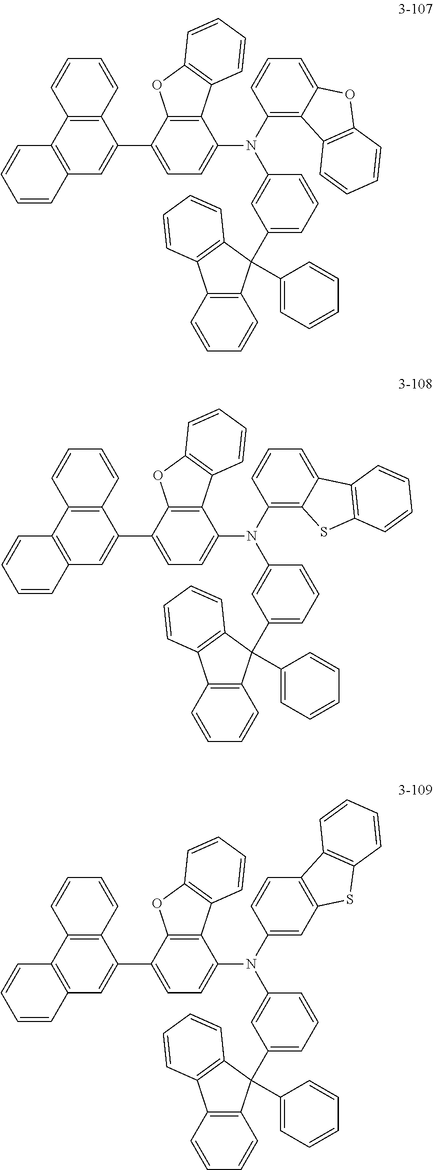 [SMILES: c1ccc(C2(c3cccc(N(c4cccc5oc6ccccc6c45)c4ccc(-c5cc6ccccc6c6ccccc56)c5oc6ccccc6c45)c3)c3ccccc3-c3ccccc32)cc1 ; c1ccc(C2(c3cccc(N(c4cccc5c4sc4ccccc45)c4ccc(-c5cc6ccccc6c6ccccc56)c5oc6ccccc6c45)c3)c3ccccc3-c3ccccc32)cc1 ; c1ccc(C2(c3cccc(N(c4ccc5c(c4)sc4ccccc45)c4ccc(-c5cc6ccccc6c6ccccc56)c5oc6ccccc6c45)c3)c3ccccc3-c3ccccc32)cc1]

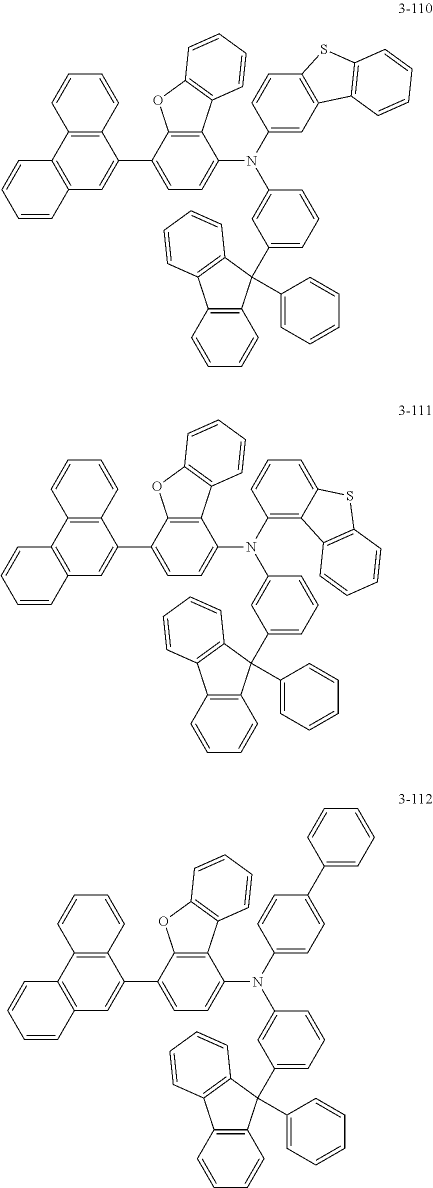 [SMILES: c1ccc(C2(c3cccc(N(c4ccc5sc6ccccc6c5c4)c4ccc(-c5cc6ccccc6c6ccccc56)c5oc6ccccc6c45)c3)c3ccccc3-c3ccccc32)cc1 ; c1ccc(C2(c3cccc(N(c4cccc5sc6ccccc6c45)c4ccc(-c5cc6ccccc6c6ccccc56)c5oc6ccccc6c45)c3)c3ccccc3-c3ccccc32)cc1 ; c1ccc(-c2ccc(N(c3cccc(C4(c5ccccc5)c5ccccc5-c5ccccc54)c3)c3ccc(-c4cc5ccccc5c5ccccc45)c4oc5ccccc5c34)cc2)cc1]

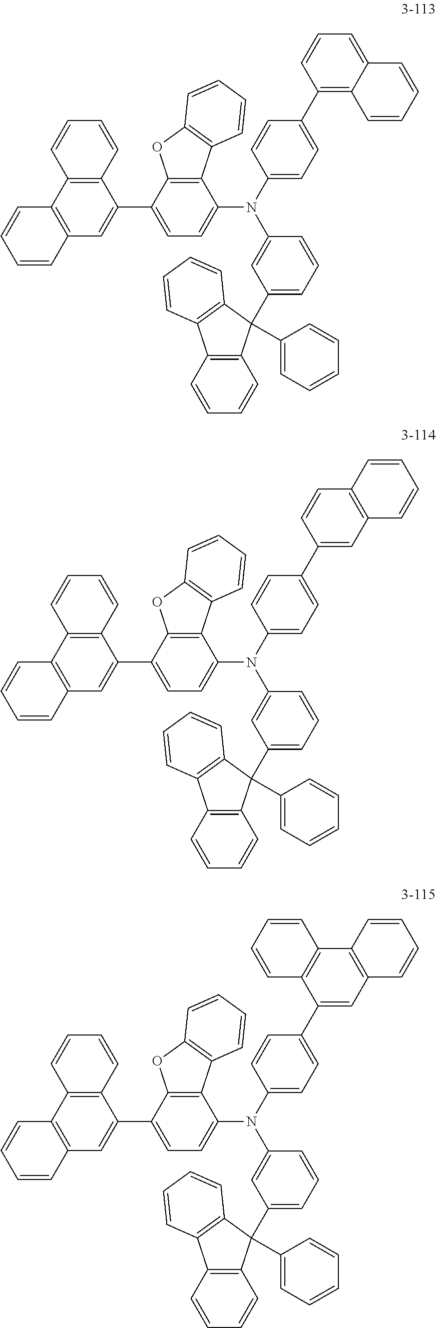 [SMILES: c1ccc(C2(c3cccc(N(c4ccc(-c5cccc6ccccc56)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5ccc6ccccc6c5)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5cc6ccccc6c6ccccc56)cc4)c4ccc(-c5cc6ccccc6c6ccccc56)c5oc6ccccc6c45)c3)c3ccccc3-c3ccccc32)cc1]

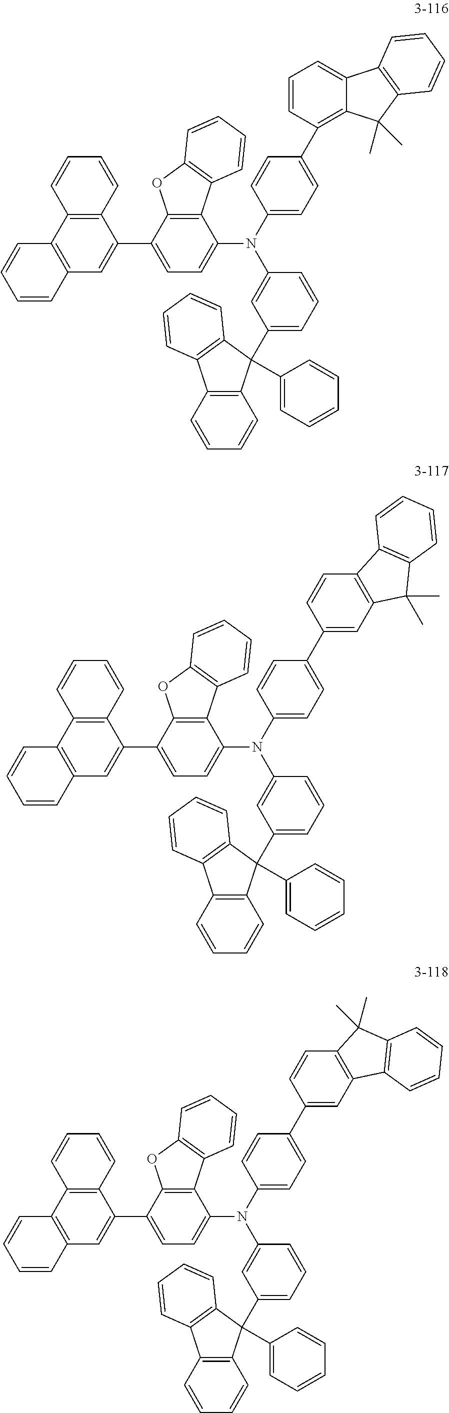 [SMILES: CC1(C)c2ccccc2-c2cccc(-c3ccc(N(c4cccc(C5(c6ccccc6)c6ccccc6-c6ccccc65)c4)c4ccc(-c5cc6ccccc6c6ccccc56)c5oc6ccccc6c45)cc3)c21 ; CC1(C)c2ccccc2-c2ccc(-c3ccc(N(c4cccc(C5(c6ccccc6)c6ccccc6-c6ccccc65)c4)c4ccc(-c5cc6ccccc6c6ccccc56)c5oc6ccccc6c45)cc3)cc21 ; CC1(C)c2ccccc2-c2cc(-c3ccc(N(c4cccc(C5(c6ccccc6)c6ccccc6-c6ccccc65)c4)c4ccc(-c5cc6ccccc6c6ccccc56)c5oc6ccccc6c45)cc3)ccc21]

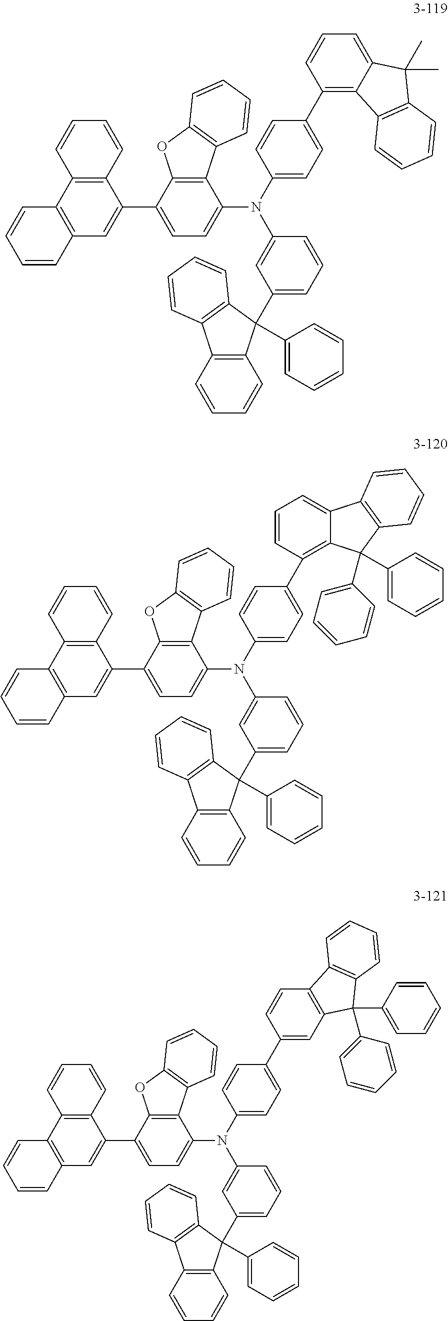 [SMILES: CC1(C)c2ccccc2-c2c(-c3ccc(N(c4cccc(C5(c6ccccc6)c6ccccc6-c6ccccc65)c4)c4ccc(-c5cc6ccccc6c6ccccc56)c5oc6ccccc6c45)cc3)cccc21 ; c1ccc(C2(c3cccc(N(c4ccc(-c5cccc6c5C(c5ccccc5)(c5ccccc5)c5ccccc5-6)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5ccc6c(c5)C(c5ccccc5)(c5ccccc5)c5ccccc5-6)cc4)c4ccc(-c5cc6ccccc6c6ccccc56)c5oc6ccccc6c45)c3)c3ccccc3-c3ccccc32)cc1]

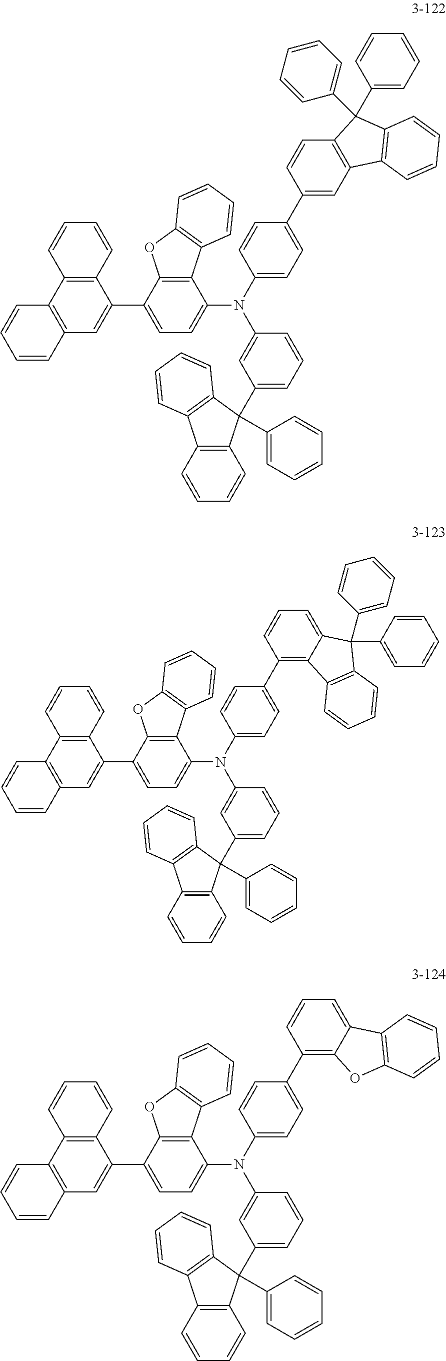 [SMILES: c1ccc(C2(c3cccc(N(c4ccc(-c5ccc6c(c5)-c5ccccc5C6(c5ccccc5)c5ccccc5)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5cccc6c5-c5ccccc5C6(c5ccccc5)c5ccccc5)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5cccc6c5oc5ccccc56)cc4)c4ccc(-c5cc6ccccc6c6ccccc56)c5oc6ccccc6c45)c3)c3ccccc3-c3ccccc32)cc1]

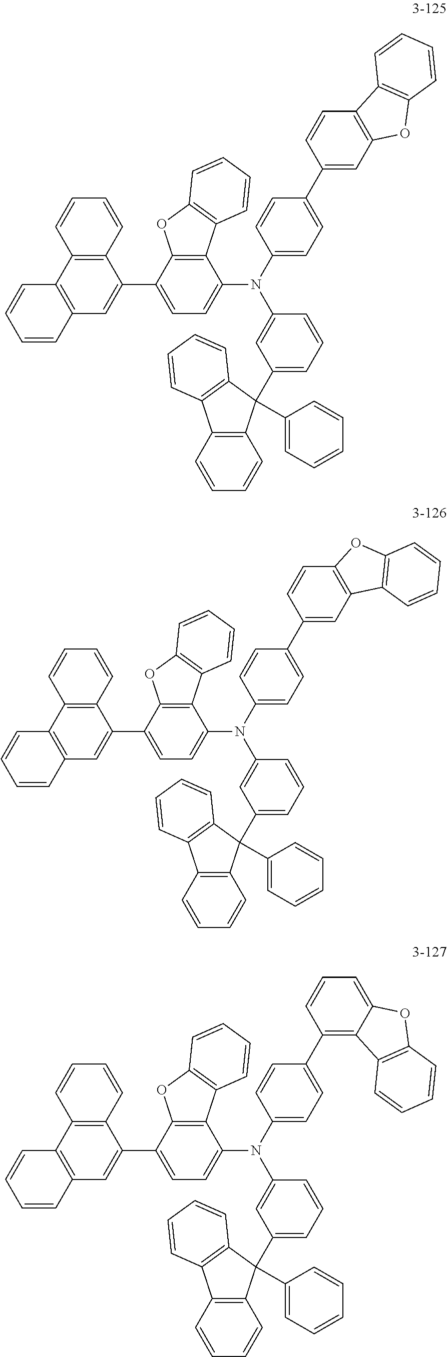 [SMILES: c1ccc(C2(c3cccc(N(c4ccc(-c5ccc6c(c5)oc5ccccc56)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5ccc6oc7ccccc7c6c5)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5cccc6oc7ccccc7c56)cc4)c4ccc(-c5cc6ccccc6c6ccccc56)c5oc6ccccc6c45)c3)c3ccccc3-c3ccccc32)cc1]

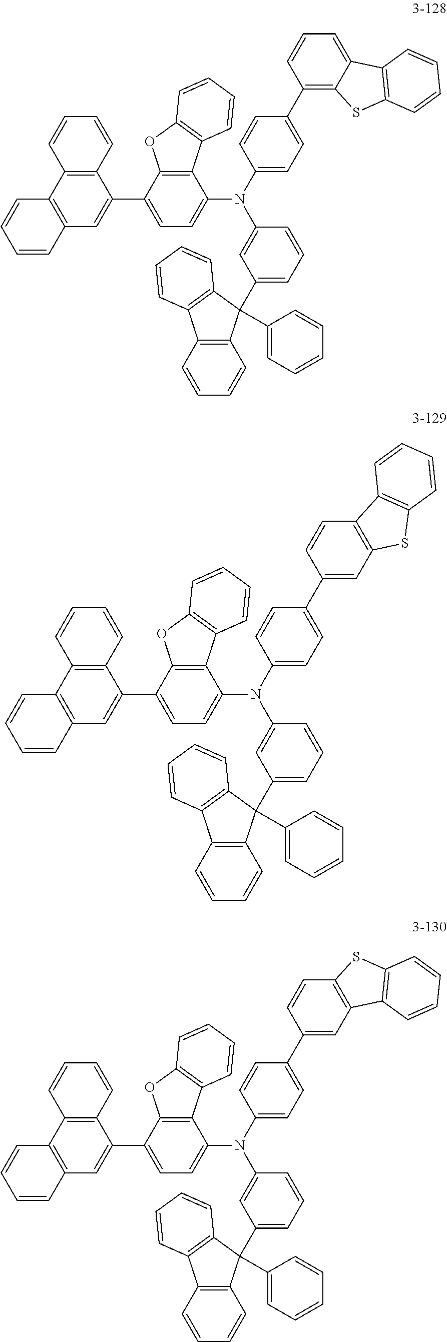 [SMILES: c1ccc(C2(c3cccc(N(c4ccc(-c5cccc6c5sc5ccccc56)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5ccc6c(c5)sc5ccccc56)cc4)c4ccc(-c5cc6ccccc6c6ccccc56)c5oc6ccccc6c45)c3)c3ccccc3-c3ccccc32)cc1 ; c1ccc(C2(c3cccc(N(c4ccc(-c5ccc6sc7ccccc7c6c5)cc4)c4ccc(-c5cc6ccccc6c6ccccc56)c5oc6ccccc6c45)c3)c3ccccc3-c3ccccc32)cc1]

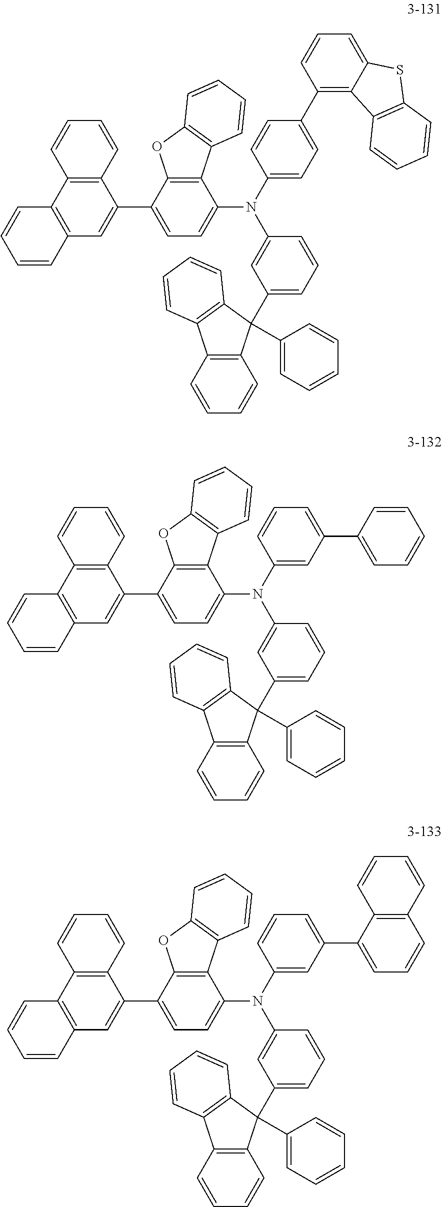 [SMILES: c1ccc(C2(c3cccc(N(c4ccc(-c5cccc6sc7ccccc7c56)cc4)c4ccc(-c5cc6ccccc6c6ccccc56)c5oc6ccccc6c45)c3)c3ccccc3-c3ccccc32)cc1 ; c1ccc(-c2cccc(N(c3cccc(C4(c5ccccc5)c5ccccc5-c5ccccc54)c3)c3ccc(-c4cc5ccccc5c5ccccc45)c4oc5ccccc5c34)c2)cc1 ; c1ccc(C2(c3cccc(N(c4cccc(-c5cccc6ccccc56)c4)c4ccc(-c5cc6ccccc6c6ccccc56)c5oc6ccccc6c45)c3)c3ccccc3-c3ccccc32)cc1]

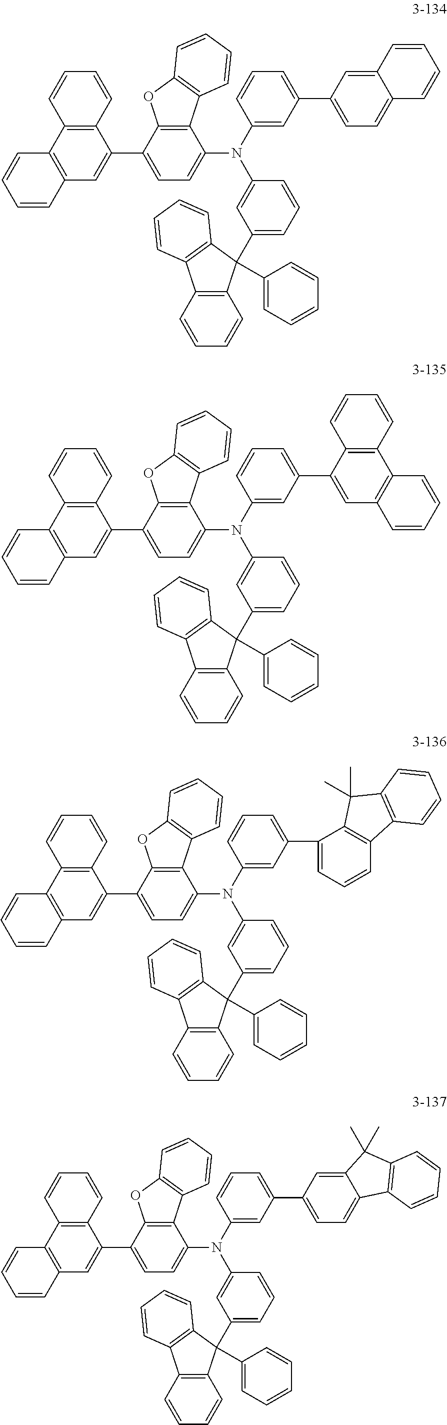 [SMILES: c1ccc(C2(c3cccc(N(c4cccc(-c5ccc6ccccc6c5)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5cc6ccccc6c6ccccc56)c4)c4ccc(-c5cc6ccccc6c6ccccc56)c5oc6ccccc6c45)c3)c3ccccc3-c3ccccc32)cc1 ; CC1(C)c2ccccc2-c2cccc(-c3cccc(N(c4cccc(C5(c6ccccc6)c6ccccc6-c6ccccc65)c4)c4ccc(-c5cc6ccccc6c6ccccc56)c5oc6ccccc6c45)c3)c21 ; CC1(C)c2ccccc2-c2ccc(-c3cccc(N(c4cccc(C5(c6ccccc6)c6ccccc6-c6ccccc65)c4)c4ccc(-c5cc6ccccc6c6ccccc56)c5oc6ccccc6c45)c3)cc21]

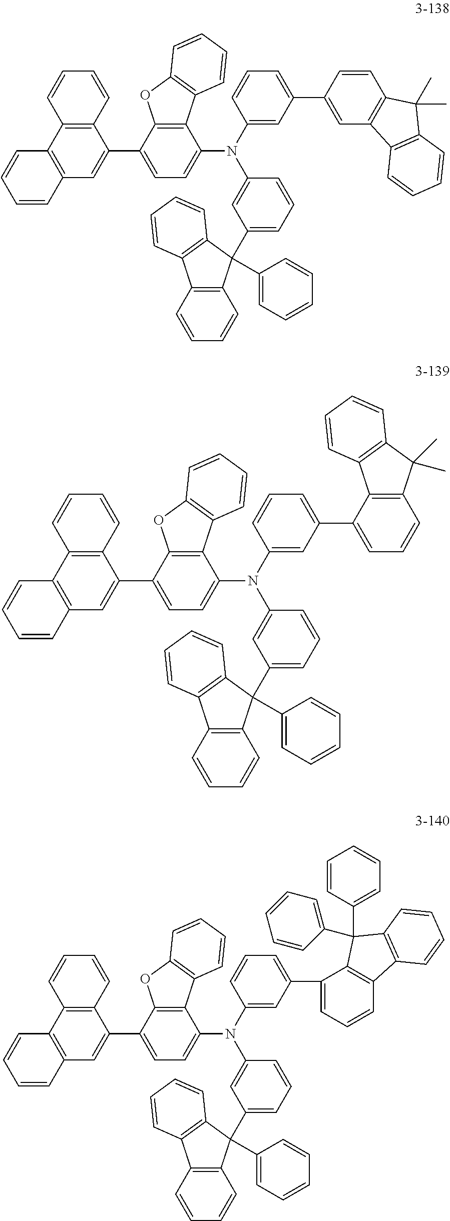 [SMILES: CC1(C)c2ccccc2-c2cc(-c3cccc(N(c4cccc(C5(c6ccccc6)c6ccccc6-c6ccccc65)c4)c4ccc(-c5cc6ccccc6c6ccccc56)c5oc6ccccc6c45)c3)ccc21 ; CC1(C)c2ccccc2-c2c(-c3cccc(N(c4cccc(C5(c6ccccc6)c6ccccc6-c6ccccc65)c4)c4ccc(-c5cc6ccccc6c6ccccc56)c5oc6ccccc6c45)c3)cccc21 ; c1ccc(C2(c3cccc(N(c4cccc(-c5cccc6c5C(c5ccccc5)(c5ccccc5)c5ccccc5-6)c4)c4ccc(-c5cc6ccccc6c6ccccc56)c5oc6ccccc6c45)c3)c3ccccc3-c3ccccc32)cc1]

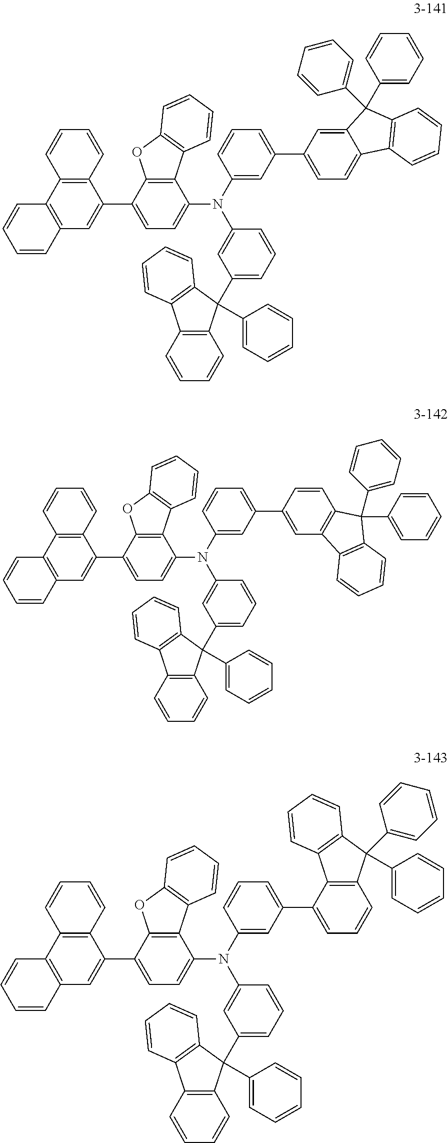 [SMILES: c1ccc(C2(c3cccc(N(c4cccc(-c5ccc6c(c5)C(c5ccccc5)(c5ccccc5)c5ccccc5-6)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5ccc6c(c5)-c5ccccc5C6(c5ccccc5)c5ccccc5)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5cccc6c5-c5ccccc5C6(c5ccccc5)c5ccccc5)c4)c4ccc(-c5cc6ccccc6c6ccccc56)c5oc6ccccc6c45)c3)c3ccccc3-c3ccccc32)cc1]

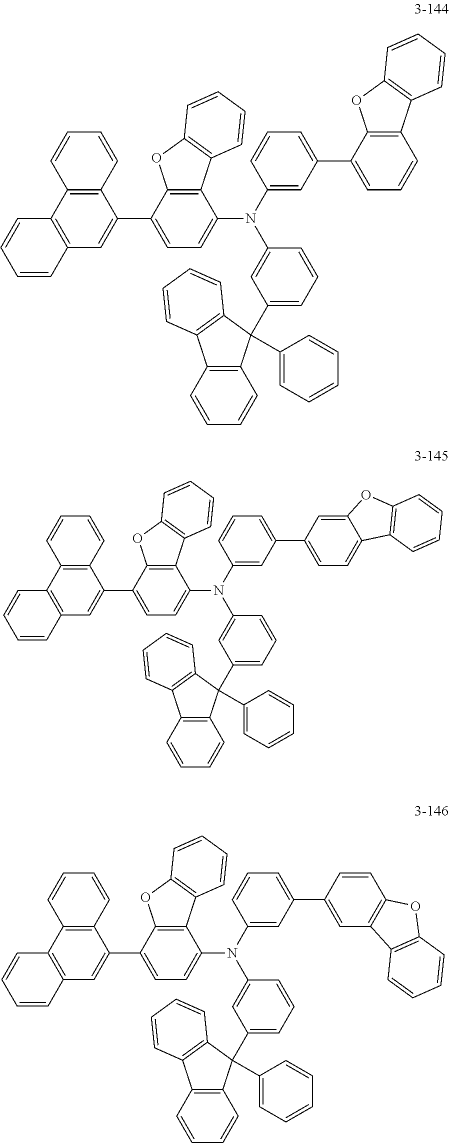 [SMILES: c1ccc(C2(c3cccc(N(c4cccc(-c5cccc6c5oc5ccccc56)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5ccc6c(c5)oc5ccccc56)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5ccc6oc7ccccc7c6c5)c4)c4ccc(-c5cc6ccccc6c6ccccc56)c5oc6ccccc6c45)c3)c3ccccc3-c3ccccc32)cc1]

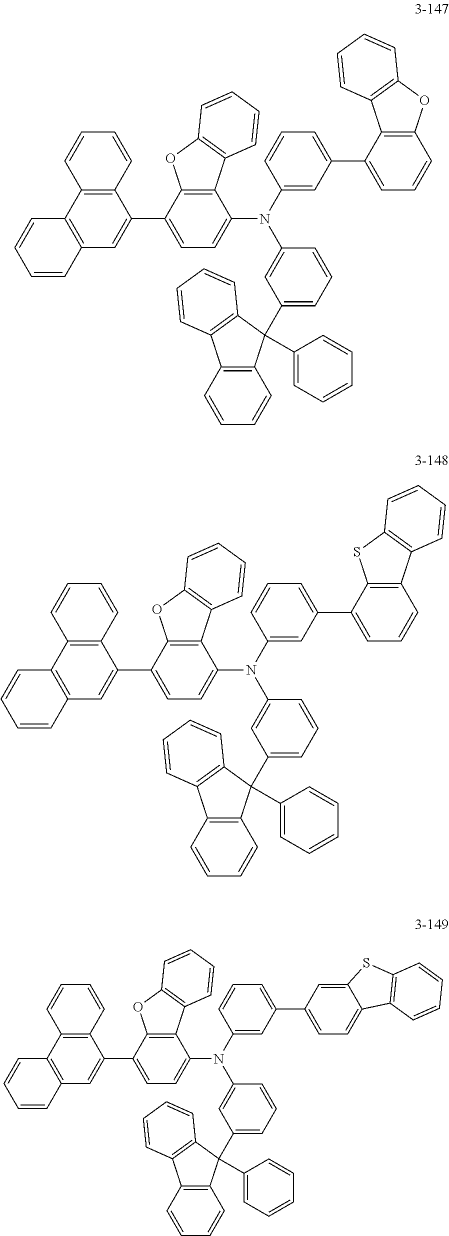 [SMILES: c1ccc(C2(c3cccc(N(c4cccc(-c5cccc6oc7ccccc7c56)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5cccc6c5sc5ccccc56)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5ccc6c(c5)sc5ccccc56)c4)c4ccc(-c5cc6ccccc6c6ccccc56)c5oc6ccccc6c45)c3)c3ccccc3-c3ccccc32)cc1]

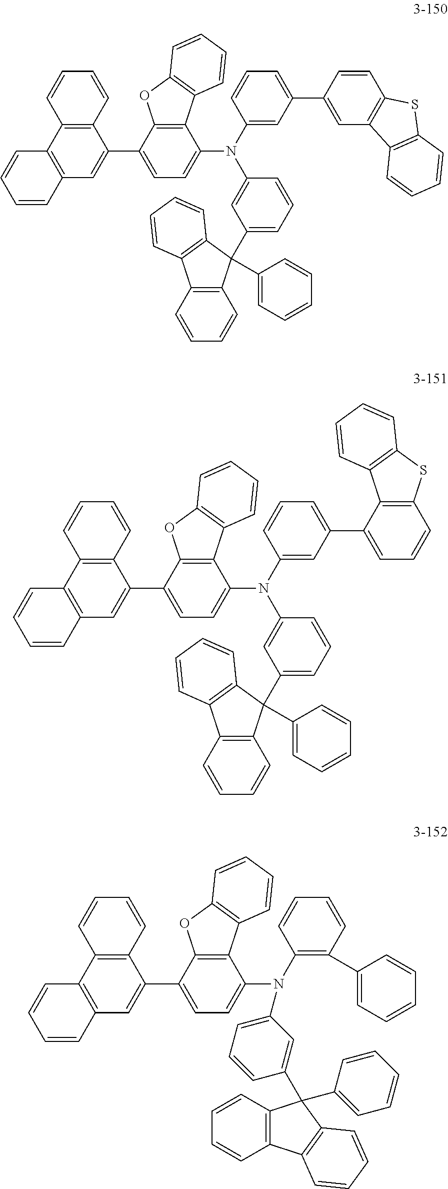 [SMILES: c1ccc(C2(c3cccc(N(c4cccc(-c5ccc6sc7ccccc7c6c5)c4)c4ccc(-c5cc6ccccc6c6ccccc56)c5oc6ccccc6c45)c3)c3ccccc3-c3ccccc32)cc1 ; c1ccc(C2(c3cccc(N(c4cccc(-c5cccc6sc7ccccc7c56)c4)c4ccc(-c5cc6ccccc6c6ccccc56)c5oc6ccccc6c45)c3)c3ccccc3-c3ccccc32)cc1 ; c1ccc(-c2ccccc2N(c2cccc(C3(c4ccccc4)c4ccccc4-c4ccccc43)c2)c2ccc(-c3cc4ccccc4c4ccccc34)c3oc4ccccc4c23)cc1]

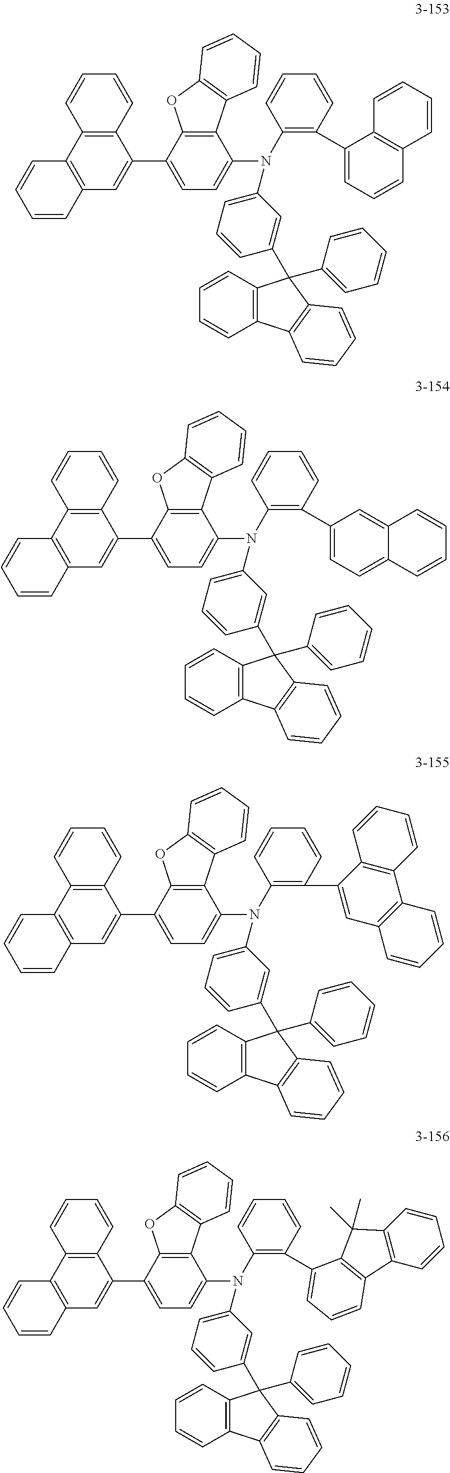 [SMILES: c1ccc(C2(c3cccc(N(c4ccccc4-c4cccc5ccccc45)c4ccc(-c5cc6ccccc6c6ccccc56)c5oc6ccccc6c45)c3)c3ccccc3-c3ccccc32)cc1 ; c1ccc(C2(c3cccc(N(c4ccccc4-c4ccc5ccccc5c4)c4ccc(-c5cc6ccccc6c6ccccc56)c5oc6ccccc6c45)c3)c3ccccc3-c3ccccc32)cc1 ; c1ccc(C2(c3cccc(N(c4ccccc4-c4cc5ccccc5c5ccccc45)c4ccc(-c5cc6ccccc6c6ccccc56)c5oc6ccccc6c45)c3)c3ccccc3-c3ccccc32)cc1 ; CC1(C)c2ccccc2-c2cccc(-c3ccccc3N(c3cccc(C4(c5ccccc5)c5ccccc5-c5ccccc54)c3)c3ccc(-c4cc5ccccc5c5ccccc45)c4oc5ccccc5c34)c21]

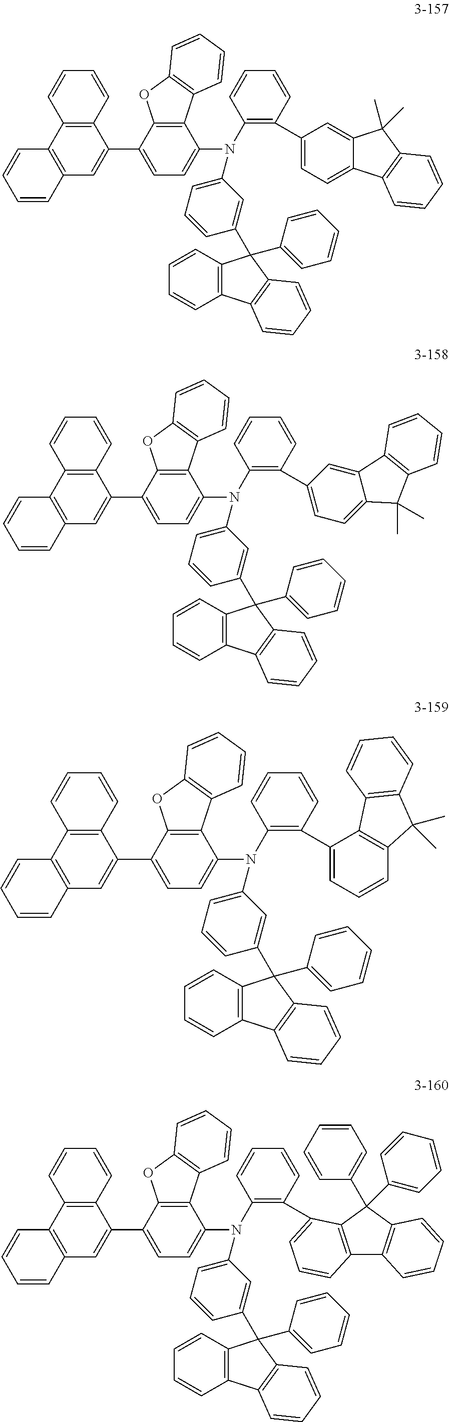

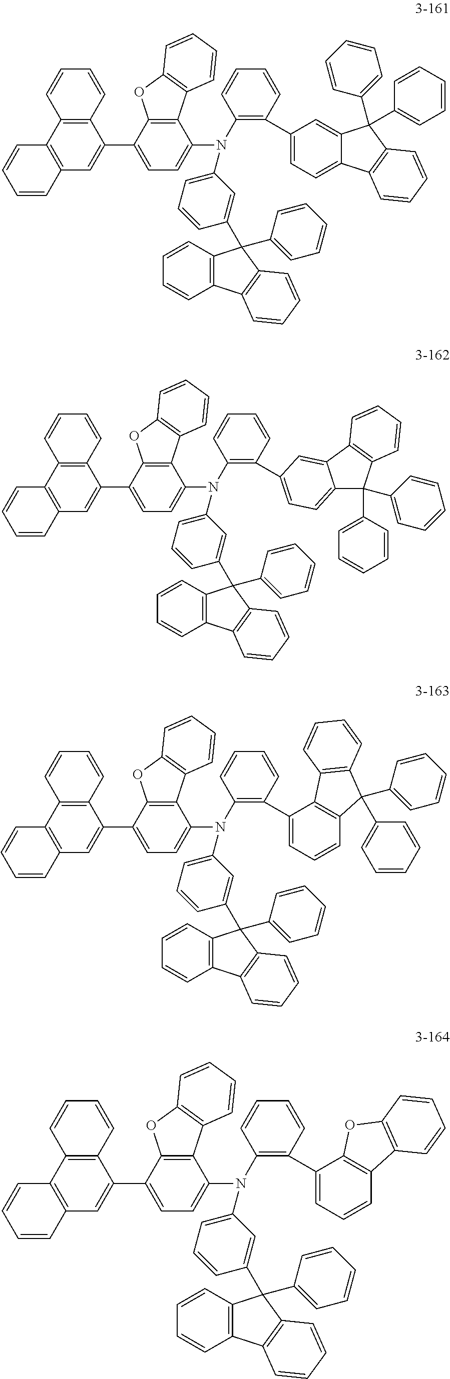

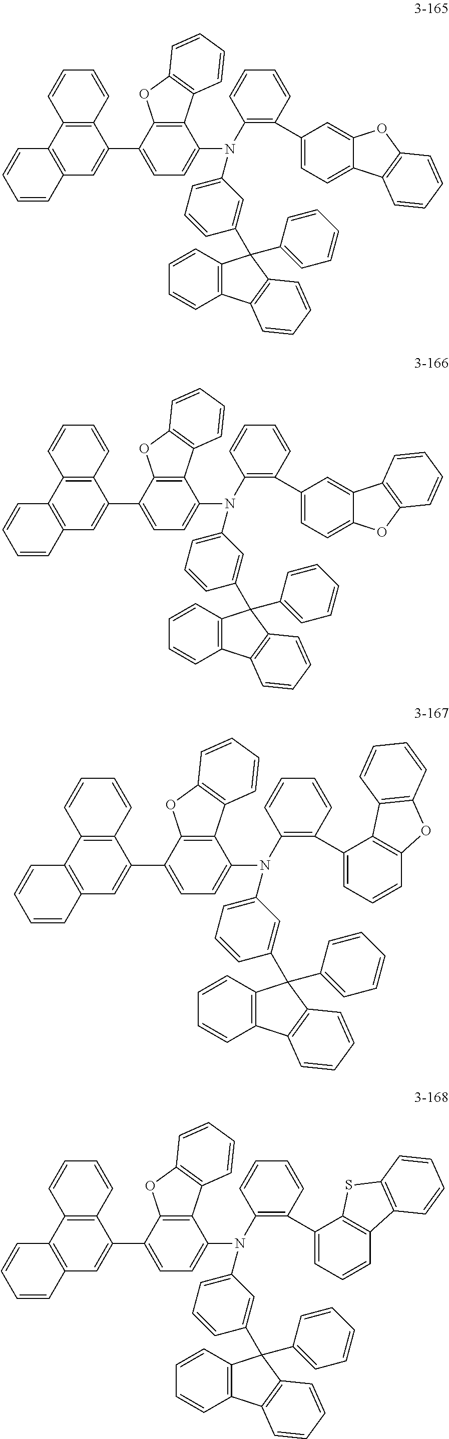 [SMILES: c1ccc(C2(c3cccc(N(c4ccccc4-c4ccc5c(c4)oc4ccccc45)c4ccc(-c5cc6ccccc6c6ccccc56)c5oc6ccccc6c45)c3)c3ccccc3-c3ccccc32)cc1 ; c1ccc(C2(c3cccc(N(c4ccccc4-c4ccc5oc6ccccc6c5c4)c4ccc(-c5cc6ccccc6c6ccccc56)c5oc6ccccc6c45)c3)c3ccccc3-c3ccccc32)cc1 ; c1ccc(C2(c3cccc(N(c4ccccc4-c4cccc5oc6ccccc6c45)c4ccc(-c5cc6ccccc6c6ccccc56)c5oc6ccccc6c45)c3)c3ccccc3-c3ccccc32)cc1 ; c1ccc(C2(c3cccc(N(c4ccccc4-c4cccc5c4sc4ccccc45)c4ccc(-c5cc6ccccc6c6ccccc56)c5oc6ccccc6c45)c3)c3ccccc3-c3ccccc32)cc1]

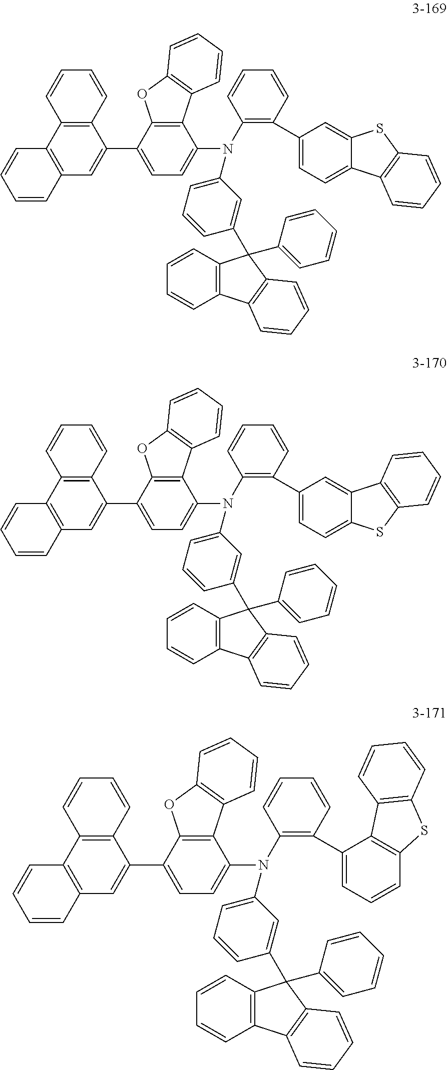 [SMILES: c1ccc(C2(c3cccc(N(c4ccccc4-c4ccc5c(c4)sc4ccccc45)c4ccc(-c5cc6ccccc6c6ccccc56)c5oc6ccccc6c45)c3)c3ccccc3-c3ccccc32)cc1 ; c1ccc(C2(c3cccc(N(c4ccccc4-c4ccc5sc6ccccc6c5c4)c4ccc(-c5cc6ccccc6c6ccccc56)c5oc6ccccc6c45)c3)c3ccccc3-c3ccccc32)cc1 ; c1ccc(C2(c3cccc(N(c4ccccc4-c4cccc5sc6ccccc6c45)c4ccc(-c5cc6ccccc6c6ccccc56)c5oc6ccccc6c45)c3)c3ccccc3-c3ccccc32)cc1]

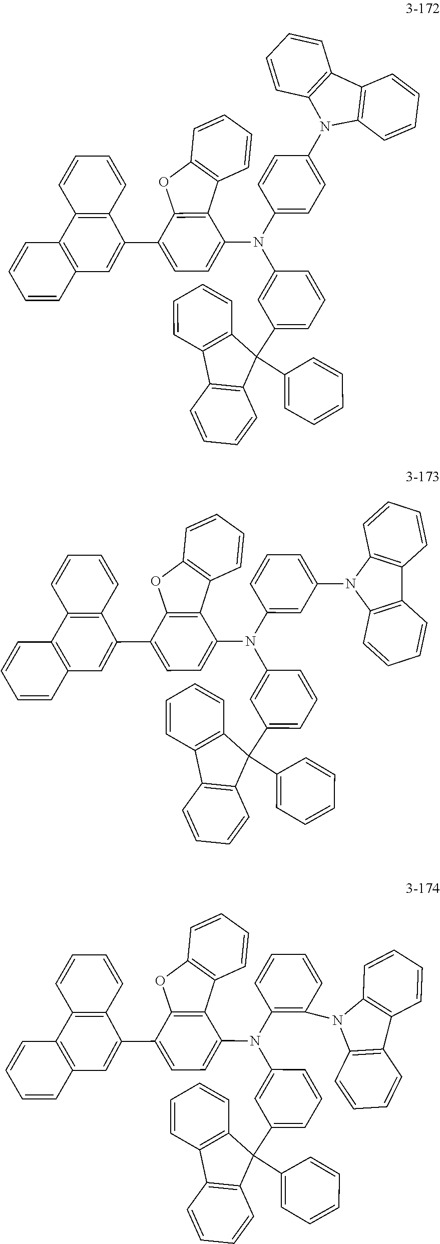 [SMILES: c1ccc(C2(c3cccc(N(c4ccc(-n5c6ccccc6c6ccccc65)cc4)c4ccc(-c5cc6ccccc6c6ccccc56)c5oc6ccccc6c45)c3)c3ccccc3-c3ccccc32)cc1 ; c1ccc(C2(c3cccc(N(c4cccc(-n5c6ccccc6c6ccccc65)c4)c4ccc(-c5cc6ccccc6c6ccccc56)c5oc6ccccc6c45)c3)c3ccccc3-c3ccccc32)cc1 ; c1ccc(C2(c3cccc(N(c4ccccc4-n4c5ccccc5c5ccccc54)c4ccc(-c5cc6ccccc6c6ccccc56)c5oc6ccccc6c45)c3)c3ccccc3-c3ccccc32)cc1]

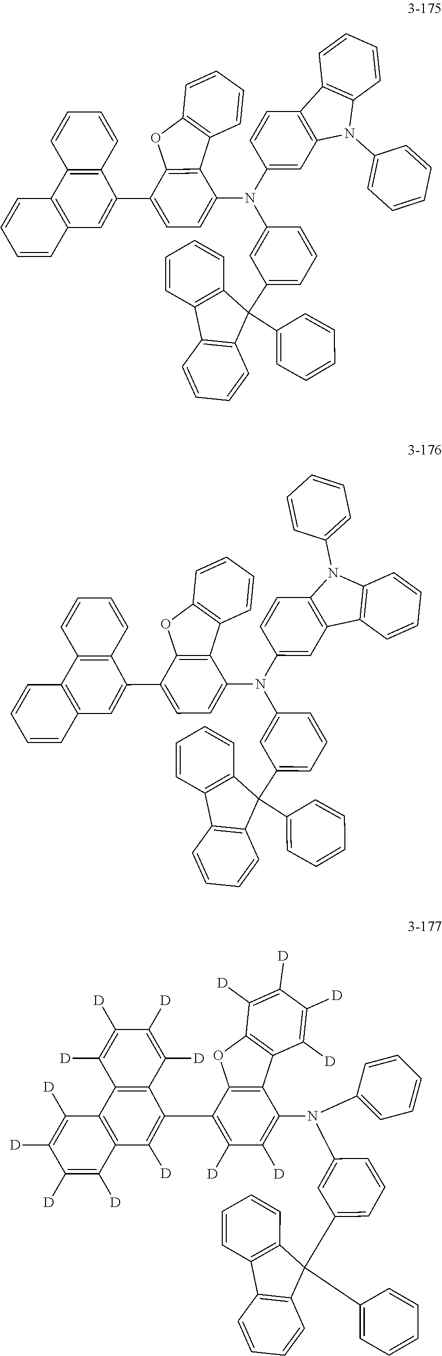 [SMILES: c1ccc(-n2c3ccccc3c3ccc(N(c4cccc(C5(c6ccccc6)c6ccccc6-c6ccccc65)c4)c4ccc(-c5cc6ccccc6c6ccccc56)c5oc6ccccc6c45)cc32)cc1 ; c1ccc(-n2c3ccccc3c3cc(N(c4cccc(C5(c6ccccc6)c6ccccc6-c6ccccc65)c4)c4ccc(-c5cc6ccccc6c6ccccc56)c5oc6ccccc6c45)ccc32)cc1 ; [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(N(c4ccccc4)c4cccc(C5(c6ccccc6)c6ccccc6-c6ccccc65)c4)c32)c1[2H]]

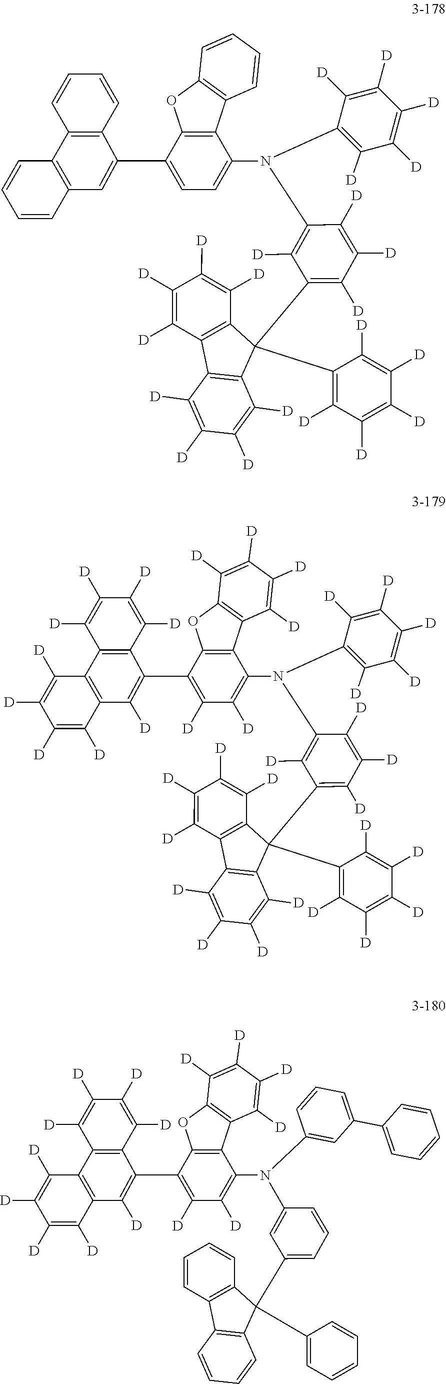

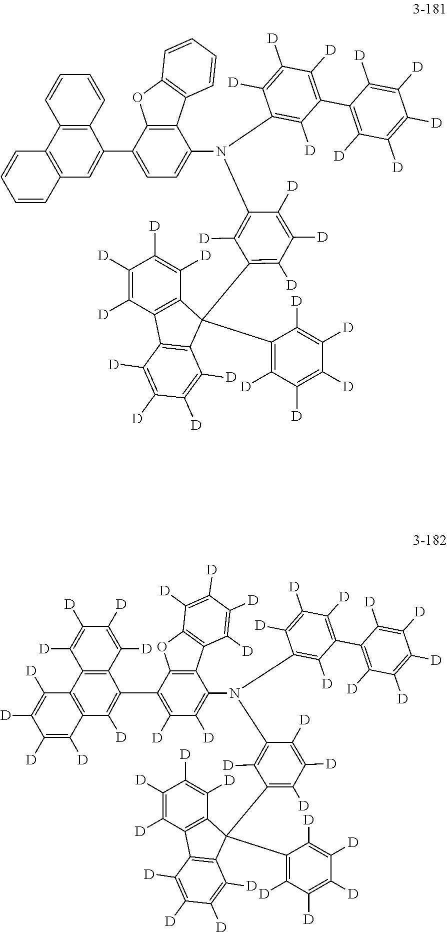

An organic light-emitting diode according to one exemplary embodiment of the present invention includes a first electrode (anode) and a second electrode (cathode) facing the first electrode, and may include one or more organic material layers which are disposed between the first electrode and the second electrode.

At least one of the one or more layers of the organic material layers may include an organic compound represented by Formula 1.

The organic material layer may include one or more layers among a hole injection layer (HIL), a hole transport layer (HTL), a hole transport auxiliary layer, an emission layer (EML), an electron transport auxiliary layer, an electron transport layer (ETL), and an electron injection layer (EIL).

For example, the organic light-emitting diode may have a structure in which a first electrode, a hole injection layer (HIL), a hole transport layer (HTL), a hole transport auxiliary layer, an emission layer (EML), an electron transport layer (ETL), an electron injection layer (EIL), and a second electrode are sequentially laminated.

In addition, the organic light-emitting diode may have a structure in which a first electrode, a hole injection layer, a hole transport layer, a hole transport auxiliary layer, an emission layer (EML), an electron transport auxiliary layer, an electron transport layer, an electron injection layer, and a second electrode are sequentially laminated.

Here, an organic material layer comprising the compound represented by Formula 1 according to one exemplary embodiment of the present invention may be a hole transport layer (HTL) or a hole transport auxiliary layer.

The one or more organic material layers may further include one or more selected from the group consisting of a hole injection layer, an emission layer, an electron transport auxiliary layer, an electron transport layer, and an electron injection layer.

For example, in a case where an organic compound represented by Formula 1 is used as a substance for a hole transport auxiliary layer, it is possible to provide an energy level that is suitable for a hole transport auxiliary layer to take a role in transferring holes from the hole transport layer to the light emission layer and blocking electrons coming from the light emission layer.

The organic light-emitting diode according to one exemplary embodiment of the present invention makes it possible to excellently realize the targeted color coordinates even in a case where a hole transport layer and/or a hole transport auxiliary layer, which contains the organic compound represented by Formula 1 according to the present invention, is combined with a light emission layer of any color.

The first electrode may be an anode, and the first electrode may contain a substance such as indium tin oxide (ITO), indium zinc oxide (IZO), tin oxide (SnO₂), or zinc oxide (ZnO), which is transparent and has excellent conductivity.

The second electrode may be a cathode, and the second electrode may contain a substance such as lithium (Li), aluminum (Al), aluminum-lithium (Al—Li), calcium (Ca), magnesium (Mg), magnesium-indium (Mg—In), or magnesium-silver (Mg—Ag). In addition, in a case of an organic light-emitting diode of a top emission type, indium tin oxide (ITO) or indium zinc oxide (IZO) may be used to form a transparent second electrode through which light may transmit.

A capping layer (CPL) may be formed on the surface of the second electrode with a composition for forming a capping layer.

In addition, a protective film (encapsulation layer or protecting layer) may be additionally disposed on the capping layer to protect the organic light-emitting diode from moisture and oxygen. Such a protective film may be formed of a curable adhesive composition comprising an inorganic hygroscopic agent.

The compound for the hole injection layer or the hole transport layer is not particularly limited, and any compound may be used as long as it is generally used as the compound for the hole injection layer or the hole transport layer. Non-limiting examples of the compound for the hole injection layer or the hole transport layer include a phthalocyanine derivative, a porphyrin derivative, a triarylamine derivative, an indolocarbazole derivative, and the like. Examples thereof include 1,4,5,8,9,11-hexaazatriphenylene-hexacarbonitrile (HAT-CN), copper phthalocyanine (CuPc), 4,4′,4′-tris(3-methylphenylamino)triphenyl amine (m-MTDATA), 4,4′,4′-tris(3-methylphenylamino)phenoxybenzene (m-MTDAPB), 4,4′-tri(N-carbazolyl)triphenylamine (TCTA), 4,4′-tris(N-(2-naphthyl)-N-phenylamino)-triphenylamine (2-TNATA), N4,N4,N4′,N4′-tetra([1,1′-biphenyl]-4-yl)-[1,1′-biphenyl]-4,4′-diamine, bis(N-(1-naphthyl-n-phenyl))benzidine (α-NPD), N,N′-di(naphthalene-1-yl)-N,N′-biphenyl-benzidine (NPB), N,N′-biphenyl-N,N′-bis(3-methylphenyl)-1,1′-biphenyl-4,4′-diamine (TPD), and the like.

The compound contained in the emission layer is not particularly limited, and any compound may be used as long as it is generally used as a compound for an emission layer. A single-type light emitting compound or a light emitting host compound may be used.

Examples of the light emitting compound for the emission layer include a compound that can emit light through phosphorescence, fluorescence, thermally activated delayed fluorescence, that is, TADF (also referred to as E-type delayed fluorescence), triplet-triplet extinction, or a combination of these processes; however, the light emitting compound is not limited thereto. The light emitting compound can be selected from various materials according to the desired color of the emitted light. Non-limiting examples of light emitting compound include a condensed ring derivative such as phenanthrene, anthracene, pyrene, tetracene, pentacene, perylene, naphthopyrene, dibenzopyrene, rubrene, or chrysene, a benzoxazole derivative, a benzothiazole derivative, a benzimidazole derivative, a benzotriazole derivative, an oxazole derivative, an oxadiazole derivative, a thiazole derivative, an imidazole derivative, a thiadiazole derivative, a triazole derivative, a pyrazoline derivative, a stilbene derivative, a thiophene derivative, a tetraphenylbutadiene derivative, a cyclopentadiene derivative, a bistyryl derivative, a bistyrylarylene derivative, a diazaindacene derivative, a furan derivative, a benzofuran derivative, an isobenzofuran derivative, a dibenzofuran derivative, a coumarin derivative, a dicyanomethylenepyran derivative, a dicyanomethylenethiopyran derivative, a polymethine derivative, a cyanine derivative, an oxobenzanthracene derivative, a xanthene derivative, a rhodamine derivative, a fluorescein derivative, a pyrylium derivative, a carbostyril derivative, an acridine derivative, an oxazine derivative, a phenylene oxide derivative, a quinacridone derivative, a quinazoline derivative, a pyrrolopyridine derivative, a furopyridine derivative, a 1,2,5-thiadiazolopyrene derivative, a pyrromethene derivative, a perinone derivative, a pyrrolopyrrole derivative, a squarylium derivative, a violanthrone derivative, a phenazine derivative, an acridone derivative, a deazaflavin derivative, a fluorene derivative, a benzofluorene derivative, an aromatic boron derivative, an aromatic nitrogen boron derivative, and a metal complex (for example, a complex in which a metal such as Ir, Pt, Au, Eu, Ru, Re, Ag, or Cu is bonded to a heteroaromatic ring ligand), and the like. Examples thereof include N1,N1,N6,N6-tetrakis(4-(1-silyl)phenyl)pyrene-1,6-diamine, 2,12-di-tut-butyl-5,9-bis(4-(tut-butyl)phenyl)-7-(3,5-di-tut-butyl)phenyl-5,9-dihydro-5,9-diaza-13b-boranaphto[3,2,1-de]anthracene (t-DABNA-dtB), platinum octaethylporphyrin (PtOEP), Ir(ppy)₃, Ir(ppy)₂(acac), Ir(mppy)₃, Ir(PPy)₂(m-bppy), BtpIr(acac), Ir(btp)₂(acac), Ir(2-phq)₃, Hex-Ir(phq)₃, Ir(fbi)₂(acac), fac-Tris(2-(3-p-xylyl)phenyl)pyridine iridium(III), Eu(dbm)₃(Phen), Ir(piq)₃, Ir(piq)₂(acac), Ir(Fliq)₂(acac), Ir(Flq)₂(acac), Ru(dtb-bpy)₃·2(PF₆), Ir(BT)₂(acac), Ir(DMP)₃, Ir(Mphq)₃IR(phq)₂tpy, fac-Ir(ppy)₂Pc, Ir(dp)PQ₂, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, and the like.

As the host compound of the emission layer, a host having light emitting properties, a host having hole transportability, a host having electron transportability, or a combination thereof can be be used. Non-limiting examples of the host compound having light emitting properties include a condensed ring derivative such as anthracene or pyrene, a bistyryl derivative such as a bistyryl anthracene derivative or a distyrylbenzene derivative, a tetraphenylbutadiene derivative, a cyclopentadiene derivative, a fluorene derivative, a benzofluorene derivative, an n-phenylcarbazole (9-phenylcarbazole) derivative, a carbazolylnitrile derivative, and the like. Non-limiting examples of the host substance having hole transportability include a carbazole derivative, a dibenzofuran derivative, a dibenzothiophene derivative, a triarylamine derivative, an indolocarbazole derivative, and a benzoxazinophenoxazine derivative. Non-limiting examples of the host substance having electron transportability include a pyridine derivative, a triazine derivative, a phosphine oxide derivative, a benzofuropyridine derivative, and a dibenzoxacillin derivative. Examples thereof include 9,10-bis(2-naphthyl)anthracene (ADN), tris(8-hydroxyquinolinato)aluminum (Alq₃), 8-hydroxyquinoline beryllium salt (BAlq), 4,4′-biphenylethenyl)-1,1′-biphenyl (DPVBi) series, spiro-4,4′-bis(2,2-biphenylethenyl)-1,1′-biphenyl (spiro-DPVBi) series, 2-(2-benzoxazolyl)-phenol lithium salt (LiPBO), bis(biphenylvinyl)benzene, an aluminum-quinoline metal complex, a metal complex of imidazole, thiazole, or oxazole, and the like.

The compound for the electron injection layer or the electron transport layer compound is not particularly limited, and any compound may be used as long as it is generally used as a compound for the electron injection layer or the electron transport layer. Non-limiting examples of the compound for the electron injection layer or the electron transport layer include a pyridine derivative, a naphthalene derivative, an anthracene derivative, a phenanthroline derivative, a perinone derivative, a coumarin derivative, a naphthalimide derivative, an anthraquinone derivative, a diphenoquinone derivative, a diphenylquinone derivative, a perylene derivative, an oxadiazole derivative, a thiophene derivative, a thiazole derivative, a thiadiazole derivative, a metal complex of an oxine derivative, a quinolinol-based metal complex, a quinoxaline derivative, a polymer of a quinoxaline derivative, benzazole compounds, a gallium complex, a pyrazole derivative, a perfluorinated phenylene derivative, a triazine derivative, a pyrazine derivative, a benzoquinoline derivative, an imidazopyridine derivative, a borane derivative, a benzimidazole derivative, a benzoxazole derivative, a benzothiazole derivative, a quinoline derivative, an oligopyridine derivative such as terpyridine, a bipyridine derivative, a terpyridine derivative, a naphthyridine derivative, an aldazine derivative, a carbazole derivative, an indole derivative, a phosphine oxide derivative, a bistyryl derivative, a quinolinol-based metal complex, a hydroxazole-based metal complex, an azomethine-based metal complex, a tropolone-based metal complex, a flavonol-based metal complex, a benzoquinoline-based metal complex, a metal salt, and the like. These materials are used alone; however, they may be used in combination with other materials. For example, substances such as 2-(4-(9,10-di(naphthalene-2-yl)anthracene-2-yl)phenyl)-1-phenyl-1H-benzo[d]imidazole, tris(8-hydroxyquinolinato)aluminum (Alq₃), LiF, Liq, Li₂O, BaO, NaCl, and CsF may be included.

The compound for the electron transport auxiliary layer positioned between the electron transport layer and the emission layer is not particularly limited, and any compound may be used as long as it is generally used as a compound for the electron transport auxiliary layer. For example, the electron transport auxiliary layer may contain a pyrimidine derivative and the like.

The organic light-emitting diode according to one exemplary embodiment of the present invention may belong to a top light emitting-type or may belong to a bottom light emitting-type.

The organic light-emitting diode according to one exemplary embodiment of the present invention may be used in a display device.

The organic light-emitting diode according to one exemplary embodiment of the present invention may be applied to a transparent display device, a mobile display device, a flexible display device, and the like; however, the organic light-emitting diode according to one exemplary embodiment of the present invention is not limited thereto.

The organic light-emitting diode according to one exemplary embodiment of the present invention may include a tandem structure including a plurality of light emitting stacks between the anode and the cathode.

Hereinafter, a method of synthesizing the above-described compound will be described with reference to a representative example. However, the synthesis method for the compounds according to the present invention is not limited to the method exemplified below, or the implementation of the present invention is not limited to the following examples and experimental examples.

### Synthesis Example

Representatively, a synthesis example for Compound 2-15 (P22) is described, and compounds according to the present invention, represented by Formula 1, may be synthesized in a manner similar to the reaction for Compound 2-15 (P22).

In the following Reaction Scheme, a solvent, a catalyst, a protective group, a release group, a reaction temperature, a reaction time, an equivalent of a reactant, and the like are representative examples, and all the solvent, the catalyst, the protective group, the release group, the reaction temperature, the reaction time, the equivalent of a reactant, and the like, which are equivalent to those described above, may be used.

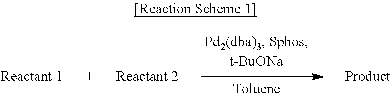

Under nitrogen flow, a reactant 1 (33.29 mmol) of P22, a reactant 2 (32.64 mmol) of P22, t-BuONa (65.28 mmol), Pd₂(dba)₃ (0.49 mmol), Sphos (0.98 mmol), and toluene were added to a reaction flask and subjected to reflux while stirring. After completion of the reaction, the organic layer was extracted using toluene and water. The extracted solution was treated with MgSO₄ to remove residual moisture, subjected to concentration under reduced pressure, and then subjected to purification using a column chromatography method and then recrystallization to obtain a product of P22. The synthesis results of the product of P22 are shown in Table 1 below.

TABLE 1

| Item | Reactant 1 | Reactant 2 |
|---|---|---|
| P1 | 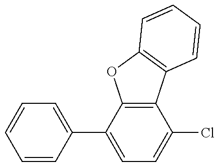 [SMILES: Clc1ccc(-c2ccccc2)c2oc3ccccc3c12] | 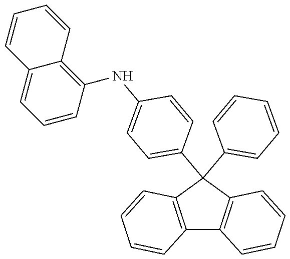 [SMILES: c1ccc(C2(c3ccc(Nc4cccc5ccccc45)cc3)c3ccccc3-c3ccccc32)cc1] |
| P2 | 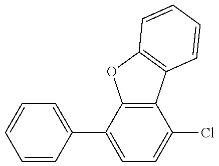 [SMILES: Clc1ccc(-c2ccccc2)c2oc3ccccc3c12] | 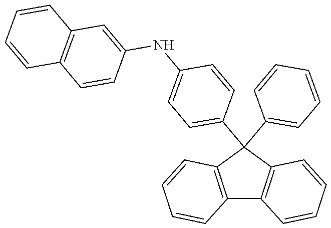 [SMILES: c1ccc(C2(c3ccc(Nc4ccc5ccccc5c4)cc3)c3ccccc3-c3ccccc32)cc1] |
| P3 | 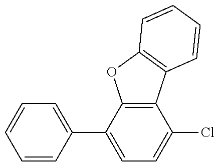 [SMILES: Clc1ccc(-c2ccccc2)c2oc3ccccc3c12] | 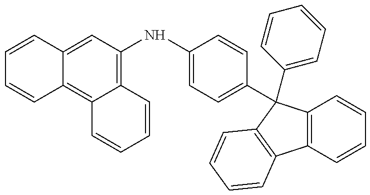 [SMILES: c1ccc(C2(c3ccc(Nc4cc5ccccc5c5ccccc45)cc3)c3ccccc3-c3ccccc32)cc1] |
| P4 | 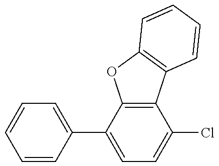 [SMILES: Clc1ccc(-c2ccccc2)c2oc3ccccc3c12] | 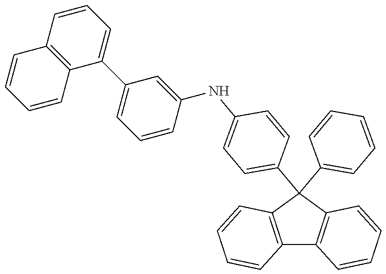 [SMILES: c1ccc(C2(c3ccc(Nc4cccc(-c5cccc6ccccc56)c4)cc3)c3ccccc3-c3ccccc32)cc1] |
| P5 | 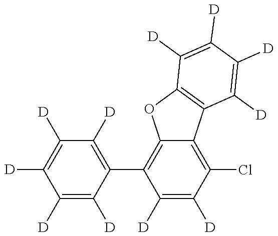 [SMILES: [2H]c1c([2H])c([2H])c(-c2c([2H])c([2H])c(Cl)c3c2oc2c([2H])c([2H])c([2H])c([2H])c23)c([2H])c1[2H]] | 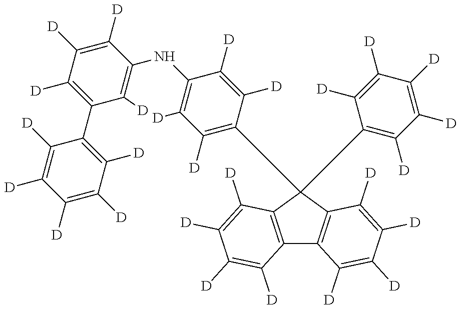 [SMILES: [2H]c1c([2H])c([2H])c(-c2c([2H])c([2H])c([2H])c(Nc3c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c([2H])c3[2H])c2[2H])c([2H])c1[2H]] |
| P6 | 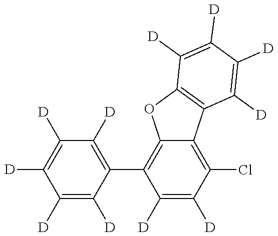 [SMILES: [2H]c1c([2H])c([2H])c(-c2c([2H])c([2H])c(Cl)c3c2oc2c([2H])c([2H])c([2H])c([2H])c23)c([2H])c1[2H]] | 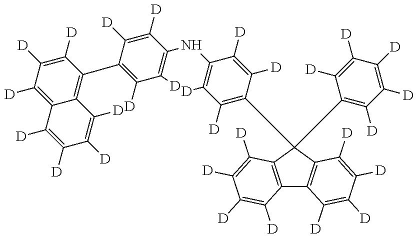 [SMILES: [2H]c1c([2H])c([2H])c(C2(c3c([2H])c([2H])c(Nc4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c([2H])c([2H])c3-c3c([2H])c([2H])c([2H])c([2H])c32)c([2H])c1[2H]] |
| P7 | 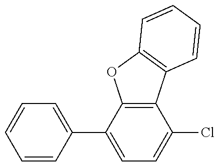 [SMILES: Clc1ccc(-c2ccccc2)c2oc3ccccc3c12] | 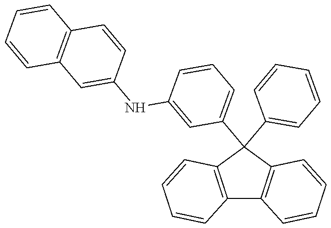 [SMILES: c1ccc(C2(c3cccc(Nc4ccc5ccccc5c4)c3)c3ccccc3-c3ccccc32)cc1] |
| P8 | 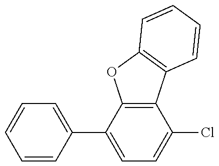 [SMILES: Clc1ccc(-c2ccccc2)c2oc3ccccc3c12] | 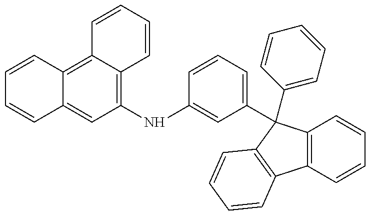 [SMILES: c1ccc(C2(c3cccc(Nc4cc5ccccc5c5ccccc45)c3)c3ccccc3-c3ccccc32)cc1] |
| P9 | 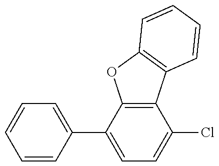 [SMILES: Clc1ccc(-c2ccccc2)c2oc3ccccc3c12] | 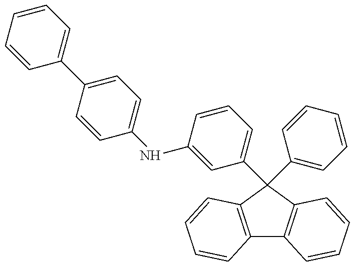 [SMILES: c1ccc(-c2ccc(Nc3cccc(C4(c5ccccc5)c5ccccc5-c5ccccc54)c3)cc2)cc1] |
| P10 | 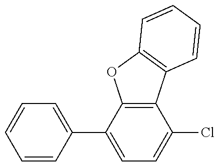 [SMILES: Clc1ccc(-c2ccccc2)c2oc3ccccc3c12] | 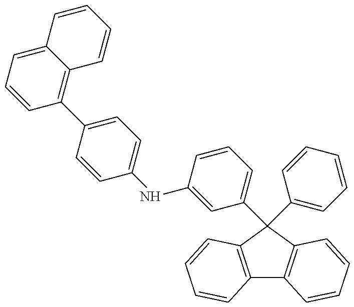 [SMILES: c1ccc(C2(c3cccc(Nc4ccc(-c5cccc6ccccc56)cc4)c3)c3ccccc3-c3ccccc32)cc1] |
| P11 | 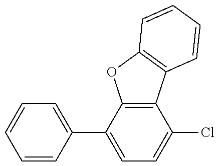 [SMILES: Clc1ccc(-c2ccccc2)c2oc3ccccc3c12] | 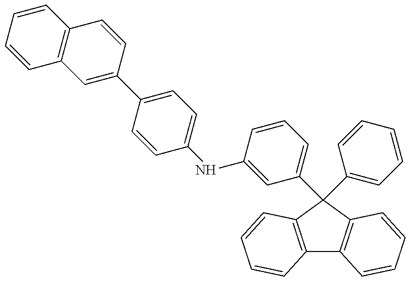 [SMILES: c1ccc(C2(c3cccc(Nc4ccc(-c5ccc6ccccc6c5)cc4)c3)c3ccccc3-c3ccccc32)cc1] |
| P12 | 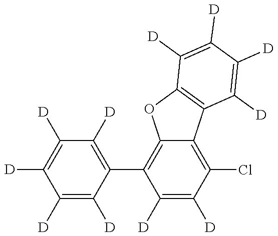 [SMILES: [2H]c1c([2H])c([2H])c(-c2c([2H])c([2H])c(Cl)c3c2oc2c([2H])c([2H])c([2H])c([2H])c23)c([2H])c1[2H]] | 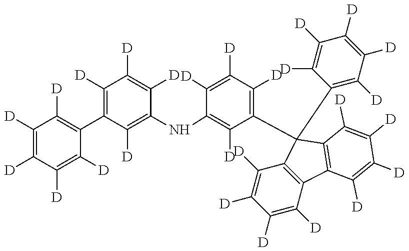 [SMILES: [2H]c1c([2H])c([2H])c(-c2c([2H])c([2H])c([2H])c(Nc3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c2[2H])c([2H])c1[2H]] |
| P13 | 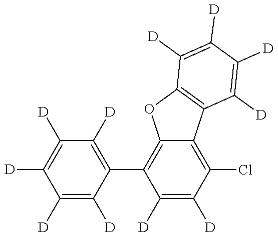 [SMILES: [2H]c1c([2H])c([2H])c(-c2c([2H])c([2H])c(Cl)c3c2oc2c([2H])c([2H])c([2H])c([2H])c23)c([2H])c1[2H]] | 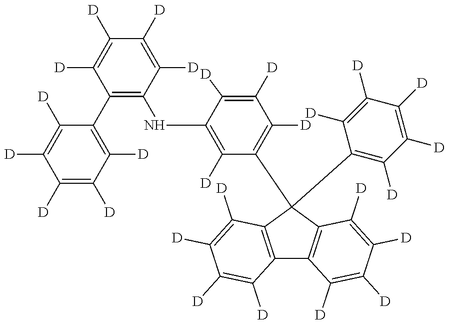 [SMILES: [2H]c1c([2H])c([2H])c(-c2c([2H])c([2H])c([2H])c([2H])c2Nc2c([2H])c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c2[2H])c([2H])c1[2H]] |
| P14 | 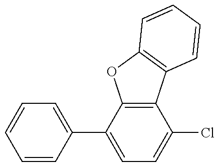 [SMILES: Clc1ccc(-c2ccccc2)c2oc3ccccc3c12] | 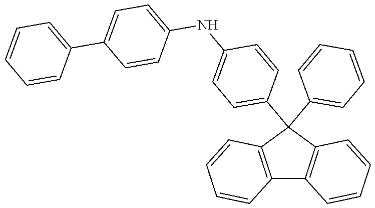 [SMILES: c1ccc(-c2ccc(Nc3ccc(C4(c5ccccc5)c5ccccc5-c5ccccc54)cc3)cc2)cc1] |
| P15 | 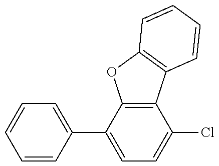 [SMILES: Clc1ccc(-c2ccccc2)c2oc3ccccc3c12] | 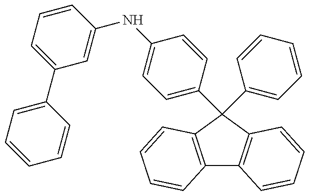 [SMILES: c1ccc(-c2cccc(Nc3ccc(C4(c5ccccc5)c5ccccc5-c5ccccc54)cc3)c2)cc1] |
| P16 | 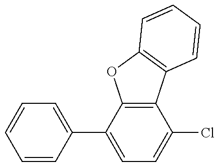 [SMILES: Clc1ccc(-c2ccccc2)c2oc3ccccc3c12] | 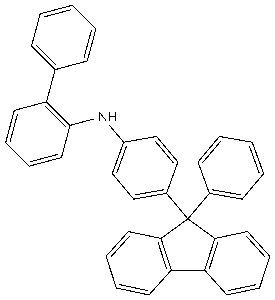 [SMILES: c1ccc(-c2ccccc2Nc2ccc(C3(c4ccccc4)c4ccccc4-c4ccccc43)cc2)cc1] |
| P17 | 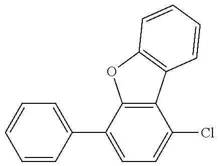 [SMILES: Clc1ccc(-c2ccccc2)c2oc3ccccc3c12] | 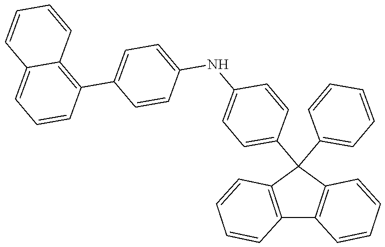 [SMILES: c1ccc(C2(c3ccc(Nc4ccc(-c5cccc6ccccc56)cc4)cc3)c3ccccc3-c3ccccc32)cc1] |
| P18 | 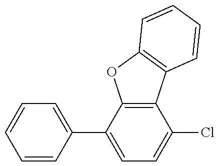 [SMILES: Clc1ccc(-c2ccccc2)c2oc3ccccc3c12] | 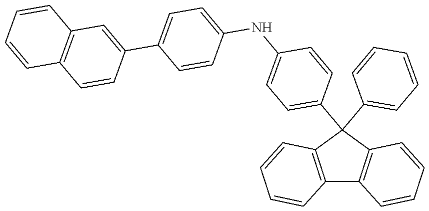 [SMILES: c1ccc(C2(c3ccc(Nc4ccc(-c5ccc6ccccc6c5)cc4)cc3)c3ccccc3-c3ccccc32)cc1] |
| P19 | 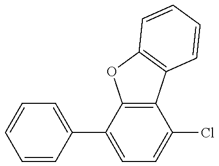 [SMILES: Clc1ccc(-c2ccccc2)c2oc3ccccc3c12] | 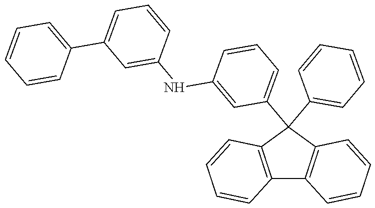 [SMILES: c1ccc(-c2cccc(Nc3cccc(C4(c5ccccc5)c5ccccc5-c5ccccc54)c3)c2)cc1] |
| P20 | 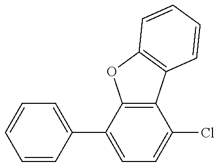 [SMILES: Clc1ccc(-c2ccccc2)c2oc3ccccc3c12] | 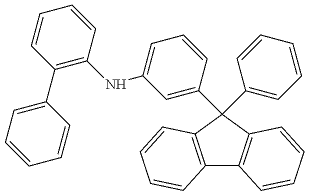 [SMILES: c1ccc(-c2ccccc2Nc2cccc(C3(c4ccccc4)c4ccccc4-c4ccccc43)c2)cc1] |
| P21 | 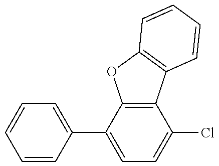 [SMILES: Clc1ccc(-c2ccccc2)c2oc3ccccc3c12] | 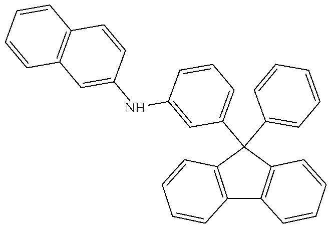 [SMILES: c1ccc(C2(c3cccc(Nc4ccc5ccccc5c4)c3)c3ccccc3-c3ccccc32)cc1] |
| P22 | 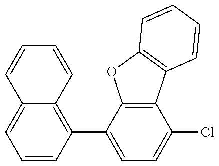 [SMILES: Clc1ccc(-c2cccc3ccccc23)c2oc3ccccc3c12] | 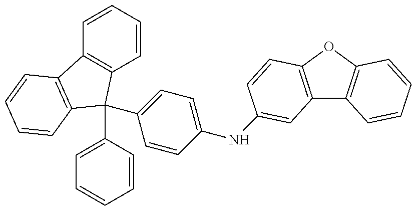 [SMILES: c1ccc(C2(c3ccc(Nc4ccc5oc6ccccc6c5c4)cc3)c3ccccc3-c3ccccc32)cc1] |
| P23 | 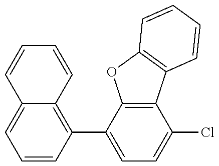 [SMILES: Clc1ccc(-c2cccc3ccccc23)c2oc3ccccc3c12] | 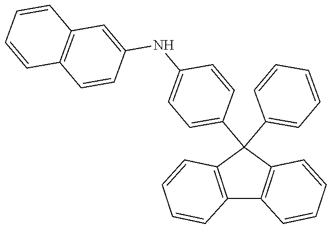 [SMILES: c1ccc(C2(c3ccc(Nc4ccc5ccccc5c4)cc3)c3ccccc3-c3ccccc32)cc1] |
| P24 | 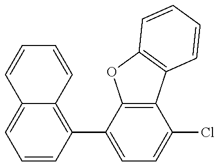 [SMILES: Clc1ccc(-c2cccc3ccccc23)c2oc3ccccc3c12] | 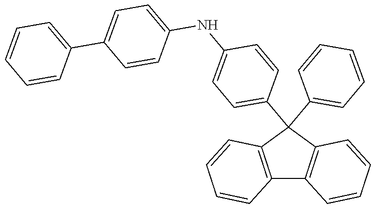 [SMILES: c1ccc(-c2ccc(Nc3ccc(C4(c5ccccc5)c5ccccc5-c5ccccc54)cc3)cc2)cc1] |
| P25 | 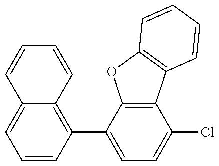 [SMILES: Clc1ccc(-c2cccc3ccccc23)c2oc3ccccc3c12] | 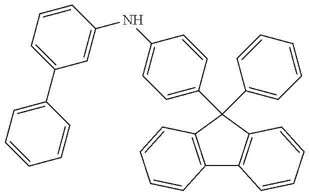 [SMILES: c1ccc(-c2cccc(Nc3ccc(C4(c5ccccc5)c5ccccc5-c5ccccc54)cc3)c2)cc1] |
| P26 | 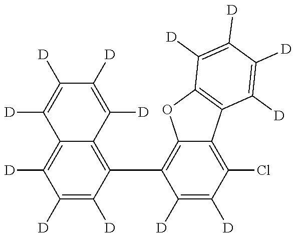 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 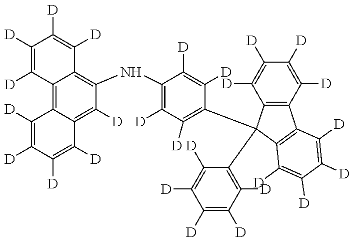 [SMILES: [2H]c1c([2H])c([2H])c(C2(c3c([2H])c([2H])c(Nc4c([2H])c5c([2H])c([2H])c([2H])c([2H])c5c5c([2H])c([2H])c([2H])c([2H])c45)c([2H])c3[2H])c3c([2H])c([2H])c([2H])c([2H])c3-c3c([2H])c([2H])c([2H])c([2H])c32)c([2H])c1[2H]] |
| P27 | 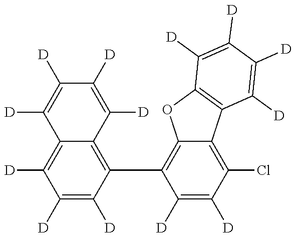 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 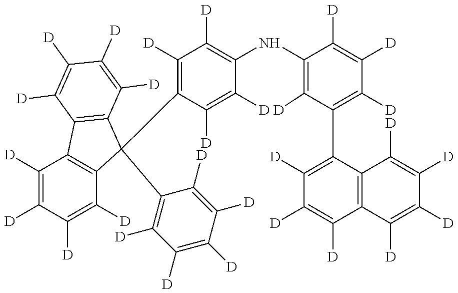 [SMILES: [2H]c1c([2H])c([2H])c(C2(c3c([2H])c([2H])c(Nc4c([2H])c([2H])c([2H])c(-c5c([2H])c([2H])c([2H])c6c([2H])c([2H])c([2H])c([2H])c56)c4[2H])c([2H])c3[2H])c3c([2H])c([2H])c([2H])c([2H])c3-c3c([2H])c([2H])c([2H])c([2H])c32)c([2H])c1[2H]] |
| P28 | 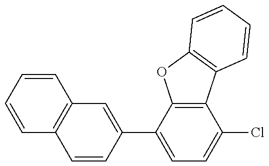 [SMILES: Clc1ccc(-c2ccc3ccccc3c2)c2oc3ccccc3c12] | 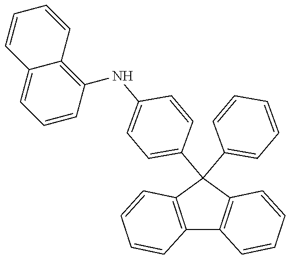 [SMILES: c1ccc(C2(c3ccc(Nc4cccc5ccccc45)cc3)c3ccccc3-c3ccccc32)cc1] |
| P29 | 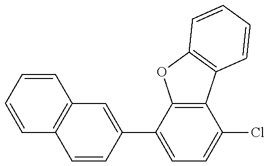 [SMILES: Clc1ccc(-c2ccc3ccccc3c2)c2oc3ccccc3c12] | 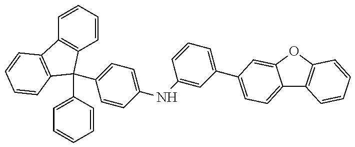 [SMILES: c1ccc(C2(c3ccc(Nc4cccc(-c5ccc6c(c5)oc5ccccc56)c4)cc3)c3ccccc3-c3ccccc32)cc1] |
| P30 | 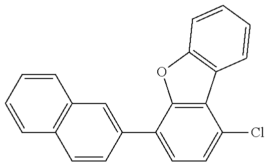 [SMILES: Clc1ccc(-c2ccc3ccccc3c2)c2oc3ccccc3c12] | 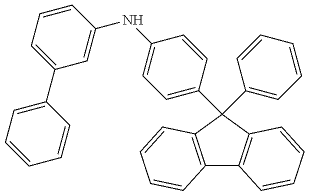 [SMILES: c1ccc(-c2cccc(Nc3ccc(C4(c5ccccc5)c5ccccc5-c5ccccc54)cc3)c2)cc1] |
| P31 | 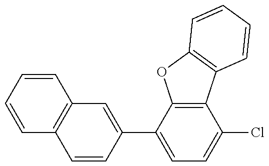 [SMILES: Clc1ccc(-c2ccc3ccccc3c2)c2oc3ccccc3c12] | 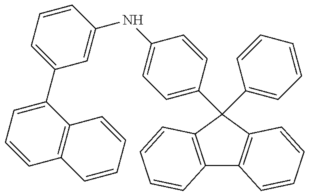 [SMILES: c1ccc(C2(c3ccc(Nc4cccc(-c5cccc6ccccc56)c4)cc3)c3ccccc3-c3ccccc32)cc1] |
| P32 | 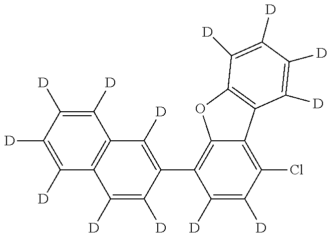 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 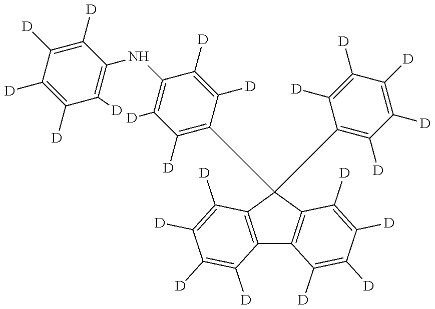 [SMILES: [2H]c1c([2H])c([2H])c(Nc2c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c([2H])c2[2H])c([2H])c1[2H]] |
| P33 | 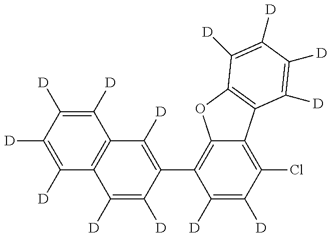 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 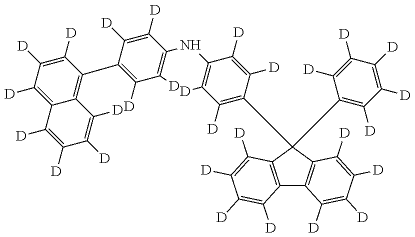 [SMILES: [2H]c1c([2H])c([2H])c(C2(c3c([2H])c([2H])c(Nc4c([2H])c([2H])c(-c5c([2H])c([2H])c([2H])c6c([2H])c([2H])c([2H])c([2H])c56)c([2H])c4[2H])c([2H])c3[2H])c3c([2H])c([2H])c([2H])c([2H])c3-c3c([2H])c([2H])c([2H])c([2H])c32)c([2H])c1[2H]] |
| P34 | 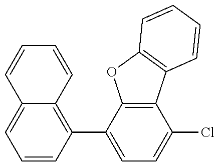 [SMILES: Clc1ccc(-c2cccc3ccccc23)c2oc3ccccc3c12] | 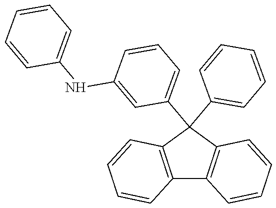 [SMILES: c1ccc(Nc2cccc(C3(c4ccccc4)c4ccccc4-c4ccccc43)c2)cc1] |
| P35 | 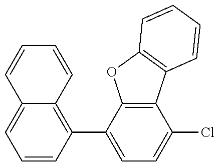 [SMILES: Clc1ccc(-c2cccc3ccccc23)c2oc3ccccc3c12] | 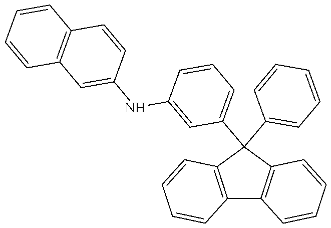 [SMILES: c1ccc(C2(c3cccc(Nc4ccc5ccccc5c4)c3)c3ccccc3-c3ccccc32)cc1] |
| P36 | 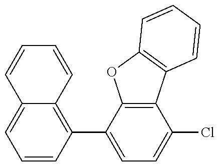 [SMILES: Clc1ccc(-c2cccc3ccccc23)c2oc3ccccc3c12] | 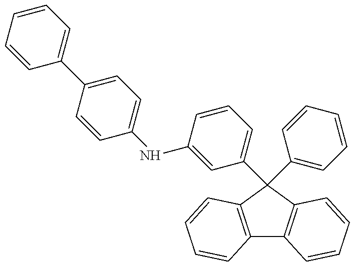 [SMILES: c1ccc(-c2ccc(Nc3cccc(C4(c5ccccc5)c5ccccc5-c5ccccc54)c3)cc2)cc1] |
| P37 | 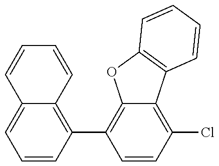 [SMILES: Clc1ccc(-c2cccc3ccccc23)c2oc3ccccc3c12] | 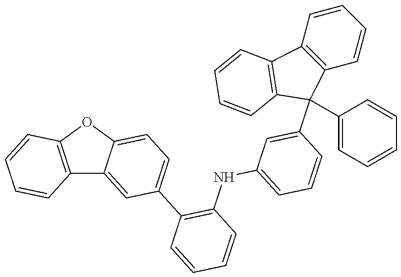 [SMILES: c1ccc(C2(c3cccc(Nc4ccccc4-c4ccc5oc6ccccc6c5c4)c3)c3ccccc3-c3ccccc32)cc1] |
| P38 | 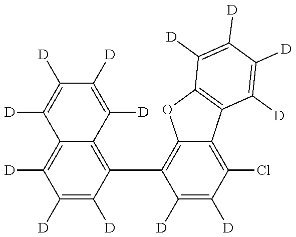 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 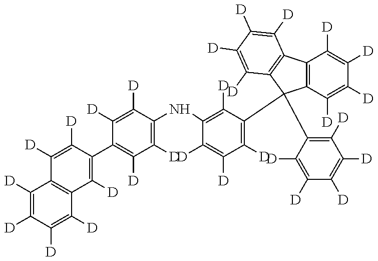 [SMILES: [2H]c1c([2H])c([2H])c(C2(c3c([2H])c([2H])c([2H])c(Nc4c([2H])c([2H])c(-c5c([2H])c([2H])c6c([2H])c([2H])c([2H])c([2H])c6c5[2H])c([2H])c4[2H])c3[2H])c3c([2H])c([2H])c([2H])c([2H])c3-c3c([2H])c([2H])c([2H])c([2H])c32)c([2H])c1[2H]] |
| P39 | 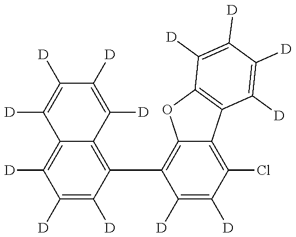 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 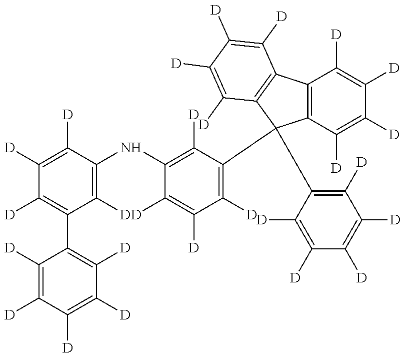 [SMILES: [2H]c1c([2H])c([2H])c(-c2c([2H])c([2H])c([2H])c(Nc3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c2[2H])c([2H])c1[2H]] |
| P40 | 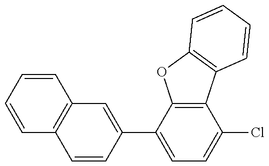 [SMILES: Clc1ccc(-c2ccc3ccccc3c2)c2oc3ccccc3c12] | 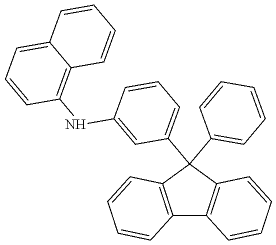 [SMILES: c1ccc(C2(c3cccc(Nc4cccc5ccccc45)c3)c3ccccc3-c3ccccc32)cc1] |
| P41 | 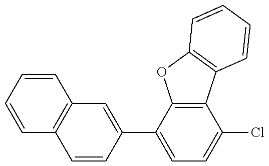 [SMILES: Clc1ccc(-c2ccc3ccccc3c2)c2oc3ccccc3c12] | 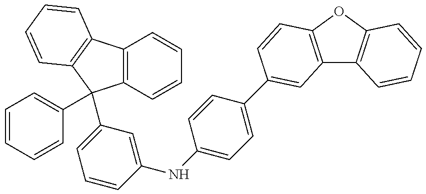 [SMILES: c1ccc(C2(c3cccc(Nc4ccc(-c5ccc6oc7ccccc7c6c5)cc4)c3)c3ccccc3-c3ccccc32)cc1] |
| P42 | 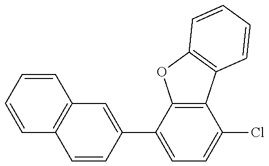 [SMILES: Clc1ccc(-c2ccc3ccccc3c2)c2oc3ccccc3c12] | 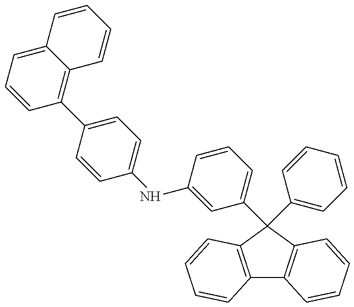 [SMILES: c1ccc(C2(c3cccc(Nc4ccc(-c5cccc6ccccc56)cc4)c3)c3ccccc3-c3ccccc32)cc1] |
| P43 | 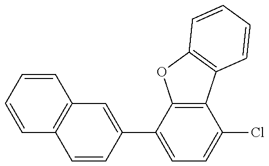 [SMILES: Clc1ccc(-c2ccc3ccccc3c2)c2oc3ccccc3c12] | 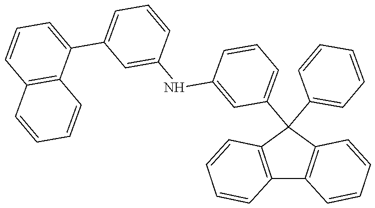 [SMILES: c1ccc(C2(c3cccc(Nc4cccc(-c5cccc6ccccc56)c4)c3)c3ccccc3-c3ccccc32)cc1] |
| P44 | 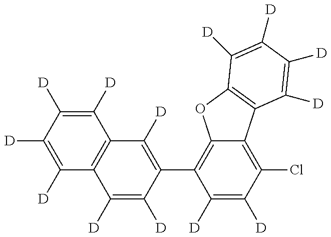 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 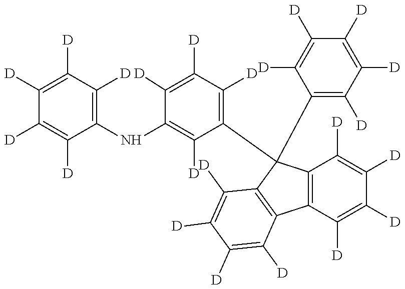 [SMILES: [2H]c1c([2H])c([2H])c(Nc2c([2H])c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c2[2H])c([2H])c1[2H]] |
| P45 | 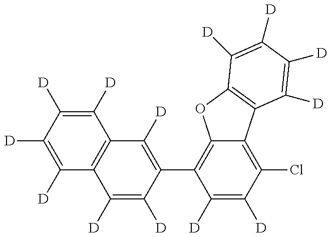 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 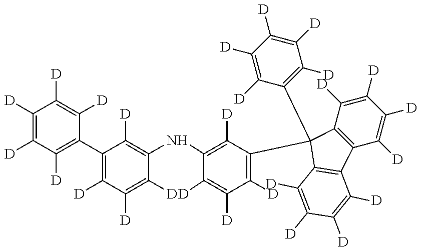 [SMILES: [2H]c1c([2H])c([2H])c(-c2c([2H])c([2H])c([2H])c(Nc3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c2[2H])c([2H])c1[2H]] |
| P46 | 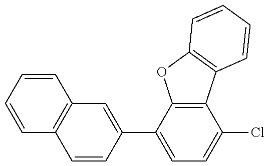 [SMILES: Clc1ccc(-c2ccc3ccccc3c2)c2oc3ccccc3c12] | 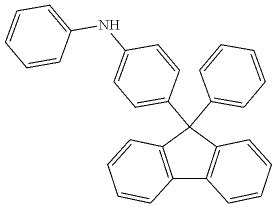 [SMILES: c1ccc(Nc2ccc(C3(c4ccccc4)c4ccccc4-c4ccccc43)cc2)cc1] |
| P47 | 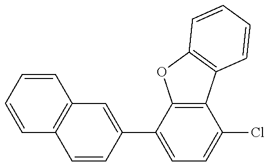 [SMILES: Clc1ccc(-c2ccc3ccccc3c2)c2oc3ccccc3c12] | 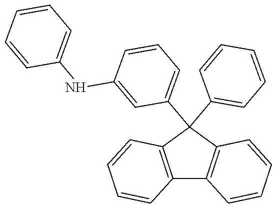 [SMILES: c1ccc(Nc2cccc(C3(c4ccccc4)c4ccccc4-c4ccccc43)c2)cc1] |
| P48 | 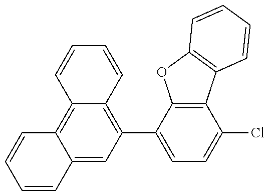 [SMILES: Clc1ccc(-c2cc3ccccc3c3ccccc23)c2oc3ccccc3c12] | 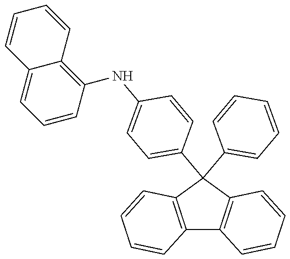 [SMILES: c1ccc(C2(c3ccc(Nc4cccc5ccccc45)cc3)c3ccccc3-c3ccccc32)cc1] |
| P49 | 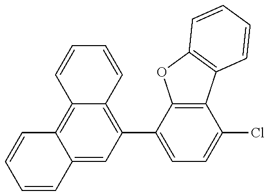 [SMILES: Clc1ccc(-c2cc3ccccc3c3ccccc23)c2oc3ccccc3c12] | 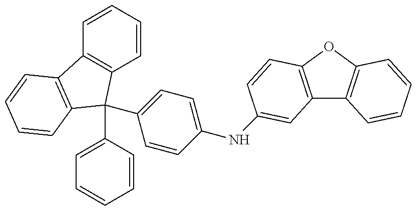 [SMILES: c1ccc(C2(c3ccc(Nc4ccc5oc6ccccc6c5c4)cc3)c3ccccc3-c3ccccc32)cc1] |
| P50 | 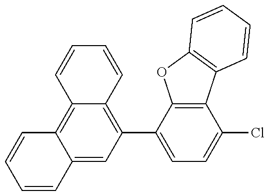 [SMILES: Clc1ccc(-c2cc3ccccc3c3ccccc23)c2oc3ccccc3c12] | 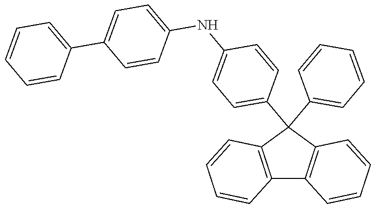 [SMILES: c1ccc(-c2ccc(Nc3ccc(C4(c5ccccc5)c5ccccc5-c5ccccc54)cc3)cc2)cc1] |
| P51 | 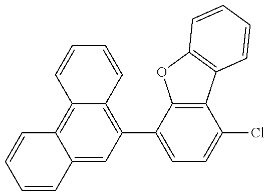 [SMILES: Clc1ccc(-c2cc3ccccc3c3ccccc23)c2oc3ccccc3c12] | 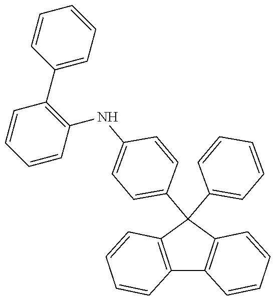 [SMILES: c1ccc(-c2ccccc2Nc2ccc(C3(c4ccccc4)c4ccccc4-c4ccccc43)cc2)cc1] |
| P52 | 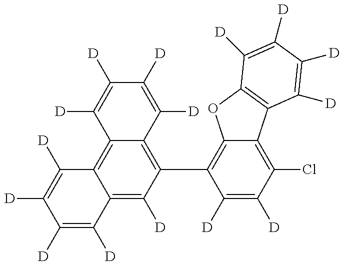 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 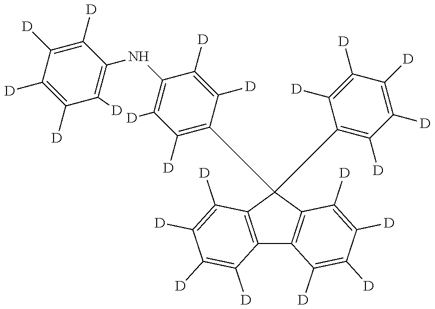 [SMILES: [2H]c1c([2H])c([2H])c(Nc2c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c([2H])c2[2H])c([2H])c1[2H]] |
| P53 | 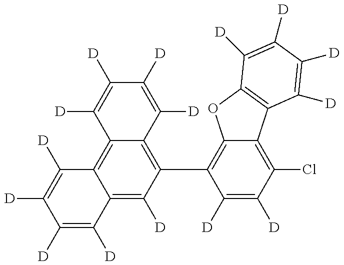 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 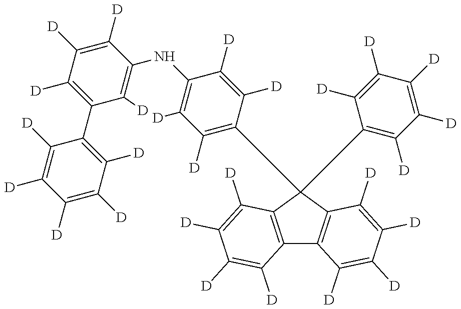 [SMILES: [2H]c1c([2H])c([2H])c(-c2c([2H])c([2H])c([2H])c(Nc3c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c([2H])c3[2H])c2[2H])c([2H])c1[2H]] |
| P54 | 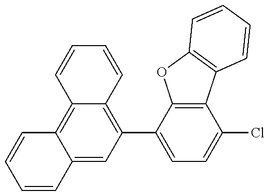 [SMILES: Clc1ccc(-c2cc3ccccc3c3ccccc23)c2oc3ccccc3c12] | 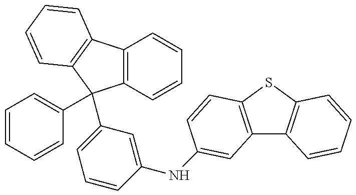 [SMILES: c1ccc(C2(c3cccc(Nc4ccc5sc6ccccc6c5c4)c3)c3ccccc3-c3ccccc32)cc1] |
| P55 | 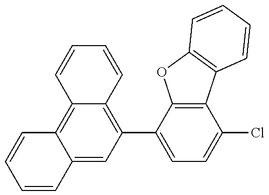 [SMILES: Clc1ccc(-c2cc3ccccc3c3ccccc23)c2oc3ccccc3c12] | 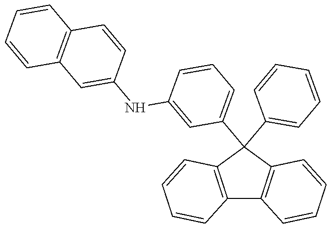 [SMILES: c1ccc(C2(c3cccc(Nc4ccc5ccccc5c4)c3)c3ccccc3-c3ccccc32)cc1] |
| P56 | 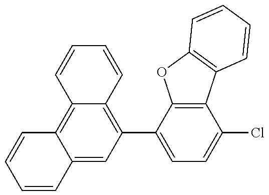 [SMILES: Clc1ccc(-c2cc3ccccc3c3ccccc23)c2oc3ccccc3c12] | 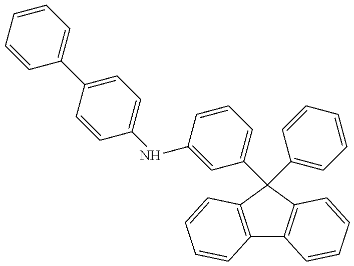 [SMILES: c1ccc(-c2ccc(Nc3cccc(C4(c5ccccc5)c5ccccc5-c5ccccc54)c3)cc2)cc1] |
| P57 | 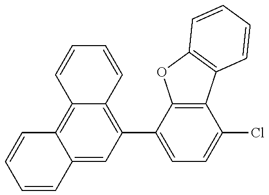 [SMILES: Clc1ccc(-c2cc3ccccc3c3ccccc23)c2oc3ccccc3c12] | 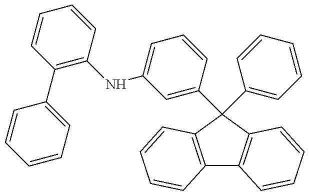 [SMILES: c1ccc(-c2ccccc2Nc2cccc(C3(c4ccccc4)c4ccccc4-c4ccccc43)c2)cc1] |
| P58 | 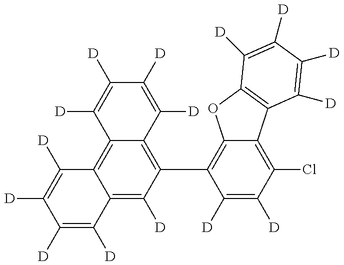 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 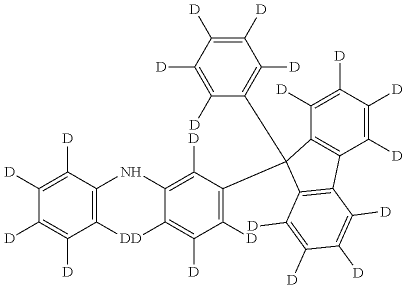 [SMILES: [2H]c1c([2H])c([2H])c(Nc2c([2H])c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c2[2H])c([2H])c1[2H]] |
| P59 | 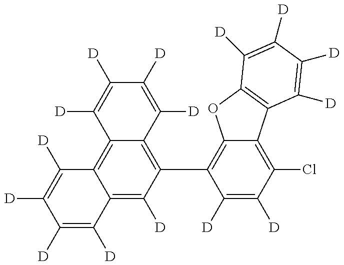 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 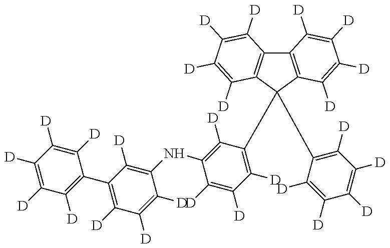 [SMILES: [2H]c1c([2H])c([2H])c(-c2c([2H])c([2H])c([2H])c(Nc3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c2[2H])c([2H])c1[2H]] |
| P60 | 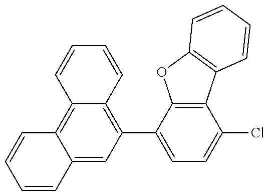 [SMILES: Clc1ccc(-c2cc3ccccc3c3ccccc23)c2oc3ccccc3c12] | 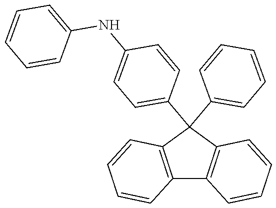 [SMILES: c1ccc(Nc2ccc(C3(c4ccccc4)c4ccccc4-c4ccccc43)cc2)cc1] |
| P61 | 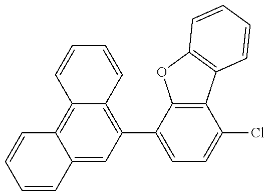 [SMILES: Clc1ccc(-c2cc3ccccc3c3ccccc23)c2oc3ccccc3c12] | 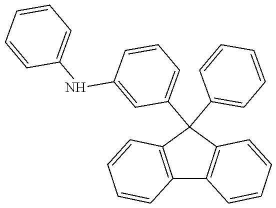 [SMILES: c1ccc(Nc2cccc(C3(c4ccccc4)c4ccccc4-c4ccccc43)c2)cc1] |
| P62 | 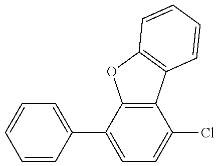 [SMILES: Clc1ccc(-c2ccccc2)c2oc3ccccc3c12] | 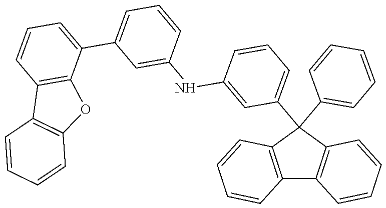 [SMILES: c1ccc(C2(c3cccc(Nc4cccc(-c5cccc6c5oc5ccccc56)c4)c3)c3ccccc3-c3ccccc32)cc1] |

| | | Amount | |
|---|---|---|---|
| | | of yield | |
| | | (percent | |
| Item | | of yield) | [M + H]⁺ |
| P1 | 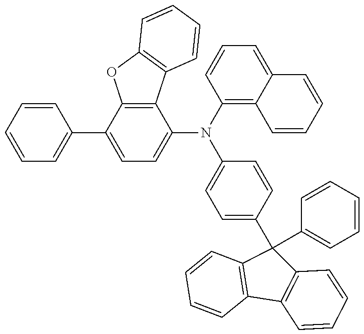 [SMILES: c1ccc(-c2ccc(N(c3ccc(C4(c5ccccc5)c5ccccc5-c5ccccc54)cc3)c3cccc4ccccc34)c3c2oc2ccccc23)cc1] | 17.4 g (76%) | 701.87 |
| | 1-2 | | |
| P2 | 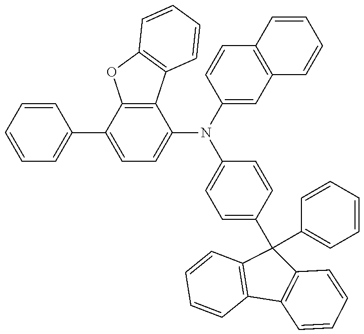 [SMILES: c1ccc(-c2ccc(N(c3ccc(C4(c5ccccc5)c5ccccc5-c5ccccc54)cc3)c3ccc4ccccc4c3)c3c2oc2ccccc23)cc1] | 16.3 g (71%) | 701.87 |
| | 1-3 | | |
| P3 | 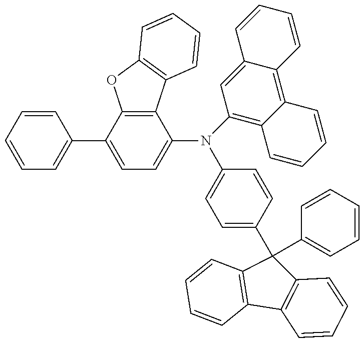 [SMILES: c1ccc(-c2ccc(N(c3ccc(C4(c5ccccc5)c5ccccc5-c5ccccc54)cc3)c3cc4ccccc4c4ccccc34)c3c2oc2ccccc23)cc1] | 15.7 g (71%) | 751.93 |
| | 1-4 | | |
| P4 | 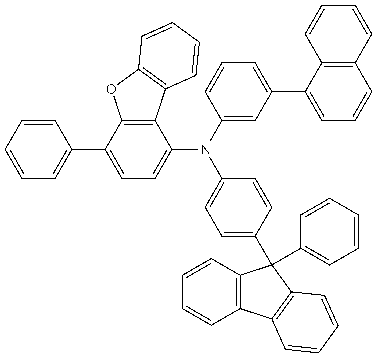 [SMILES: c1ccc(-c2ccc(N(c3ccc(C4(c5ccccc5)c5ccccc5-c5ccccc54)cc3)c3cccc(-c4cccc5ccccc45)c3)c3c2oc2ccccc23)cc1] | 16.1 g (74%) | 777.97 |
| | 1-26 | | |
| P5 | 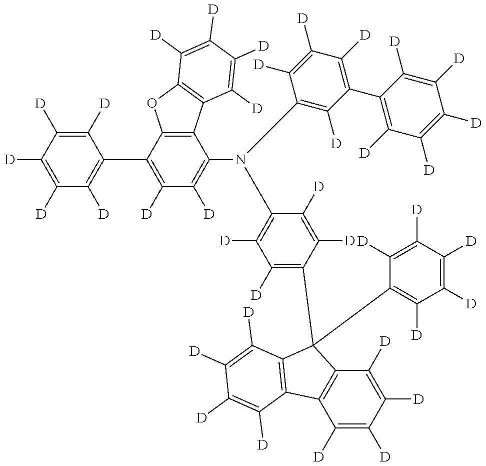 [SMILES: [2H]c1c([2H])c([2H])c(-c2c([2H])c([2H])c([2H])c(N(c3c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c([2H])c3[2H])c3c([2H])c([2H])c(-c4c([2H])c([2H])c([2H])c([2H])c4[2H])c4oc5c([2H])c([2H])c([2H])c([2H])c5c34)c2[2H])c([2H])c1[2H]] | 15.7 g (70%) | 765.13 |
| | 1-71 | | |
| P6 | 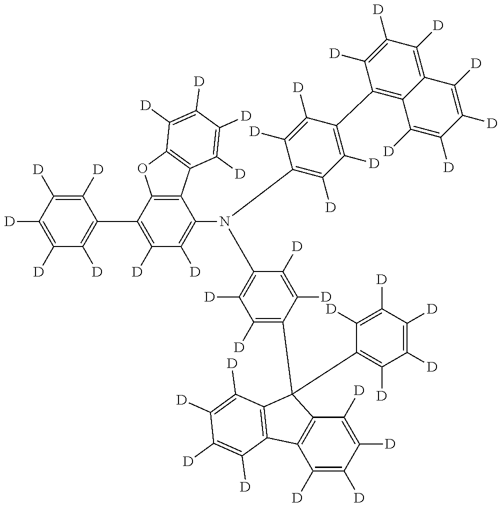 [SMILES: [2H]c1c([2H])c([2H])c(-c2c([2H])c([2H])c(N(c3c([2H])c([2H])c(-c4c([2H])c([2H])c([2H])c5c([2H])c([2H])c([2H])c([2H])c45)c([2H])c3[2H])c3c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c([2H])c3[2H])c3c2oc2c([2H])c([2H])c([2H])c([2H])c23)c([2H])c1[2H]] | 14.8 g (68%) | 817.20 |
| | 1-68 | | |
| P7 | 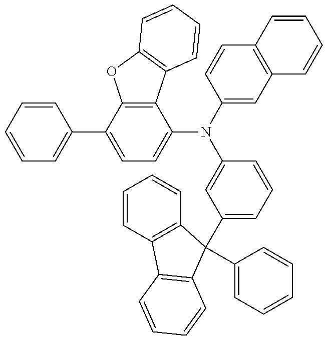 [SMILES: c1ccc(-c2ccc(N(c3cccc(C4(c5ccccc5)c5ccccc5-c5ccccc54)c3)c3ccc4ccccc4c3)c3c2oc2ccccc23)cc1] | 16.3 g (71%) | 701.87 |
| | 1-77 | | |
| P8 | 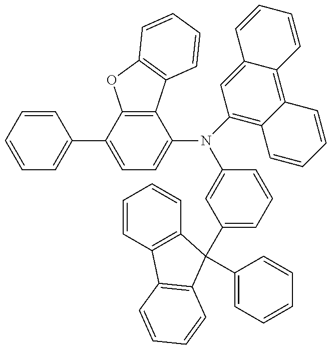 [SMILES: c1ccc(-c2ccc(N(c3cccc(C4(c5ccccc5)c5ccccc5-c5ccccc54)c3)c3cc4ccccc4c4ccccc34)c3c2oc2ccccc23)cc1] | 15.5 g (70%) | 751.93 |
| | 1-78 | | |
| P9 | 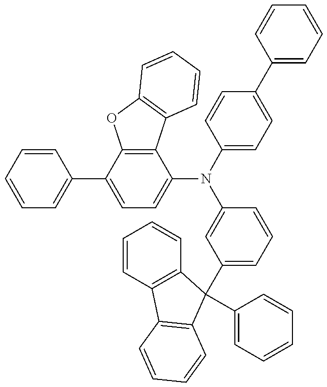 [SMILES: c1ccc(-c2ccc(N(c3cccc(C4(c5ccccc5)c5ccccc5-c5ccccc54)c3)c3ccc(-c4ccccc4)c4oc5ccccc5c34)cc2)cc1] | 16.4 g (73%) | 727.91 |
| | 1-87 | | |
| P10 | 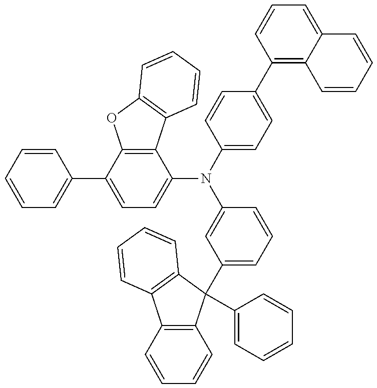 [SMILES: c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1] | 15.9 g (73%) | 777.97 |
| | 1-88 | | |
| P11 | 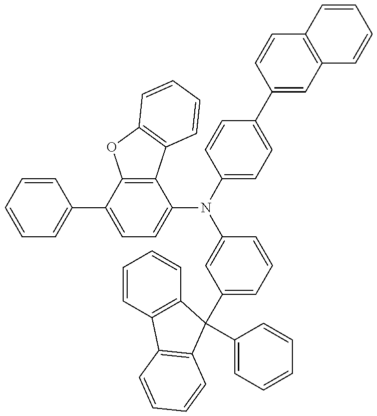 [SMILES: c1ccc(-c2ccc(N(c3ccc(-c4ccc5ccccc5c4)cc3)c3cccc(C4(c5ccccc5)c5ccccc5-c5ccccc54)c3)c3c2oc2ccccc23)cc1] | 16.3 g (75%) | 777.97 |
| | 1-89 | | |
| P12 | 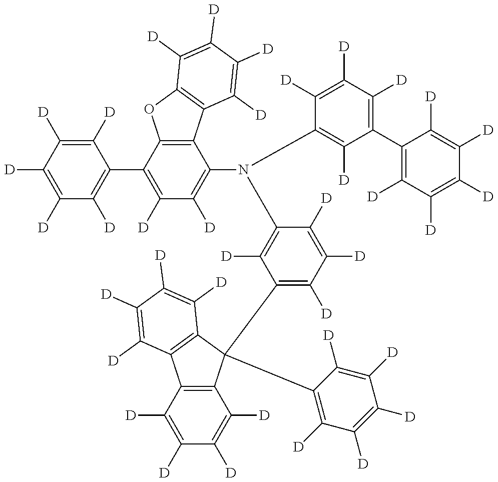 [SMILES: [2H]c1c([2H])c([2H])c(-c2c([2H])c([2H])c([2H])c(N(c3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c3c([2H])c([2H])c(-c4c([2H])c([2H])c([2H])c([2H])c4[2H])c4oc5c([2H])c([2H])c([2H])c([2H])c5c34)c2[2H])c([2H])c1[2H]] | 15.5 g (69%) | 765.13 |
| | 1-148 | | |
| P13 | 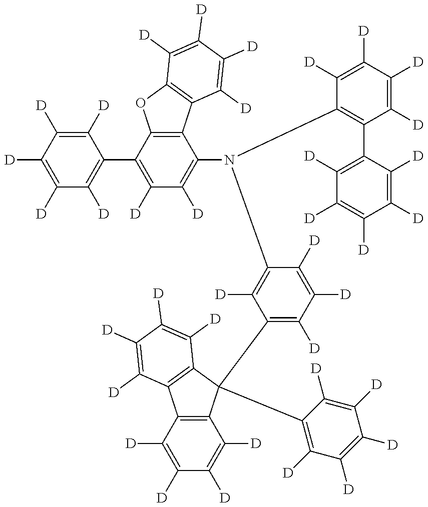 [SMILES: [2H]c1c([2H])c([2H])c(-c2c([2H])c([2H])c([2H])c([2H])c2N(c2c([2H])c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c2[2H])c2c([2H])c([2H])c(-c3c([2H])c([2H])c([2H])c([2H])c3[2H])c3oc4c([2H])c([2H])c([2H])c([2H])c4c23)c([2H])c1[2H]] | 16.1 g (72%) | 765.13 |
| | 1-151 | | |
| P14 | 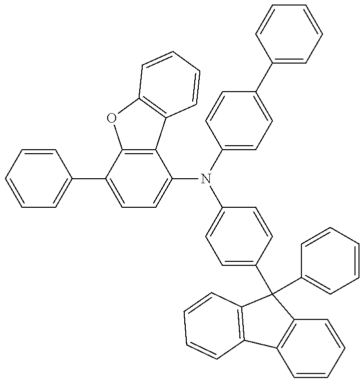 [SMILES: c1ccc(-c2ccc(N(c3ccc(C4(c5ccccc5)c5ccccc5-c5ccccc54)cc3)c3ccc(-c4ccccc4)c4oc5ccccc5c34)cc2)cc1] | 16.1 g (72%) | 727.91 |
| | 1-13 | | |
| P15 | 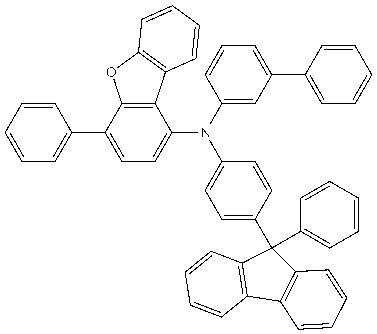 [SMILES: c1ccc(-c2cccc(N(c3ccc(C4(c5ccccc5)c5ccccc5-c5ccccc54)cc3)c3ccc(-c4ccccc4)c4oc5ccccc5c34)c2)cc1] | 16.4 g (73%) | 727.91 |
| | 1-25 | | |
| P16 | 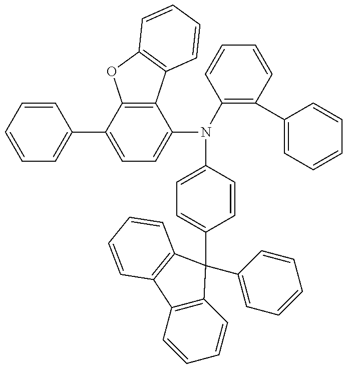 [SMILES: c1ccc(-c2ccccc2N(c2ccc(C3(c4ccccc4)c4ccccc4-c4ccccc43)cc2)c2ccc(-c3ccccc3)c3oc4ccccc4c23)cc1] | 16.1 g (72%) | 765.13 |
| | 1-37 | | |
| P-17 | 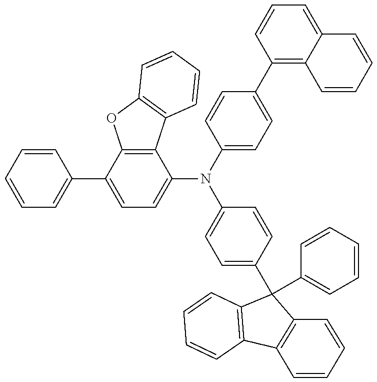 [SMILES: c1ccc(-c2ccc(N(c3ccc(-c4cccc5ccccc45)cc3)c3ccc(C4(c5ccccc5)c5ccccc5-c5ccccc54)cc3)c3c2oc2ccccc23)cc1] | 15.7 g (72%) | 777.97 |
| | 1-14 | | |
| P-18 | 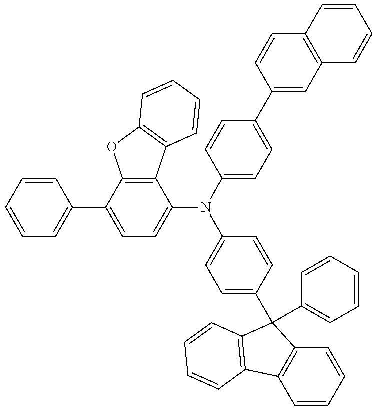 [SMILES: c1ccc(-c2ccc(N(c3ccc(-c4ccc5ccccc5c4)cc3)c3ccc(C4(c5ccccc5)c5ccccc5-c5ccccc54)cc3)c3c2oc2ccccc23)cc1] | 15.2 g (70%) | 777.97 |
| | 1-15 | | |
| P19 | 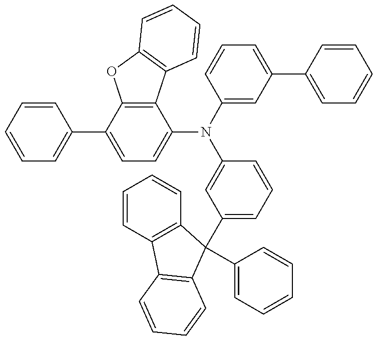 [SMILES: c1ccc(-c2cccc(N(c3cccc(C4(c5ccccc5)c5ccccc5-c5ccccc54)c3)c3ccc(-c4ccccc4)c4oc5ccccc5c34)c2)cc1] | 16.2 g (72%) | 727.91 |
| | 1-99 | | |
| P20 | 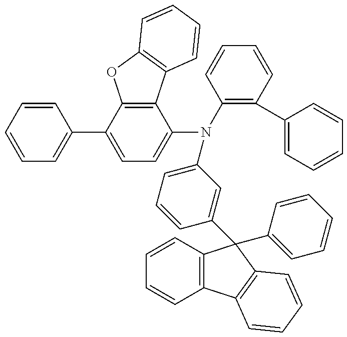 [SMILES: c1ccc(-c2ccccc2N(c2cccc(C3(c4ccccc4)c4ccccc4-c4ccccc43)c2)c2ccc(-c3ccccc3)c3oc4ccccc4c23)cc1] | 16.9 g (71%) | 727.91 |
| | 1-111 | | |
| P21 | 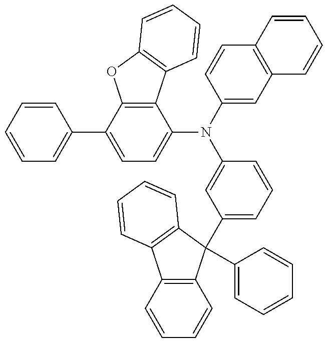 [SMILES: c1ccc(-c2ccc(N(c3cccc(C4(c5ccccc5)c5ccccc5-c5ccccc54)c3)c3ccc4ccccc4c3)c3c2oc2ccccc23)cc1] | 16.5 g (72%) | 701.87 |
| | 1-77 | | |
| P22 | 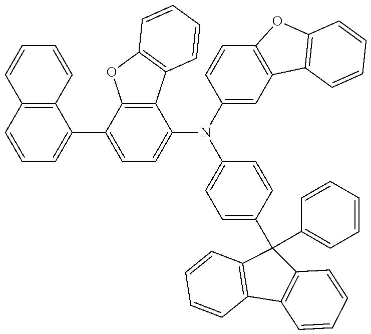 [SMILES: c1ccc(C2(c3ccc(N(c4ccc5oc6ccccc6c5c4)c4ccc(-c5cccc6ccccc56)c5oc6ccccc6c45)cc3)c3ccccc3-c3ccccc32)cc1] | 17.6 g (74%) | 791.95 |

2-15

| | | | |
|---|---|---|---|
| P23 | 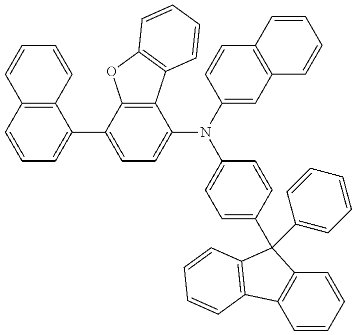 [SMILES: c1ccc(C2(c3ccc(N(c4ccc5ccccc5c4)c4ccc(-c5cccc6ccccc56)c5oc6ccccc6c45)cc3)c3ccccc3-c3ccccc32)cc1] | 17.4 g (71%) | 751.93 |
| | 2-3 | | |
| P24 | 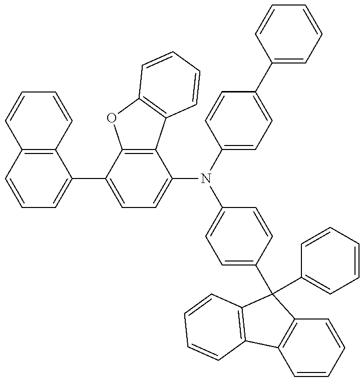 [SMILES: c1ccc(-c2ccc(N(c3ccc(C4(c5ccccc5)c5ccccc5-c5ccccc54)cc3)c3ccc(-c4cccc5ccccc45)c4oc5ccccc5c34)cc2)cc1] | 16.8 g (70%) | 777.97 |
| | 2-21 | | |
| P25 | 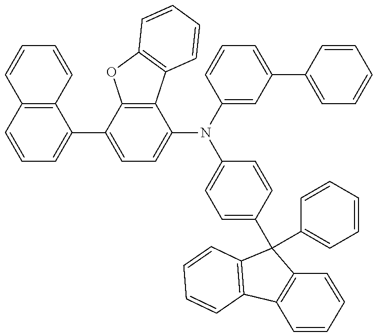 [SMILES: c1ccc(-c2cccc(N(c3ccc(C4(c5ccccc5)c5ccccc5-c5ccccc54)cc3)c3ccc(-c4cccc5ccccc45)c4oc5ccccc5c34)c2)cc1] | 17.5 g (73%) | 777.97 |
| | 2-41 | | |
| P26 | 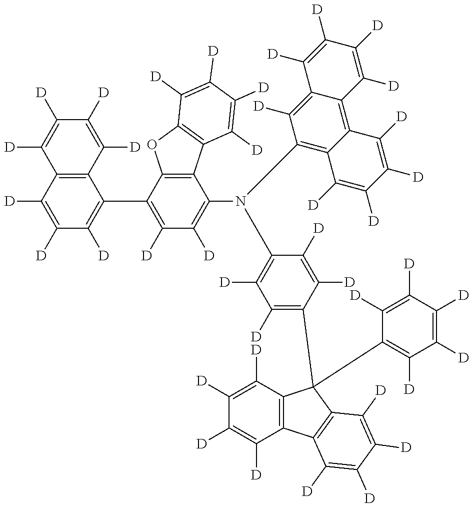 [SMILES: [2H]c1c([2H])c([2H])c(C2(c3c([2H])c([2H])c(N(c4c([2H])c5c([2H])c([2H])c([2H])c([2H])c5c5c([2H])c([2H])c([2H])c([2H])c45)c4c([2H])c([2H])c(-c5c([2H])c([2H])c([2H])c6c([2H])c([2H])c([2H])c([2H])c56)c5oc6c([2H])c([2H])c([2H])c([2H])c6c45)c([2H])c3[2H])c3c([2H])c([2H])c([2H])c([2H])c3-c3c([2H])c([2H])c([2H])c([2H])c32)c([2H])c1[2H]] | 16.0 g (68%) | 841.23 |
| | 2-83 | | |
| P27 | 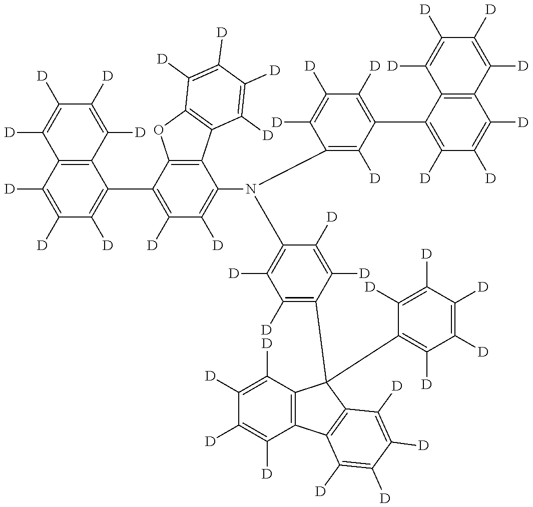 [SMILES: [2H]c1c([2H])c([2H])c(C2(c3c([2H])c([2H])c(N(c4c([2H])c([2H])c([2H])c(-c5c([2H])c([2H])c([2H])c6c([2H])c([2H])c([2H])c([2H])c56)c4[2H])c4c([2H])c([2H])c(-c5c([2H])c([2H])c([2H])c6c([2H])c([2H])c([2H])c([2H])c56)c5oc6c([2H])c([2H])c([2H])c([2H])c6c45)c([2H])c3[2H])c3c([2H])c([2H])c([2H])c([2H])c3-c3c([2H])c([2H])c([2H])c([2H])c32)c([2H])c1[2H]] | 16.6 g (72%) | 869.28 |
| | 2-86 | | |
| P28 | 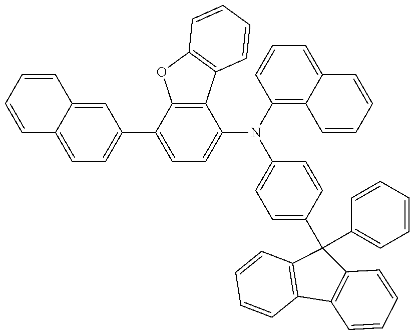 [SMILES: c1ccc(C2(c3ccc(N(c4cccc5ccccc45)c4ccc(-c5ccc6ccccc6c5)c5oc6ccccc6c45)cc3)c3ccccc3-c3ccccc32)cc1] | 18.9 g (77%) | 751.93 |
| | 2-88 | | |
| P29 | 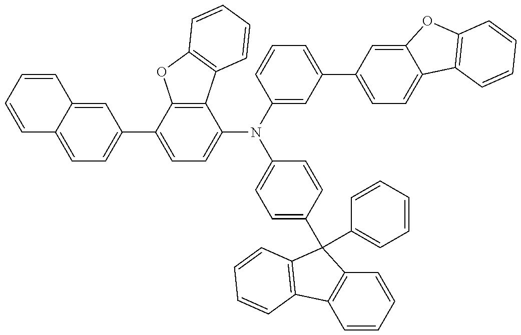 [SMILES: c1ccc(C2(c3ccc(N(c4cccc(-c5ccc6c(c5)oc5ccccc56)c4)c4ccc(-c5ccc6ccccc6c5)c5oc6ccccc6c45)cc3)c3ccccc3-c3ccccc32)cc1] | 17.0 g (75%) | 868.05 |
| | 2-140 | | |
| P30 | 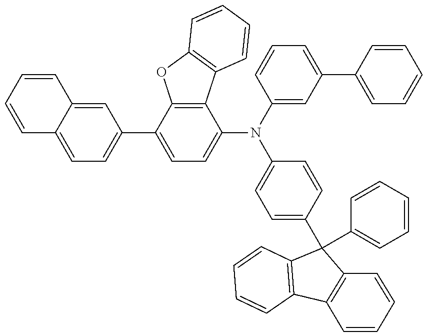 [SMILES: c1ccc(-c2cccc(N(c3ccc(C4(c5ccccc5)c5ccccc5-c5ccccc54)cc3)c3ccc(-c4ccc5ccccc5c4)c4oc5ccccc5c34)c2)cc1] | 17.1 g (71%) | 777.97 |
| | 2-127 | | |
| P31 | 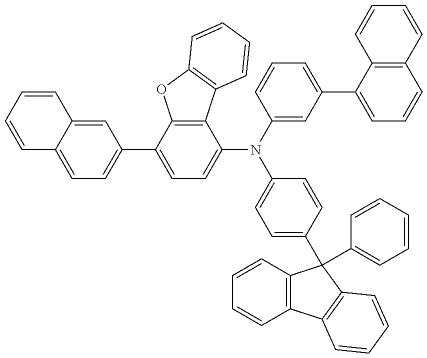 [SMILES: c1ccc(C2(c3ccc(N(c4cccc(-c5cccc6ccccc56)c4)c4ccc(-c5ccc6ccccc6c5)c5oc6ccccc6c45)cc3)c3ccccc3-c3ccccc32)cc1] | 15.5 g (67%) | 828.03 |
| | 2-128 | | |
| P32 | 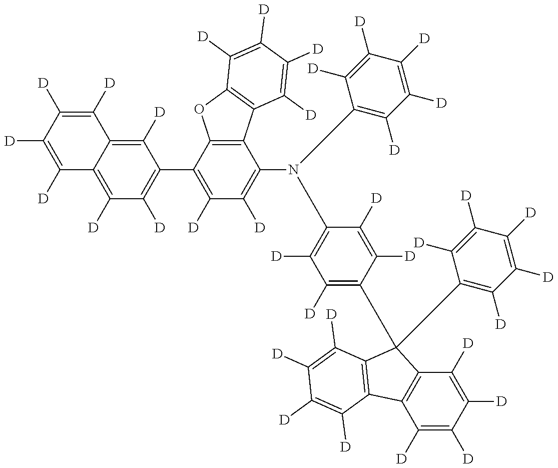 [SMILES: [2H]c1c([2H])c([2H])c(N(c2c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c([2H])c2[2H])c2c([2H])c([2H])c(-c3c([2H])c([2H])c4c([2H])c([2H])c([2H])c([2H])c4c3[2H])c3oc4c([2H])c([2H])c([2H])c([2H])c4c23)c([2H])c1[2H]] | 17.9 g (74%) | 737.08 |
| | 2-169 | | |
| P33 | 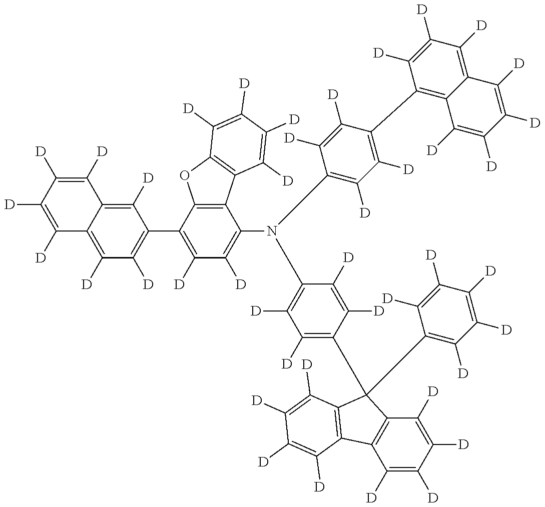 [SMILES: [2H]c1c([2H])c([2H])c(C2(c3c([2H])c([2H])c(N(c4c([2H])c([2H])c(-c5c([2H])c([2H])c([2H])c6c([2H])c([2H])c([2H])c([2H])c56)c([2H])c4[2H])c4c([2H])c([2H])c(-c5c([2H])c([2H])c6c([2H])c([2H])c([2H])c([2H])c6c5[2H])c5oc6c([2H])c([2H])c([2H])c([2H])c6c45)c([2H])c3[2H])c3c([2H])c([2H])c([2H])c([2H])c3-c3c([2H])c([2H])c([2H])c([2H])c32)c([2H])c1[2H]] | 18.9 g (70%) | 869.28 |
| | 2-172 | | |
| P34 | 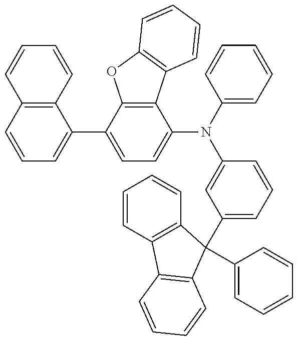 [SMILES: c1ccc(N(c2cccc(C3(c4ccccc4)c4ccccc4-c4ccccc43)c2)c2ccc(-c3cccc4ccccc34)c3oc4ccccc4c23)cc1] | 18.8 g (73%) | 701.87 |
| | 2-173 | | |
| P35 | 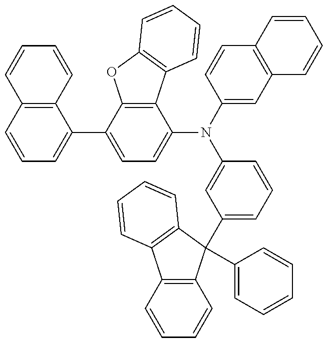 [SMILES: c1ccc(C2(c3cccc(N(c4ccc5ccccc5c4)c4ccc(-c5cccc6ccccc56)c5oc6ccccc6c45)c3)c3ccccc3-c3ccccc32)cc1] | 17.2 g (70%) | 751.93 |
| | 2-175 | | |
| P36 | 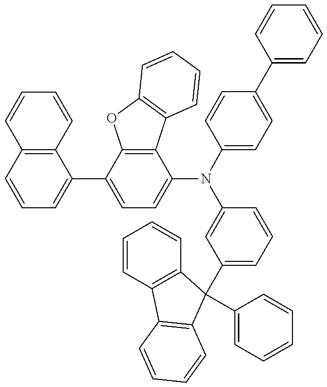 [SMILES: c1ccc(-c2ccc(N(c3cccc(C4(c5ccccc5)c5ccccc5-c5ccccc54)c3)c3ccc(-c4cccc5ccccc45)c4oc5ccccc5c34)cc2)cc1] | 17.8 g (74%) | 777.97 |
| | 2-193 | | |
| P37 | 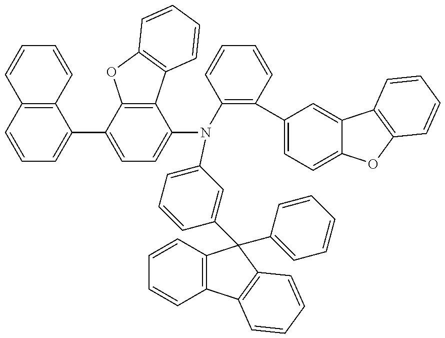 [SMILES: c1ccc(C2(c3cccc(N(c4ccccc4-c4ccc5oc6ccccc6c5c4)c4ccc(-c5cccc6ccccc56)c5oc6ccccc6c45)c3)c3ccccc3-c3ccccc32)cc1] | 16.1 g (71%) | 868.05 |
| | 2-247 | | |
| P38 | 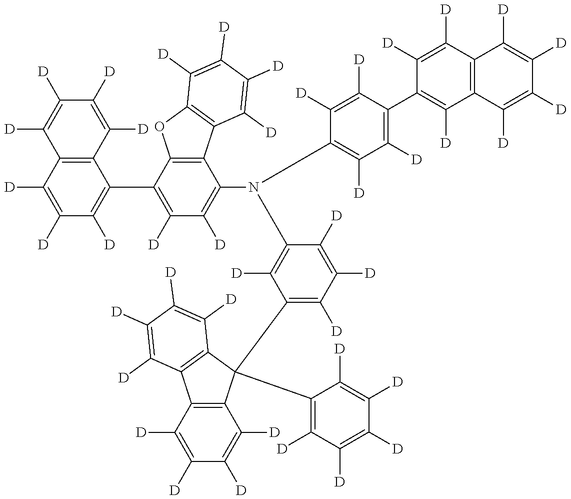 [SMILES: [2H]c1c([2H])c([2H])c(C2(c3c([2H])c([2H])c([2H])c(N(c4c([2H])c([2H])c(-c5c([2H])c([2H])c6c([2H])c([2H])c([2H])c([2H])c6c5[2H])c([2H])c4[2H])c4c([2H])c([2H])c(-c5c([2H])c([2H])c([2H])c6c([2H])c([2H])c([2H])c([2H])c56)c5oc6c([2H])c([2H])c([2H])c([2H])c6c45)c3[2H])c3c([2H])c([2H])c([2H])c([2H])c3-c3c([2H])c([2H])c([2H])c([2H])c32)c([2H])c1[2H]] | 15.5 g (67%) | 869.28 |
| | 2-255 | | |
| P39 | 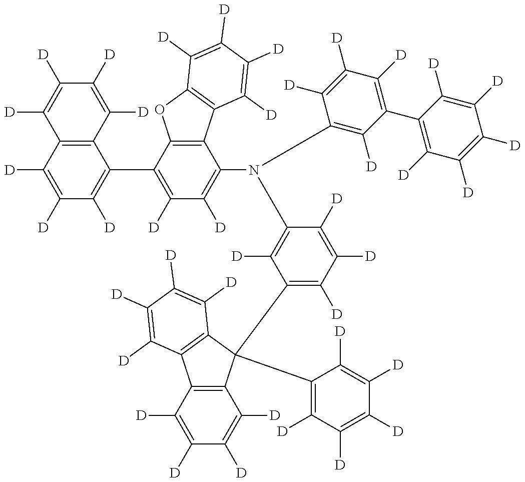 [SMILES: [2H]c1c([2H])c([2H])c(-c2c([2H])c([2H])c([2H])c(N(c3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c2[2H])c([2H])c1[2H]] | 17.2 g (72%) | 817.2 |
| | 2-258 | | |
| P40 | 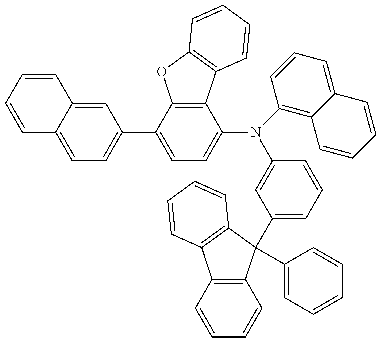 [SMILES: c1ccc(C2(c3cccc(N(c4cccc5ccccc45)c4ccc(-c5ccc6ccccc6c5)c5oc6ccccc6c45)c3)c3ccccc3-c3ccccc32)cc1] | 18.7 g (76%) | 751.93 |
| | 2-260 | | |
| P41 | 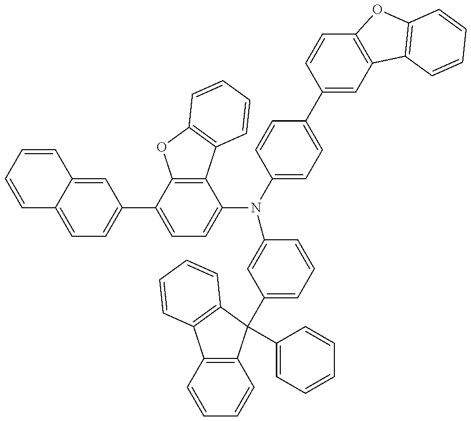 [SMILES: c1ccc(C2(c3cccc(N(c4ccc(-c5ccc6oc7ccccc7c6c5)cc4)c4ccc(-c5ccc6ccccc6c5)c5oc6ccccc6c45)c3)c3ccccc3-c3ccccc32)cc1] | 17.0 g (75%) | 868.05 |
| | 2-293 | | |
| P42 | 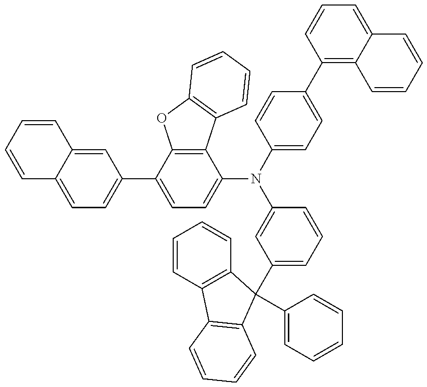 [SMILES: c1ccc(C2(c3cccc(N(c4ccc(-c5cccc6ccccc56)cc4)c4ccc(-c5ccc6ccccc6c5)c5oc6ccccc6c45)c3)c3ccccc3-c3ccccc32)cc1] | 16.7 g (72%) | 828.03 |
| | 2-280 | | |
| P43 | 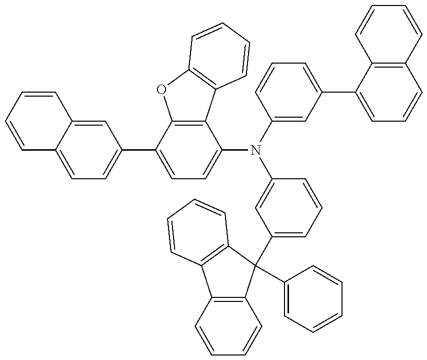 [SMILES: c1ccc(C2(c3cccc(N(c4cccc(-c5cccc6ccccc56)c4)c4ccc(-c5ccc6ccccc6c5)c5oc6ccccc6c45)c3)c3ccccc3-c3ccccc32)cc1] | 17.2 g (74%) | 828.03 |
| | 2-300 | | |
| P44 | 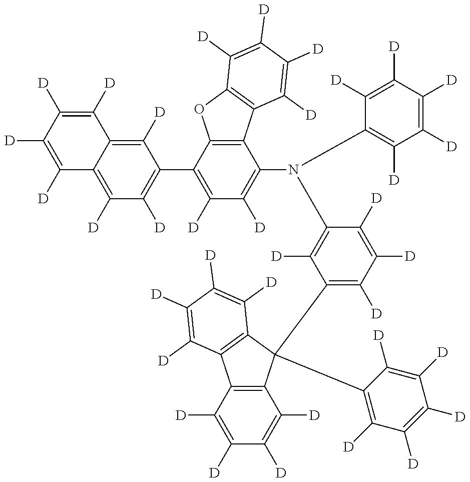 [SMILES: [2H]c1c([2H])c([2H])c(N(c2c([2H])c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c2[2H])c2c([2H])c([2H])c(-c3c([2H])c([2H])c4c([2H])c([2H])c([2H])c([2H])c4c3[2H])c3oc4c([2H])c([2H])c([2H])c([2H])c4c23)c([2H])c1[2H]] | 17.9 g (70%) | 737.08 |
| | 2-341 | | |
| P45 | 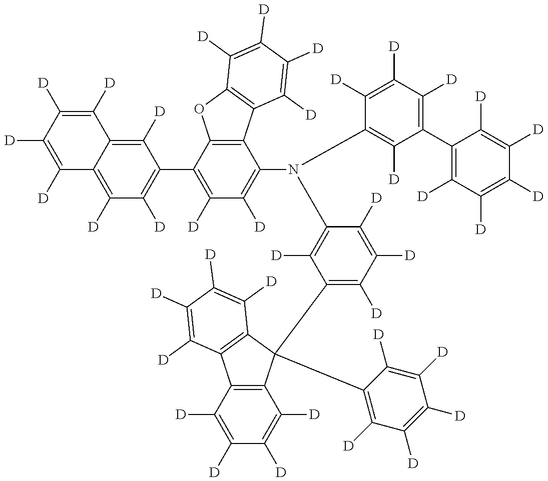 [SMILES: [2H]c1c([2H])c([2H])c(-c2c([2H])c([2H])c([2H])c(N(c3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c3c([2H])c([2H])c(-c4c([2H])c([2H])c5c([2H])c([2H])c([2H])c([2H])c5c4[2H])c4oc5c([2H])c([2H])c([2H])c([2H])c5c34)c2[2H])c([2H])c1[2H]] | 16.3 g (68%) | 817.2 |
| | 2-344 | | |
| P46 | 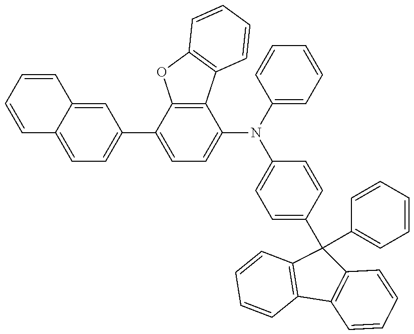 [SMILES: c1ccc(N(c2ccc(C3(c4ccccc4)c4ccccc4-c4ccccc43)cc2)c2ccc(-c3ccc4ccccc4c3)c3oc4ccccc4c23)cc1] | 19.5 g (70%) | 701.87 |
| | 2-87 | | |
| P47 | 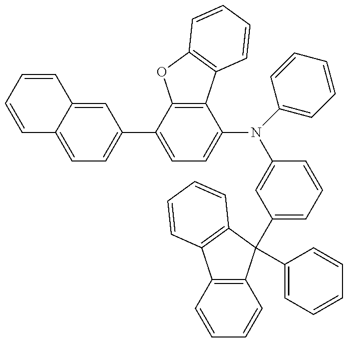 [SMILES: c1ccc(N(c2cccc(C3(c4ccccc4)c4ccccc4-c4ccccc43)c2)c2ccc(-c3ccc4ccccc4c3)c3oc4ccccc4c23)cc1] | 18.3 g (70%) | 701.87 |
| | 2-259 | | |
| P48 | 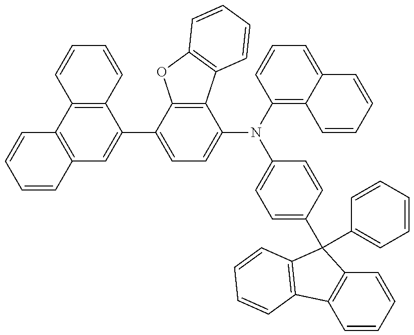 [SMILES: c1ccc(C2(c3ccc(N(c4cccc5ccccc45)c4ccc(-c5cc6ccccc6c6ccccc56)c5oc6ccccc6c45)cc3)c3ccccc3-c3ccccc32)cc1] | 19.1 g (73%) | 801.99 |
| | 3-2 | | |
| P49 | 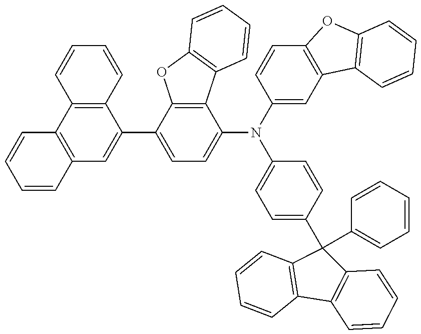 [SMILES: c1ccc(C2(c3ccc(N(c4ccc5oc6ccccc6c5c4)c4ccc(-c5cc6ccccc6c6ccccc56)c5oc6ccccc6c45)cc3)c3ccccc3-c3ccccc32)cc1] | 17.9 g (71%) | 842.01 |
| | 3-15 | | |
| P50 | 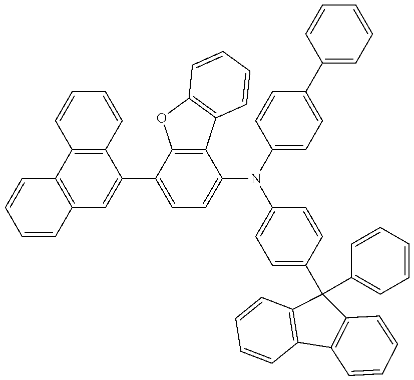 [SMILES: c1ccc(-c2ccc(N(c3ccc(C4(c5ccccc5)c5ccccc5-c5ccccc54)cc3)c3ccc(-c4cc5ccccc5c5ccccc45)c4oc5ccccc5c34)cc2)cc1] | 18.9 g (74%) | 828.03 |
| | 3-21 | | |
| P51 | 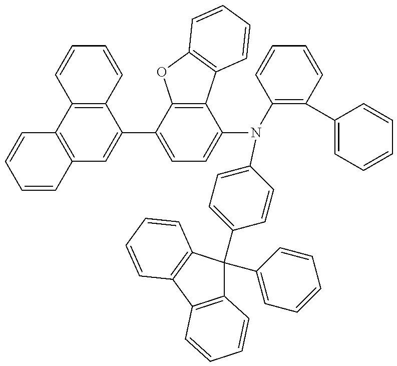 [SMILES: c1ccc(-c2ccccc2N(c2ccc(C3(c4ccccc4)c4ccccc4-c4ccccc43)cc2)c2ccc(-c3cc4ccccc4c4ccccc34)c3oc4ccccc4c23)cc1] | 18.2 g (71%) | 828.03 |
| | 3-61 | | |
| P52 | 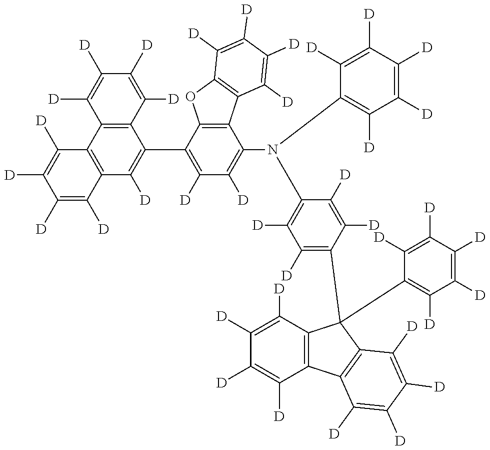 [SMILES: [2H]c1c([2H])c([2H])c(N(c2c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c([2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 19.2 g (70%) | 789.15 |
| | 3-88 | | |
| P53 | 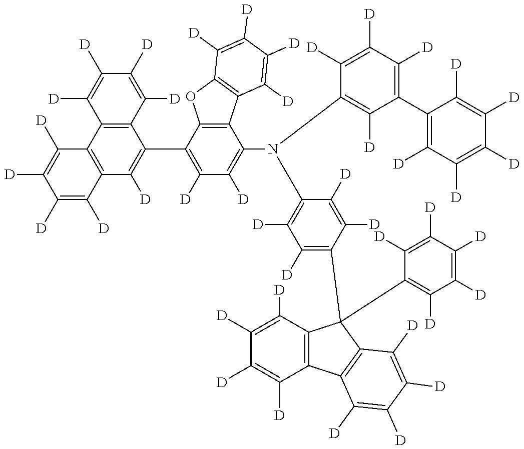 [SMILES: [2H]c1c([2H])c([2H])c(-c2c([2H])c([2H])c([2H])c(N(c3c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c([2H])c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c2[2H])c([2H])c1[2H]] | 17.1 g (67%) | 869.28 |
| | 3-91 | | |
| P54 | 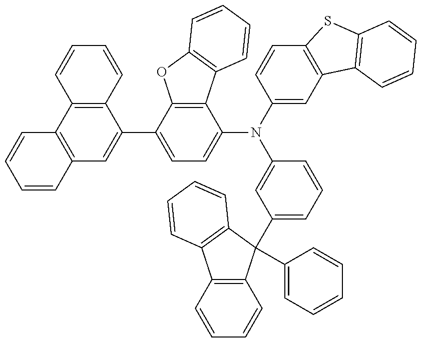 [SMILES: c1ccc(C2(c3cccc(N(c4ccc5sc6ccccc6c5c4)c4ccc(-c5cc6ccccc6c6ccccc56)c5oc6ccccc6c45)c3)c3ccccc3-c3ccccc32)cc1] | 18.5 g (74%) | 858.07 |
| | 3-110 | | |
| P55 | 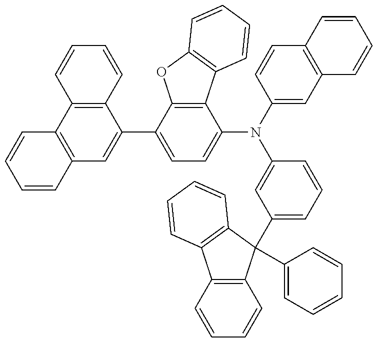 [SMILES: c1ccc(C2(c3cccc(N(c4ccc5ccccc5c4)c4ccc(-c5cc6ccccc6c6ccccc56)c5oc6ccccc6c45)c3)c3ccccc3-c3ccccc32)cc1] | 19.9 g (76%) | 801.99 |
| | 3-94 | | |
| P56 | 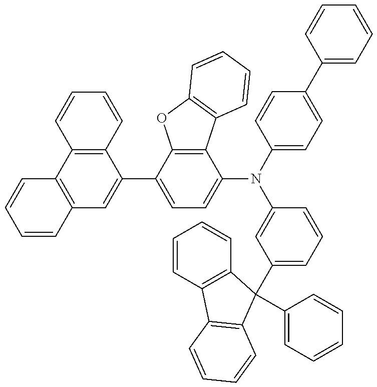 [SMILES: c1ccc(-c2ccc(N(c3cccc(C4(c5ccccc5)c5ccccc5-c5ccccc54)c3)c3ccc(-c4cc5ccccc5c5ccccc45)c4oc5ccccc5c34)cc2)cc1] | 18.4 g (72%) | 828.03 |
| | 3-112 | | |
| P57 | 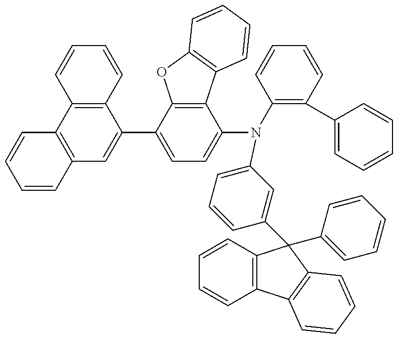 [SMILES: c1ccc(-c2ccccc2N(c2cccc(C3(c4ccccc4)c4ccccc4-c4ccccc43)c2)c2ccc(-c3cc4ccccc4c4ccccc34)c3oc4ccccc4c23)cc1] | 19.2 g (75%) | 828.03 |
| | 3-152 | | |
| P58 | 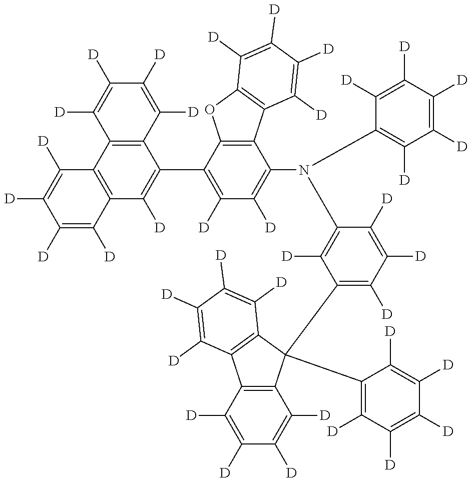 [SMILES: [2H]c1c([2H])c([2H])c(N(c2c([2H])c([2H])c([2H])c(C3(c4c([2H])c([2H])c([2H])c([2H])c4[2H])c4c([2H])c([2H])c([2H])c([2H])c4-c4c([2H])c([2H])c([2H])c([2H])c43)c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 19.2 g (70%) | 789.15 |
| | 3-179 | | |
| P59 | 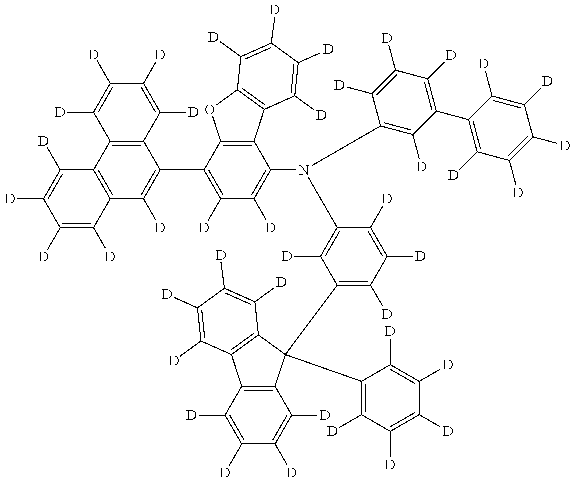 [SMILES: [2H]c1c([2H])c([2H])c(-c2c([2H])c([2H])c([2H])c(N(c3c([2H])c([2H])c([2H])c(C4(c5c([2H])c([2H])c([2H])c([2H])c5[2H])c5c([2H])c([2H])c([2H])c([2H])c5-c5c([2H])c([2H])c([2H])c([2H])c54)c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c2[2H])c([2H])c1[2H]] | 18.1 g (71%) | 869.28 |
| | 3-182 | | |
| P60 | 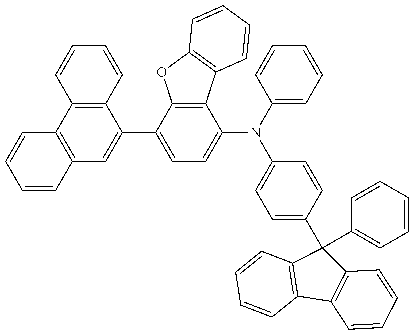 [SMILES: c1ccc(N(c2ccc(C3(c4ccccc4)c4ccccc4-c4ccccc43)cc2)c2ccc(-c3cc4ccccc4c4ccccc34)c3oc4ccccc4c23)cc1] | 20.1 g (73%) | 751.93 |
| | 3-1 | | |
| P61 | 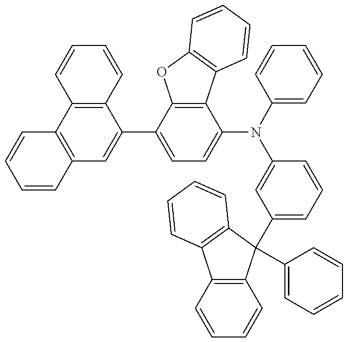 [SMILES: c1ccc(N(c2cccc(C3(c4ccccc4)c4ccccc4-c4ccccc43)c2)c2ccc(-c3cc4ccccc4c4ccccc34)c3oc4ccccc4c23)cc1] | 19.8 g (72%) | 751.93 |
| | 3-92 | | |
| P62 | 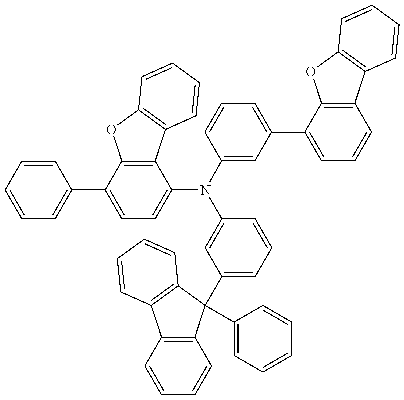 [SMILES: c1ccc(-c2ccc(N(c3cccc(-c4cccc5c4oc4ccccc45)c3)c3cccc(C4(c5ccccc5)c5ccccc5-c5ccccc54)c3)c3c2oc2ccccc23)cc1] | 20.3 g (76%) | 817.30 |
| | 1-103 | | |

### [Experimental Example 1] Measurement of HOMO and LUMO

The effect of the compound according to the present invention was checked through the following experiments, which are merely representative examples, and the experimental examples are not limited thereto.

### Experimental Example 1: Simulation Result of Hole Transport Auxiliary Layer

The hole transport auxiliary layer serves to reduce the accumulation of holes at the emission layer interface due to the difference in the HOMO level between the hole transport layer and the emission layer, and for this purpose, it is desirable that the HOMO energy difference with respect to the emission layer is smaller than the HOMO energy difference with respect to the hole transport layer. In addition, the hole transport auxiliary layer should have a higher LUMO energy level than the emission layer in order to minimize the leakage of electrons from the emission layer to the hole transport layer.

In order to check whether the compound according to the present invention, which is represented by Formula 1, is suitable as a substance for a hole transport auxiliary layer, the HOMO energy level (eV) and the LUMO energy level (eV) were calculated using Spartan software (B3LYP DFT 6-31G* by spartan' 16), which are shown in Table 2 below.

TABLE 2

| Compound | HOMO (calculation) | LUMO (calculation) |
|---|---|---|
| 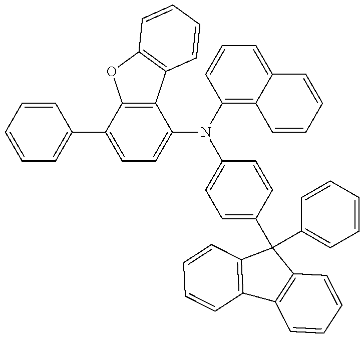 [SMILES: c1ccc(-c2ccc(N(c3ccc(C4(c5ccccc5)c5ccccc5-c5ccccc54)cc3)c3cccc4ccccc34)c3c2oc2ccccc23)cc1] | −4.94 | −1.30 |
| 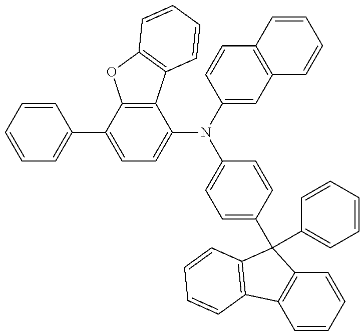 [SMILES: c1ccc(-c2ccc(N(c3ccc(C4(c5ccccc5)c5ccccc5-c5ccccc54)cc3)c3ccc4ccccc4c3)c3c2oc2ccccc23)cc1] | −4.92 | −1.20 |
| 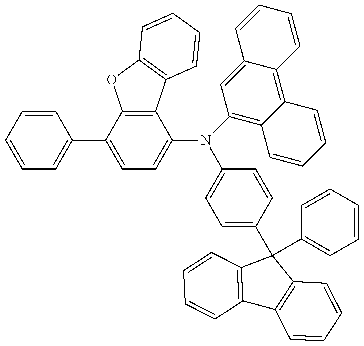 [SMILES: c1ccc(-c2ccc(N(c3ccc(C4(c5ccccc5)c5ccccc5-c5ccccc54)cc3)c3cc4ccccc4c4ccccc34)c3c2oc2ccccc23)cc1] | −4.94 | −1.31 |
| 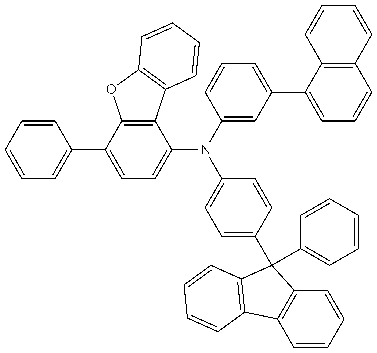 [SMILES: c1ccc(-c2ccc(N(c3ccc(C4(c5ccccc5)c5ccccc5-c5ccccc54)cc3)c3cccc(-c4cccc5ccccc45)c3)c3c2oc2ccccc23)cc1] | −5.01 | −1.17 |
| 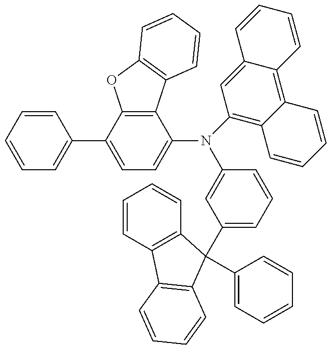 [SMILES: c1ccc(-c2ccc(N(c3cccc(C4(c5ccccc5)c5ccccc5-c5ccccc54)c3)c3cc4ccccc4c4ccccc34)c3c2oc2ccccc23)cc1] | −4.94 | −1.29 |
| 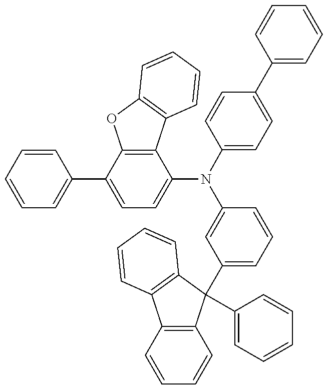 [SMILES: c1ccc(-c2ccc(N(c3cccc(C4(c5ccccc5)c5ccccc5-c5ccccc54)c3)c3ccc(-c4ccccc4)c4oc5ccccc5c34)cc2)cc1] | −4.99 | −1.13 |
| 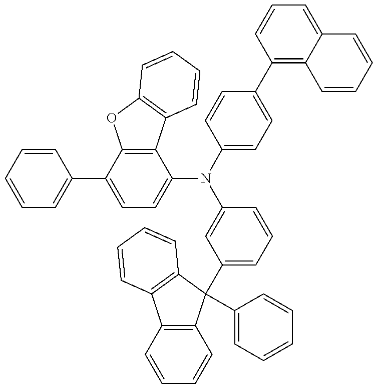 [SMILES: c1ccc(-c2ccc(N(c3ccc(-c4cccc5ccccc45)cc3)c3cccc(C4(c5ccccc5)c5ccccc5-c5ccccc54)c3)c3c2oc2ccccc23)cc1] | −5.01 | −1.13 |
| 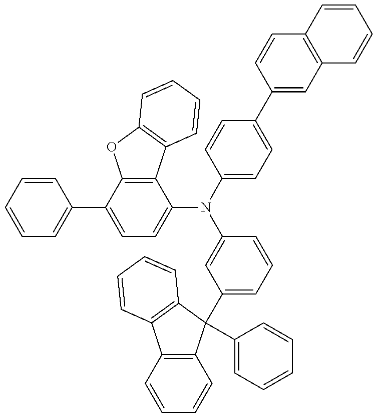 [SMILES: c1ccc(-c2ccc(N(c3ccc(-c4ccc5ccccc5c4)cc3)c3cccc(C4(c5ccccc5)c5ccccc5-c5ccccc54)c3)c3c2oc2ccccc23)cc1] | −5.00 | −1.23 |
| 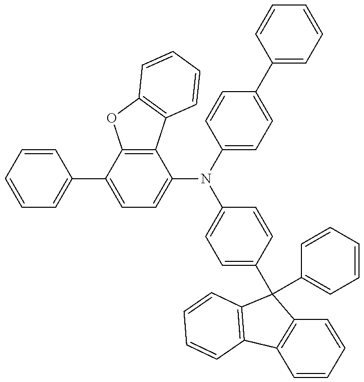 [SMILES: c1ccc(-c2ccc(N(c3ccc(C4(c5ccccc5)c5ccccc5-c5ccccc54)cc3)c3ccc(-c4ccccc4)c4oc5ccccc5c34)cc2)cc1] | −4.98 | −1.15 |
| 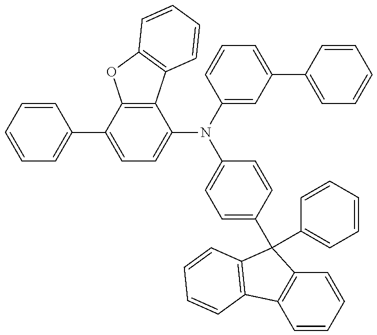 [SMILES: c1ccc(-c2cccc(N(c3ccc(C4(c5ccccc5)c5ccccc5-c5ccccc54)cc3)c3ccc(-c4ccccc4)c4oc5ccccc5c34)c2)cc1] | −5.00 | −1.15 |
| 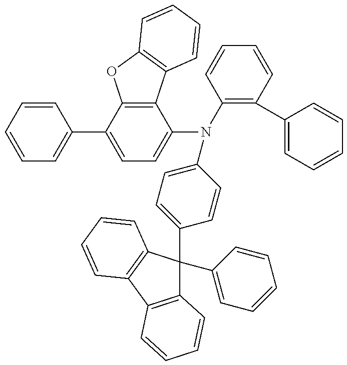 [SMILES: c1ccc(-c2ccccc2N(c2ccc(C3(c4ccccc4)c4ccccc4-c4ccccc43)cc2)c2ccc(-c3ccccc3)c3oc4ccccc4c23)cc1] | −4.98 | −1.06 |
| 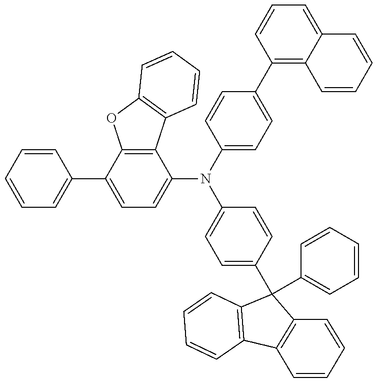 [SMILES: c1ccc(-c2ccc(N(c3ccc(-c4cccc5ccccc45)cc3)c3ccc(C4(c5ccccc5)c5ccccc5-c5ccccc54)cc3)c3c2oc2ccccc23)cc1] | −5.00 | −1.15 |
| 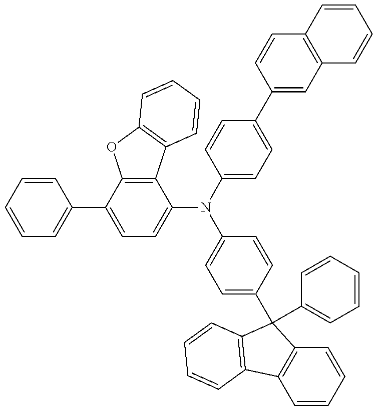 [SMILES: c1ccc(-c2ccc(N(c3ccc(-c4ccc5ccccc5c4)cc3)c3ccc(C4(c5ccccc5)c5ccccc5-c5ccccc54)cc3)c3c2oc2ccccc23)cc1] | −4.97 | −1.23 |
| 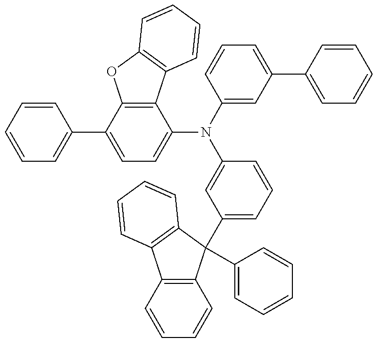 [SMILES: c1ccc(-c2cccc(N(c3cccc(C4(c5ccccc5)c5ccccc5-c5ccccc54)c3)c3ccc(-c4ccccc4)c4oc5ccccc5c34)c2)cc1] | −4.99 | −1.11 |
| 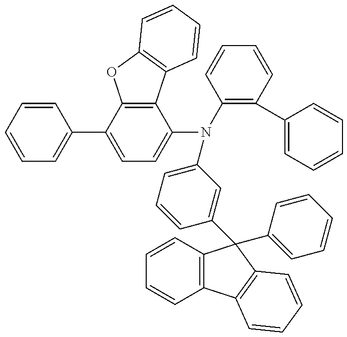 [SMILES: c1ccc(-c2ccccc2N(c2cccc(C3(c4ccccc4)c4ccccc4-c4ccccc43)c2)c2ccc(-c3ccccc3)c3oc4ccccc4c23)cc1] | −4.96 | −1.01 |
| 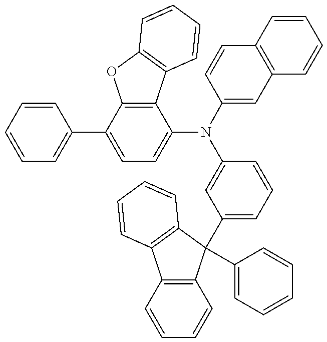 [SMILES: c1ccc(-c2ccc(N(c3cccc(C4(c5ccccc5)c5ccccc5-c5ccccc54)c3)c3ccc4ccccc4c3)c3c2oc2ccccc23)cc1] | −4.92 | −1.22 |
| 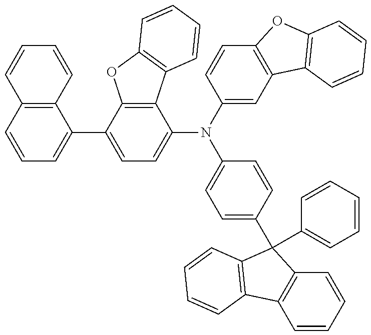 [SMILES: c1ccc(C2(c3ccc(N(c4ccc5oc6ccccc6c5c4)c4ccc(-c5cccc6ccccc56)c5oc6ccccc6c45)cc3)c3ccccc3-c3ccccc32)cc1] | −4.96 | −1.15 |
| 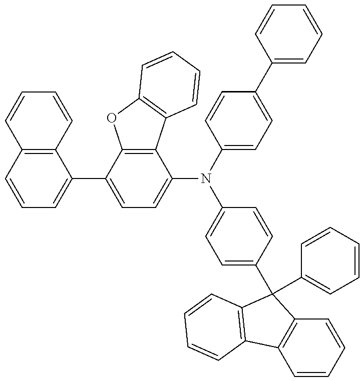 [SMILES: c1ccc(-c2ccc(N(c3ccc(C4(c5ccccc5)c5ccccc5-c5ccccc54)cc3)c3ccc(-c4cccc5ccccc45)c4oc5ccccc5c34)cc2)cc1] | −4.99 | −1.16 |
| 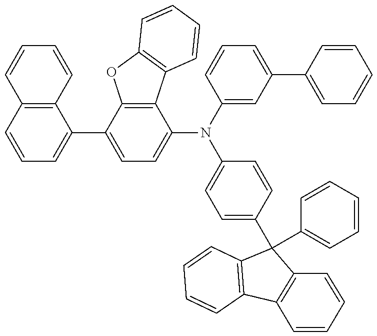 [SMILES: c1ccc(-c2cccc(N(c3ccc(C4(c5ccccc5)c5ccccc5-c5ccccc54)cc3)c3ccc(-c4cccc5ccccc45)c4oc5ccccc5c34)c2)cc1] | −5.02 | −1.15 |
| 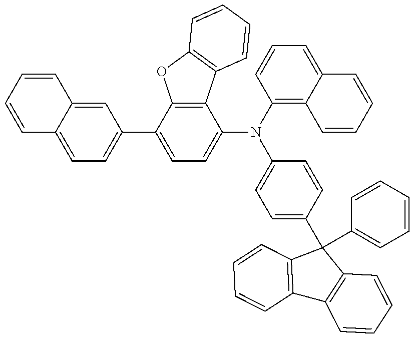 [SMILES: c1ccc(C2(c3ccc(N(c4cccc5ccccc45)c4ccc(-c5ccc6ccccc6c5)c5oc6ccccc6c45)cc3)c3ccccc3-c3ccccc32)cc1] | −4.93 | −1.35 |
| 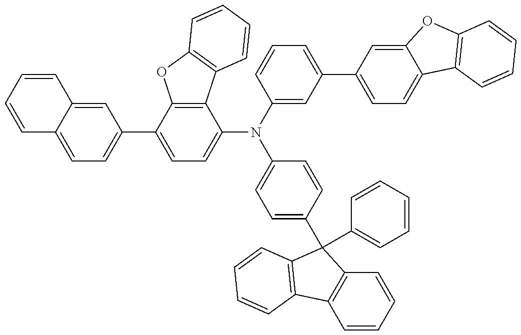 [SMILES: c1ccc(C2(c3ccc(N(c4cccc(-c5ccc6c(c5)oc5ccccc56)c4)c4ccc(-c5ccc6ccccc6c5)c5oc6ccccc6c45)cc3)c3ccccc3-c3ccccc32)cc1] | −5.01 | −1.31 |
| 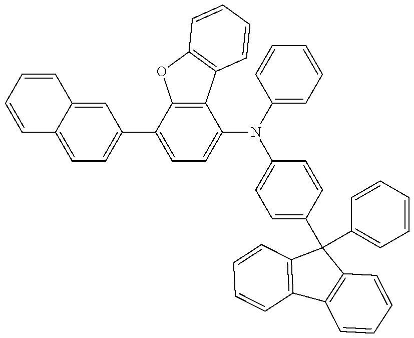 [SMILES: c1ccc(N(c2ccc(C3(c4ccccc4)c4ccccc4-c4ccccc43)cc2)c2ccc(-c3ccc4ccccc4c3)c3oc4ccccc4c23)cc1] | −5.01 | −1.35 |
| 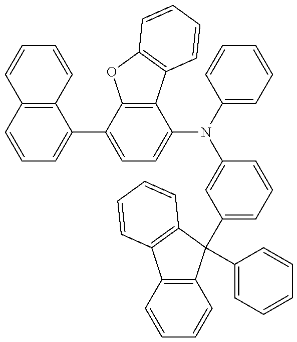 [SMILES: c1ccc(N(c2cccc(C3(c4ccccc4)c4ccccc4-c4ccccc43)c2)c2ccc(-c3cccc4ccccc34)c3oc4ccccc4c23)cc1] | −5.02 | −1.19 |
| 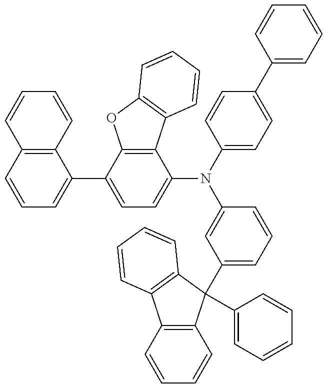 [SMILES: c1ccc(-c2ccc(N(c3cccc(C4(c5ccccc5)c5ccccc5-c5ccccc54)c3)c3ccc(-c4cccc5ccccc45)c4oc5ccccc5c34)cc2)cc1] | −5.00 | −1.14 |
| 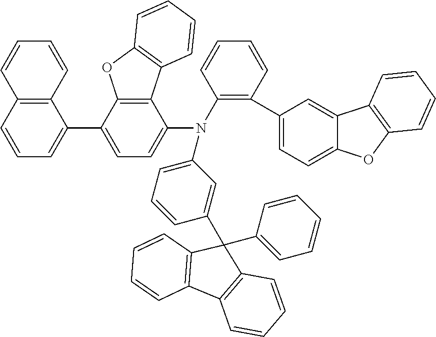 [SMILES: c1ccc(C2(c3cccc(N(c4ccccc4-c4ccc5oc6ccccc6c5c4)c4ccc(-c5cccc6ccccc56)c5oc6ccccc6c45)c3)c3ccccc3-c3ccccc32)cc1] | −4.98 | −1.10 |
| 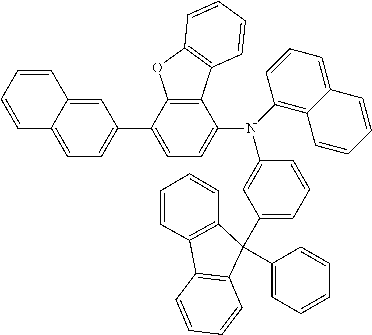 [SMILES: c1ccc(C2(c3cccc(N(c4cccc5ccccc45)c4ccc(-c5ccc6ccccc6c5)c5oc6ccccc6c45)c3)c3ccccc3-c3ccccc32)cc1] | −4.97 | −1.35 |
| 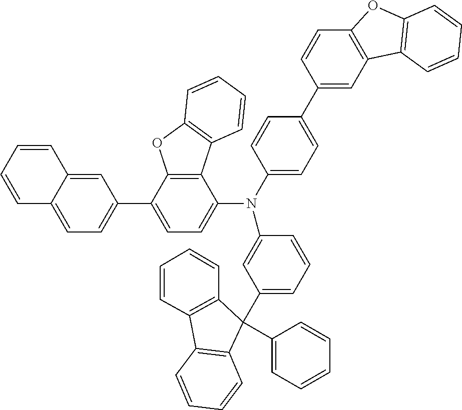 [SMILES: c1ccc(C2(c3cccc(N(c4ccc(-c5ccc6oc7ccccc7c6c5)cc4)c4ccc(-c5ccc6ccccc6c5)c5oc6ccccc6c45)c3)c3ccccc3-c3ccccc32)cc1] | −4.98 | −1.30 |
| 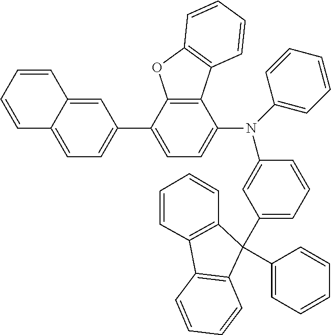 [SMILES: c1ccc(N(c2cccc(C3(c4ccccc4)c4ccccc4-c4ccccc43)c2)c2ccc(-c3ccc4ccccc4c3)c3oc4ccccc4c23)cc1] | −5.01 | −1.35 |
| 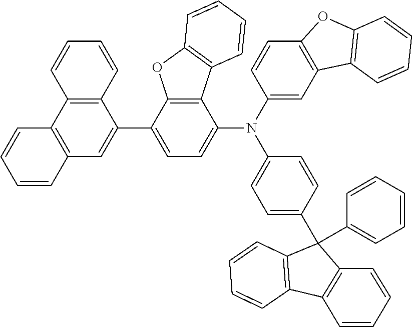 [SMILES: c1ccc(C2(c3ccc(N(c4ccc5oc6ccccc6c5c4)c4ccc(-c5cc6ccccc6c6ccccc56)c5oc6ccccc6c45)cc3)c3ccccc3-c3ccccc32)cc1] | −4.97 | −1.18 |
| 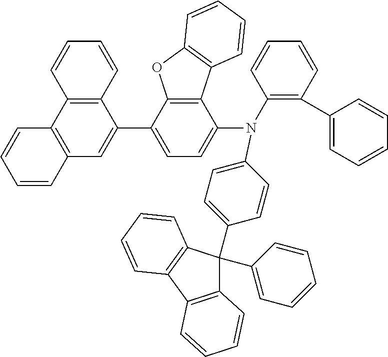 [SMILES: c1ccc(-c2ccccc2N(c2ccc(C3(c4ccccc4)c4ccccc4-c4ccccc43)cc2)c2ccc(-c3cc4ccccc4c4ccccc34)c3oc4ccccc4c23)cc1] | −5.01 | −1.14 |
| 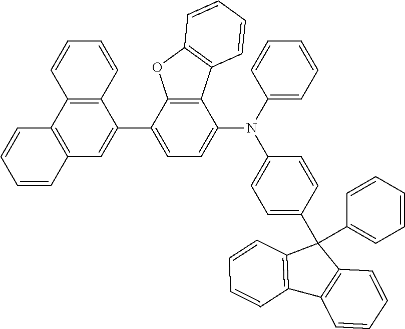 [SMILES: c1ccc(N(c2ccc(C3(c4ccccc4)c4ccccc4-c4ccccc43)cc2)c2ccc(-c3cc4ccccc4c4ccccc34)c3oc4ccccc4c23)cc1] | −5.02 | −1.17 |
| 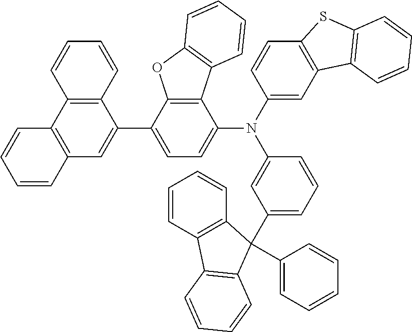 [SMILES: c1ccc(C2(c3cccc(N(c4ccc5sc6ccccc6c5c4)c4ccc(-c5cc6ccccc6c6ccccc56)c5oc6ccccc6c45)c3)c3ccccc3-c3ccccc32)cc1] | −4.96 | −1.24 |
| 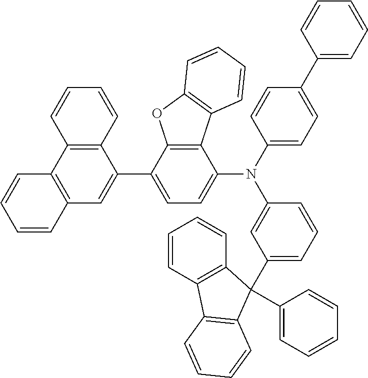 [SMILES: c1ccc(-c2ccc(N(c3cccc(C4(c5ccccc5)c5ccccc5-c5ccccc54)c3)c3ccc(-c4cc5ccccc5c5ccccc45)c4oc5ccccc5c34)cc2)cc1] | −5.04 | −1.20 |
| 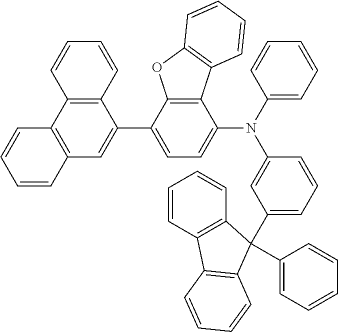 [SMILES: c1ccc(N(c2cccc(C3(c4ccccc4)c4ccccc4-c4ccccc43)c2)c2ccc(-c3cc4ccccc4c4ccccc34)c3oc4ccccc4c23)cc1] | −5.06 | −1.19 |

### [Example 1] Manufacturing of Organic Light-Emitting Diode (Blue Emission Layer)

A substrate, on which ITO (100 nm) as the first electrode (anode) of the organic light-emitting diode had been laminated, was subjected to patterning by dividing the substrate into a region of the second electrode (cathode), a region of the first electrode (anode), and an insulating layer through a photo-lithography process, and then subjected to a UV-ozone treatment and a surface treatment with an O₂:N₂ plasma for the intended purpose of increasing the work function and cleaning the first electrode (ITO).

Next, a mixture obtained by mixing NDP-9 (2-(7-dicyanomethylene-1,3,4,5,6,8,9,10-octafluoro-7H-pyren-2-ylidene)-malononitrile) and N4,N4,N4′,N4′-tetra([1,1′-biphenyl]-4-yl)-[1,1′-biphenyl]-4,4′-diamine at a ratio of 3:97 was deposited on the anode, thereby forming a hole injection layer (HIL) to have a thickness of 10 nm. Subsequently, N4,N4,N4′,N4′-tetra([1,1′-biphenyl]-4-yl)-[1,1′-biphenyl]-4,4′-diamine was subjected to vacuum deposition on the upper part of the hole injection layer to form a hole transport layer having a 100 nm thickness, and the compound 2-1 was used to form a hole transport auxiliary layer to have a thickness of 15 nm, on the upper part of the hole transport layer (HTL).

9,10-bis(2-naphthyl)anthracene (ADN) was used as a host, and (2,12-di-tert-butyl-5,9-bis(4-(tert-butyl)phenyl)-7-(3,5-di-tert-butylphenyl)-5,9-dihydro5,9-diaza-13b-boranaphtho[3,2,1-de]anthracene (t-DABNA-dtB) was used as a dopant to deposit a blue emission layer to have a thickness of 25 nm on the upper part of the hole transport auxiliary layer, where the mixing ratio (based on weight) of host:dopant was 97:3. (2-(4-(9,10-di(naphthalene-2-yl)anthracene-2-yl)phenyl)-1-phenyl-1H-benzo[d]imidazole and Liq were mixed at a weight ratio of 1:1 and then deposited to have a thickness of 25 nm, thereby forming an electron transport layer (ETL) on the blue emission layer. An electron injection layer (Liq) was deposited to have a thickness of 1 nm on the upper part of the electron transport layer ETL, and a mixture obtained by mixing magnesium and silver at a weight ratio of 1:4 was deposited, thereby forming a cathode to have a thickness of 16 nm. N4,N4′-bis [4-[bis(3-methyl phenyl)amino]phenyl]-N4,N4′-diphenyl-[1,1′-biphenyl]-4,4′-diamine (DNTPD) was deposited on the cathode, thereby forming a capping layer to have a thickness of 60 nm. By bonding a seal cap comprising a hygroscopic agent onto the capping layer with a UV curable adhesive, A protective film (encapsulation layer or protecting layer) was formed to protect an organic light-emitting diode from oxygen or moisture in the atmosphere, thereby forming an organic light-emitting diode.

### Examples 2 to 74

Each of the organic light-emitting diodes of Examples 2 to 74 was manufactured in the same manner as in Example 1, except that the material of the compound 2-1 used as the material for the hole transport auxiliary layer in Example 1 was changed to the material shown in Table 3 below.

### Comparative Examples 1 to 5

Each of the organic light-emitting diodes of Comparative Examples 1 to 5 was manufactured in the same manner as in Example 1, except that the material of the compound 2-1 used as the material for the hole transport auxiliary layer in Example 1 was changed to a compound A, a compound B, a compound C, a compound D, or a compound E below.

### [Experimental Example 2] Performance Evaluation of Organic Light-Emitting Diode (Blue Element)

A current of 10 mA/cm² was applied to each of the organic light-emitting diodes prepared in Examples 1 to 74 and Comparative Examples 1 to 5 with a CS-2000, manufactured by KONICA MINOLTA, INC., thereby measuring a operation voltage V and an external quantum efficiency (EQE) (%). In addition, the lifetime (LT95) (hrs) was measured according to a method in which the time for brightness reduction from the initial brightness to 95% by driving at a constant current of 10 mA/cm² was checked with M6000 manufactured by McScience Inc. The measurement results are shown in Table 3 below.

TABLE 3

| Example/ | Compound for hole | | | |
|---|---|---|---|---|
| Comparative | transport auxiliary | Operation | EQE | Lifetime (LT95) |
| Example | layer | voltage (V) | (%) | (hrs) |
| Example 1 | 2-1 | 3.33 | 22.5 | 476 |
| Example 2 | 2-42 | 3.38 | 21.8 | 465 |
| Example 3 | 2-7 | 3.35 | 22.3 | 474 |
| Example 4 | 2-13 | 3.46 | 20.8 | 433 |
| Example 5 | 2-16 | 3.46 | 20.7 | 433 |
| Example 6 | 2-20 | 3.37 | 20.6 | 431 |
| Example 7 | 2-21 | 3.39 | 21.6 | 463 |
| Example 8 | 2-33 | 3.54 | 20.1 | 422 |
| Example 9 | 2-41 | 3.38 | 21.8 | 466 |
| Example 10 | 2-53 | 3.51 | 20.5 | 425 |
| Example 11 | 2-61 | 3.40 | 21.5 | 461 |
| Example 12 | 2-86 | 3.38 | 21.8 | 535 |
| Example 13 | 2-87 | 3.59 | 19.5 | 409 |
| Example 14 | 2-92 | 3.59 | 19.4 | 408 |
| Example 15 | 2-99 | 3.67 | 18.2 | 373 |
| Example 16 | 2-106 | 3.67 | 18.1 | 371 |
| Example 17 | 2-107 | 3.62 | 19.0 | 400 |
| Example 18 | 2-122 | 3.70 | 17.6 | 361 |
| Example 19 | 2-127 | 3.60 | 19.1 | 403 |
| Example 20 | 2-128 | 3.61 | 19.1 | 402 |
| Example 21 | 2-147 | 3.63 | 19.0 | 398 |
| Example 22 | 2-169 | 3.58 | 19.5 | 470 |
| Example 23 | 2-173 | 3.30 | 23.0 | 480 |
| Example 24 | 2-178 | 3.31 | 22.8 | 478 |
| Example 25 | 2-185 | 3.45 | 21.1 | 438 |
| Example 26 | 2-188 | 3.45 | 21.1 | 437 |
| Example 27 | 2-192 | 3.45 | 21.0 | 435 |
| Example 28 | 2-193 | 3.39 | 21.7 | 465 |
| Example 29 | 2-194 | 3.39 | 21.6 | 464 |
| Example 30 | 2-208 | 3.53 | 20.3 | 424 |
| Example 31 | 2-213 | 3.37 | 22.0 | 470 |
| Example 32 | 2-216 | 3.37 | 21.9 | 468 |
| Example 33 | 2-233 | 3.39 | 21.6 | 463 |
| Example 34 | 2-258 | 3.37 | 22.0 | 541 |
| Example 35 | 2-259 | 3.57 | 19.9 | 414 |
| Example 36 | 2-264 | 3.56 | 19.8 | 413 |
| Example 37 | 2-271 | 3.65 | 18.4 | 380 |
| Example 38 | 2-102 | 3.66 | 18.3 | 374 |
| Example 39 | 2-278 | 3.65 | 18.4 | 378 |
| Example 40 | 2-279 | 3.61 | 19.1 | 402 |
| Example 41 | 2-282 | 3.61 | 19.1 | 401 |
| Example 42 | 2-294 | 3.69 | 17.7 | 365 |
| Example 43 | 2-299 | 3.60 | 19.3 | 405 |
| Example 44 | 2-301 | 3.60 | 19.2 | 404 |
| Example 45 | 2-311 | 3.68 | 17.8 | 369 |
| Example 46 | 2-319 | 3.63 | 19.0 | 396 |
| Example 47 | 2-341 | 3.56 | 20.0 | 476 |
| Example 48 | 2-344 | 3.60 | 19.2 | 466 |
| Example 49 | 3-1 | 3.73 | 17.3 | 351 |
| Example 50 | 3-6 | 3.73 | 17.3 | 349 |
| Example 51 | 3-13 | 3.81 | 16.4 | 338 |
| Example 52 | 3-16 | 3.81 | 16.4 | 338 |
| Example 53 | 3-20 | 3.83 | 16.3 | 336 |
| Example 54 | 3-21 | 3.78 | 17.0 | 347 |
| Example 55 | 3-33 | 3.89 | 16.1 | 332 |
| Example 56 | 3-45 | 3.76 | 17.1 | 348 |
| Example 57 | 3-61 | 3.79 | 16.9 | 345 |
| Example 58 | 3-88 | 3.75 | 17.4 | 404 |
| Example 59 | 3-91 | 3.77 | 17.1 | 400 |
| Example 60 | 3-92 | 3.71 | 17.5 | 352 |
| Example 61 | 3-97 | 3.71 | 17.5 | 352 |
| Example 62 | 3-104 | 3.81 | 16.6 | 340 |
| Example 63 | 3-111 | 3.80 | 16.6 | 341 |
| Example 64 | 3-112 | 3.77 | 17.0 | 347 |
| Example 65 | 3-132 | 3.76 | 17.1 | 348 |
| Example 66 | 3-152 | 3.77 | 17.0 | 346 |
| Example 67 | 3-182 | 3.76 | 17.1 | 400 |
| Example 68 | 3-179 | 3.71 | 17.5 | 405 |
| Example 69 | 2-8 | 3.35 | 22.2 | 473 |
| Example 70 | 2-174 | 3.31 | 22.9 | 478 |
| Example 71 | 2-175 | 3.31 | 22.8 | 478 |
| Example 72 | 2-176 | 3.32 | 22.6 | 477 |
| Example 73 | 2-179 | 3.31 | 22.8 | 479 |
| Example 74 | 2-347 | 3.29 | 23.1 | 552 |
| Comparative Example 1 | Compound A 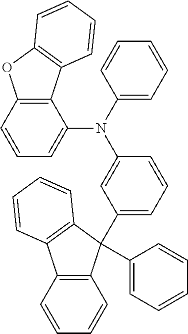 [SMILES: c1ccc(N(c2cccc(C3(c4ccccc4)c4ccccc4-c4ccccc43)c2)c2cccc3oc4ccccc4c23)cc1] | 4.01 | 15.1 | 311 |
| Comparative Example 2 | Compound B 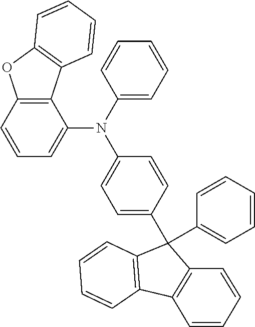 [SMILES: c1ccc(N(c2ccc(C3(c4ccccc4)c4ccccc4-c4ccccc43)cc2)c2cccc3oc4ccccc4c23)cc1] | 4.05 | 14.8 | 307 |
| Comparative Example 3 | Compound C 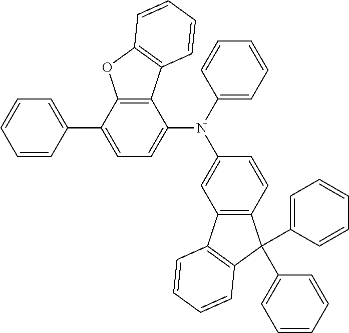 [SMILES: c1ccc(-c2ccc(N(c3ccccc3)c3ccc4c(c3)-c3ccccc3C4(c3ccccc3)c3ccccc3)c3c2oc2ccccc23)cc1] | 4.05 | 13.4 | 297 |
| Comparative Example 4 | Compound D 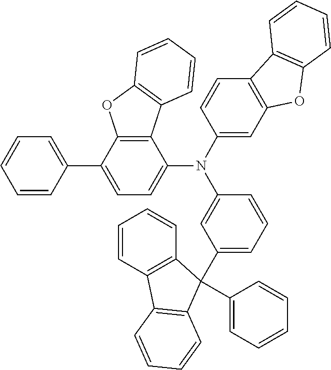 [SMILES: c1ccc(-c2ccc(N(c3cccc(C4(c5ccccc5)c5ccccc5-c5ccccc54)c3)c3ccc4c(c3)oc3ccccc34)c3c2oc2ccccc23)cc1] | 4.03 | 13.5 | 298 |
| Comparative Example 5 | Compound E 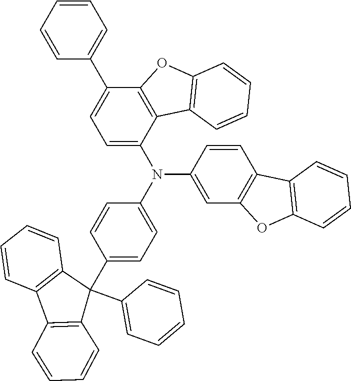 [SMILES: c1ccc(-c2ccc(N(c3ccc(C4(c5ccccc5)c5ccccc5-c5ccccc54)cc3)c3ccc4c(c3)oc3ccccc34)c3c2oc2ccccc23)cc1] | 4.03 | 13.6 | 300 |

As shown in Table 3 above, it has been confirmed that in the organic compound according to the present invention, which is represented by Formula 1, since the polycyclic compound represented by Formulae 3 to 5 is introduced into Ar₂, the stability of the compound itself is high, the hole mobility is also excellent, and thus the charge balance of the entire element is improved, and as a result, the characteristics of low voltage, long lifespan, and high efficiency are exhibited.

As described above, the exemplary embodiments according to the present specification have been described in more detail. However, the present specification is not necessarily limited to such embodiments and may be variously modified without departing from the technical idea of the present specification.

Accordingly, exemplary embodiments disclosed in the present specification are not intended to limit the technical idea of the present specification but to describe the technical idea of the present specification, and the scope of the technical idea of the present specification is not limited by such exemplary embodiments. Therefore, it should be understood that the examples described above should be understood as exemplary and be not limited thereto.

Although the organic compound and the organic light-emitting diode including the same have been described with reference to the specific embodiments, they are not limited thereto. Therefore, it will be readily understood by those skilled in the art that various modifications and changes can be made thereto without departing from the spirit and scope of the present invention defined by the appended claims.

What is claimed is:

1. An organic compound represented by Formula 1 below: 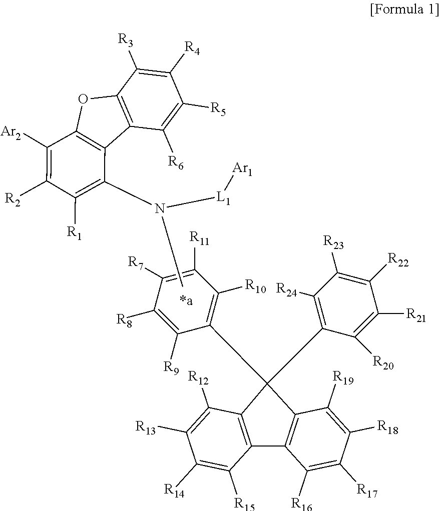
where, in Formula 1 above,
L₁ is selected from the group consisting of a single bond, a substituted or unsubstituted arylene group having 6 to 30 carbon atoms, and a substituted or unsubstituted heteroarylene group having 1 to 30 carbon atoms,
Ar₁ is a substituted or unsubstituted aryl group having 6 to 30 carbon atoms or a substituted or unsubstituted heteroaryl group having 1 to 30 carbon atoms,
Ar₂ is selected from as one group among Formula 3, Formula 4, and Formula 5 below, 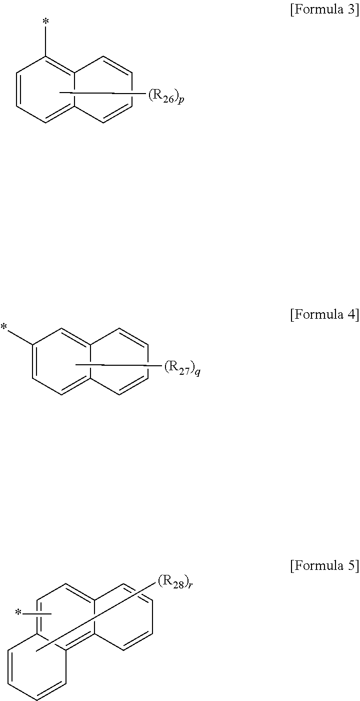
R₁ to R₂₄ and R₂₆ to R₂₈ are the same as or different from each other and are each independently one selected from the group consisting of a hydrogen atom, a deuterium atom, a substituted or unsubstituted aryl group having 6 to 30 carbon atoms, and a substituted or unsubstituted heteroary group having 1 to 30 carbon atoms,
p is an integer of 1 to 7,
q is an integer of 1 to 7,
r is an integer of 1 to 9, and
* means a moiety for bonding,
when L₁, Ar₁, Ar₂, R₁ to R₂₄, and R₂₆ to R₂₈ are substituted, substituents are the same as or different from each other and may be each independently one or more selected from the group consisting of a deuterium atom, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, an heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms, and when the substituents are plural, the substituents are the same as or different from each other, and
* a represents a bonding position on a phenylene, and one of R₇ to R₁₁ represents a single bond bonded to *a.

2. The organic compound of claim 1, wherein Formula 1 represents a compound selected from the group consisting of compounds represented by Formula 6 or Formula 7 below, 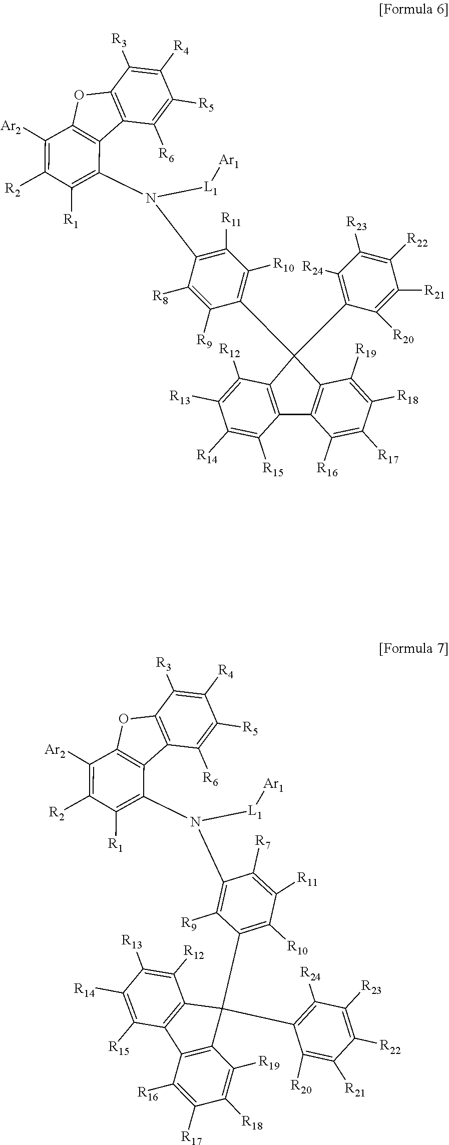
wherein, in Formula 6 or Formula 7 above,
definitions of L₁, Ar₁, Ar₂, and R₁ to R₂₄ and substituents thereof are the same as those described in Formula 1 above.

3. The organic compound of claim 1, wherein L₁ is a single bond or is selected from the group consisting of substituents represented by F1 to F3 below: 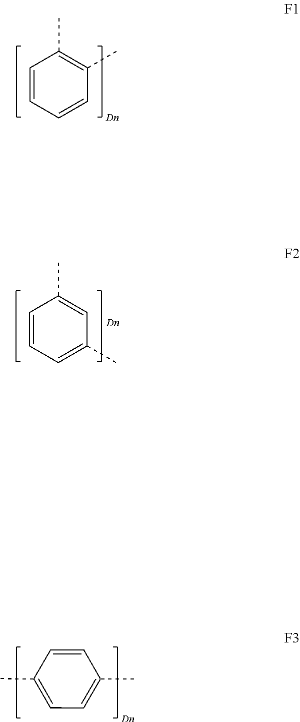
wherein, Dn means the number of substituted deuterium atoms in structures of F1 to F3 above.

4. The organic compound of claim 1, wherein Ar₁ represents a group selected from the group consisting of compounds represented by Formula 8 to Formula 11 below: 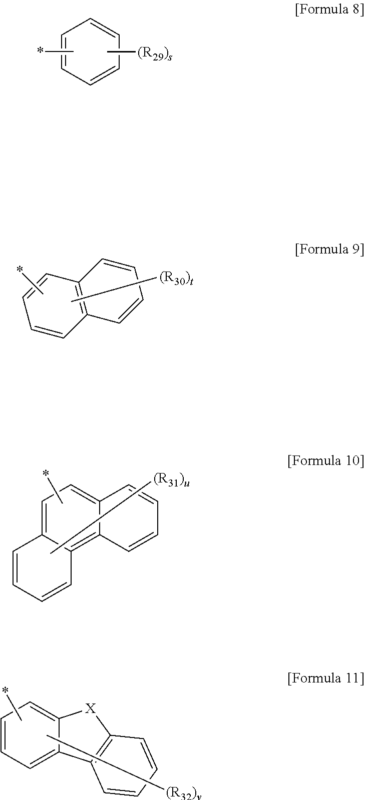
wherein, in Formula 8 to Formula 11,
R₂₉ to R₃₂ are the same as or different from each other and are each independently one selected from the group consisting of a hydrogen atom, a deuterium atom, a substituted or unsubstituted aryl group having 6 to 30 carbon atoms, and a substituted or unsubstituted heteroaryl group having 1 to 30 carbon atoms,
s is an integer in a range of 1 to 5,
t is an integer in a range of 1 to 7,
v is an integer in a range of 1 to 7, and
u is an integer in a range of 1 to 9,
in Formula 11,
X is 0, S, NR₃₃, or CR₃₄R₃₅,
R₃₃ to R₃₅ are the same as or different from each other and are each independently selected from the group consisting of a hydrogen atom, a deuterium atom, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms, and
* means a moiety for bonding.

5. An organic light-emitting diode comprising:
a first electrode;
a second electrode facing the first electrode; and
one or more organic material layers disposed between the first electrode and the second electrode,
wherein at least one of the one or more organic material layers is a hole transport layer or a hole transport auxiliary layer, which contains the organic compound of claim 1.

6. A display device comprising the organic light-emitting diode of claim 5.
